SEQUENCE LISTING (79 sequences)

SEQ ID NO: 1 - protein, 751 aa, synthetic construct
  REGION 1..751: note = Synthetic
MSNPIRDIQDRLKTAKFDNKDDMMNLASSLYKYEKQLMDSSEATLCQQGLSNRPNSFSQLSQFRDSDIQSKAGGQTGKFWQNEYEACKNFQTHKERRETLEQIIRFLQNGAEEKDADDLLLKTLARAYFHRGLLYRPKGFSVPARKVEAMKKAIAYCEIILDKNEEESEALRIWLYAAMELRRCGEEYPENFAEKLFYLANDGFISELYDIRLFLEYTEREEDNNFLDMILQENQDRERLFELCLYKARACFHLNQLNDVRIYGESAIDNAPGAFADPFWDELVEFIRMLRNKKSELWKEIAIKAWDKCREKEMKVGNNIYLSWYWARQRELYDLAFMAQDGIEKKTRIADSLKSRTTLRIQELNELRKDAHRKQNRRLEDKLDRIIEQENEARDGAYLRRNPPCFTGGKREEIPFARLPQNWIAVHFYLNELESHEGGKGGHALIYDPQKAEKDQWQDKSFDYKELHRKFLEWQENYILNEEGSADFLVTLCREIEKAMPFLFKSEVIPEDRPVLWIPHGFLHRLPLHAAMKSGNNSNIEIFWERHASRYLPAWHLFDPAPYSREESSTLLKNFEEYDFQNLENGEIEVYAPSSPKKVKEAIRENPAILLLLCHGEADMTNPFRSCLKLKNKDMTIFDLLTVEDVRLSGSRILLGACESDMVPPLEFSVDEHLSVSGAFLSHKAGEIVAGLWTVDSEKVDECYSYLVEEKDFLRNLQEWQMAETENFRSENDSSLFYKIAPFRIIGFPAE

SEQ ID NO: 2 - protein, 565 aa, synthetic construct
  REGION 1..565: note = Synthetic
MNTTTYNTTTDALLEWGKVYFQKEDFSEFLDNLEAYISDAGDSLKDELESGVEKLVLGIKSAEAVIFGEAVIGTTPENEAWYDAEESFLTLDCAVWLSQALDRVVRRQDASLADSLIARLDEAINRVAEKLYADNLSPLRFSSLNEIRRSALEATDEKYHYLFPWHGAACDVDENILLILTEEYHLIGADKAGANLSEELRGDLPFIFAELERDEVLRAYVEKENALSLALENTMREHWAFGLLEAARDEGYNHPYPADVGMRIHQVARAVFSQTNLSPAERLAVAIAGACFTPEISEDRRLEILLDCEERVCEIEAPTGDDTSVRVIKDLKALADHRVRHEIPAESLVSLWFEQIEAAGTDFDTKTPMDELVLRMLSDNVITLSVDRKAASQTETDDVKPQKGKIIPFPVPDIANDEVEYQKAVGMKKDKKAANDSKVKFPGLLEIQGCRDGDKAILLEDTDDAAANHRKLFSILKAGKLNSAFFIQSDDGEWVESESKPTMEDNRIILHDSHHSSFVWILDTGSMQLRQSVKCVKDALNKKTGSAKKLKPKTMIVWVTIPQEG

SEQ ID NO: 3 - protein, 80 aa, synthetic construct
  REGION 1..80: note = Synthetic
LWFEQIEAAGTDFDTKTPMDELVLRMLSDNVITLSVDRKAASQTETDDVKPQKGKIIPFPVPDIANDEVEYQKAVGMKKD

SEQ ID NO: 4 - protein, 129 aa, synthetic construct
  REGION 1..129: note = Synthetic
MDLPDDHYLSTQTILSKDLNSGLRSGSGLEMEVSALEKEVSALEKEVSALEKEVSALEKEVSALEKEKRDHMVLLEYVTAAGITDASENLYFQSKVSALKEKVSALKEKVSALKEKVSALKEKVSALKE

SEQ ID NO: 5 - protein, 89 aa, synthetic construct
  REGION 1..89: note = Synthetic
MDLPDDHYLSTQTILSKDLNSGLRSGSGLEMEVSALEKEVSALEKEVSALEKEVSALEKEVSALEKEKRDHMVLLEYVTAAGITDASGS

SEQ ID NO: 6 - protein, 89 aa, synthetic construct; identical to SEQ ID NO: 5
  REGION 1..89: note = Synthetic

SEQ ID NO: 7 - protein, 751 aa, synthetic construct; identical to SEQ ID NO: 1
  REGION 1..751: note = Synthetic

SEQ ID NO: 8 - protein, 1601 aa, synthetic construct
  REGION 1..1601: note = Synthetic
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 9 - RNA, 55 nt, synthetic construct
  misc_feature 1..55: note = Synthetic
  source 1..55: mol_type = other RNA
gaaacaccgttgatgtcacggaacagctgtacaccacgttgtggttgctcttgta

SEQ ID NO: 10 - RNA, 55 nt, synthetic construct
  misc_feature 1..55: note = Synthetic
  source 1..55: mol_type = other RNA
gaaacaccgttgatgtcacggaactacaagagcaaccacaacgtggtgtacagct

SEQ ID NO: 11 - protein, 565 aa, synthetic construct; identical to SEQ ID NO: 2
  REGION 1..565: note = Synthetic

SEQ ID NO: 12 - protein, 566 aa, synthetic construct
  REGION 1..566: note = Synthetic
MNTTTYNTTTDALLEWGKVYFQKEDFSEFLDNLEAYISDAGDSLKDELESGVEKLVLGIKSAEAVIFGEAVIGTTPENEAWYDAEESFLTLDCAVWLSQALDRVVRRQDASLADSLIARLDEAINRVAEKLYADNLSPLRFSSLNEIRRSALEATDDEKYHYLFPWHGAACDVDENILLILTEEYHLIGADKAGANLSEELRGDLPFIFAELERDEVLRAYVEKENALSLALENTMREHWAFGLLEAARDEGYNHPYPADVGMRIHQVARAVFSQTNLSPAERLAVAIAGACFTPEISEDRRLEILLDCEERVCEIEAPTGDDTSVRVIKDLKALADHRVRHEIPAESLVSLWFEQIEAAGTDFDTKTPMDELVLRMLSDNVITLSVDRKAASQTETDDVKPQKGKIIPFPVPDIANDEVEYQKAVGMKKDKKAANSSKVKFPGLLEIQGCRDGDKAILLEDTDDAAANHRKLFSILKAGKLNSAFFIQSDDGEWVESESKPTMEDNRIILHDSHHSSFVWILDTGSMQLRQSVKCVKDALNKKTGSAKKLKPKTMIVWVTIPQEG

SEQ ID NO: 13 - protein, 566 aa, synthetic construct
  REGION 1..566: note = Synthetic
MNTTTYNTTTDALLEWGKVYFQKEDFSEFLDNLEAYISDAGDSLKDELESGVEKLVLGIKSAEAVIFGEAVIGTTPENEAWYDAEESFLTLDCAVWLSQALDRVVRRQDASLADSLIARLDEAINRVAEKLYADNLSPLRFSSLNEIRRSALEATDEKYHYLFPWHGAACDVDENILLILTEEYHLIGADKAGANLSEELRGDLPFIFAELERDEVLRAYVEKENALSLALENTMREHWAFGLLEAARDEGYNHPYPADVGMRIHQVARAVFSQTNLSPAERLAVAIAGACFTPEISEDRRLEILLDCEERVCEIEAPTGDDTSVRVIKDLKALADHRVRHEIPAESLVSLWFEQIEAAGTDFDTKTPMDELVLRMLSDNVITLSVDRKAASQTETTDDVKPQKGKIIPFPVPDIANDEVEYQKAVGMKKDKKAANDSKVKFPGLLEIQGCRDGDKAILLEDTDDAAANHRKLFSILKAGKLNSAFFIQSDDGEWVESESKPTMEDNRIILHDSHHSSFVWILDTGSMQLRQSVKCVKDALNKKTGSAKKLKPKTMIVWVTIPQEG

SEQ ID NO: 14 - protein, 565 aa, synthetic construct; identical to SEQ ID NO: 2
  REGION 1..565: note = Synthetic

SEQ ID NO: 15 - protein, 565 aa, synthetic construct; identical to SEQ ID NO: 2
  REGION 1..565: note = Synthetic

SEQ ID NO: 16 - protein, 4 aa, synthetic construct
  REGION 1..4: note = Synthetic
ASQT

SEQ ID NO: 17 - protein, 4 aa, synthetic construct
  REGION 1..4: note = Synthetic
AAAA

SEQ ID NO: 18 - protein, 4 aa, synthetic construct
  REGION 1..4: note = Synthetic
ETDD

SEQ ID NO: 19 - protein, 4 aa, synthetic construct
  REGION 1..4: note = Synthetic
VKPQ

SEQ ID NO: 20 - protein, 4 aa, synthetic construct
  REGION 1..4: note = Synthetic
KGKI

SEQ ID NO: 21 - protein, 4 aa, synthetic construct
  REGION 1..4: note = Synthetic
IPFP

SEQ ID NO: 22 - protein, 4 aa, synthetic construct
  REGION 1..4: note = Synthetic
ANDE

SEQ ID NO: 23 - protein, 4 aa, synthetic construct
  REGION 1..4: note = Synthetic
VEYQ

SEQ ID NO: 24 - protein, 4 aa, synthetic construct
  REGION 1..4: note = Synthetic
KAVG

SEQ ID NO: 25 - protein, 4 aa, synthetic construct
  REGION 1..4: note = Synthetic
MKKD

SEQ ID NO: 26 - protein, 4 aa, synthetic construct
  REGION 1..4: note = Synthetic
KKAA

SEQ ID NO: 27 - protein, 4 aa, synthetic construct
  REGION 1..4: note = Synthetic
NDSK

SEQ ID NO: 28 - protein, 4 aa, synthetic construct
  REGION 1..4: note = Synthetic
VKFP

SEQ ID NO: 29 - RNA, 31 nt, synthetic construct
  misc_feature 1..31: note = Synthetic
  source 1..31: mol_type = other RNA
agctgtacaccacgttgtggttgctcttgta

SEQ ID NO: 30 - RNA, 31 nt, synthetic construct
  misc_feature 1..31: note = Synthetic
  source 1..31: mol_type = other RNA
cacagtcgaggctgatcagcgggtttaaacg

SEQ ID NO: 31 - protein, 7 aa, synthetic construct
  REGION 1..7: note = Synthetic
MKKDKKA

SEQ ID NO: 32 - protein, 48 aa, synthetic construct
  REGION 1..48: note = Synthetic
GKIIPFPVPDIANDEVEYQKAVGMKKDKKAANDSKVKFPGLLEIQGCR

SEQ ID NO: 33 - protein, 4 aa, synthetic construct
  REGION 1..4: note = Synthetic
VPDI

SEQ ID NO: 34 - protein, 19 aa, synthetic construct
  REGION 1..19: note = Synthetic
LCCMRRTKQVEKNDEDQKI

SEQ ID NO: 35 - protein, 13 aa, synthetic construct
  REGION 1..13: note = Synthetic
GCVCSSNPENNNN

SEQ ID NO: 36 - protein, 18 aa, synthetic construct
  REGION 1..18: note = Synthetic
GSSKSKPKDPSQRRNNNN

SEQ ID NO: 37 - protein, 158 aa, synthetic construct
  REGION 1..158: note = Synthetic
ISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 38 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 39 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 40 - protein, 5 aa, synthetic construct
  REGION 1..5: note = Synthetic
VITYF

SEQ ID NO: 41 - protein, 5 aa, synthetic construct
  REGION 1..5: note = Synthetic
VVVVV

SEQ ID NO: 42 - protein, 7 aa, synthetic construct
  REGION 1..7: note = Synthetic
KPWWPRR

SEQ ID NO: 43 - protein, 9 aa, synthetic construct
  REGION 1..9: note = Synthetic
NIVNVSLVK

SEQ ID NO: 44 - protein, 5 aa, synthetic construct
  REGION 1..5: note = Synthetic
LPFFD

SEQ ID NO: 45 - protein, 4 aa, synthetic construct
  REGION 1..4: note = Synthetic
KLVF

SEQ ID NO: 46 - protein, 7 aa, synthetic construct
  REGION 1..7: note = Synthetic
PGKLVYA

SEQ ID NO: 47 - protein, 7 aa, synthetic construct
  REGION 1..7: note = Synthetic
LVEALYL

SEQ ID NO: 48 - protein, 6 aa, synthetic construct
  REGION 1..6: note = Synthetic
RGFFYT

SEQ ID NO: 49 - protein, 7 aa, unidentified
  source 1..7: note = Simian virus 40
PKKKRKV

SEQ ID NO: 50 - protein, 10 aa, unidentified
  source 1..10: note = Simian virus 40
PKKKRKVEAS

SEQ ID NO: 51 - protein, 16 aa, Homo sapiens
KRPAATKKAGQAKKKK

SEQ ID NO: 52 - protein, 9 aa, Homo sapiens
PAAKRVKLD

SEQ ID NO: 53 - protein, 11 aa, Homo sapiens
RQRRNELKRSP

SEQ ID NO: 54 - protein, 38 aa, Homo sapiens
NQSSNFGPMKGGNFGGRSSGPYGGGGQYFAKPRNQGGY

SEQ ID NO: 55 - protein, 42 aa, Homo sapiens
RMRIZFKNKGKDTAELRRRRVEVSVELRKAKKDEQILKRRNV

SEQ ID NO: 56 - protein, 8 aa, Homo sapiens
VSRKRPRP

SEQ ID NO: 57 - protein, 8 aa, Homo sapiens
PPKKARED

SEQ ID NO: 58 - protein, 8 aa, Homo sapiens
PQPKKKPL

SEQ ID NO: 59 - protein, 12 aa, Mus sp.
SALIKKKKKMAP

SEQ ID NO: 60 - protein, 5 aa, unidentified
  source 1..5: note = Influenza virus
DRLRR

SEQ ID NO: 61 - protein, 7 aa, unidentified
  source 1..7: note = Influenza virus
PKQKKRK

SEQ ID NO: 62 - protein, 10 aa, unidentified
  source 1..10: note = Hepatitis D virus
RKLKKKIKKL

SEQ ID NO: 63 - protein, 10 aa, Mus sp.
REKKKFLKRR

SEQ ID NO: 64 - protein, 20 aa, Homo sapiens
KRKGDEVDGVDEVAKKKSKK

SEQ ID NO: 65 - protein, 17 aa, Homo sapiens
RKCLQAGMNLEARKTKK

SEQ ID NO: 66 - RNA, 12 nt, synthetic construct
  misc_feature 1..12: note = Synthetic
  misc_feature 1: note = Biotin Tag = /5Biosg/
  misc_feature 12: note = 3IAbRQSp = Iowa black quencher
  source 1..12: mol_type = other RNA
tctcgtacgttc

SEQ ID NO: 67 - RNA, 24 nt, synthetic construct
  misc_feature 1..24: note = Synthetic
  misc_feature 1: note = Biotin Tag = /5Biosg/
  misc_feature 24: note = 3IAbRQSp = Iowa black quencher
  source 1..24: mol_type = other RNA
tctcgtacgttctctcgtacgttc

SEQ ID NO: 68 - protein, 10 aa, synthetic construct
  REGION 1..10: note = Synthetic
  VARIANT 2..4: note = Xaa can be any naturally occurring amino acid
  VARIANT 6..7: note = Xaa can be any naturally occurring amino acid
  VARIANT 9: note = Xaa can be any naturally occurring amino acid
LXXXLXXLXL

SEQ ID NO: 69 - protein, 4 aa, synthetic construct
  REGION 1..4: note = Synthetic
KDEL

SEQ ID NO: 70 - protein, 15 aa, synthetic construct
  REGION 1..15: note = Synthetic
GGGGGGGGGGGGGGG

SEQ ID NO: 71 - protein, 15 aa, synthetic construct
  REGION 1..15: note = Synthetic
GGGGSGGGGSGGGGS

SEQ ID NO: 72 - protein, 5 aa, synthetic construct
  REGION 1..5: note = Synthetic
TEFCA

SEQ ID NO: 73 - protein, 5 aa, synthetic construct
  REGION 1..5: note = Synthetic
APRPG

SEQ ID NO: 74 - protein, 4 aa, synthetic construct
  REGION 1..4: note = Synthetic
GALA

SEQ ID NO: 75 - protein, 8 aa, synthetic construct
  REGION 1..8: note = Synthetic
GPLGIAGQ

SEQ ID NO: 76 - DNA, 10 bp, synthetic construct
  misc_feature 1..10: note = Synthetic
  source 1..10: mol_type = other DNA
tatatatata

SEQ ID NO: 77 - DNA, 15 bp, synthetic construct
  misc_feature 1..15: note = Synthetic
  source 1..15: mol_type = other DNA
gtcgtcgtcgtcgtc

SEQ ID NO: 78 - protein, 24 aa, synthetic construct
GSKVSSALKEKVSALKEKVSALKE

SEQ ID NO: 79 - protein, 518 aa, synthetic construct
GSKVSALKEKVSALKEKVSALKEKVSALKEKVSALKEDIEGRGSLLTCGDVEENPGPKLATMRKGEELFTGIVPILVELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPGGRSREGRGSLLTCGDVEENPGPELMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

(19) United States
(12) Patent Application Publication
(10) Pub. No.: US 2025/0129355 A1
(43) Pub. Date: Apr. 24, 2025

(54) PROGRAMMABLE NUCLEASE-PEPTIDASE COMPOSITIONS

(71) Applicants: The Broad Institute, Inc., Cambridge, MA (US); Massachusetts Institute of Technology, Cambridge, MA (US)

(72) Inventors: Feng Zhang, Cambridge, MA (US); Jonathan Strecker, Cambridge, MA (US)

(21) Appl. No.: 18/932,763

(22) Filed: Oct. 31, 2024

Related U.S. Application Data
(63) Continuation of application No. PCT/US2023/066501, filed on May 2, 2023.
(60) Provisional application No. 63/337,386, filed on May 2, 2022.
(60) Provisional application No. 63/358,306, filed on Jul. 5, 2022.

(51) Int. Cl. C12N 9/64; A61K 38/00; C12N 9/22; G01N 33/58

(52) U.S. Cl. CPC C12N 9/6424; C12N 9/22; G01N 33/581; A61K 38/00; C12N 2310/20; C12Y 304/21111

(57) ABSTRACT

Described in certain example embodiments herein are programmable nuclease-peptidase compositions, systems, and methods for the manipulation of nucleic acids and/or polypeptides. In some embodiments, the programmable nuclease-peptidase composition comprises a repeat-associated mysterious protein (RAMP) polypeptide; a guide molecule capable of forming a RAMP-guide molecule complex with the RAMP polypeptide and directing sequence specific binding of the complex to a target polynucleotide; and a peptidase capable of binding to the RAMP polypeptide, the guide molecule, or further complexing with the RAMP-guide molecule complex, wherein binding of the RAMP-guide molecule complex to the target polynucleotide initiates binding and/or interaction of the peptidase with a target polypeptide.

### CROSS-REFERENCE TO RELATED APPLICATIONS

This application is a continuation application of PCT/US2023/066501, filed May 2, 2023, which claims the benefit of and priority to U.S. Provisional Patent Application No. 63/337,386, filed on May 2, 2022, and U.S. Provisional Patent Application No. 63/358,306, filed on Jul. 5, 2022, the contents of which are incorporated by reference herein in their entireties.

### STATEMENT REGARDING FEDERALLY SPONSORED RESEARCH

This invention was made with government support under Grant No. HL141201 awarded by the National Institutes of Health. The government has certain rights in the invention.

### SEQUENCE LISTING

This application contains a sequence listing filed in electronic form as an xml file entitled “BROD-5545US_ST26”, created on Nov. 19, 2024, and having a size of 103,156 bytes. The content of the sequence listing is incorporated herein in its entirety.

### TECHNICAL FIELD

The subject matter disclosed herein is generally directed to programmable nuclease compositions, systems, and methods. In particularly, the present disclosure describes programmable nuclease-peptidase compositions, systems, and methods.

### BACKGROUND

While there are genome-editing techniques available for producing targeted genome perturbations, there remains a pressing need for new and alternative genome engineering technologies that employ robust novel strategies and molecular mechanisms and are affordable, easy to set up, scalable, and amenable to targeting multiple positions within the genome. The CRISPR-Cas systems of bacterial and archaeal adaptive immunity are some such systems that show extreme diversity of protein composition and genomic loci architecture. These additional desirable tools in genome engineering and biotechnology would further advance the art.

Citation or identification of any document in this application is not an admission that such a document is available as prior art to the present invention.

### SUMMARY

Described in certain example embodiments herein are programmable nuclease-peptidase compositions comprising a repeat-associated mysterious protein (RAMP) polypeptide; a guide molecule capable of forming a RAMP-guide molecule complex with the RAMP polypeptide and directing sequence specific binding of the complex to a target polynucleotide; and a peptidase capable of binding to the RAMP polypeptide, the guide molecule, or further complexing with the RAMP-guide molecule complex, wherein binding of the RAMP-guide molecule complex to the target polynucleotide initiates binding and/or interaction of the peptidase with a target polypeptide.

In certain example embodiments, target polypeptide interaction and/or binding occurs at, or in effective proximity to, a peptidase recognition motif in the target polypeptide.

In certain example embodiments, the peptidase recognition motif comprises or consists of SEQ ID NO: 3 or a sequence therein optionally MKKD (SEQ ID NO: 25).

In certain example embodiments, the peptidase is a TPR-CHAT peptidase.

In certain example embodiments, the TPR-CHAT peptidase is derived from Desulfonema ishimotonii, or a homolog, ortholog, or variant thereof.

In certain example embodiments, the RAMP polypeptide is derived from Desulfonema ishimotonii, or a homolog, ortholog or variant thereof.

In certain example embodiments, the RAMP polypeptide comprises a Cas11 domain and multiple Cas7 domains.

In certain example embodiments, the RAMP polypeptide further comprises a Csm3, Csm4, or Csm6 domain.

In certain example embodiments, the RAMP polypeptide is a Type III-E Cas polypeptide.

In certain example embodiments, the target polypeptide comprises, consists of, or is coupled to an effector, wherein the effector is optionally a reporter polypeptide; a signal amplification polypeptide; an engineered prodrug; a cargo polypeptide; or a pathogenic polypeptide.

In certain example embodiments, the target polypeptide is a disease-associated polypeptide.

In certain example embodiments, the disease-associated polypeptide is associated with a neurodegenerative disease, a metabolic disease, an autoimmune disease, or a cancer.

Described in certain example embodiments herein are polynucleotides that encode a programmable nuclease-peptidase composition or component thereof as described herein.

In certain example embodiments, the polynucleotides further comprise one or more regulatory elements and wherein the polynucleotide encoding a programmable nuclease-peptidase composition or component thereof is operatively coupled to one or more of the one or more regulatory elements.

Described in certain example embodiments herein are vectors or vector systems comprising one or more polynucleotides that encode a programmable nuclease-peptidase composition or component thereof described herein.

In certain example embodiments, the vector or vector system is a viral vector or vector system, optionally an adeno-associated virus vector or vector system.

Described in certain example embodiments herein are cells or cell populations that comprise a programmable nuclease-peptidase composition described herein.

Described in certain example embodiments herein are pharmaceutical formulations comprising a programmable nuclease-peptidase composition or component thereof as described herein, a target polypeptide, a target polynucleotide, a nucleic acid and/or polypeptide detection composition or component thereof as described herein, an engineered composition or component thereof as described herein, a polynucleotide as described herein, a vector or vector system as described herein, a cell or cell population as described herein, or any combination thereof; and a pharmaceutically acceptable carrier.

Described in certain example embodiments herein are methods of modifying a polypeptide comprising introducing into a sample having one or more target polynucleotide and target polypeptides, the programmable nuclease-peptidase composition described herein; and activating the peptidase via sequence specific binding of the complex to the one or more target polynucleotides such that the peptidase then binds or interacts with the one or more target polypeptides resulting in modification of the one or more target polypeptides.

In certain example embodiments, the target polypeptide modification is cleavage of the target polypeptide.

In certain example embodiments, introducing into the sample comprises in vitro, ex vivo, or in vivo delivery of the programable nuclease-peptidase composition into a cell or cell population.

In certain example embodiments, the one or more target polypeptides are proenzymes and the modification results in conversion of the proenzyme into an active enzyme.

In certain example embodiments, modification of the one or more target polypeptides results in activation or deactivation of one or more cell-signaling proteins.

In certain example embodiments, the one or more target polynucleotides are a specific transcript or set of transcripts and wherein modification of the one or more target polypeptides triggers cell death upon activating the peptidase in response to binding of the nuclease-peptidase to the specific transcript or set of transcripts.

In certain example embodiments, the guide molecule is configured to detect one or more mutations in the specific transcript or set of transcripts.

Described in certain example embodiments herein are detection compositions comprising (i) a RAMP polypeptide; (ii) a guide molecule capable of forming a RAMP-guide molecule complex with the RAMP polypeptide and directing sequence-specific binding of the complex to a target polynucleotide; (iii) a peptidase capable of binding the RAMP polypeptide, the guide molecule, or further complexing with the RAMP-guide complex; and (iv) a detection construct, wherein binding of the RAMP-guide molecule complex to the target polynucleotide initiates peptidase mediated modification of the detection construct resulting in generation of a detectable signal.

In certain example embodiments, the detection construct comprises a peptidase recognition motif recognized by the peptidase.

In certain example embodiments, the peptidase recognition motif comprises or consists of SEQ ID NO: 3 or a sequence therein, optionally MKKD (SEQ ID NO: 25).

In certain example embodiments, the peptidase is a TPR-CHAT peptidase. In certain example embodiments, the TPR-CHAT peptidase is derived from Desulfonema ishimotonii or a homolog, ortholog, or variant thereof.

In certain example embodiments, the RAMP polypeptide is derived from Desulfonema ishimotonii, or a homolog, ortholog or variant thereof.

In certain example embodiments, the RAMP polypeptide comprises a Cas11 domain and multiple Cas7 domains.

In certain example embodiments, the RAMP polypeptide further comprises a Csm3, Csm4, or Csm6 domain.

In certain example embodiments, the RAMP polypeptide is a Type III-E Cas polypeptide.

In certain example embodiments, the detection construct comprises a polypeptide comprising a protease recognition motif recognized by the peptidase.

In certain example embodiments, the polypeptide is a fluorescent protein protease reporter.

Described in certain example embodiments herein are polynucleotides encoding one or more elements (e.g., (i)-(iv) above) of the detection composition described herein.

Described in certain example embodiments herein are vector systems comprising one or more vectors encoding one or more of elements (i)-(iv) of the detection composition described herein.

Described in certain example embodiments herein are engineered cells modified to express elements (i) and (iii) of the detection composition described herein. In certain example embodiments, where the engineered cells are further modified to express element (iv) of the detection composition described herein. In certain example embodiments, where the engineered cells are further modified to express element (ii) of the detection composition described herein.

Described in certain example embodiments herein are methods for screening cell perturbations comprising introducing a perturbation to a cell population comprising engineered cells of the present invention described herein, such as those modified to express a programmable nuclease-peptidase composition or component thereof and/or detection composition or component thereof described herein, along with any elements of the detection composition not already expressed by the engineered cells, and wherein the guide molecules are configured to detect one or more target transcripts associated with a specific cell type or cell state; activating the peptidase via binding of the complex to one or more target polynucleotides such that the detection construct is modified by the activated peptidase to produce a detectable product and/or signal; and detecting an ability of the perturbation to modify expression of the one or more target transcripts by measuring a change in the detectable product and/or signal relative to a control.

Described in certain example embodiments herein are methods of detecting target polynucleotides in samples comprising combining a sample or a component thereof with the detection composition as described herein; and activating the peptidase via binding of the complex to one or more target polynucleotides such that the detection construct is modified by the activated peptidase such that a detectable product and/or signal is produced, thereby detecting the target polynucleotide in the sample. In certain example embodiments, the method further comprises amplifying and/or enriching the target polynucleotide. In certain example embodiments, activating the peptidase further results in activation or generation of one or more signal amplification molecules.

Described in certain example embodiments herein are methods of labeling cells comprising introducing the detection composition as described herein into a population of cells, wherein the guide molecule is configured to detect one or more target transcripts associated with a particular cell type or cell state; activating the peptidase via binding of the complex to the one or more target transcripts such that the detection construct is modified by the activated peptidase such that a detectable product and/or signal is generated, thereby labeling cells within the cell population expressing the one or more target transcripts.

Described in certain embodiments herein are methods of in vivo effector activation or delivery comprising introducing a programmable nuclease system of the present invention into a cell comprising a substrate of the peptidase, wherein the substrate of the peptidase is optionally tethered to a cellular structure and wherein the substrate the peptidase is coupled to an effector. In certain example embodiments, the effector is capable of producing a detectable signal when activated, is a therapeutic molecule or prodrug, is a genetic modifying molecule, or any combination thereof. In certain example embodiments, the effector is inactive when coupled to an uncleaved substrate. In certain example embodiments, the effector is inactive when coupled to a cleaved substrate portion. In certain example embodiments, the method further comprises cleaving the substrate by the peptidase in response to a target RNA and activation of the peptidase of the programmable nuclease system. In certain example embodiments, the target RNA is endogenous to the cell or is exogenous to the cell. In certain example embodiments, the substrate is tethered to a cell membrane or a nuclear membrane.

In certain example embodiments herein labeled cells are further sorted or isolated based on production of the detectable product and/or signal. These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 (SEQ ID NO: 1)—Shows a 3D ribbon model of the predicted structure of a D. ishimotonii CHAT domain containing protein.

FIG. 2 (SEQ ID NOS: 2-3)—Shows a 3D ribbon model of the predicted structure of a D. ishimotonii CHAT domain containing protein showing a natural target substrate of the CHAT domain containing protein of FIG. 1 with the predicted cleavage site and/or binding motif region shaded and underlined (SEQ ID NO: 3).

FIG. 3 (SEQ ID NOS: 4-5, 78)—Shows a Flip protease reporter assay that can include a substrate of a CHAT domain containing protein. The Flip protease reporter assay can be used to examine substrates of a CHAT domain containing protein. Candidate substrates are incorporated within the flip reporter protein at the position labeled “substrate linker”.

FIG. 4 (SEQ ID NOS: 6-10, 79)—Shows amino acid and polynucleotide sequences associated with various components of the Flip reporter assay for candidate substrates. Candidate substrates are incorporated within the flip reporter protein at the position labeled “substrate linker”.

FIG. 5—Shows a representative SDS-PAGE gel demonstrating in vitro reconstitution of RNA-guided protein cleavage. A gRAMP-protease-crRNA complex was purified from E. coli and incubated with purified WP_124327587.1 protein. Reactions were incubated at 37 C for 1 hour in the presence of Mg2+ and ATP.

FIGS. 6A-6B—Show representative SDS-PAGE gels demonstrating reconstitution of protein substrate cleavage following RNA targeting by the gRAMP-CHAT complex in HEK-293 cells transfected with separate gRAMP and CHAT expression plasmids or a combination of the two proteins with a T2A linker, a targeting or non-targeting crRNA, a plasmid expressing the target RNA, and an HA-tagged protein substrate on the N-terminus (FIG. 6A) or C-terminus (FIG. 6B). Immunoblot analysis using an anti-HA-antibody of the cell lysates was performed after 3 days of incubation. Cleavage of substrate occurred in a manner dependent on a targeting crRNA.

FIGS. 7A-7E (SEQ ID NO: 11)—Demonstrate the gRAMP-CHAT locus from Desulfonema ishimotonii strain Tokyo 01 and that Upstream protein 1 (Up1, WP_12327587.1) is cleaved by the gRAMP-CHAT in response to target RNA. The gRAMP-CHAT complex exhibited protease activity across a wide range of temperatures ranging from 4-50 degrees C. Further, RNA cleavage by gRAMP is not required for protease activity as inactivating the nuclease with the D429A/D654A mutations has no effect on protease activity. Without being bound by theory, this can facilitate applications for sensing RNA without their destruction.

FIGS. 8A-8D (SEQ ID NOS: 12-15)—Show enzyme digest mapping of peptides from the two fragments (N-terminal and C-terminal) produced from Up1 cleavage with the Desulfonema ishimotonii strain Tokyo 01 gRAMP-CHAT. Without being bound by theory, enzyme digest mapping revealed an approximate breakage point around M427-D430.

FIGS. 9A-9B (SEQ ID NOS: 16-28)—Demonstrate that the C-terminal end of Up1 is required for cleavage but that the N-terminal end can be truncated. Smaller versions of Up1 containing amino acids 296-565 retained full activity for processing and can be used in applications to reduce the size of the protein substrate.

FIGS. 10A-10B—Show alanine substitution mutations in the Up1 protein substrate and their effect on protein cleavage. No single alanine mutation blocks CHAT protease activity, which suggested that cleavage is not dependent on a specific residue and potentially that the shape of the substrate is being recognized.

FIG. 11 (SEQ ID NOS: 29-30)—Shows Data from human cells that demonstrates processing of 3×HA-tagged Up1 which is dependent on gRAMP, CHAT, and a targeting crRNA. This activity is abolished in the C658A and H615A CHAT mutations, which disrupted the catalytic site. Consistent with the in vitro data, inactivating the gRAMP nuclease residues with D429A/D654A mutations does not prevent cleavage of Up1 indicating that target RNA binding alone is required. This work was performed with two separate spacer sequences as shown.

FIG. 12—Shows an exemplary schematic for an in vitro nucleic acid detection with gRAMP-CHAT. A gRAMP-CHAT substrate (e.g., Up1) containing an N-terminal avidin tag, which can be biotinylated, and a C-terminal FAM. Cleavage of the biotin-Up1-FAM substrate in response to target RNA can allow for visual detection on a standard biotin/FAM flow strip.

FIG. 13—Shows an exemplary schematic for an in vivo effector system in which proteins are tethered to a cell membrane using transmembrane domains (e.g., gap43: LCCMRRTKQVEKNDEDQKI (SEQ ID NO: 34), L10: GCVCSSNPENNNN (SEQ ID NO: 35), S15: GSSKSKPKDPSQRRNNNN (SEQ ID NO: 36)) with a linker sequence containing a minimal Up1 substrate (amino acids 297-565). Following RNA detection and Up1 cleavage, the effector domain can move into the nucleus and perform different biological activities. For example, dCas9-VPR effector can be used to allow for the activation of genes, and a Cre effector to activate GFP expression.

FIG. 14—Shows an exemplary schematic for a degron in which a degron tag is fused to an effector of interest via a linker sequence containing a minimal Up1 substrate (297-565). For example, a dihydrofolate reductase (DHFR) sequence (ISLIAALAVDHVIGMETVMPWNLPADLAWFKRNTLNKPVIMGRHTWESIGRPLPGR KNIILSSQPSTDDRVTWVKSVDEAIAACGDVPEIMVIGGGRVYEQFLPKAQKLYLTHI DAEVEGDTHFPDYEPDDWESVFSEFHDADAQNSHSYCFEILERR (SEQ ID NO: 37)), which destabilizes the protein resulting in degradation. Following RNA detection and Up1 cleavage, the degron tag is removed from the effector thereby stabilizing the effector and allowing for its activity. Exemplary effectors include reporters (e.g., fluorescent proteins (e.g., GFP)), a Cas (e.g., Cas 9), Cre, and others. Such an approach can be applied to any effector of interest.

FIG. 15A-15C—A type III-E CRISPR-associated protease cleaves Up1 in response to target RNA. FIG. 15A. Schematic of selected CRISPR loci and three conserved upstream genes adjacent to gRAMP and the TPR-CHAT protease. FIG. 15B. A gRAMP-CHAT-crRNA complex cleaves purified Up1 protein in response to target RNA. FIG. 15C. Up1 cleavage requires target RNA and the CHAT protease catalytic residues, but not catalytic residues of gRAMP. Panels FIG. 15B and FIG. 15C are SDS-PAGE gels stained with Coomassie.

FIG. 16A-16G. Requirements of Up1 proteolytic processing and function. FIG. 16A, Schematic of Up1 and the cleavage site as determined by mass spectrometry. FIG. 16B, Alphafold2 structural prediction of Up1 highlighting the cleavage site and putative C-terminal effector domain. FIG. 16C, Analysis of gRAMP-CHAT activity on truncated Up1 proteins. FIG. 16D (SEQ ID NOS: 17, 25, 31), Western blot analysis of Up1 mutants generated by cell free transcription-translation. FIG. 16E, gRAMP-CHAT binds to Up1 in the absence of target RNA. Pulldown of TwinStrep-Up1 mutants and the elution of bound proteins. FIG. 16F, Pulldown of HIS-Up3 in the presence of untagged Up1 yields a Up1-Up3 complex that is cleaved by gRAMP-CHAT. FIG. 16G, Model for potential three-pronged capability of CASP systems in defense against foreign genetic elements. Panels FIG. 16C, FIG. 16E, and FIG. 16F are SDS-PAGE gels stained with Coomassie.

FIG. 17A-17F—RNA sensing applications with DiCASP in vitro and in human cells. FIG. 17A, Schematic of Up1 substrates for diagnostic applications. FIG. 17B, RNA detection using an engineered Up1 reporter across target RNA concentration. FIG. 17C, Immunoblot analysis of Up1 protein cleavage in HEK293T human cells transfected with DiCASP. FIG. 17D—Immunoblot analysis of Up1 cleavage in response to detection of endogenous transcripts at different levels of expression in HEK293T cells (low: 1-10 TPM, medium: 10-100 TPM, high: 100-1000 TPM). FIG. 17E, Schematic of engineered membrane tethered proteins containing Up1 and an effector domain in human cells. FIG. 17F, Flow cytometry of DiCASP activity in Neuro2A loxP:GFP cells using a Chrm3-Up₂₅₀₋₅₆₅⁻Cre reporter. Error bars represent standard deviation from the mean.

FIG. 18A-18E—FIG. 18A, Immunoblot analysis of in vitro reactions with 3×HA tagged Up1-3 and gRAMP-CHAT. FIG. 18B, Time course of Up1 cleavage upon addition of target RNA. FIG. 18C, Dilution series of gRAMP-CHAT relative to Up1 concentration. FIG. 18D, Up1 cleavage across dilution series of target RNA. FIG. 18E, Up1 cleavage across a temperature range. Panels FIG. 18B-18E are SDS-PAGE gels stained with Coomassie.

FIG. 19A-19B—FIG. 19A, Mass spectrometry analysis of Up1 processed fragments following trypsin and chymotrypsin digests. FIG. 19B (SEQ ID NO: 32), Unique peptides detected by mass spectrometry around the Up1 cleavage site.

FIG. 20A-20C—FIG. 20A, In vitro cleavage of truncated Up1 proteins. SDS-PAGE gel stained with Coomassie. FIG. 20B-20C (SEQ ID NOS: 17-28, 33), Immunoblot analysis of in vitro reactions with 3×HA-Up1 mutants produced by cell-free transcription-translation.

FIG. 21A-21E—FIG. 21A, Thin layer chromatography of cell wall components following incubation with full length or cleaved Up1. FIG. 21B, Growth curves of E. coli overexpressing Up1N or Up1C in combination with Up2. FIG. 21C, Growth curves of E. coli overexpressing Up1N or Up1C combined with cellular stresses. FIG. 21D, Schematic of Up1 and Up3 and an Alphafold2 prediction of a Up1-Up3 interaction. FIG. 21E, Confocal microscopy of msGFP-Up1 and msGFP-Up3 in live E. coli.

FIG. 22A-22D—FIG. 22A, Schematic of an engineered Up1 substrate for diagnostic applications and labeling strategy. FIG. 22B, Immunoblot analysis of HA-tagged Up1 truncation mutants in HEK293T cells. FIG. 22C, Correlation between Up1 cleavage efficiency in FIG. 3d and RNA expression level. FIG. 22D, Flow cytometry of DiCASP activity in Neuro2A loxP:GFP cells using a Gap43-Up250-565 Cre reporter. Error bars represent standard deviation from the mean.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide programmable nuclease-peptidase compositions that can have CRISPR-activated peptidase (or protease) activity. In general, such compositions include a repeat-associated mysterious protein (RAMP) polypeptide, that like traditional CRISPR-Cas based systems, is capable of binding or otherwise activating an associated peptidase upon RAMP activation by complexing with a guide and/or target polynucleotide. Such compositions can have various applications, including detection of target polynucleotides, modification of target polypeptides, activation of proenzymes and prodrugs, labeling of cells, among others.

### Programmable Nuclease-Peptidase Compositions

Described in certain example embodiments herein are programmable nuclease-peptidase compositions comprising a repeat-associated mysterious protein (RAMP) polypeptide; a guide molecule capable of forming a RAMP-guide molecule complex with the RAMP polypeptide and directing sequence specific binding of the complex to a target polynucleotide; and a peptidase capable of binding to the RAMP polypeptide, the guide molecule, or further complexing with the RAMP-guide molecule complex, wherein binding of the RAMP-guide molecule complex to the target polynucleotide initiates binding and/or interaction of the peptidase with a target polypeptide.

The target polypeptide may be, but is not limited to, a reporter polypeptide; a signal amplification polypeptide; an engineered prodrug; a cleavable linker; a cargo polypeptide; or a pathogenic polypeptide.

Also described in certain example embodiments herein are detection compositions that comprise one or more components of the programmable nuclease-peptidase compositions described herein. In some embodiments, a detection composition comprises (i) a RAMP polypeptide; (ii) a guide molecule capable of forming a RAMP-guide molecule complex with the RAMP polypeptide and directing sequence-specific binding of the complex to a target polynucleotide; (iii) a peptidase capable of binding the RAMP polypeptide, the guide molecule, or further complexing with the RAMP-guide complex; and (iv) a detection construct, wherein binding of the RAMP-guide molecule complex to the target polynucleotide initiates peptidase mediated modification of the detection construct resulting in generation of a detectable signal.

### Peptidases

Generally, the programmable nuclease-peptidase composition described herein includes a peptidase or functional domain thereof that is capable of binding, interacting with, or otherwise associating with or complexing with a RAMP polypeptide. RAMP polypeptides are described in greater detail elsewhere herein. In some embodiments, the peptidase or functional domain thereof is activated upon biding of the composition to a target nucleic acid, thereby exhibiting polypeptide cleavage activity. The polypeptide cleavage activity may be a peptidase activity, e.g., an endopeptidase or exopeptidase activity. The peptidase, or functional domain thereof, may be a caspase polypeptide or functional domain thereof. In some embodiments, the peptidase is a Caspase HetF Associated with Tprs (TPR-CHAT) peptidase or functional domain thereof. In certain example embodiments, the TPR-CHAT peptidase is derived from Desulfonema ishimotonii, or a homolog, ortholog, or variant thereof. A TPR-CHAT peptidase is a peptidase is a peptidase comprising a TPR-CHAT domain, also referred to as a “CHAT domain”. In some embodiments, the TPR-CHAT peptidase or TPR-CHAT domain is derived from Desulfonema ishimotonii, Candidatus jettenia caeni, Candidatus Scalindua brodae, Deltaprotobacterium, Desulfobacteraceae bacterium, or Candidatus brocadia fulgda.

The peptidase, or functional domain thereof, may be 70-100% identical to SEQ ID NO: 1, or a region of at least 5, 10 20, 30, 40, 50, 60, 70, 80, 90, 100, or more contiguous amino acids thereof. In some embodiments, the peptidase or functional domain thereof is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to/or 100% identical to identical to SEQ ID NO: 1 or region thereof of at least 5, 10 20, 30, 40, 50, 60, 70, 80, 90, 100, or more contiguous amino acids thereof.

In some embodiments, the peptidase is a multi-turnover peptidase. In some embodiments, the peptidase is capable of cleaving or otherwise processing an excess of substrate.

In some embodiments, the programmable nuclease-peptidase composition has peptidase activity at a temperature ranging from 4-50° C., such as 4° C., 4.5° C., 5° C., 5.5° C., 6° C., 6.5° C., 7° C., 7.5° C., 8° C., 8.5° C., 9° C., 9.5° C., 10° C., 10.5° C., 11° C., 11.5° C., 12° C., 12.5° C., 13° C., 13.5° C., 14° C., 14.5° C., 15° C., 15.5° C., 16° C., 16.5° C., 17° C., 17.5° C., 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., 40° C., 40.5° C., 41° C., 41.5° C., 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., or 50° C. In some embodiments the programmable nuclease-peptidase composition has peptidase activity at a temperature of about 37° C. to about 45° C.

In some embodiments, the programmable nuclease-peptidase composition lacks nucleic acid cleavage activity but is otherwise capable of recognizing, complexing and/or binding a target nucleic acid and has peptidase activity. In some embodiments, the programmable nuclease-peptidase composition is engineered to lack nucleic acid cleavage activity and retain target nucleic acid recognition, complexing, and/or binding activity and peptidase activity.

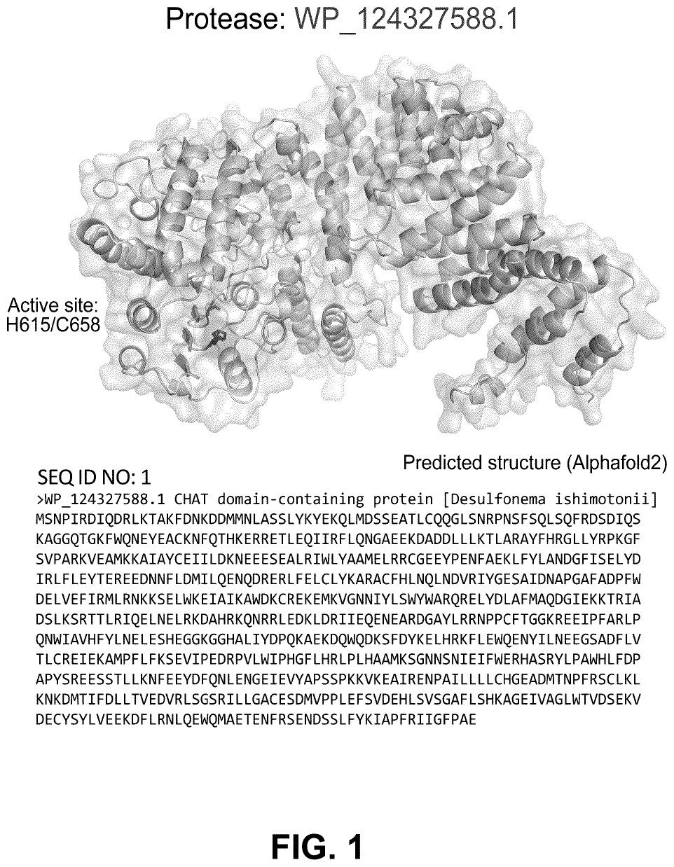

(FIG. 1)
SEQ ID NO: 1
>WP_124327588.1 CHAT domain-containing protein
[Desulfonema ishimotonii]
MSNPIRDIQDRLKTAKFDNKDDMMNLASSLYKYEKQLMDSSEATLC
QQGLSNRPNSFSQLSQFRDSDIQSKAGGQTGKFWQNEYEACKNFQ
THKERRETLEQIIRFLQNGAEEKDADDLLLKTLARAYFHRGLLYR
PKGFSVPARKVEAMKKAIAYCEIILDKNEEESEALRIWLYAAMEL
RRCGEEYPENFAEKLFYLANDGFISELYDIRLFLEYTEREEDNNF
LDMILQENQDRERLFELCLYKARACFHLNQLNDVRIYGESAIDNA
PGAFADPFWDELVEFIRMLRNKKSELWKEIAIKAWDKCREKEMKV
GNNIYLSWYWARQRELYDLAFMAQDGIEKKTRIADSLKSRTTLRI
QELNELRKDAHRKQNRRLEDKLDRIIEQENEARDGAYLRRNPPCF
TGGKREEIPFARLPQNWIAVHFYLNELESHEGGKGGHALIYDPQK
AEKDQWQDKSFDYKELHRKFLEWQENYILNEEGSADFLVTLCREI
EKAMPFLFKSEVIPEDRPVLWIPHGFLHRLPLHAAMKSGNNSNIE
IFWERHASRYLPAWHLFDPAPYSREESSTLLKNFEEYDFQNLENG
EIEVYAPSSPKKVKEAIRENPAILLLLCHGEADMINPFRSCLKLK
NKDMTIFDLLTVEDVRLSGSRILLGACESDMVPPLEFSVDEHLSV
SGAFLSHKAGEIVAGLWTVDSEKVDECYSYLVEEKDFLRNLQEWQ
MAETENFRSENDSSLFYKIAPFRIIGFPAE

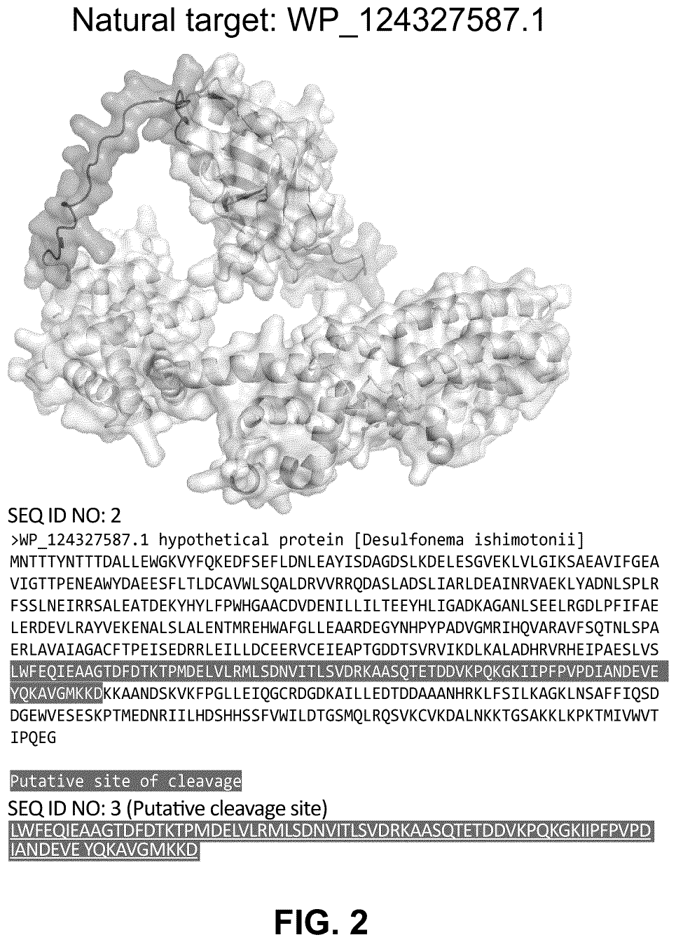

The peptidase or functional domain thereof is capable of binding, interacting with, associating with, or otherwise complexing with and/or cleaving a polypeptide having a peptide sequence according to SEQ ID NO: 2 or 3 (see e.g., FIG. 2), or a sequence therein. In some embodiments, the peptidase or functional domain thereof is capable of binding, interacting with, associating with, or otherwise complexing with and/or cleaving a polypeptide having a peptide sequence having an N-terminal truncation of SEQ ID NO: 2. In some embodiments, the N-terminal truncation is a truncation of amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, or 395 of an Up1 polypeptide, such as SEQ ID NO: 2.

In some embodiments, the substrate of the peptidase is 80-100 percent (e.g., 80, 81, 82, 83, 84, 85, 86, 87, 88, 89, 90, 91, 92, 93, 94, 95, 96, 97, 98, 99, 100 percent) identical to the C-terminus of an Up1 polypeptide (e.g., residues 396-565 of SEQ ID NO: 2).

The peptidase or functional domain thereof may also be capable of specifically binding and/or cleaving a polypeptide having a peptide sequence as in SEQ ID NO: 3 or a region therein, optionally MKKD (SEQ ID NO: 25). In some embodiments, the peptidase or functional domain thereof may also be capable of specifically binding and/or cleaving a polypeptide having 3-70 or more contiguous amino acids of the peptide sequence according to SEQ ID NO: 3.

The peptidase can be engineered to have reduce or eliminated peptidase activity, e.g., polypeptide cleavage activity. The peptidase can also be engineered to recognize, bind, cleave, or otherwise interact or associate with a different substrate than its native substrate. In some embodiments, the peptidase is engineered to recognize, bind, cleave, or otherwise interact or associate with a different substrate than its native substrate using a directed evolution approach, such as that utilized to evolve BoNT proteases described in e.g., Blum et al. Science. 2021. 371(6531):803-810; International Patent Application Publications WO 2019/040935, WO 2021/011579, WO 2022/133266, and/or WO 2023/010050, all of which are incorporated by reference as if expressed in their entireties herein.

In some embodiments, the peptidase is engineered to recognize, bind, cleave, or otherwise interact or associate with any one of the peptide sequences of SEQ ID NO: 2 or a sequence therein, optionally an N-terminal truncation (e.g., an N-terminal truncation of SEQ ID NO: 2 up to amino acid 295 as previously described), a peptidase recognition motif (e.g., SEQ ID NO: 3 or a sequence therein, optionally MKKD (SEQ ID NO: 25), as further described in detail elsewhere herein). In some embodiments, the peptidase is engineered to recognize, bind, cleave, or otherwise interact or associate with any one of the peptide sequences of SEQ ID NO: 3 or a region therein, optionally MKKD (SEQ ID NO: 25).

In some embodiments, the catalytic residues of the CHAT protease are modified so as to increase or otherwise modify (e.g., substrate preference) protease activity. In some embodiments, residue H615 and/or C658 relative to D. ishimotonii CHAT protease or amino acids corresponding thereto in a non-D. ishimotonii CHAT are modified.

### Peptidase Recognition Motifs

The peptidase of the programmable-nuclease composition can be capable of interacting binding, associating, complexing with and/or cleaving a target polypeptide. In certain example embodiments, target polypeptide interaction and/or binding with the peptidase occurs at, or in effective proximity to, a peptidase recognition motif in the target polypeptide. In some embodiments, the interaction is cleavage of a target polypeptide at one or more locations in a target polypeptide. In some embodiments, cleavage and/or other interaction is within the peptidase recognition motif. In some embodiments, cleavage and/or other interaction is not within the peptidase recognition motif. In some embodiments, cleavage is effective proximity to the peptidase recognition motif.

As used herein, the term “effective proximity” refers to the distance, region, number of amino acid residues, number of nucleic acids, or area surrounding a reference point, motif, sequence, or object in which a desired effect or activity occurs. In some embodiments, the desired effect or activity is cleavage of a target polypeptide. In some embodiments, the desired effect or activity is binding, complexing, or otherwise interacting or association with a target polypeptide. In some embodiments, the desired effect is modification of one or more amino acid residues of the target polypeptide.

In some embodiments, effective proximity is 0, to/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more amino acids away from the peptidase recognition motif.

In some embodiments, effective proximity is a distance of 0 Å to 100 Å or more, such as 1 Å, to/or 2 Å, 3 Å, 4 Å, 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, 80 Å, 81 Å, 82 Å, 83 Å, 84 Å, 85 Å, 86 Å, 87 Å, 88 Å, 89 Å, 90 Å, 91 Å, 92 Å, 93 Å, 94 Å, 95 Å, 96 Å, 97 Å, 98 Å, 99 Å, 100 Å, or more.

In some embodiments, the peptidase recognition motif comprises or consists of SEQ ID NO: 3 or a sequence therein, optionally MKKD (SEQ ID NO: 25). In some embodiments, the peptidase recognition motif comprises or consists of 3-70 or more contiguous amino acids of SEQ ID NO: 3.

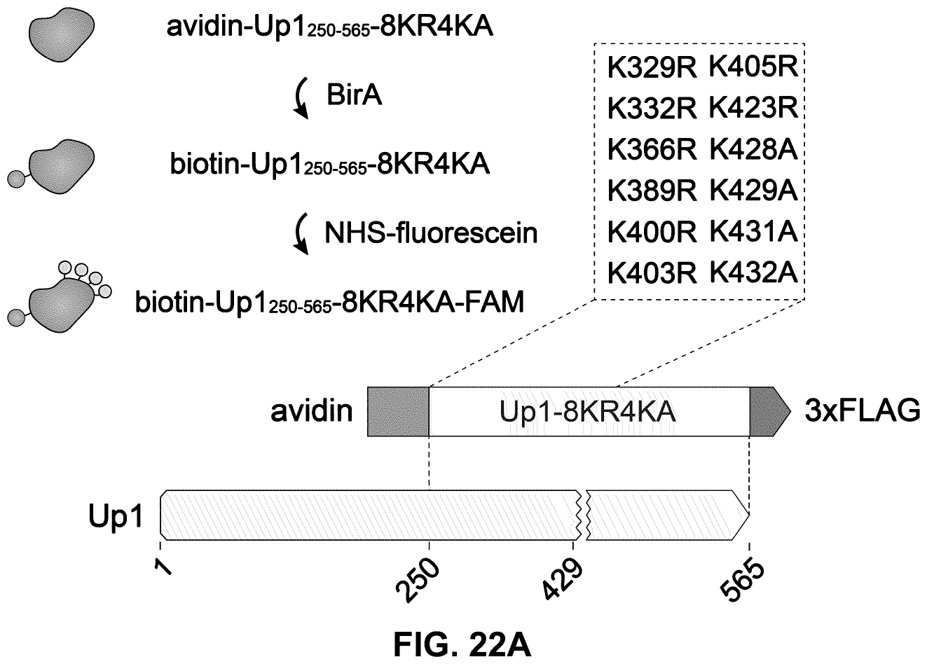

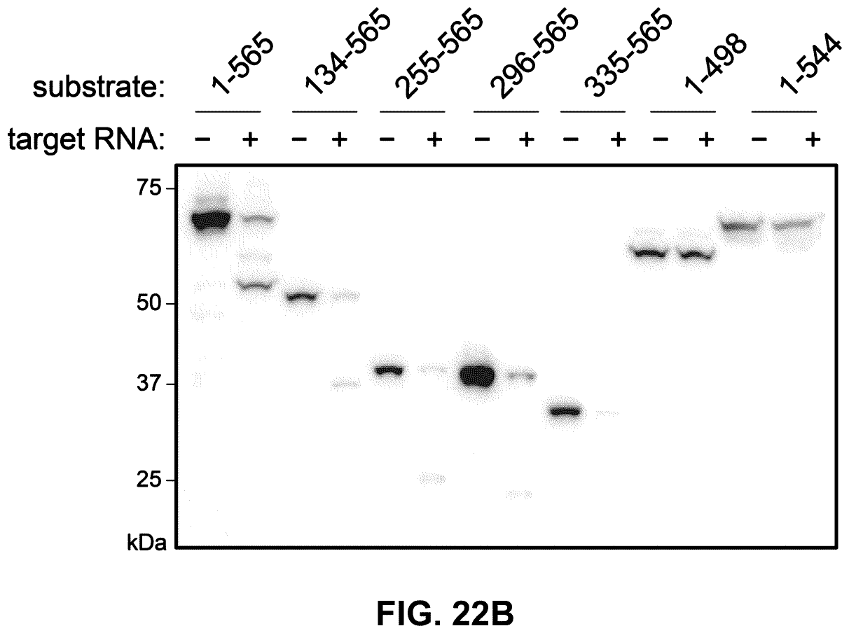

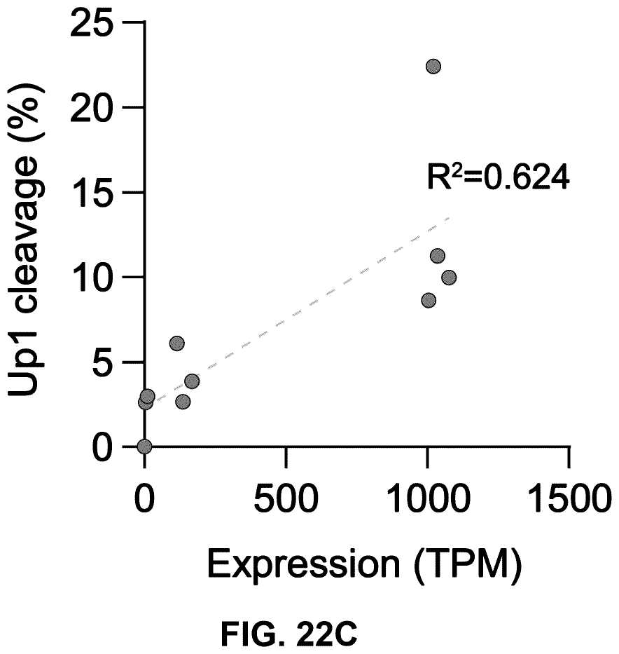

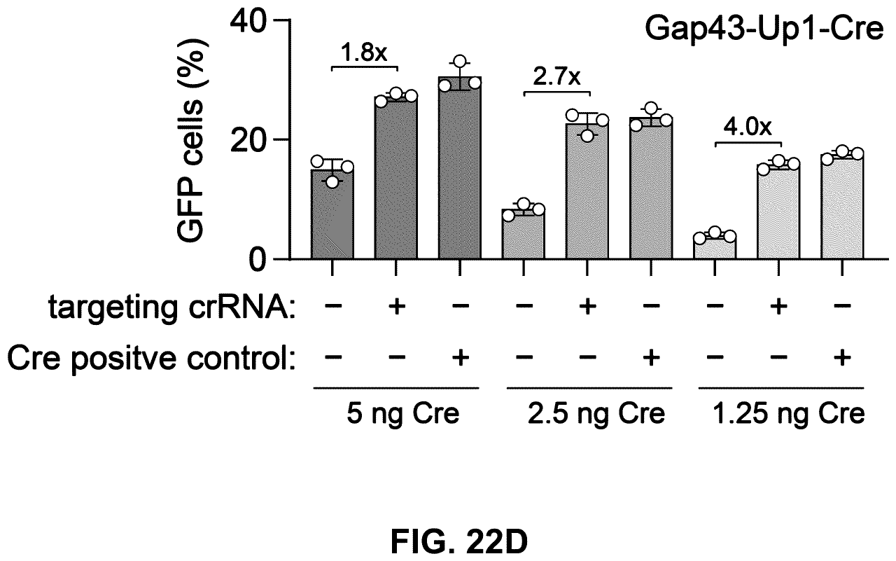

In some embodiments, the peptidase recognition motif comprises one or more mutated lysine residues. In some embodiments, the peptidase recognition motif comprises 1, 2, 3, 4, 5, 6, 7, 8, 9, 10, 11, 12, 13, 14, 15, 16, 17, 18, 19, 20, or more mutated lysine residues. In some embodiments, the one or more lysine residues are mutated to an alanine or an arginine. In some embodiments, the peptidase recognition motif comprises one more mutated lysine residues selected from K329, K332, K366, K389, K400, K403, K405, K423, K428, K429, K431, and K432. In some embodiments, the peptidase recognition motif comprises one more mutated lysine residues selected from K329, K332, K366, K389, K400, K403, K405, and K423. In some embodiments, the peptidase recognition motif comprises one more mutated lysine residues selected from K428, K429, K431, and K432. In some embodiments, the peptidase recognition motif comprises one more mutations selected from K329R, K332R, K366R, K389R, K400R, K403R, K405R, K423R, K428A, K429A, K431A, and K432A. In some embodiments, the peptidase recognition motif comprises one more mutations selected from K329R, K332R, K366R, K389R, K400R, K403R, K405R, and K423R. In some embodiments, the peptidase recognition motif comprises one more mutations selected from K428A, K429A, K431A, and K432A. In some embodiments, the peptidase recognition motif comprises a mutated K329. In some embodiments, the peptidase recognition motif comprises a mutated K332. In some embodiments, the peptidase recognition motif comprises a mutated K366. In some embodiments, the peptidase recognition motif comprises a mutated K389. In some embodiments, the peptidase recognition motif comprises a mutated K400. In some embodiments, the peptidase recognition motif comprises a mutated K403. In some embodiments, the peptidase recognition motif comprises a mutated K405. In some embodiments, the peptidase recognition motif comprises a mutated K423. In some embodiments, the peptidase recognition motif comprises a mutated K428. In some embodiments, the peptidase recognition motif comprises a mutated K429. In some embodiments, the peptidase recognition motif comprises a mutated K431. In some embodiments, the peptidase recognition motif comprises a mutated K432. It will be appreciated that the position numbering is with reference to SEQ ID NO: 2 and correspond to positions in SEQ ID NO: 3. See e.g., FIG. 22A.

### RAMP Polypeptides

The programmable nuclease-peptidase composition comprises a RAMP polypeptide (also referred to as a RAMP domain). In certain example embodiments, the RAMP polypeptide is derived from Desulfonema ishimotonii, or a homolog, ortholog or variant thereof. In some embodiments, the RAMP polypeptide contains an RNA recognition motif (RRM). In some embodiments, the RAMP polypeptide contains multiple domains. In certain example embodiments, the RAMP polypeptide comprises a Cas11 domain and multiple Cas7 domains. In some embodiments, the number of Cas7 domains is 2, 3, 4, 5, 6, or more. In some embodiments the Cas11 domain and/or Cas7 domains are derived from Desulfonema ishimotonii. In some embodiments the Cas 11 domain and/or the Cas 7 domains are derived from Desulfonema ishimotonii, Candidatus jettenia caeni, Candidatus scalindua brodae, Deltaprotobacterium, Desulfobacteraceae bacterium, Candidatus brocadia fulgda, Syntrophohabdaceae bacterium, and/or Candidatus magnebomorum.

In certain example embodiments, the RAMP polypeptide further comprises a Csm3, Csm4, or Csm6 domain. In some embodiments the Csm3, Csm4, and/or the Csm6 domains are derived from Desulfonema ishimotonii, Candidatus jettenia caeni, Candidatus scalindua brodae, Deltaprotobacterium, Desulfobacteraceae bacterium, Candidatus brocadia fulgda, Syntrophohabdaceae bacterium, and/or Candidatus magnebomorum.

In certain example embodiments, the RAMP polypeptide is a Type III-E Cas polypeptide. In some embodiments, the RAMP polypeptide is a Type III-E Cas polypeptide derived from Desulfonema ishimotonii, Candidatus jettenia caeni, Candidatus scalindua brodae, Deltaprotobacterium, Desulfobacteraceae bacterium, Candidatus brocadia fulgda, Syntrophohabdaceae bacterium, and/or Candidatus magnebomorum.

In some embodiments, the RAMP polypeptide does not contain a Cas10 and/or Cas 5 domain.

In some embodiments, the RAMP polypeptide is about 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 525 amino acids, 550 amino acids, 575 amino acids, 600 amino acids, 625 amino acids, 650 amino acids, 675 amino acids, 700 amino acids, 725 amino acids, 750 amino acids, 775 amino acids, 800 amino acids, 825 amino acids, 850 amino acids, 875 amino acids, 900 amino acids, 925 amino acids, 950 amino acids, 975 amino acids, 1000 amino acids, 1025 amino acids, 1050 amino acids, 1075 amino acids, 1100 amino acids, 1125 amino acids, 1150 amino acids, 1175 amino acids, 1200 amino acids, 1225 amino acids, 1250 amino acids, 1275 amino acids, 1300 amino acids, 1325 amino acids, 1350 amino acids, 1375 amino acids, 1400 amino acids, 1425 amino acids, 1450 amino acids, 1475 amino acids, 1500 amino acids, 1525 amino acids, 1550 amino acids, or more amino acids in length.

### Target Polypeptides

The target polypeptide can be any polypeptide that is a substrate for the peptidase within the programmable nuclease-peptidase composition. In one example embodiment, the target polypeptide comprises a peptidase recognition motif. In one example embodiment, the peptidase recognition motif comprises or consists of a peptide of SEQ ID NO: 3 or a sequence therein, optionally MKKD (SEQ ID NO: 25). In some embodiments, peptidase recognition motif comprises or consists of a peptide that is 3-70 or more contiguous amino acids of SEQ ID NO: 3.

(SEQ ID NO: 3)
LWFEQIEAAGTDFDTKTPMDELVLRMLSDNVITLSVDRKAASQTE
TDDVKPQKGKIIPFPVPDIANDEVEYQKAVGMKKD

The activity of the peptidase of the programmable nuclease-peptidase composition may cause a modification to the target polypeptide. In one example embodiment, the modification is cleavage of the target polypeptide between two amino acid residues at one or more locations in the target polypeptide. In one example embodiment, the peptidase recognition motif is at the C-terminus, N-terminus, or both the C- and N-terminus of the target polypeptide. In one example embodiment, the peptidase recognition motif is contained between the C- and N-terminus of the target peptide. In one example embodiment, the target polypeptide has peptidase recognition motifs at the C-terminus, N-terminus, both the C- and N-terminus, between the C- and N-terminus, or any combination thereof. The target polypeptide may contain 1, 2, 3, 4, 5, 6, 7, 8, 9, 10, 11, 12, 13, 14, 15, 16, 17, 18, 19, 20, or more peptidase recognition motifs.

In one example embodiment, the peptidase recognition motif(s) is/are native to a target polypeptide. The target polypeptide may also be engineered to contain one or more peptidase recognition motifs that are not native to the target polypeptide. In one example embodiment, a target polypeptide is engineered to contain one or more peptidase recognition motifs, such as any of those described elsewhere herein, fused to the C-terminus and/or N-terminus and/or between any two amino acids between the C-terminus and N-terminus of the target polypeptide. In one example embodiment, the target polypeptide is engineered to contain one or more peptidase recognition motifs linked, via one or more amino acid linkers, to the C-terminus and/or N-terminus and/or between any two amino acids between the C-terminus and N-terminus of the target polypeptide. In some embodiments, the target polypeptide is engineered to contain one or more peptidase recognition motifs linked, via one or more chemical linkers to one or more residues of the target polypeptide.

In some embodiments, activity of the peptidase of the programmable nuclease-peptidase composition causes the target polypeptide to be reversibly or irreversibly bound by the programable nuclease-peptidase composition. In some embodiments, this binding can result in a conformational change and/or block or expose an active site in the target polypeptide, which, without being bound by theory, can modify an activity of the target polypeptide. In some embodiments, this binding results in inhibition of the target polypeptide. In some embodiments, this binding results in activation of the target polypeptide.

### Example Target Polypeptides

In certain example embodiments, the target polypeptide is a reporter polypeptide; a signal amplification polypeptide; an engineered prodrug; a cleavable linker; a cargo polypeptide, pathogenic polypeptide.

As used herein, “pathogenic polypeptide” refers to a polypeptide derived from or produced by a pathogenic microorganism or virus (collectively referred to as pathogens). Exemplary pathogens are described elsewhere herein. In some embodiments, the pathogenic polypeptide can be an effector such that it can cause cell death, such as via apoptosis or lysis, upon activation and/or delivery to a cell by the programmable nuclease-protease system of the present invention.

In some embodiments, the target polypeptide is an intracellular polypeptide. In some embodiments, the target polypeptide is a disease-associated polypeptide.

As used herein “disease-associated” polypeptide is a polypeptide that is involved in the etiology of a disease, infection, or a condition or is otherwise involved in manifesting a primary or secondary symptom of a disease or condition. In some embodiments, a reduction in the amount of and/or activity of the disease-associated polypeptide treats and/or prevents a disease, condition or a symptom thereof. In some embodiments, the disease or condition that is caused by or associated with the disease associated polypeptide is a cancer, a metabolic disease or condition, an autoimmune disease, a cardiovascular disease, a neurodegenerative disease or condition, a motor neuron disease or condition, graft-versus-host disease, transplant rejection, ischemic injury (e.g., stroke, heart attack, etc.), or spinal cord injury. Exemplary neurodegenerative diseases or conditions are, without limitation, Parkinson's disease, Huntington's disease, Alzheimer's disease, amyotrophic lateral sclerosis, an epilepsy, a dementia, Bell's palsy, or Progressive supranuclear palsy. Exemplary metabolic diseases or conditions are, without limitation, diabetes mellitus (type 1 and type 2), metabolic syndrome, obesity, hyperthyroidism, hypothyroidism, and Cushing's disease or syndrome.

In some embodiments, the disease or condition is characterized by increased or undesired activity of a target protein relative to a normal or healthy state. In some embodiments, increased activity of a target disease-associated protein occurs when, in a cell, the amount and/or activity of the target disease-associated protein is about 2-fold, 3-fold, 4-fold, 5-fold, 10-fold, 25-fold, 50-fold, 100-fold, 500-fold, or 1000-fold over the amount and/or activity of the target disease-associated protein in a normal healthy cell, or extracellular environment.

In some embodiments, the target disease-associated polypeptide is a pathogenic polypeptide.

In some embodiments, the target disease-associated polypeptide is an antibody.

In some embodiments, the disease-associated polypeptide comprises a peptidase recognition motif. In some embodiments, the disease associated polypeptide comprises MDDK.

In some embodiments, the target polypeptide is any one in Table 1. In some embodiments, the disease associated polypeptide is any one in Table 1.

TABLE 1
Exemplary human proteins with MDDK (SEQ ID NO: 25) motif

| | Scientific | |
|---|---|---|
| Description | Name | Accession |
| E3 ubiquitin-protein ligase MYCBP2 [Homo sapiens] | Homo | NP_055872.4 |
| | sapiens | |
| protein associated with Myc [Homo sapiens] | Homo | AAC39928.1 |
| | sapiens | |
| MYC binding protein 2, isoform CRA_b [Homo sapiens] | Homo | EAW80565.1 |
| | sapiens | |
| MYC binding protein 2, isoform CRA_c [Homo sapiens] | Homo | EAW80566.1 |
| | sapiens | |
| KICSTOR complex protein SZT2 isoform 1 [Homo sapiens] | Homo | NP_001352928.1 |
| | sapiens | |
| KICSTOR complex protein SZT2 isoform 2 [Homo sapiens] | Homo | NP_056099.3 |
| | sapiens | |
| hCG2036582, isoform CRA_c [Homo sapiens] | Homo | EAX07097.1 |
| | sapiens | |
| hCG2036582, isoform CRA_b [Homo sapiens] | Homo | EAX07096.1 |
| | sapiens | |
| MYC binding protein 2, isoform CRA_a [Homo sapiens] | Homo | EAW80564.1 |
| | sapiens | |
| MYC binding protein 2, isoform CRA_d [Homo sapiens] | Homo | EAW80567.1 |
| | sapiens | |
| fatty acid synthase [Homo sapiens] | Homo | AAS09886.1 |
| | sapiens | |
| fatty acid synthase [Homo sapiens] | Homo | EAW89745.1 |
| | sapiens | |
| Fatty acid synthase [Homo sapiens] | Homo | AAH63242.1 |
| | sapiens | |
| fatty acid synthase [Homo sapiens] | Homo | KAI2585807.1 |
| | sapiens | |
| fatty acid synthase [Homo sapiens] | Homo | NP_004095.4 |
| | sapiens | |
| fatty acid synthase [Homo sapiens] | Homo | AAA73576.1 |
| | sapiens | |
| fatty acid synthase [Homo sapiens] | Homo | KAI4052325.1 |
| | sapiens | |
| fatty acid synthase [Homo sapiens] | Homo | KAI2585806.1 |
| | sapiens | |
| encodes region of fatty acid synthase activity; FAS; | Homo | AAC50259.1 |
| multifunctional protein [Homo sapiens] | sapiens | |
| unconventional myosin-XVIIIa isoform c [Homo sapiens] | Homo | NP_001333694.1 |
| | sapiens | |
| hCG27198, isoform CRA_i [Homo sapiens] | Homo | EAW51184.1 |
| | sapiens | |
| unconventional myosin-XVIIIa isoform a [Homo sapiens] | Homo | NP_510880.2 |
| | sapiens | |
| myosin XVIIIA [Homo sapiens] | Homo | KAI2582130.1 |
| | sapiens | |
| unconventional myosin-XVIIIa isoform d [Homo sapiens] | Homo | NP_001333695.1 |
| | sapiens | |
| unconventional myosin-XVIIIa isoform b [Homo sapiens] | Homo | NP_976063.1 |
| | sapiens | |
| hypothetical protein [Homo sapiens] | Homo | CAI45931.1 |
| | sapiens | |
| myosin XVIIIA [Homo sapiens] | Homo | KAI2582129.1 |
| | sapiens | |
| LOW QUALITY PROTEIN: myosin XVIIIA [Homo sapiens] | Homo | KAI2582127.1 |
| | sapiens | |
| unconventional myosin-XVIIIa isoform e [Homo sapiens] | Homo | NP_001333696.1 |
| | sapiens | |
| myosin XVIIIA [Homo sapiens] | Homo | KAI2582128.1 |
| | sapiens | |
| TAF1 RNA polymerase II, TATA box binding protein (TBP)- | Homo | EAX05293.1 |
| associated factor, 250 kDa, isoform CRA_c [Homo sapiens] | sapiens | |
| transcription initiation factor TFIID subunit 1 isoform X1 | Homo | XP_047298347.1 |
| [Homo sapiens] | sapiens | |
| transcription initiation factor TFIID subunit 1 isoform X2 | Homo | XP_047298348.1 |
| [Homo sapiens] | sapiens | |
| TAF1 RNA polymerase II, TATA box binding protein (TBP)- | Homo | BAG15901.1 |
| associated factor, neuron specific isoform [Homo sapiens] | sapiens | |
| TAF1 RNA polymerase II, TATA box binding protein (TBP)- | Homo | AAT38105.1 |
| associated factor, 250 kDa [Homo sapiens] | sapiens | |
| Structure of human TFIID-IIA bound to core promoter DNA | Homo | 5FUR_G |
| [Homo sapiens] | sapiens | |
| transcription initiation factor TFIID subunit 1 isoform X3 | Homo | XP_047298349.1 |
| [Homo sapiens] | sapiens | |
| transcription initiation factor TFIID subunit 1 isoform X4 | Homo | XP_047298350.1 |
| [Homo sapiens] | sapiens | |
| transcription initiation factor TFIID subunit 1 isoform 3 | Homo | NP_001273003.2 |
| [Homo sapiens] | sapiens | |
| transcription initiation factor TFIID subunit 1 isoform 1 | Homo | NP_004597.3 |
| [Homo sapiens] | sapiens | |
| transcription initiation factor TFIID subunit 1 isoform X6 | Homo | XP_047298352.1 |
| [Homo sapiens] | sapiens | |
| RecName: Full = Transcription initiation factor TFIID subunit 1; | Homo | P21675.2 |
| AltName: Full = Cell cycle gene 1 protein; AltName: Full = TBP- | sapiens | |
| associated factor 250 kDa; Short = p250; AltName: | | |
| Full = Transcription initiation factor TFIID 250 kDa subunit; | | |
| Short = TAF(II)250; Short = TAFII-250; Short = TAFII250 | | |
| [Homo sapiens] | | |
| transcription initiation factor TFIID subunit 1 isoform X7 | Homo | XP_047298353.1 |
| [Homo sapiens] | sapiens | |
| transcription initiation factor TFIID subunit 1 isoform X8 | Homo | XP_047298354.1 |
| [Homo sapiens] | sapiens | |
| TAF1 RNA polymerase II, TATA box binding protein (TBP)- | Homo | EAX05291.1 |
| associated factor, 250 kDa, isoform CRA_a [Homo sapiens] | sapiens | |
| missense breast and ovarian cancer susceptibility protein 1 | Homo | UGU37869.1 |
| [Homo sapiens] | sapiens | |
| TAF1 RNA polymerase II, TATA box binding protein (TBP)- | Homo | EAX05295.1 |
| associated factor, 250 kDa, isoform CRA_e [Homo sapiens] | sapiens | |
| transcription initiation factor TFIID subunit 1 isoform 2 [Homo | Homo | NP_620278.2 |
| sapiens] | sapiens | |
| transcription initiation factor TFIID subunit 1 isoform X10 [Homo | Homo | XP_047298356.1 |
| sapiens] | sapiens | |
| transcription initiation factor TFIID subunit 1 isoform X11 [Homo | Homo | XP_047298357.1 |
| sapiens] | sapiens | |
| transcription initiation factor TFIID subunit 1 isoform X12 [Homo | Homo | XP_047298358.1 |
| sapiens] | sapiens | |
| transcription initiation factor TFIID subunit 1-like [Homo sapiens] | Homo | NP_722516.1 |
| | sapiens | |
| transcription initiation factor TFIID subunit 1 isoform X13 [Homo | Homo | XP_047298359.1 |
| sapiens] | sapiens | |
| transcription initiation factor TFIID subunit 1 isoform X15 [Homo | Homo | XP_047298361.1 |
| sapiens] | sapiens | |
| transcription initiation factor TFIID subunit 1 isoform X14 [Homo | Homo | XP_047298360.1 |
| sapiens] | sapiens | |
| transcription initiation factor TFIID subunit 1 isoform X16 [Homo | Homo | XP_024308198.2 |
| sapiens] | sapiens | |
| Rho guanine nucleotide exchange factor (GEF) 12, isoform CRA_a | Homo | EAW67508.1 |
| [Homo sapiens] | sapiens | |
| CCG1 protein [Homo sapiens] | Homo | CAA30073.1 |
| | sapiens | |
| ARHGEF12 [Homo sapiens] | Homo | SPT35723.1 |
| | sapiens | |
| rho guanine nucleotide exchange factor 12 isoform 1 [Homo sapiens] | Homo | NP_056128.1 |
| | sapiens | |
| rho guanine nucleotide exchange factor 12 isoform 2 [Homo sapiens] | Homo | NP_001185594.1 |
| | sapiens | |
| rho guanine nucleotide exchange factor 12 isoform X1 [Homo | Homo | XP_006718868.1 |
| sapiens] | sapiens | |
| rho guanine nucleotide exchange factor 12 isoform X2 [Homo | Homo | XP_047282615.1 |
| sapiens] | sapiens | |
| rho guanine nucleotide exchange factor 12 isoform X3 [Homo | Homo | XP_047282616.1 |
| sapiens] | sapiens | |
| coiled-coil domain-containing protein 18 isoform X1 [Homo sapiens] | Homo | XP_054192287.1 |
| | sapiens | |
| coiled-coil domain-containing protein 18 isoform X1 [Homo sapiens] | Homo | XP_016856643.2 |
| | sapiens | |
| coiled-coil domain-containing protein 18 isoform X2 [Homo sapiens] | Homo | XP_054192288.1 |
| | sapiens | |
| coiled-coil domain-containing protein 18 isoform X2 [Homo sapiens] | Homo | XP_016856644.2 |
| | sapiens | |
| rho guanine nucleotide exchange factor 12 isoform X4 [Homo | Homo | XP_016872909.1 |
| sapiens] | sapiens | |
| coiled-coil domain-containing protein 18 isoform X3 [Homo sapiens] | Homo | XP_016856646.2 |
| | sapiens | |
| coiled-coil domain-containing protein 18 isoform X3 [Homo sapiens] | Homo | XP_054192291.1 |
| | sapiens | |
| coiled-coil domain-containing protein 18 isoform X4 [Homo sapiens] | Homo | XP_047275405.1 |
| | sapiens | |
| coiled-coil domain-containing protein 18 isoform X4 [Homo sapiens] | Homo | XP_054192292.1 |
| | sapiens | |
| rho guanine nucleotide exchange factor 12 isoform X5 [Homo | Homo | XP_047282617.1 |
| sapiens] | sapiens | |
| transcription initiation factor TFIID subunit 1 isoform X17 [Homo | Homo | XP_047298362.1 |
| sapiens] | sapiens | |
| rho guanine nucleotide exchange factor 12 isoform X7 [Homo | Homo | XP_047282618.1 |
| sapiens] | sapiens | |
| coiled-coil domain-containing protein 18 isoform c [Homo sapiens] | Homo | NP_001365133.1 |
| | sapiens | |
| coiled-coil domain-containing protein 18 isoform X5 [Homo sapiens] | Homo | XP_054192295.1 |
| | sapiens | |
| coiled-coil domain-containing protein 18 isoform a [Homo sapiens] | Homo | NP_001293005.1 |
| | sapiens | |
| coiled-coil domain containing 18 [Homo sapiens] | Homo | KAI4081486.1 |
| | sapiens | |
| coiled-coil domain-containing protein 18 isoform X10 [Homo | Homo | XP_054192300.1 |
| sapiens] | sapiens | |
| coiled-coil domain-containing protein 18 isoform X6 [Homo sapiens] | Homo | XP_016856648.2 |
| | sapiens | |
| coiled-coil domain-containing protein 18 isoform X6 [Homo sapiens] | Homo | XP_054192296.1 |
| | sapiens | |
| coiled-coil domain-containing protein 18 isoform X7 [Homo sapiens] | Homo | XP_047275432.1 |
| | sapiens | |
| coiled-coil domain-containing protein 18 isoform X7 [Homo sapiens] | Homo | XP_054192297.1 |
| | sapiens | |
| coiled-coil domain-containing protein 18 isoform X8 [Homo sapiens] | Homo | XP_054192298.1 |
| | sapiens | |
| coiled-coil domain-containing protein 18 isoform X8 [Homo sapiens] | Homo | XP_016856649.2 |
| | sapiens | |
| coiled-coil domain-containing protein 18 isoform X9 [Homo sapiens] | Homo | XP_054192299.1 |
| | sapiens | |
| coiled-coil domain-containing protein 18 isoform X9 [Homo sapiens] | Homo | XP_047275438.1 |
| | sapiens | |
| rho guanine nucleotide exchange factor 12 isoform 3 [Homo sapiens] | Homo | NP_001288013.1 |
| | sapiens | |
| ATP-binding cassette, sub-family C (CFTR/MRP), member 5, | Homo | EAW78310.1 |
| isoform CRA_e [Homo sapiens] | sapiens | |
| coiled-coil domain-containing protein 18 isoform X11 [Homo | Homo | XP_054192303.1 |
| sapiens] | sapiens | |
| coiled-coil domain-containing protein 18 isoform X11 [Homo | Homo | XP_047275448.1 |
| sapiens] | sapiens | |
| coiled-coil domain-containing protein 18 isoform X12 [Homo | Homo | XP_054192304.1 |
| sapiens] | sapiens | |
| ABC transporter MOAT-C [Homo sapiens] | Homo | AAD04169.1 |
| | sapiens | |
| coiled-coil domain-containing protein 18 isoform X12 [Homo | Homo | XP_047275449.1 |
| sapiens] | sapiens | |
| ATP-binding cassette sub-family C member 5 isoform 1 [Homo | Homo | NP_005679.2 |
| sapiens] | sapiens | |
| ABC protein [Homo sapiens] | Homo | AAD37716.1 |
| | sapiens | |
| MRP5 [Homo sapiens] | Homo | BAA76608.1 |
| | sapiens | |
| Multidrug resistance-associated protein 5 variant [Homo sapiens] | Homo | BAD92691.1 |
| | sapiens | |

In some embodiments, the target polypeptide, such as a disease associated polypeptide, is cleaved by the programmable nuclease-peptidase. In some embodiments, activity of the programmable nuclease-peptidase is controlled via temporal and/or spatial control of the gRNA or one or more other components of the programmable nuclease-peptidase of the present invention.

In some embodiments, the target polypeptide is a SNAP Receptor (SNARE) protein. In some embodiments, the target polypeptide is a vehicle associated membrane protein (VAMP). In some embodiments, the VAMP proteins is VAMP1, VAMP2, VAMP3, VAMP4, VAMP5, VAMP7, and/or VAMP8. In some embodiments, the target polypeptide is SNAP23 and/or SNAP25. In some embodiments, the target polypeptide is phosphatase and tensin homolog (PTEN).

In one example embodiment, the target polypeptide is a reporter polypeptide. Generally, reporter polypeptides are polypeptides that can be readily identified, such as by an optical signal they produce, reaction they catalyze, epitopes, activity they have, and/or a phenotype thy confer. Reporter polypeptides include, but are not limited to, optically active polypeptides, enzymes, and others. Without being bound by theory, inclusion of a protease recognition motif in a reporter polypeptide can provide a signal when acted upon by the programmable nuclease-peptidase system described herein. The reporter can be configured to produce a positive signal upon interaction with (such as cleavage by) a programmable nuclease-peptidase system described herein. In some embodiments, the reporter can be configured to produce a positive signal absent interaction with a programmable nuclease-peptidase system described herein and produce a loss of signal upon interaction with (such as cleavage by) the programmable nuclease-peptidase system described herein Exemplary reporter polypeptides include, without limitation, GUS; fluorescent proteins such as green fluorescent protein (GFP), cyan fluorescent protein (CFP), yellow fluorescent protein (YFP), red (RFP) fluorescent protein, HcRed, DsRed, and auto-fluorescent proteins including blue fluorescent protein (BFP), luciferase, cell surface proteins, polypeptides that provide resistance to antibiotics, such as, spectinomycin, ampicillin, kanamycin, tetracycline, Basta, neomycin phosphotransferase II (NEO), hygromycin phosphotransferase (HPT)) and/or the like, auxotrophic markers, epitope tags (FLAG-tag, tag, Myc-tag, influenza hemagglutinin (HA)-tag and NE-tag, and/or the like), glutathione-S-transferase (GST), horseradish peroxidase (HRP), chloramphenicol acetyltransferase (CAT) beta-galactosidase, beta-glucuronidase, polypeptides having methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, or nucleic acid binding activity, and/or any combination thereof.

In one example embodiment, the reporter polypeptide is configured as a FLIP reporter (see e.g., Zhang et al. 2019. JACS. 2019. March 20; 141(11):4526-4530. doi: 10.1021/jacs.8b13042).

Generally, signal amplification polypeptides are target polypeptides that can be included in, e.g., a detection reaction, that can amplify the signal generated during a detection reaction. The signal amplification polypeptide can be secondary to a first target polypeptide that is part of a detection construct. Signal amplification polypeptides can be spiked within a detection reaction. In some embodiments, the signal amplification polypeptides result directly in generation of the detectable signal of the detection reaction, thus boosting signal generation in response to activation of the detection composition described herein. In some embodiments, signal amplification polypeptides are configured to, when acted upon by an activated detection composition of the present invention, activate a CRISPR-Cas based detection system to result in signal amplification. Further details of signal amplification polypeptides are provided elsewhere herein.

In some embodiments, the target polypeptide is an engineered prodrug or a component of an engineered prodrug. Generally, prodrugs are agents that are provided in first, typically inactive form or prodrug, that are modified in one or more ways, to from a second, typically active, form. For example, a polypeptide prodrug can be provided as a polypeptide that is inactive or less active until cleaved to release the active peptide and/or polypeptide component(s) of the longer polypeptide prodrug. In some embodiments, one or more components of the prodrug facilitate uptake into the body (e.g., across the brushborder membrane of the small or large intestine, the blood brain barrier, and/or the like) or into a target cell via interaction with a cell surface receptor that are not directly related to the therapeutic action but increase bioavailability of the active component. Once inside the body these portions can be cleaved to release the therapeutically active portion of the prodrug. In some embodiments, a peptide or polypeptide can be coupled to a chemical or small molecule active agent, such as via an amid bond, to form a prodrug. In some embodiments, the engineered prodrug comprises or consists of a target polypeptide. Without being bound by theory, a prodrug having a target polypeptide can be modulated from an inactive form to an active form by being exposed to a programmable nuclease-peptidase system described herein. For example, cleavage of the target polypeptide can release an active portion (e.g., polypeptide, peptide, or small molecule agent) of an engineered prodrug. Spatial and/or temporal release of an active component of a prodrug can be tuned and/or further controlled by controlling activation of the protease of the programmable nuclease-peptidase system, such as by controlling where and/or when the guide molecule complexes with a programmable nuclease of the system so as to activate the system in the presence of a target polynucleotide.

In some embodiments, the target polypeptide is a cleavable linker. Generally cleavable linkers are agents that can connect or link two or more components, such as two or more peptides, polypeptides, small molecules, and/or the like, or any combination thereof together. Without being bound by theory, when an activated programmable nuclease-peptidase system interacts with the cleavable linker it can cleave the cleavable linker. In some embodiments, cleavable linker contains only the protease recognition motif. The cleavable linker can be a flexible linker. The cleavable linker can be a rigid linker. Spatial and/or temporal cleavage of a cleavable linker can be tuned and/or further controlled by controlling activation of the protease of the programmable nuclease-peptidase system, such as by controlling where and/or when the guide molecule complexes with a programmable nuclease of the system so as to activate the system in the presence of a target polynucleotide. In some embodiments, a linker that can include a protease domain is a gly-ser linker containing one or more protease recognition domains, such as any of these described in greater detail elsewhere herein.

In one example embodiments, the target polypeptide is a cargo polypeptide. In one example embodiment, the cargo polypeptide or portion thereof is released, such as from a delivery vector, particle, vesicle, molecule and/or the like that the cargo polypeptide is associated when an activated programmable nuclease-peptidase system described herein interacts with (such as cleaves) the cargo polypeptide. In some embodiments, a cargo polypeptide is activated (or deactivated) when an activated programmable nuclease-peptidase system described herein interacts with (such as cleaves) the cargo polypeptide.

### Example Cargo Polypeptides

### Polynucleotide Modifying System Polypeptides

In some embodiments, the cargo is a polynucleotide modifying system polypeptide thereof. In some embodiments the polynucleotide modifying system is a gene modifying system polypeptide.

In some embodiments, the gene editing system is an RNA-guided nuclease or other programmable nuclease. In some embodiments, the gene editing system polypeptide is a CRISPR-Cas system polypeptide, such as a Cas polypeptide.

In some embodiments, the gene modification system polynucleotide is a zinc finger nuclease. In some embodiments, the gene modification system polynucleotide is a meganuclease.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In general, were a Cas-based system (including specialized Cas-based systems) polypeptide is a cargo polypeptide, it will be appreciated that such a peptide can be complexed with a guide polynucleotide or other polynucleotide component where relevant such as a donor template.

### Class 1 Systems

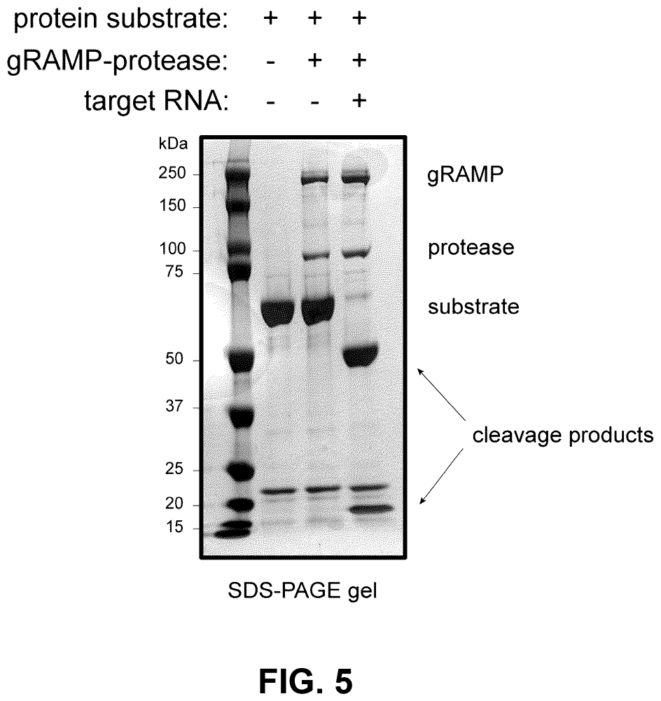

In some embodiments, the CRISPR-Cas system polypeptide is a Class 1 CRISPR polypeptide. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374:20180087, DOI: 10.1098/rstb.2018.0087. In one aspect, Class 1 systems are characterized by the signature protein Cas3. The cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one embodiment, the Type I CRISPR polypeptide comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I—F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al, the CRISPR Journal, v. 1, n5, FIG. 5.

### Class 2 Systems

In some embodiments, the CRISPR-Cas polypeptide is Class 2 CRISPR-Cas system polypeptide. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system polypeptide is a Type II system polypeptide. In some embodiments, the Type II CRISPR-Cas system polypeptide is a II-A CRISPR-Cas system polypeptide. In some embodiments, the Type II CRISPR-Cas system polypeptide is a II-B CRISPR-Cas system polypeptide. In some embodiments, the Type II CRISPR-Cas system polypeptide is a II-C1 CRISPR-Cas system polypeptide. In some embodiments, the Type II CRISPR-Cas system polypeptide is a II-C2 CRISPR-Cas system polypeptide. In some embodiments, the Type II system polypeptide is a Cas9 system. In some embodiments, the Type II system polypeptide includes a Cas9.

In some embodiments, the Class 2 system polypeptide is a Type V system polypeptide. In some embodiments, the Type V CRISPR-Cas system polypeptide is a V-A CRISPR-Cas system polypeptide. In some embodiments, the Type V CRISPR-Cas system polypeptide is a V-B1 CRISPR-Cas system polypeptide. In some embodiments, the Type V CRISPR-Cas system polypeptide is a V-B2 CRISPR-Cas system polypeptide. In some embodiments, the Type V CRISPR-Cas system polypeptide is a V-C CRISPR-Cas system polypeptide. In some embodiments, the Type V CRISPR-Cas system polypeptide is a V-D CRISPR-Cas system polypeptide. In some embodiments, the Type V CRISPR-Cas system polypeptide is a V-E CRISPR-Cas system polypeptide. In some embodiments, the Type V CRISPR-Cas system polypeptide is a V-F1 CRISPR-Cas system polypeptide. In some embodiments, the Type V CRISPR-Cas system polypeptide is a V-F1 (V-U3) CRISPR-Cas system polypeptide. In some embodiments, the Type V CRISPR-Cas system polypeptide is a V-F2 CRISPR-Cas system polypeptide. In some embodiments, the Type V CRISPR-Cas system polypeptide is a V-F3 CRISPR-Cas system polypeptide. In some embodiments, the Type V CRISPR-Cas system polypeptide is a V-G CRISPR-Cas system polypeptide. In some embodiments, the Type V CRISPR-Cas system polypeptide is a V-H CRISPR-Cas system polypeptide. In some embodiments, the Type V CRISPR-Cas system polypeptide is a V-I CRISPR-Cas system polypeptide. In some embodiments, the Type V CRISPR-Cas system polypeptide is a V-K (V-U5) CRISPR-Cas system polypeptide. In some embodiments, the Type V CRISPR-Cas system polypeptide is a V-U1 CRISPR-Cas system polypeptide. In some embodiments, the Type V CRISPR-Cas system polypeptide is a V-U2 CRISPR-Cas system polypeptide. In some embodiments, the Type V CRISPR-Cas system polypeptide is a V-U4 CRISPR-Cas system polypeptide. In some embodiments, the Type V CRISPR-Cas system polypeptide includes a Cas12a (Cpf1), Cas12b (C2c1), Cas12c (C2c3), Cas12d (CasY), Cas12e (CasX), Cas14, and/or CasΦ.

In some embodiments the Class 2 system polypeptide is a Type VI system. In some embodiments, the Type VI CRISPR-Cas system polypeptide is a VI-A CRISPR-Cas system polypeptide. In some embodiments, the Type VI CRISPR-Cas system polypeptide is a VI-B1 CRISPR-Cas system polypeptide. In some embodiments, the Type VI CRISPR-Cas system polypeptide is a VI-B2 CRISPR-Cas system polypeptide. In some embodiments, the Type VI CRISPR-Cas system polypeptide is a VI-C CRISPR-Cas system polypeptide. In some embodiments, the Type VI CRISPR-Cas system polypeptide is a VI-D CRISPR-Cas system polypeptide. In some embodiments, the Type VI CRISPR-Cas system polypeptide includes a Cas13a (C2c2), Cas13b (Group 29/30), Cas13c, and/or Cas13d.

### Specialized Cas-System Polypeptides

In some embodiments, the system is a Cas-based system polypeptide that is capable of performing a specialized function or activity or lacks one or more activities as compared to a wild-type polypeptide. In some embodiments, the Cas-system polypeptide is a catalytically deadCas (dCas) polypeptide, which has nickase activity. In some embodiments, a dCas contains one or more additional functional domains such as a nuclear localization signal (NLS) domain, a nuclear export signal (NES) domain, a translational activation domain, a transcriptional activation domain (e.g., VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. In some embodiments, the one or more functional domains have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. The one or more functional domain(s) may be positioned at, near, and/or in proximity to a terminus of the dCas. When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other. Other suitable functional domains can be found, for example, in International Patent Publication No. WO 2019/018423. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

### Split CRISPR-Cas System Polypeptides

In some embodiments, the CRISPR-Cas system polypeptide is a split CRISPR-Cas system polypeptide. See e.g., Zetche et al., 2015. Nat. Biotechnol. 33(2):139-142 and International Patent Publication WO 2019/018423, which are incorporated by reference herein. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing System Polypeptides

In some embodiments, the cargo polypeptide is a DNA or RNA base editing system polypeptide. DNA or RNA base editing system polypeptides include a Cas, such as a dCas polypeptide connected or fused to a nucleotide deaminase. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be connected or fused to a DNA binding Cas protein such as, but not limited to, Class 2 Type II and Type V systems polypeptides, which are described in greater detail elsewhere herein. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, cargo polypeptide is a CBE or an ABE.

In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems polypeptides are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system polypeptide. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. Example Type VI RNA-base editing system polynucleotides are described in Cox et al. 2017. Science 358:1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system polypeptide that may be adapted for RNA base editing purposes is described in International Patent Publication No. WO 2016/106236, which is incorporated herein by reference.

### Prime Editor Polypeptides

In some embodiments, the cargo polypeptide is a prime editor system polypeptide. Prime Editor systems and polypeptides are generally described in e.g., Anzalone et al. 2019. Nature. 576:149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, a prime editing system can be composed of a Cas polypeptide having nickase activity, a reverse transcriptase, and a guide molecule. In some embodiments, the cargo polypeptide(s) can be a Cas polypeptide having nickase activity and a reverse transcriptase. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

### CRISPR Associated Transposase (CAST) Systems

In some embodiments, the cargo polypeptide is a CAST system polypeptide. CAST system polypeptides include Cas proteins that are catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi: 10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### TALE Nucleases (TALENs)

In some embodiments, the cargo polypeptide is a TALEN system polypeptide. In some embodiments, the TALEN system polypeptide is a TALEN. In some embodiments, the TALEN comprises a TALE monomer or half monomers as a part of their organizational structure that enable the targeting of nucleic acid sequences with improved efficiency and expanded specificity. Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. As used herein, the term “polypeptide monomers”, “TALE monomers” or “monomers” is used to refer to the highly conserved repetitive polypeptide sequences within the TALE nucleic acid binding domain and the term “repeat variable di-residues” or “RVD” is used to refer to the highly variable amino acids at positions 12 and 13 of the polypeptide monomers. The amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 Of 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅) z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

In some embodiments, the TALEN polypeptides are isolated, non-naturally occurring, recombinant or engineered nucleic acid-binding proteins that have nucleic acid or DNA binding regions containing polypeptide monomer repeats that are designed to target specific nucleic acid sequences.

As described herein, TALE polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 38)
M D P I R S R T P S P A R E L L S G P Q P D
G V Q P T A D R G V S P P A G G P L D G L P
A R R T M S R T R L P S P P A P S P A F S A
D S F S D L L R Q F D P S L F N T S L F D S
L P P F G A H H T E A A T G E W D E V Q S G
L R A A D A P P P T M R V A V T A A R P P R
A K P A P R R R A A Q P S D A S P A A Q V D
L R T L G Y S Q Q Q Q E K I K P K V R S T V
A Q H H E A L V G H G F T H A H I V A L S Q
H P A A L G T V A V K Y Q D M I A A L P E A
T H E A I V G V G K Q W S G A R A L E A L L
T V A G E L R G P P L Q L D T G Q L L K I A
K R G G V T A V E A V H A W R N A L T G A P
L N

An exemplary amino acid sequence of a C-terminal capping region is:

(SEQ ID NO: 39)
R P A L E S I V A Q L S R P D P A L A A L T
N D H L V A L A C L G G R P A L D A V K K G
L P H A P A L I K R T N R R I P E R T S H R
V A D H A Q V V R V L G F F Q C H S H P A Q
A F D D A M T Q F G M S R H G L L Q L F R R
V G V T E L E A R S G T L P P A S Q R W D R
I L Q A S G M K R A K P S P T S T Q T P D Q
A S L H A F A D S L E R D L D A P S P M H E
G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs.

These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nuclease System Polypeptides

In some embodiments, the cargo polypeptide is a zinc finger nuclease. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, the cargo polypeptide is a meganuclease. Meganucleases, are endodeoxyribonucleases characterized by a large recognition site (double-stranded DNA sequences of 12 to 40 base pairs). Exemplary meganucleases methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Transposon System Polypeptides

In some embodiments, the cargo polypeptide is a transposon system polypeptide. In some embodiments, the cargo polypeptide is a Class I transposon system polypeptide. In some embodiments, the cargo polypeptide is a Class II transposon system polypeptide. As used herein, “transposon” (also referred to as transposable element) refers to a polynucleotide sequence that is capable of moving form location in a genome to another. There are several classes of transposons. Transposons include retrotransposons (Class I transposons) and DNA transposons (Class II transposons). Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Suitable Class I transposon system polypeptides any of those in, without limitation, LTR and non-LTR retrotransposon systems. Exemplary systems and system polypeptides include, without limitation, CRE, R2, R4, L1, RTE, Tad, R1, LOA, I, Jockey, CR1 polypeptides. See e.g., Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties.

Suitable Class II transposon system polypeptides include any of those in, without limitation, the following transposon systems: Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4):1531-1536), Tol2 (superfamily hAT), Frog Prince (Tc1/mariner superfamily) (see e.g. Miskey et al. 2003 Nucleic Acid Res. 31(23):6873-6881) and variants thereof.

In some embodiments, the Class II transposon polypeptide is a DD [E/D] transposon or transposon polypeptide. In some embodiments, the Class II transposon polypeptide is a Tc1/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized in the context of the present invention can also be and are not limited to those described in e.g., and without limitation, Han et al., 2013. BMC Genomics. 14:71, doi: 10.1186/1471-2164-14-71, Lopez and Garcia-Perez. 2010. Curr. Genomics. 11(2):115-128; Wessler. 2006. PNAS. 103(47):176000-17601; Gao et al., 2017. Marine Genomics. 34:67-77; Bradic et al. 2014. Mobile DNA. 5(12) doi: 10.1186/1759-8753-5-12; Li et al., 2013. PNAS. 110(25)E2279-E2287; Kebriaei et al. 2017. Trends in Genetics. 33(11):852-870); Miskey et al. 2003. Nucleic Acid res. 31(23):6873-6881; Nicolas et al. 2015. Microbiol Spectr. 3(4) doi: 10.1128/microbiolspec.MDNA3-0060-2014); W. S. Reznikoff. 1993. Annu Rev. Microbiol. 47:945-963; Rubin et al. 2001. Genetics. 158(3):949-957; Wicker et al. 2003. Plant Physiol. 132(1):52-63; Majumdar and Rio. 2015. Microbiol. Spectr. 3(2) doi 10.1128/microbiolspec.MDNA3-0004-2014; D. Lisch. 2002. Trends in Plant Sci. 7(11):498-504; Sinzelle et al. 2007. PNAS. 105(12):4715-4720; Han et al. 2014; Genome Biol. Evol. 6(7):1748-1757; Grzebelus et al. 2006; Mol. Genet. Genomics. 275(5):450-459; Zhang et al. 2004. Genetics. 166(2):971-986; Chen and Li. 2008. Gene. 408(1-2):51-63; and C. Feschotte. 2004. Mol. Biol. Evol. 21(9):1769-1780.

### Antibodies

In some embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments. As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric f-rm, and IgA antibodies exist in monomeric, dimeric or multimeric form.

The term “IgG subclass” refers to the four subclasses of immunoglobulin class IgG-IgG1“IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., “comprising 3 to 4 peptide loops”) stabilized, for example, by β pleated sheet and/or intrachain disulfide bond. Domains are further referred to herein as “constant” or “variable”, based on the relative lack of sequence variation within the domains of various class members in the case of ““constant” domain, or the significant variation within the domains of various class members in the case of a “variable” domain. Antibody or polypeptide “domains” are often referred to interchangeably in the of an antibody or polypeptide “region”. The “constant” domains of an antibody light chain are referred to interchangeably as “light chain constant regions”, “light chain constant domains”, “CL” regions or “CL” domains” The “constant” domains of an antibody heavy chain are referred to interchangeably as “heavy chain constant regions”, “heavy chain constant domains”, “CH” regions or “CH” domains)” The “variable” domains of an antibody light chain are referred to interchangeably as “light chain variable regions”, “light chain variable domains”, “VL” regions or “VL” domains)” The “variable” domains of an antibody heavy” chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10^{th} extracellular domain of human fibronectin III (10Fn3), which adopts an Ig-like beta-sandwich fold (94 residues) with 2-3 exposed loops but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin “peptides (Kolmar” Alternative binding proteins: biological activity and therapeutic potential of cystine-knot miniproteins. FEBS J 2008, 275:2684-2690).

“Specific binding” of an antibody” means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of luM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, “1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the “inetic BIACORE™ met” od. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal “antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding′ fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which ′ is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii)′isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423(1988); and Huston et al., 85 PNAS 5879(1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain “polypeptides, form a pair of antigen” binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/4028 1; U.S. Pat. No. 5,811,097; Deng et al., Blood 92(6):1981-1988 (19 98); Chen et al., Cancer Res. 58(16):3668-3678 (19 98); Harrop et al., J. Immunol. 161(4):1786-1794 (199 8); Zhu et al., Cancer Res. 58(15):3209-321 4 (1998); Yoon et al., J. Immunol. 160(7):3170-3179(1998); Pr at et al., J. Cell. Sci. III (Pt2):237-2 47 (1998); Pitard et al., J. Immunol. Methods 205 (2):177-190 (19 97); Liautard et al., Cytokine 9(4):233-241 (199 7); Carlson et al., J. Biol. Che m. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Secretory Proteins

In certain example embodiments, the one or more cargo polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In embodiments, the protein is secreted by the secretory pathway. In embodiments, the proteins are exocrine secretion proteins or peptides, comprising enzymes in the digestive tract. In embodiments, the protein is endocrine secretion protein or peptide, for example, insulin and other hormones released into the blood stream. In other embodiments, the protein is involved in signaling between or within cells via secreted signaling molecules, for example, paracrine, autocrine, endocrine or neuroendocrine. In embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g., deoxyribonucleases and ribonucleases), peptidases enzyme inhibitors such as serpins (e.g., α1-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a preferred embodiment, the one or more polynucleotides is inserted in the middle of the c-peptide. In some embodiments, the secretory protein is GLP-1, glucagon, betatrophin, pancreatic amylase, pancreatic lipase, carboxypeptidase, secretin, CCK, a PPAR (e.g., PPAR-alpha, PPAR-gamma, PPAR-delta or a precursor thereof (e.g., preprotein or preproprotein). In aspects, the secretory protein is fibronectin, a clotting factor protein (e.g., Factor VII, VIII, IX, etc.), α2-macroglobulin, α1-antitrypsin, antithrombin III, protein S, protein C, plasminogen, α2-antiplasmin, complement components (e.g., complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cell” in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4⁺, CD8⁺, effector Th, memory Th, regulatory Th, CD4⁺/CD8⁺ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific anly response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th 17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins are immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-β, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more cargo polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR) or may include one or more PPARs. In some embodiments, the protein is PPARα, PPAR γ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and/or Transplant Survival Functions

In certain example embodiments, the one or more cargo polypeptides may comprise a proteins that proteins that promote tissue regeneration and/or transplant survival functions. In some cases, such proteins may induce and/or up-regulate the expression of genes for pancreatic β cell regeneration. In some cases, the proteins that promote transplant survival and functions include the products of genes for pancreatic β cell regeneration. Such genes may include proislet peptides that are proteins or peptides derived from such proteins that stimulate islet cell neogenesis. Examples of genes for pancreatic β cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfal, Mafa, Pax6, Pax4, Nkx6.1, Nkx2.2, PDGF, vglycin, placental lactogens (somatomammotropins, e.g. CSH1, CHS2), isoforms thereof, homologs thereof, and orthologs thereof. In certain embodiments, the protein promoting pancreatic B cell regeneration is a cytokine, myokine, and/or adipokine.

### Peptide/Polypeptide Hormones

In certain embodiments, the one or more polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), 59tilizes 59 stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g., CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoiet 60 tilizes 60 izatios (HeGFs), platelet-derived endothelial cell growth factor (PD-ECGF), angiopoietin, vascular endothelial growth factor (VEGF) family, and glucocorticoidds. In a particular embodiment, the hormone is insulin or incretins such as exenatide, GLP-1.

### Neurohormones

In embodiments, the cargo peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more cargo polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is α-defensin HD-6, HNP-1 and β-defensin hBD-3, lysozyme, cathelcidin LL-37, C-type lectin RegIIIalpha, for example. See, e.g., Wang, “Human Antimicrobial Peptide and Proteins” Pharma, May 2014, 7(5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some embodiments, the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed with. In some embodiments, the anti-fibrillating agent is P4 (VITYF (SEQ ID NO: 40)), P5 (VVVVV (SEQ ID NO: 41)), KR7 (KPWWPRR (SEQ ID NO: 42)), NK9 (NIVNVSLVK (SEQ ID NO: 43)), iAb5p (Leu-Pro-Phe-Phe-Asp (SEQ ID NO: 44)), KLVF (SEQ ID NO: 45) and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3: e01681, D-(PGKLVYA (SEQ ID NO: 46)), RI-OR2-TAT, cyclo(17, 21)-(Lys17, Asp21) A_(1-28), SEN304, SEN1576, D3, R8-Aβ (25-35), human yD-crystallin (HGD), poly-lysine, heparin, poly-Asp, polyGl, poly-L-lysine, poly-L-glutamic acid, LVEALYL (SEQ ID NO: 47), RGFFYT (SEQ ID NO: 48), a peptide set forth or as designed/generated by the method set forth in U.S. Pat. No. 8,754,034, and combinations thereof. In aspects, the anti-fibrillating agent is a D-peptide. In aspects, the anti-fibrillating agent is an L-peptide. In aspects, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In aspects, the retro-inverso modified peptide is derived from a natural or synthetic Aβ peptide. In some embodiments, the polynucleotide encodes a fibrillation resistant protein. In some embodiments, the fibrillation resistant protein is a modified insulin, see e.g., U.S. Pat. No. 8,343,914.

### Signaling and Localization Polypeptides

In some embodiments, the cargo polypeptide contains a signaling or localization sequence. In some embodiments, the signaling or localization sequence is contained at the C-terminus, N-terminus, or both. In some embodiments, the signaling or localization polypeptide directs a function (e.g., secretion, folding, etc.) and/or trafficking to a particular location within a cell (e.g., nucleus, Golgi, lysosome, peroxisome, cytoplasm, membrane, chloroplast, vacuole, mitochondria, etc.). In some embodiments, the signaling and/or localization molecule(s) is/are incorporated in a polynucleotide, such as a cargo polynucleotide, such that it is at the C-terminus, N-terminus, or one or more positions between the C-terminus and N-terminus of a polypeptide encoded by the polynucleotide.

In some embodiments, a polynucleotide of the present invention includes a polynucleotide sequence that is or encodes one or more signal peptides, leucine rich repeat (LRR) sequences, nuclear localization signals, a Type IX secretion system (T9SS) substrate, secretion signal peptide, an amino acid sequence capable of directing clearance from a cell or organism, an Fc receptor directing binding to a dendritic cell, and/or directing antigen processing, an F-box domain or polypeptide, a subcellular localization sequence, a TOM70, TOM20, or TOM22 binding polypeptide, a stromal import sequence, a thylakoid targeting sequence, a peroxisome targeting signal 1 sequence, a peroxisome targeting signal 2 sequence, an endoplasmic reticulum signaling sequence.

Exemplary nuclear localization molecules are described in e.g., Lu et al., Cell Communication and Signaling. 2021. 19(60): 1-10 (particularly at Table 1 therein), which can be adapted for use with the present invention. Other non-limiting examples of NLSs include an NLS sequence derived from: the NLS of the SV40 virus large T-antigen, having the amino acid sequence PKKKRKV (SEQ ID NO: 49) or PKKKRKVEAS (SEQ ID NO: 50); the NLS from nucleoplasmin (e.g., the nucleoplasmin bipartite NLS with the sequence KRPAATKKAGQAKKKK (SEQ ID NO: 51); the c-myc NLS having the amino acid sequence PAAKRVKLD (SEQ ID NO: 52) or RQRRNELKRSP (SEQ ID NO: 53); the hRNPA1 M9 NLS having the sequence NQSSNFGPMKGGNFGGRSSGPYGGGGQYFAKPRNQGGY (SEQ ID NO: 54); the sequence RMRIZFKNKGKDTAELRRRRVEVSVELRKAKKDEQILKRRNV (SEQ ID NO: 55) of the IBB domain from importin-alpha; the sequences VSRKRPRP (SEQ ID NO: 56) and PPKKARED (SEQ ID NO: 57) of the myoma T protein; the sequence PQPKKKPL (SEQ ID NO: 58) of human p53; the sequence SALIKKKKKMAP (SEQ ID NO: 59) of mouse c-abl IV; the sequences DRLRR (SEQ ID NO: 60) and PKQKKRK (SEQ ID NO: 61) of the influenza virus NS1; the sequence RKLKKKIKKL (SEQ ID NO: 62) of the Hepatitis virus delta antigen; the sequence REKKKFLKRR (SEQ ID NO: 63) of the mouse Mx1 protein; the sequence KRKGDEVDGVDEVAKKKSKK (SEQ ID NO: 64) of the human poly(ADP-ribose) polymerase; and the sequence RKCLQAGMNLEARKTKK (SEQ ID NO: 65) of the steroid hormone receptors (human) glucocorticoid.

Exemplary signal peptides are described in e.g., Owji et al., European J Cell Biol. 2018. 97(6):422-441, which can be adapted for use with the present invention. Exemplary peroxisome targeting sequences are described in e.g., Baerends et al., 2000. FEMS Microbiol Rev. 24(3):291-301, which can be adapted for use with the present invention. Exemplary endoplasmic reticulum signaling molecules are described in e.g., Walter et al., J Cell Biol. 1981. 91(2 Pt. 1):545-50 doi: 10.1083/jcb.91.2.545, which can be adapted for use with the present invention. Exemplary lysosomal and endosomal signaling molecules are described in e.g., Bonifacino and Traub. 2003. Ann. Rev. Biochem. 72:395-447, which can be adapted for use with the present invention. Exemplary endoplasmic reticulum signaling sequences are described in e.g., J Cell Biol. 1996 Jul. 2; 134(2):269-278, which can be adapted for use with the present invention. Exemplary Golgi signaling sequences are described in e.g., Gleeson et al., 1994. Glycoconjugat J. 11:381-394, which can be adapted for use with the present invention.

Exemplary nuclear export signals include, without limitation, HIV Rev NES and MAPK NES.

The number of signaling or localization polypeptides can range from 0-10 or more, such as 0, to/or 1, 2, 3, 4, 5, 6, 7, 8, 9, 10 or more.

### Guide Molecules

The programmable nuclease-peptidase composition, CRISPR-Cas, and/or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a programmable nuclease-peptidase or CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting nuclease-peptidase or CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9(1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

Further embodiments are illustrated in the following Examples which are given for illustrative purposes only and are not intended to limit the scope of the invention.

### Target Sequences

In the context of formation of a CRISPR complex, such as a complex formed by the programmable nuclease-peptidase composition of the present invention, “target sequence” refers to a sequence in a polynucleotide to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, the target polynucleotide is associated with a disease, infection, or condition. In some embodiments, detection of the target polynucleotide can be used to detect and/or diagnose a disease, infection, or condition. In some embodiments, the disease or condition that is caused by or associated with the disease associated polypeptide is a cancer, a metabolic disease or condition, an autoimmune disease, a cardiovascular disease, a neurodegenerative disease or condition, a motor neuron disease or condition, graft-versus-host disease, transplant rejection, ischemic injury (e.g., stroke, heart attack, etc.), or spinal cord injury. Exemplary neurodegenerative diseases or conditions are, without limitation, Parkinson's disease, Huntington's disease, Alzheimer's disease, amyotrophic lateral sclerosis, an epilepsy, a dementia, Bell's palsy, or Progressive supranuclear palsy. Exemplary metabolic diseases or conditions are, without limitation, diabetes mellitus (type 1 and type 2), metabolic syndrome, obesity, hyperthyroidism, hypothyroidism, and Cushing's disease or syndrome.

In some embodiments, the target polynucleotide encodes a SNAP Receptor (SNARE) protein. In some embodiments, the target polynucleotide encodes is a vehicle associated membrane protein (VAMP). In some embodiments, the VAMP proteins is VAMP1, VAMP2, VAMP3, VAMP4, VAMP5, VAMP7, and/or VAMP8. In some embodiments, the target polynucleotide encodes a SNAP23 polypeptide and/or SNAP25 polypeptide. In some embodiments, the target polynucleotide encodes phosphatase and tensin homolog (PTEN).

In some embodiments, the target polynucleotide encodes an antibody associated with an autoimmune disease. In some embodiments, the target polynucleotide encodes an autoantigen.

### Signaling and Localization Sequences

Polypeptides of the programmable nuclease-peptidase composition described herein can include one or more signaling and/or localization sequences. Such sequences can be included at the C-terminus and/or N-terminus of the programmable nuclease-peptidase composition polypeptide(s). In some embodiments, the signaling and/or localization sequence is a nuclear localization sequence (NLS). Exemplary signaling and localization sequences are described elsewhere herein (see e.g., “Target polypeptides” section herein).

### Detection Compositions

As previously mentioned, also described herein are detection compositions that comprise one or more of the components of a programmable nuclease-peptidase composition or system described herein. In some embodiments, the target polypeptide is or is included in a detection construct of the detection composition. In some embodiments, a detection composition comprises (i) a RAMP polypeptide; (ii) a guide molecule capable of forming a RAMP-guide molecule complex with the RAMP polypeptide and directing sequence-specific binding of the complex to a target polynucleotide; (iii) a peptidase capable of binding the RAMP polypeptide, the guide molecule, or further complexing with the RAMP-guide complex; and (iv) a detection construct, wherein binding of the RAMP-guide molecule complex to the target polynucleotide initiates peptidase mediated modification of the detection construct resulting in generation of a detectable signal.

Described in certain example embodiments herein are detection compositions comprising (i) a RAMP polypeptide; (ii) a guide molecule capable of forming a RAMP-guide molecule complex with the RAMP polypeptide and directing sequence-specific binding of the complex to a target polynucleotide; (iii) a peptidase capable of binding the RAMP polypeptide, the guide molecule, or further complexing with the RAMP-guide complex; and (iv) a detection construct, wherein binding of the RAMP-guide molecule complex to the target polynucleotide initiates peptidase mediated modification of the detection construct resulting in generation of a detectable signal.

In certain example embodiments, the RAMP polypeptide is derived from Desulfonema ishimotonii, or a homolog, ortholog or variant thereof. RAMP polypeptides are further described in greater detail elsewhere herein. In certain example embodiments, the RAMP polypeptide comprises a Cas11 domain and multiple Cas7 domains. Cas 11 and Cas 7 domains are described in greater detail elsewhere herein. In certain example embodiments, the RAMP polypeptide further comprises a Csm3, Csm4, or Csm6 domain. Csm3, Csm4, and Csm6 domains are described in greater detail elsewhere herein. In certain example embodiments, the RAMP polypeptide is a Type III-E Cas polypeptide.

### Detection Construct

The detection composition can include a detection construct. In some embodiments, the detection construct comprises a polypeptide (e.g., a target polypeptide) that contains one or more peptidase recognition motifs. As used herein, a “detection construct” refers to a molecule that can be cleaved or otherwise deactivated by an activated programmable nuclease-peptidase composition or system effector protein described herein. The detection construct can be capable of producing one or more detectable signals. The detection construct can exist in an unmodified state and when modified (e.g., cleaved) by an activated effector (e.g., a peptidase), the detection construct can produce one or more detectable signals to indicate the presence of a target (e.g., a target polynucleotide). In some embodiments, one or more of the detectable signals can be an assay control. In certain example embodiments, the detection construct comprises a peptidase recognition motif recognized by the peptidase. Peptidase recognition motifs are described in greater detail elsewhere herein. In certain example embodiments, the peptidase recognition motif comprises or consists of SEQ ID NO: 3 or a sequence therein. In certain example embodiments, the peptidase is a TPR-CHAT peptidase. In certain example embodiments, the TPR-CHAT peptidase is derived from Desulfonema ishimotonii or a homolog, ortholog, or variant thereof. Other TPR-CHAT peptidases are described elsewhere herein. In certain example embodiments, the detection construct comprises a polypeptide comprising a peptidase recognition motif recognized by the peptidase. In certain example embodiments, the polypeptide is a fluorescent protein protease reporter. Other suitable reporters are described elsewhere herein e.g., with respect to cargo target polypeptides. In some embodiments, cleavage of the polypeptide containing a peptidase recognition motif of the detection construct releases agents or produces conformational changes that allow a detectable signal to be produced. It will be appreciated that a detectable signal can be generation of a positive signal (e.g., a gain of function) or a loss of a signal (e.g., a loss of function). In some embodiments, prior to cleavage, or when the detection construct is in an ‘active’ state, the detection construct blocks the generation or detection of a positive detectable signal.

It will be understood that in certain example embodiments a minimal background signal may be produced in the presence of an active detection construct. A positive detectable signal may be any signal that can be detected using optical, fluorescent, chemiluminescent, electrochemical, functional assay, or other detection methods known in the art. The term “positive detectable signal” is used to differentiate from other detectable signals that may be detectable in the presence of the detection construct. For example, in certain embodiments a first signal may be detected when the masking agent is present or when a composition or system of the present invention is not activated (i.e., a negative detectable signal), which then converts to a second signal (e.g., the positive detectable signal) upon detection of the target molecules and cleavage or deactivation of the masking agent, or upon activation of the effector protein of the composition or system of the present invention. The positive detectable signal, then, is a signal detected upon activation of the effector protein of the composition or system of the present invention, and may be, in a colorimetric or fluorescent assay, a decrease in fluorescence or color relative to a control or an increase in fluorescence or color relative to a control, depending on the configuration. In some embodiments, it also depends on the configuration of a lateral flow substrate, and as described further herein.

In certain example embodiments, the detection construct may suppress generation of a gene product. The gene product may be encoded by a reporter construct that is added to the sample. The detection construct may be an interfering RNA involved in an RNA interference pathway, such as a short hairpin RNA (shRNA) or small interfering RNA (siRNA). The detection construct may also comprise microRNA (miRNA). While present, the detection construct suppresses expression of the gene product. The gene product may be a fluorescent protein or other RNA transcript or proteins that would otherwise be detectable by a labeled probe, aptamer, or antibody but for the presence of the detection construct. Upon activation of the effector protein the detection construct is cleaved or otherwise silenced allowing for expression and detection of the gene product as the positive detectable signal. In preferred embodiments, the detection construct comprise two or more detectable signals, for example, fluorescent signals, that can be read on different channels of a fluorimeter.

In specific embodiments, the detection construct comprises a silencing RNA that suppresses generation of a gene product encoded by a reporting construct, wherein the gene product generates the detectable positive signal when expressed.

In certain example embodiments, the detection construct may sequester one or more reagents needed to generate a detectable positive signal such that release of the one or more reagents from the detection construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA or DNA aptamers are degraded.

In certain example embodiments, the detection construct may be immobilized on a substrate, such as a solid substrate, in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized detection construct is a or comprises a target polypeptide that can be cleaved by the activated effector protein of the composition or system of the present invention upon detection of a target molecule (e.g., a target nucleic acid).

In certain other example embodiments, the detection construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the detection construct is cleaved to a degree sufficient to interfere with the ability of the detection construct to bind the reagent thereby allowing the labeled binding partner to bind to the immobilized reagent. Thus, the labeled binding partner remains after the wash step indicating the presence of the target molecule in the sample. In certain aspects, the detection construct that binds the immobilized reagent is a DNA or RNA aptamer. The immobilized reagent may be a protein and the labeled binding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the detection construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In some embodiments, the detection construct may be or include a ribozyme that generates a negative detectable signal, and wherein a positive detectable signal is generated when the ribozyme is deactivated. In some embodiment such a ribozyme can contain a peptidase recognition motif.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal until the detection construct is activated by an effector protein of the composition or system of the present invention. In some embodiments, the protein is bound by a substrate or antibody or other polypeptide that when bound sequesters/inhibits the protein such that it cannot generate the detectable signal. The substrate or antibody can include a peptidase recognition motif such that, when the composition or system of the present invention is activated, an effector cleaves the substrate or antibody, thus removing the inhibition/sequestration of the protein and allows a detectable signal to be produced. In some embodiments the sequestered/inhibited protein is thrombin. When the sequestration/inhibition is removed thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. The same approach may be used for horseradish peroxidase (HRP), beta-galactosidase, or calf alkaline phosphatase (CAP) and within the general principals laid out above.

In certain embodiments, peptidase activity is detected colorimetrically via cleavage of polypeptide inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to peptidase activity, colorimetric enzyme and inhibitor pairs can be engineered into peptidase sensors. The colorimetric peptidase sensor based upon small-molecule inhibitors involves three components: the colorimetric enzyme, the inhibitor, and a bridging polypeptide that is covalently linked to both the inhibitor and enzyme, tethering the inhibitor to the enzyme. In the uncleaved configuration, the enzyme is inhibited by the increased local concentration of the small molecule; when the bridging polypeptide is cleaved (e.g., by peptidase activity of the compositions or systems of the present invention), the inhibitor will be released and the colorimetric enzyme will be activated.

In certain embodiments, a polypeptide-tethered inhibitor may sequester an enzyme, wherein the enzyme generates a detectable signal upon release from the polypeptide-tethered inhibitor by acting upon a substrate. In some embodiments, the polypeptide-tethered inhibitor may inhibit an enzyme and prevents the enzyme from catalyzing generation of a detectable signal from a substance. In some embodiments, the polypeptide-tethered inhibitor may inhibit an enzyme and may prevent the enzyme from catalyzing generation of a detectable signal from a substrate. The polypeptide-tethered inhibitor can be a target polypeptide for the peptidase of the compositions or systems of the present invention.

In certain example embodiments, the detection construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized detection construct is a polypeptide that can be cleaved by the activated effector protein upon detection of a target molecule.

In one example embodiment, the detection construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises a target polypeptide of the compositions or systems of the present invention. Upon activation of the effector proteins disclosed herein, the target polypeptide portion of the bridge molecule is cleaved allowing the detection agent to disperse and resulting in the corresponding change in color. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Al³⁺, Ru³⁺, Zn²⁺, Fe³⁺, Ni²⁺ and Ca²⁺ ions.

When the polypeptide bridge is cut by the activated effector of the composition or system of the present invention (e.g., a peptidase), the aforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate.

In certain other example embodiments, the detection construct may comprise a target polypeptide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the target polypeptide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the target polypeptide is cleaved thereby severing the proximity between the fluorophore and quencher needed to maintain the contact quenching effect. Accordingly, detection of the fluorophore may be used to determine the presence of a target molecule in a sample.

In certain other example embodiments, the detection construct may comprise one or more target polypeptides to which are attached one or more metal nanoparticles, such as gold nanoparticles. In some embodiments, the detection construct comprises a plurality of metal nanoparticles crosslinked by a plurality of target polypeptides forming a closed loop. In one embodiment, the v comprises three gold nanoparticles crosslinked by three target polypeptides forming a closed loop. In some embodiments, the cleavage of the target polypeptides by the effector protein leads to a detectable signal produced by the metal nanoparticles.

In certain other example embodiments, the detection construct may comprise one or more target polypeptides to which are attached one or more quantum dots. In some embodiments, the cleavage of the target polypeptides by the effector protein leads to a detectable signal produced by the quantum dots.

In some embodiments, the detection construct may comprise a quantum dot. The quantum dot may have multiple linker molecules attached to the surface. At least a portion of the linker molecule comprises a polypeptides. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art. Upon activation of the effector proteins disclosed herein, the polypeptide portion of the linker molecule is cleaved thereby eliminating the proximity between the quantum dot and one or more quenchers needed to maintain the quenching effect. In certain example embodiments the quantum dot is streptavidin conjugated. Polypeptides can be attached via biotin or other suitable linkers and recruit quenching molecules with the sequences /5Biosg/UCUCGUACGUUC/3IAbRQSp/ (SEQ ID NO: 66) or /5Biosg/UCUCGUACGUUCUCUCGUACGUUC/3IAbRQSp/ (SEQ ID NO: 67) where /5Biosg/ is a biotin tag and /31AbRQSp/ is an Iowa black quencher (Iowa Black FQ). Upon cleavage, by the activated effectors disclosed herein the quantum dot will fluoresce visibly.

In specific embodiments, the detectable ligand may be a fluorophore and the detection construct may be a quencher molecule.

In a similar fashion, fluorescence energy transfer (FRET) may be used to generate a detectable positive signal. FRET is a non-radiative process by which a photon from an energetically excited fluorophore (i.e., “donor fluorophore”) raises the energy state of an electron in another molecule (i.e., “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the detection construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the detection construct suppresses generation of a detectable positive signal until cleaved or modified by an activated effector protein of the compositions or systems of the present invention. In some embodiments, the detection construct may suppress generation of a detectable positive signal by masking the detectable positive signal or generating a detectable negative signal instead.

### Amplification Reagents

In certain example embodiments, the composition further comprises one or more nucleic acid amplification reagents. The amplification reagent(s) included can be capable of amplifying a target polynucleotide and/or a detectable signal. Exemplary amplification reagents are discussed in greater detail elsewhere herein.

Effector Systems Incorporating the Programmable Nucleacse-Peptidase Composition and/or Substrate

The programmable nuclease-peptidase composition (e.g., gRAMP-CHAT peptidase or functional domain(s) thereof), complex thereof (e.g., complexed with a target nucleic acid binding molecule and/or target nucleic acid), and/or substrate thereof (e.g., target polypeptide, Up 1 or domain thereof containing a gRAMP-CHAT cleavage site) can be incorporated into a system that includes an effector of interest that is coupled to and/or is activated or otherwise modified by cleavage of a programmable nuclease-peptidase composition substrate by the programmable nuclease-peptidase composition in response to binding, complexing and/or cleaving a target nucleic acid. In some embodiments, the substrate is or comprises Up1 or domain thereof having a gRAMP-CHAT recognition and/or cleavage site (e.g., a peptidase recognition motif described elsewhere herein). In some embodiments, the substrate is a target polypeptide.

In some embodiments, the programmable nuclease-peptidase composition substrate is coupled to or otherwise associated with an effector of interest within the system such that when the peptidase of the programmable nuclease-peptidase composition is activated (such as by cleaving, binding, and/or otherwise complexing with a target nucleic acid) it acts on the substrate to cleave or otherwise modify the substrate, which in turn activates, releases, and/or otherwise modifies the effector of interest such that the effector of interest performs a function or imparts an effect. In some embodiments, effector system is configured for in vitro (e.g., cell free) applications. For example, and as described in greater detail elsewhere herein, the effector system can be configured as an in vitro diagnostic system. In some embodiments, the effector system is configured for ex vivo or in vivo applications, such as systems for triggering biological activities, controlled delivery/activation of effectors of interest.

Exemplary and non-limiting effector systems are described below and elsewhere herein.

Exemplary Effector Systems In vitro Nucleic Acid Detection

In some embodiments, the programmable nuclease-peptidase composition substrate (e.g., a polypeptide or peptide that is or comprises Up1 or domain thereof of containing a peptidase (e.g., gRAMP-CHAT) recognition and/or cleavage site) and/or programmable nuclease-peptidase composition or component(s) thereof can be incorporated into an in vitro nucleic acid detection system and assay. In some embodiments, the peptidase (e.g., a gRAMP-CHAT) substrate (e.g., Up1 or domain thereof of containing gRAMP-CHAT cleavage site) can include at one or more different tags, each placed at a different position within the substrate. In some embodiments, a first tag is fused to or otherwise coupled to the N- or at the C-terminus of the substrate. In some embodiments that include a second tag, the second tag is fused or otherwise coupled to a different terminus than the first tag. Thus, in some embodiments, a first tag is fused to or is otherwise coupled to the N-terminus of the substrate and a second tag is fused to or is otherwise coupled to the C-terminus of the substrate. In other embodiments, a first tag is fused to or is otherwise coupled to the C-terminus of the substrate and a second tag is fused to or is otherwise coupled to the N-terminus of the substrate. In some embodiments, cleavage of the substrate by a peptidase (e.g., a gRAMP-CHAT or functional domain(s) thereof) of the programmable nuclease-peptidase composition that is/are activated by binding, complexing, and/or cleaving a target nucleic acid (e.g., a target RNA) results in release one or modification of one both portions of the tagged substrate and/or tag(s). The released portion(s) in turn activate or otherwise with a detection construct capable of reacting with one or both tags so as to produce a signal indicative of target nucleic acid detection. In some embodiments, all or components of the effector system are contained in a device or on a substrate such as a lateral flow strip. Detections constructs capable of producing a signal can be present at discrete locations along the lateral flow strip or other substrate separate from or within the same discrete location as the peptidase, substrate. When the released or otherwise activated tagged portion containing the appropriate tag is present in the same discrete location as the corresponding detection construct a signal can be produced indicating detection of a target nucleic acid. Devices and other configurations are described in greater detail elsewhere herein and can be adapted for use with an effector system.

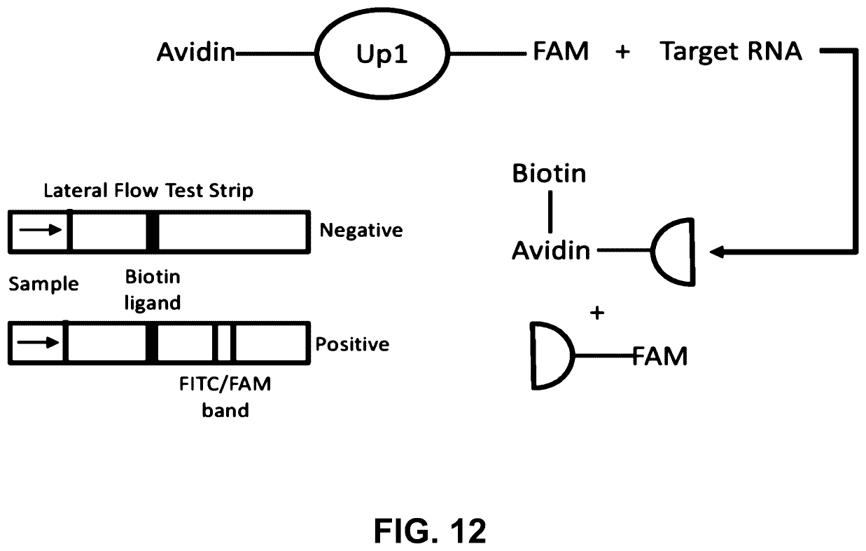

As shown in FIG. 12, in some embodiments, the peptidase substrate can be tagged with an N-terminal avidin tag, which can be biotinylated, and a C-terminal FAM tag. Cleavage of the biotin-Up1-FAM substrate in response to the gRAMP-CHAT complexing with a target RNA and being activated results in release of one or both tagged portions of the Up1 substrate. The released tagged portion(s) of the Up1 substrate can travel along a lateral flow strip and contact FAM and/or biotin detection constructs located at discrete locations along the flow strip whereby a reaction or interaction between the tag and detection construct results in a visual signal thus allowing visual detection on a standard biotin/FAM flow strip.

### In Vivo/Ex Vivo Effector Systems

In some embodiments, the effector system is configured for in vivo/ex vivo applications. In general, an effector of interest is coupled to (e.g., via direct fusion or via a linker) to a peptidase substrate of the programmable nuclease-peptidase composition disclosed herein. In some embodiments, the peptidase substrate (e.g., a target polypeptide) is cleaved by the peptidase upon activation of the peptidase by complexing with a target nucleic acid and/or target nucleic acid binding molecule. Cleavage of the peptidase substrate results, either directly or indirectly, in effector function.

Effectors of interest can be any desired effector molecule capable of performing a desired function, such as a biological function or otherwise cause a biological effect. Exemplary biological functions and/or effects include, without limitation, nucleic acid and genome modification (e.g. gene editing, base editing, and/or the like), programmed cell death (including but not limited to apoptosis), epigenetic modification (e.g., histone modification (e.g., methylation and acetylation), DNA methylation/unmethylation), RNAi), transcription and/or translation modulation, DNA replication modulation, cell signaling and/or transduction modulation, inflammatory modulation, cell cycle modulation, cell proliferation modulation, immunomodulation, cell growth modulation, antioxidant, anti-neoplastic, anti-pyretic, antimicrobial, antiviral, antifungal, analgesic, reporter (e.g., fluorescence or other signal), radiation sensitizing, anxiolytic, antipsychotic, psychedelic, dissociative, stimulant, depressive, ion or other channel modulation, phosphorylation/dephosphorylation, ubiquination, methylation/demethylation, acetylation/deacetylation, metabolic modulation, and/or the like, and any combination thereof.

Exemplary effectors of interest include, without limitation, peptides, proteins, nucleic acids (DNA, RNA or combinations thereof), lipids, small molecule chemical compounds (e.g., small molecule therapeutic compounds), or any combination thereof. Exemplary effectors of interest include, without limitation, genetic modifiers (e.g., CRISPR-Cas systems or components thereof, IscB systems or components thereof, recombinases, transposases, and/or the like), antibodies, aptamers, ribozymes, guide sequences for ribozymes, hormones, immunomodulators, antipyretics, anxiolytics, antipsychotics, analgesics, antispasmodics, anti-inflammatoires, anti-histamines, anti-infectives, radiation sensitizers, psychedelics, dissociatives, hallucinogenics, and chemotherapeutics, stimulants, depressives, and any combination thereof.

Other exemplary effectors of interest are described in greater detail elsewhere herein and/or will be appreciated by those of ordinary skill in the art in view of the description herein and are within the scope of the present disclosure.

In some embodiments, the peptidase substrate is tethered, such as via an anchor molecule, to a cell membrane or organelle. In some embodiments the peptidase substrate is coupled to an anchor molecule (e.g., via fusion or a linker). In some embodiments, the cell membrane is the nuclear membrane. In some embodiments, the cell membrane is the cytoplasmic membrane. In some embodiments, the organelle is the mitochondria, endoplasmic reticulum (rough or smooth), Golgi apparatus, lysosome, vacuole, chloroplast, and/or microtubule. Anchor molecules can be any molecule or complex that attaches (reversibly or irreversibly) an uncleaved or portion of a cleaved peptidase substrate to a cell membrane or organelle. Anchor molecules can be proteins, peptides, lipids, nucleic acids, sugars, and/or the like and any combination thereof. Exemplary anchor molecules include, but are not limited to, transmembrane proteins or transmembrane domain(s) thereof, binding partners (e.g., ligands, antibodies, aptamers, receptors, and/or the like) for cell membrane or organelle bound ligands, molecules, receptors, and/or the like, lipid-linked proteins (also referred to as lipid-anchored proteins), glycoslyphosphatidlinositol (GPIs), an isoprenoid containing 15 or 20 carbons attached to an optionally methylated cysteine residue at a C-terminus of the peptidase substrate via a suitable liker (e.g., a thioester linker), a myristic acid attached to a glycine residue at the N terminus of the peptidase substrate via an amid linkage, a palmitic acid attached to a cysteine residue at or close to the N- or C-terminus of the peptidase substrate via a suitable linker (e.g., thioester linker) or an internal serine and/or threonine residues of the peptidase substrate via a suitable linkage (e.g., ester linkage), a fatty acid or 1, 2, diaculglycerol attached to an N-terminal cysteine via a suitable linker or linkage (e.g., amide or thioether), and combinations thereof.

In some embodiments, the peptidase substrate can be tethered to the cell membrane via an electrostatic interaction. Phospholipids found in biological membranes can have a negative charge. In some embodiments, the peptidase substrate can contain one or more regions of excess of positively charged amino acids that can be attracted to the negative charge of the phospholipid cell membrane thus tethering the peptidase substrate or portion thereof to the cell membrane.

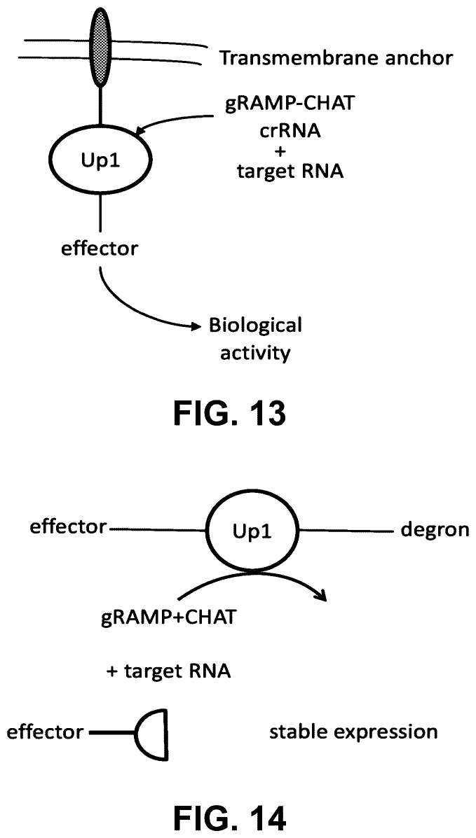

In certain exemplary embodiments, a gRAMP-CHAT substrate (e.g., Up1) and/or gRAMP-CHAT can be incorporated into an in vivo effector system. FIG. 13 shows an exemplary schematic for an in vivo effector system in which proteins are tethered to a cell membrane using transmembrane domains (e.g., gap43: LCCMRRTKQVEKNDEDQKI (SEQ ID NO: 34), L10: GCVCSSNPENNNN (SEQ ID NO: 35), S15: GSSKSKPKDPSQRRNNNN (SEQ ID NO: 36)) with a linker sequence containing a minimal Up1 substrate (amino acids 297-565). Following RNA detection and Up1 cleavage, the effector domain can move into the nucleus and perform different biological activities. For example, dCas9-VPR effector can be used to allow for the activation of genes, and a Cre effector to activate GFP expression.

In some embodiments, the peptidase substrate is coupled to (e.g., fused with attached via a linker) to a degron as well as the effector of interest. Degron is a term of art that generally refers to protein or peptide elements that confer metabolic instability or degradation. So long as the effector of interest is coupled to the degron via the peptidase substrate, the activity of the effector of interest is inhibited via its degradation. Upon cleavage of the peptidase substrate by a peptidase of a programmable nuclease-peptidase composition that is activated by binding, complexing, and/or cleaving with a target nucleic acid, the effector of interest is decoupled from to the degron. Without being bound by theory, once the effector of interest is disassociated/uncoupled from the degron, expression of the effector of interest is stabilized and thus the function of the effector of interest is no longer inhibited.

In some embodiments, the degron is a constitutive degron. In some embodiments, the degron is an inducible degron. Suitable degrons that can be included in some embodiments of the effector system are generally known, and include without limitation, tripartite degrons (Guharoy et al., 2016. Nat. comm. 7:10239), N-degrons and C-degrons (see e.g., Varshavsky, A. 2019. PNAS. 116(2)358-366), synthetic and modular degrons (see e.g., Chassin et al., 2019. Nat. Comm. 10:2013), a bacterial degron (see e.g., Izert et al., Front. Mol. Biosci. 2021. https://doi.org/10.3389/fmolb.2021.669762, particularly at Table 1), inducible degrons (see e.g., Yesbolatova et al. 2020. Nat. Comm. 11:5701; Dohmen et al. Scienc. 263(5151):1273-1276; and Murawska et al., ACS Chem Biol. 2022. 17(1):24-31). In some embodiments the degron is a dihdrofolate reductase or domain thereof.

FIG. 14 shows an exemplary schematic for a degron in which a degron tag is fused to an effector of interest via a linker sequence containing a minimal Up1 substrate (297-565). For a dihydrofolate example, reductase (DHFR) sequence (ISLIAALAVDHVIGMETVMPWNLPADLAWFKRNTLNKPVIMGRHTWESIGRPLPGR KNIILSSQPSTDDRVTWVKSVDEAIAACGDVPEIMVIGGGRVYEQFLPKAQKLYLTHI DAEVEGDTHFPDYEPDDWESVFSEFHDADAQNSHSYCFEILERR (SEQ ID NO: 37)), which destabilizes the protein resulting in degradation. Following RNA detection and Up1 cleavage, the degron tag is removed from the effector thereby stabilizing the effector and allowing for its activity.

### Polynucleotides and Vectors

Described herein are polynucleotides encoding one or more components (e.g., polypeptides and/or guide polynucleotides) of the programmable nuclease-protease composition or system (such as a detection composition or system) comprising the programmable nuclease-protease composition. Also described herein are vectors and vector systems containing one or more programmable nuclease-protease composition or system encoding polynucleotides. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Polynucleotides

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

### Codon Optimization

In some embodiments, the polynucleotide can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292(2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257 (6):3026-31. As to codon usage in plants including algae, reference is made to Codon usage in higher plants, green algae, and cyanobacteria, Campbell and Gowri, Plant Physiol. 1990 January; 92(1):1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46 (4):449-59.

The polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a polynucleotide coding sequence encoding one or more elements of the programmable nuclease-protease composition or system described herein is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate.

### Vectors and Vector Systems

Also provided herein are vectors and vector system that can contain one or more of the programable nuclease-protease composition or system polynucleotides (such as an encoding polynucleotide) described herein. In certain embodiments, the vector can contain one or more polynucleotides encoding one or more elements of a CRISPR-Cas system described herein. The vectors can be useful in producing bacterial, fungal, yeast, plant cells, animal cells, and transgenic animals that can express one or more components of the programable nuclease-protease composition or system described herein. Within the scope of this disclosure are vectors containing one or more of the polynucleotide sequences described herein. One or more of the polynucleotides that are part of the programable nuclease-protease composition or system described herein can be included in a vector or vector system. The vectors and/or vector systems can be used, for example, to express one or more of the polynucleotides in a cell, such as a producer cell, to produce programable nuclease-protease composition or system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the programable nuclease-protease composition or system described herein. In some embodiments, expression of elements of the programable nuclease-protease composition or system described herein can be driven by the CBh promoter or other ubiquitous promoter. Where the element of the programable nuclease-protease composition or system is an RNA, its expression can be driven by a Pol III promoter, such as a U6 promoter. In some embodiments, the two are combined.

In some embodiments, a vector capable of delivering an effector protein and optionally at least one guide RNA to a cell can be composed of or contain a minimal promoter operably linked to a polynucleotide sequence encoding the effector protein and a second minimal promoter operably linked to a polynucleotide sequence encoding at least one guide RNA, wherein the length of the vector sequence comprising the minimal promoters and polynucleotide sequences is less than 4.4 Kb. In an embodiment, the vector can be a viral vector. In certain embodiments, the viral vector is an adeno-associated virus (AAV) or an adenovirus vector.

In some embodiments, the vector capable of delivering a lentiviral vector for an effector protein and at least one guide RNA to a cell can be composed of or contain a promoter operably linked to a polynucleotide sequence encoding a RAMP, a target polypeptide, a peptidase and a second promoter operably linked to a polynucleotide sequence encoding at least one guide RNA, wherein the polynucleotide sequences are in reverse orientation.

In one embodiment, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the programable nuclease-protease composition or system complex to the one or more target sequence(s) in a eukaryotic cell, wherein the programable nuclease-protease composition or system complex comprises a RAMP polypeptide and/or peptidase polypeptide complexed with the one or more guide sequence(s) that is hybridized to the one or more target sequence(s); and (b) a second regulatory element operably linked to an enzyme-coding sequence encoding said RAMP polypeptide and/or peptidase, preferably comprising at least one nuclear localization sequence and/or at least one NES; wherein components (a) and (b) are located on the same or different vectors of the system. Where applicable, a tracr sequence may also be provided. In some embodiments, component (a) further comprises two or more guide sequences operably linked to the first regulatory element, wherein when expressed, each of the two or more guide sequences direct sequence specific binding of a programable nuclease-protease composition or system complex to a different target sequence in a eukaryotic cell. In some embodiments, the programable nuclease-protease composition or system complex comprises one or more nuclear localization sequences and/or one or more NES of sufficient strength to drive accumulation of said programable nuclease-protease composition or system complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

These and others are further detailed and described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the programable nuclease-protease composition or system described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited, to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSecl (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of a CRISPR-Cas system so as to drive expression of the one or more elements of the CRISPR-Cas system described herein.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of a programable nuclease-protease composition or system described herein are introduced into a host cell such that expression of the elements of the engineered delivery system described herein direct formation a programable nuclease-protease composition or system complex at one or more target sites. For example, a programable nuclease-protease composition or system effector protein describe herein and a nucleic acid component (e.g., a guide polynucleotide) can each be operably linked to separate regulatory elements on separate vectors. RNA(s) of different elements of programable nuclease-protease composition or system described herein can be delivered to an animal, plant, microorganism or cell thereof to produce an animal (e.g., a mammal, reptile, avian, etc.), plant, microorganism or cell thereof that constitutively, inducibly, or conditionally expresses different elements of the programable nuclease-protease composition or system described herein that incorporates one or more elements of the programable nuclease-protease composition or system described herein or contains one or more cells that incorporates and/or expresses one or more elements of the programable nuclease-protease composition or system described herein.

In some embodiments, two or more of the elements expressed from the same or different regulatory element(s), can be combined in a single vector, with one or more additional vectors providing any components of the system not included in the first vector. In some embodiments, the specific regulator elements used are chosen to reduce or eliminate regulatory element competition, such as promoter competition. Programable nuclease-protease composition or system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more programable nuclease-protease composition or system proteins, embedded within one or more intron sequences (e.g., each in a different intron, two or more in at least one intron, or all in a single intron). In some embodiments, the programable nuclease-protease composition or system polynucleotides can be operably linked to and expressed from the same promoter.

### Cell-Free Vector and Polynucleotide Expression

In some embodiments, the polynucleotide encoding one or more features of the programable nuclease-protease composition or system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the programable nuclease-protease composition or system polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC)), MYL2 (MLC-2v), TNI3 (cTnl), NPPA (ANF), Slc8a1 (Ncx1), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters (e.g., ITGAM, CD43 promoter, CD14 promoter, CD45 promoter, CD68 promoter), urogenital cell specific promoters (e.g., Pbsn, Upk2, Sbp, Fer114), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the CRISPR-Cas system described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the programable nuclease-protease composition or system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the programable nuclease-protease composition or system are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include, but is not limited to, sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more elements of the programable nuclease-protease composition or system described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the polynucleotide, vector or system thereof can include one or more elements capable of translocating and/or expressing a programable nuclease-protease composition or system polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/ and Negi et al., 2015. Database. 2015: bav003; doi: 10.1093/database/bav003), nuclear export signals (e.g., LXXXLXXLXL (SEQ ID NO: 68) and others described elsewhere herein), endoplasmic reticulum localization/retention signals (e.g., KDEL (SEQ ID NO: 69), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLOS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g., (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http: minimotifminer.org, http://mitominer.mrc-mbu.cam.ac.uk/release-4.0/embodiment.do?name=Protein % 20MTS), LocDB (see above), PTSs predictor ( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the programable nuclease-protease composition or system polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the programable nuclease-protease composition or system polynucleotide such that the selectable marker polypeptide, when translated, is inserted between two amino acids between the N- and C-terminus of the programable nuclease-protease composition or system polypeptide or at the N- and/or C-terminus of the programable nuclease-protease composition or system polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the programable nuclease-protease composition or system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the CRISPR-Cas system described herein via suitable linker, such as a glycine or glycine serine linkers as short as GS or GG up to (GGGGG)₃ (SEQ ID NO: 70) or (GGGGS)₃ (SEQ ID NO: 71). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the programable nuclease-protease composition or system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated programable nuclease-protease composition or system polynucleotide(s) to specific cells, tissues, organs, etc.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more elements of a programmable nuclease-peptidase composition or system described herein are as used in the foregoing documents, such as International Patent Publication No. WO 2014/093622 (PCT/US2013/074667) and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a programmable nuclease-peptidase polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the programmable nuclease-peptidase composition or system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more components of the programmable nuclease-peptidase composition or system, including but not limited to a peptidase and/or RAMP effector, is external to the capsid or virus particle. In the sense that it is not inside the capsid (enveloped or encompassed with the capsid), but is externally exposed so that it can contact the target genomic DNA. In some embodiments, the viral vector is configured such that all the carog(s) are contained within the capsid after packaging.

### Split Viral Vector Systems

When the programmable nuclease-peptidase composition or system viral vector or vector system (be it a retroviral (e.g., AAV) or lentiviral vector) is designed so as to position the cargo(s) (e.g., one or more programmable nuclease-peptidase composition or system components) at the internal surface of the capsid once formed, the cargo(s) will fill most or all of internal volume of the capsid. In other embodiments, the effector protein may be modified or divided so as to occupy a less of the capsid internal volume. Accordingly, in certain embodiments, the programmable nuclease-peptidase composition or system or component thereof (e.g., a RAMP or peptidase effector protein) can be divided in two portions, one portion comprises in one viral particle or capsid and the second portion comprised in a second viral particle or capsid. In certain embodiments, by splitting the programmable nuclease-peptidase composition or system or component thereof in two portions, space is made available to link one or more heterologous domains to one or both programmable nuclease-peptidase composition or system component (e.g., RAMP or peptidase protein) portions. Such systems can be referred to as “split vector systems” or in the context of the present disclosure a “split programmable nuclease-peptidase composition or system” a “split programmable nuclease-peptidase composition or system polypeptide”, a “split RAMP protein” and the like. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of the programmable nuclease-peptidase composition or system that can be achieved with a split system or split protein design.

Split programmable nuclease-peptidase composition or system polypeptides that can be incorporated into the AAV or other vectors described herein are set forth elsewhere herein and in documents incorporated herein by reference in further detail herein. In certain embodiments, each part of a split programmable nuclease-peptidase composition or system polypeptides are attached to a member of a specific binding pair, and when bound with each other, the members of the specific binding pair maintain the parts of the programmable nuclease-peptidase composition or system polypeptide in proximity. In certain embodiments, each part of a split programmable nuclease-peptidase composition or system polypeptide is associated with an inducible binding pair. An inducible binding pair is one which is capable of being switched “on” or “off” by a protein or small molecule that binds to both members of the inducible binding pair. In general, according to the invention, programmable nuclease-peptidase composition or system polypeptides may preferably split between domains, leaving domains intact. Preferred, non-limiting examples of such programmable nuclease-peptidase composition or system polypeptides include, without limitation, RAMP polypeptides, peptidase polypeptide, sCas protein, and orthologues.

In some embodiments, any AAV serotype is preferred. In some embodiments, the VP2 domain associated with the programmable nuclease-peptidase composition or system polypeptide is an AAV serotype 2 VP2 domain. In some embodiments, the VP2 domain associated with the programmable nuclease-peptidase composition or system polypeptide is an AAV serotype 8 VP2 domain. The serotype can be a mixed serotype as is known in the art.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the CRISPR-Cas systems can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIA V vectors have been used to mediate expression, packaging, and/or delivery in other contexts, such as for ocular gene therapy (see, e.g., Balagaan, J Gene Med 2006; 8:275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the programmable nuclease-peptidase composition or system described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the programmable nuclease-peptidase composition or system of the present invention.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124:1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLOS Pathog. 12(e1005461); and Friedrich et al. 2013. Mol. Ther. 2013. 21:849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA (SEQ ID NO: 72)) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA (SEQ ID NO: 72) can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA (SEQ ID NO: 72) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver a programmable nuclease-peptidase composition or system polynucleotide described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g. antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus, and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

Adenoviral vectors, Helper-dependent Adenoviral vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more CRISPR-Cas polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the programmable nuclease-peptidase composition or system polynucleotides described herein. In some embodiments, the polynucleotide to be delivered via the viral particle produced from a helper-dependent adenoviral vector or system thereof can be up to about 37 kb. Thus, in some embodiments, a adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5):2964-2971; Zhang et al. 2013. PloS One. 8(10) e76771; and Cooney et al. 2015. Mol. Ther. 23(4):667-674), whose techniques and vectors described therein can be modified and adapted for use in the programmable nuclease-peptidase composition or system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the programmable nuclease-peptidase composition or system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the programmable nuclease-peptidase composition or system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82:5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the programmable nuclease-peptidase composition or system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In another embodiment, the invention provides a non-naturally occurring or engineered programmable nuclease-peptidase composition or system protein associated with Adeno Associated Virus (AAV), e.g., an AAV comprising a programmable nuclease-peptidase composition or system protein as a fusion, with or without a linker, to or with an AAV capsid protein such as VP1, VP2, and/or VP3; and, for shorthand purposes, such a non-naturally occurring or engineered programmable nuclease-peptidase composition or system protein is herein termed a “AAV-programmable nuclease-peptidase composition or system protein” More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi: 10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a programmable nuclease-peptidase composition or system-protein. Likewise, these can be fusions, with the protein, e.g., large payload protein such as a programmable nuclease-peptidase composition or system-protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that large payload proteins can be fused to the AAV capsid. Applicants provide AAV capsid programmable nuclease-peptidase composition or system R protein (e.g., RAMP, peptidase, etc.) fusions and those AAV-capsid programmable nuclease-peptidase composition or system protein fusions can be a recombinant AAV that contains nucleic acid molecule(s) encoding or providing programmable nuclease-peptidase composition or system or complex RNA guide(s), whereby the programmable nuclease-peptidase composition or system protein fusion delivers a programmable nuclease-peptidase composition or system complex by the fusion, e.g., VP1, VP2, or VP3 fusion, and the guide RNA is provided by the coding of the recombinant virus, whereby in vivo, in a cell, the programmable nuclease-peptidase composition or system is assembled from the nucleic acid molecule(s) of the recombinant providing the guide RNA and the outer surface of the virus providing the programmable nuclease-peptidase composition or system polypeptide. Accordingly, the instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus of within the family Parvoviridae or the genus Dependoparvovirus or any of the other foregoing genera within Parvoviridae is contemplated as within the invention with discussion herein as to AAV applicable to such other viruses.

In some embodiments, the programmable nuclease-peptidase composition or system polypeptide is external to the capsid or virus particle. In the sense that it is not inside the capsid (enveloped or encompassed with the capsid), but is externally exposed so that it can contact the target genomic DNA). In some embodiments, the programmable nuclease-peptidase composition or system polypeptide is associated with the AAV VP2 domain by way of a fusion protein. In some embodiments, the association may be considered to be a modification of the VP2 domain. Where reference is made herein to a modified VP2 domain, then this will be understood to include any association discussed herein of the VP2 domain and the programmable nuclease-peptidase composition or system polypeptide. In some embodiments, the AAV VP2 domain may be associated (or tethered) to the programmable nuclease-peptidase composition or system polypeptide via a connector protein, for example using a system such as the streptavidin-biotin system. In an embodiment, the present invention provides a polynucleotide encoding the present programmable nuclease-peptidase composition or system polypeptide and associated AAV VP2 domain. In one embodiment, the invention provides a non-naturally occurring modified AAV having a VP2-programmable nuclease-peptidase composition or system polypeptide capsid protein, wherein the programmable nuclease-peptidase composition or system polypeptide is part of or tethered to the VP2 domain. In some preferred embodiments, the programmable nuclease-peptidase composition or system polypeptide is fused to the VP2 domain so that, in another embodiment, the invention provides a non-naturally occurring modified AAV having a VP2-programmable nuclease-peptidase composition or system polypeptide fusion capsid protein. Thus, reference herein to a VP2-programmable nuclease-peptidase composition or system polypeptide capsid protein may also include a VP2-programmable nuclease-peptidase composition or system polypeptide fusion capsid protein. In some embodiments, the VP2-programmable nuclease-peptidase composition or system polypeptide capsid protein further comprises a linker, whereby the VP2-programmable nuclease-peptidase composition or system polypeptide is distanced from the remainder of the AAV. In some embodiments, the VP2-programmable nuclease-peptidase composition or system polypeptide capsid protein further comprises at least one protein complex, e.g., programmable nuclease-peptidase composition or system polypeptide complex, such as a programmable nuclease-peptidase composition or system polypeptide complex guide RNA that targets a particular DNA, TALE, etc. A programmable nuclease-peptidase composition or system polypeptide complex, such as programmable nuclease-peptidase composition or system comprising the VP2-programmable nuclease-peptidase composition or system polypeptide capsid protein and at least one programmable nuclease-peptidase composition or system polypeptide complex, such as a programmable nuclease-peptidase composition or system polypeptide complex guide RNA that targets a particular DNA, is also provided in one embodiment.

In one embodiment, the invention provides a non-naturally occurring or engineered composition comprising a programmable nuclease-peptidase composition or system polypeptide which is part of or tethered to an AAV capsid domain, i.e., VP1, VP2, or VP3 domain of Adeno-Associated Virus (AAV) capsid. In some embodiments, part of or tethered to an AAV capsid domain includes associated with associated with a AAV capsid domain. In some embodiments, the programmable nuclease-peptidase composition or system polypeptide may be fused to the AAV capsid domain. In some embodiments, the fusion may be to the N-terminal end of the AAV capsid domain. As such, in some embodiments, the C-terminal end of the programmable nuclease-peptidase composition or system polypeptide is fused to the N-terminal end of the AAV capsid domain. In some embodiments, an NLS and/or a linker (such as a GlySer linker) may be positioned between the C-terminal end of the programmable nuclease-peptidase composition or system polypeptide and the N-terminal end of the AAV capsid domain. In some embodiments, the fusion may be to the C-terminal end of the AAV capsid domain. In some embodiments, this is not preferred due to the fact that the VP1, VP2 and VP3 domains of AAV are alternative splices of the same RNA and so a C-terminal fusion may affect all three domains. In some embodiments, the AAV capsid domain is truncated. In some embodiments, some or all of the AAV capsid domain is removed. In some embodiments, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the CRISPR protein. A branched linker may be used, with the programmable nuclease-peptidase composition or system polypeptide fused to the end of one of the branches. This allows for some degree of spatial separation between the capsid and the programmable nuclease-peptidase composition or system polypeptide. In this way, the programmable nuclease-peptidase composition or system polypeptide is part of (or fused to) the AAV capsid domain.

In other embodiments, the CRISPR enzyme may be fused in frame within, i.e., internal to, the AAV capsid domain. Thus, in some embodiments, the AAV capsid domain again preferably retains its N-terminal and C-terminal ends. In this case, a linker is preferred, in some embodiments, either at one or both ends of the programmable nuclease-peptidase composition or system polypeptide. In this way, the programmable nuclease-peptidase composition or system polypeptide is again part of (or fused to) the AAV capsid domain. In certain embodiments, the positioning of the programmable nuclease-peptidase composition or system polypeptide is such that the programmable nuclease-peptidase composition or system polypeptide is at the external surface of the viral capsid once formed. In one embodiment, the invention provides a non-naturally occurring or engineered composition comprising a programmable nuclease-peptidase composition or system polypeptide associated with a AAV capsid domain of Adeno-Associated Virus (AAV) capsid. Here, associated may mean in some embodiments fused, or in some embodiments bound to, or in some embodiments tethered to. The programmable nuclease-peptidase composition or system polypeptide may, in some embodiments, be tethered to the VP1, VP2, or VP3 domain. This may be via a connector protein or tethering system such as the biotin-streptavidin system. In one example, a biotinylation sequence (15 amino acids) could therefore be fused to the programmable nuclease-peptidase composition or system polypeptide. When a fusion of the AAV capsid domain, especially the N-terminus of the AAV AAV capsid domain, with streptavidin is also provided, the two will therefore associate with very high affinity. Thus, in some embodiments, provided is a composition or system comprising a programmable nuclease-peptidase composition or system polypeptide-biotin fusion and a streptavidin-AAV capsid domain arrangement, such as a fusion. The programmable nuclease-peptidase composition or system polypeptide-biotin and streptavidin-AAV capsid domain forms a single complex when the two parts are brought together. NLSs may also be incorporated between the programmable nuclease-peptidase composition or system polypeptide and the biotin; and/or between the streptavidin and the AAV capsid domain.

As such, provided is a fusion of a programmable nuclease-peptidase composition or system polypeptide with a connector protein specific for a high affinity ligand for that connector, whereas the AAV VP2 domain is bound to said high affinity ligand. For example, streptavidin may be the connector fused to the programmable nuclease-peptidase composition or system polypeptide, while biotin may be bound to the AAV VP2 domain. Upon co-localization, the streptavidin will bind to the biotin, thus connecting the programmable nuclease-peptidase composition or system polypeptide to the AAV VP2 domain. The reverse arrangement is also possible. In some embodiments, a biotinylation sequence (15 amino acids) could therefore be fused to the AAV VP2 domain, especially the N-terminus of the AAV VP2 domain. A fusion of the programmable nuclease-peptidase composition or system polypeptide with streptavidin is also preferred, in some embodiments. In some embodiments, the biotinylated AAV capsids with streptavidin-programmable nuclease-peptidase composition or system polypeptide are assembled in vitro. This way the AAV capsids should assemble in a straightforward manner and the programmable nuclease-peptidase composition or system polypeptide-streptavidin fusion can be added after assembly of the capsid. In other embodiments a biotinylation sequence (15 amino acids) could therefore be fused to the programmable nuclease-peptidase composition or system polypeptide, together with a fusion of the AAV VP2 domain, especially the N-terminus of the AAV VP2 domain, with streptavidin. For simplicity, a fusion of the programmable nuclease-peptidase composition or system polypeptide and the AAV VP2 domain is preferred in some embodiments. In some embodiments, the fusion may be to the N-terminal end of the programmable nuclease-peptidase composition or system polypeptide. In other words, in some embodiments, the AAV and programmable nuclease-peptidase composition or system polypeptide are associated via fusion. In some embodiments, the AAV and programmable nuclease-peptidase composition or system polypeptide are associated via fusion including a linker. Suitable linkers are discussed herein, but include Gly Ser linkers. Fusion to the N-term of AAV VP2 domain is preferred, in some embodiments. In some embodiments, the programmable nuclease-peptidase composition or system polypeptide comprises at least one Nuclear Localization Signal (NLS). In a further embodiment, the present invention provides compositions comprising the programmable nuclease-peptidase composition or system polypeptide and associated AAV VP2 domain or the polynucleotides or vectors described herein. Such compositions and formulations are discussed elsewhere herein.

An alternative tether may be to fuse or otherwise associate the AAV capsid domain to an adaptor protein which binds to or recognizes to a corresponding RNA sequence or motif. In some embodiments, the adaptor is or comprises a binding protein which recognizes and binds (or is bound by) an RNA sequence specific for said binding protein. In some embodiments, a preferred example is the MS2 (see Konermann et al. December 2014, cited infra, incorporated herein by reference) binding protein which recognizes and binds (or is bound by) an RNA sequence specific for the MS2 protein.

With the AAV capsid domain associated with the adaptor protein, the CRISPR protein may, in some embodiments, be tethered to the adaptor protein of the AAV capsid domain. The programmable nuclease-peptidase composition or system polypeptide may, in some embodiments, be tethered to the adaptor protein of the AAV capsid domain via the CRISPR enzyme being in a complex with a modified guide, see Konermann et al. The modified guide is, in some embodiments, a sgRNA. In some embodiments, the modified guide comprises a distinct RNA sequence; see, e.g., International Patent Application No. PCT/US14/70175, incorporated herein by reference.

In some embodiments, distinct RNA sequence is an aptamer. Thus, corresponding aptamer-adaptor protein systems are preferred. One or more functional domains may also be associated with the adaptor protein. An example of a preferred arrangement would be: [AAV AAV capsid domain-adaptor protein]-[modified guide-programmable nuclease-peptidase composition or system polypeptide]

In certain embodiments, the positioning of the programmable nuclease-peptidase composition or system polypeptide is such that the programmable nuclease-peptidase composition or system polypeptide is at the internal surface of the viral capsid once formed. In one embodiment, the invention provides a non-naturally occurring or engineered composition comprising a programmable nuclease-peptidase composition or system polypeptide associated with an internal surface of an AAV capsid domain. Here again, associated may mean in some embodiments fused, or in some embodiments bound to, or in some embodiments tethered to. The programmable nuclease-peptidase composition or system polypeptide may, in some embodiments, be tethered to the VP1, VP2, or VP3 domain such that it locates to the internal surface of the viral capsid once formed. This may be via a connector protein or tethering system such as the biotin-streptavidin system as described above and/or elsewhere herein.

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiment the programmable nuclease-peptidase composition or system polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the CRISPR-Cas system of the present invention.

### Poxvirus Vectors

In some embodiments, the vector can be a poxvirus vector or system thereof. In some embodiments, the poxvirus vector can result in cytoplasmic expression of one or more programmable nuclease-peptidase composition or system polynucleotides of the present invention. In some embodiments the capacity of a poxvirus vector or system thereof can be about 25 kb or more. In some embodiments, a poxvirus vector or system thereof can include one or more programmable nuclease-peptidase composition or system polynucleotides described herein.

### Viral Vectors for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a programmable nuclease-peptidase composition or system polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the CRISPR-Cas system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g. the CRISPR-Cas system polynucleotide(s)) between 2 ITRs; (2) a vector that carries the AAV Rep-Cap encoding polynucleotides; and (3) helper polynucleotides. One of skill in the art will appreciate various methods and variations thereof that are both helper and -helper free and as well as the different advantages of each system.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating programmable nuclease-peptidase composition or system polynucleotide(s) and delivering said programmable nuclease-peptidase composition or system polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments one or more programmable nuclease-peptidase composition or system polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the programmable nuclease-peptidase composition or system polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the programmable nuclease-peptidase composition or system polynucleotide(s) of the present invention. In some embodiments, the naked polynucleotide can contain other nucleic acids and/or polynucleotides in addition to the programmable nuclease-peptidase composition or system polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the programmable nuclease-peptidase composition or system polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g. Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more CRISPR-Cas system polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the programmable nuclease-peptidase composition or system polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the programmable nuclease-peptidase composition or system polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the programmable nuclease-peptidase composition or system polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, Sleeping Beauty transposon system (Tc1/mariner superfamily) (see e.g., Ivics et al. 1997. Cell. 91(4):501-510), piggyBac (piggyBac superfamily) (see e.g., Li et al. 2013 110(25): E2279-E2287 and Yusa et al. 2011. PNAS. 108(4):1531-1536), Tol2 (superfamily hAT), Frog Prince (Tc1/mariner superfamily) (see e.g., Miskey et al. 2003 Nucleic Acid Res. 31(23):6873-6881) and variants thereof.

### Delivery of the Polynucleotides, Vectors, and Vector Systems

The polynucleotides, vectors, and/or vector systems can be delivered, such as to a cell or cells, by any suitable method or technique. In some embodiments, delivery can include association or otherwise incorporating the polynucleotides, vectors and/or vector systems with one or more delivery vehicles. Exemplary delivery methods and vehicles are discussed in greater detail below.

### Physical Delivery

In some embodiments, the polynucleotides, vectors, and vector systems or any delivery vehicle containing the same may be introduced to cells by physical delivery methods. Examples of physical methods include microinjection, electroporation, and hydrodynamic delivery. Both nucleic acid and proteins may be delivered using such methods. For example, proteins of the present invention may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for proteins of the programmable nuclease-peptidase composition or system and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and programmable nuclease-peptidase composition or system polypeptide-encoding mRNA to the cytoplasm, e.g., facilitating translation and shuttling of said polypeptides or polynucleotides to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the programmable nuclease-peptidase composition or system polypeptide or polynucleotides and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the programmable nuclease-peptidase composition or system polypeptides and/or polynucleotides, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The programmable nuclease-peptidase composition or system polypeptides and/or polynucleotides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The programmable nuclease-peptidase composition or system polypeptides and/or polynucleotides can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer be used to transduce cells.

### Biolistics

The programmable nuclease-peptidase composition or system polypeptides and/or polynucleotides can be introduced to cells using a biolistic method or technique. The term of art “biolistic”, as used herein refers to the delivery of nucleic acids to cells by high-speed particle bombardment. In some embodiments, the cargo(s) can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a programmable nuclease-peptidase composition or system polypeptides and/or polynucleotides described herein. Various implantable devices are described in the art, and include any device, graft, or other composition that can be implanted into a subject.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles in accordance with the present invention may a greatest dimension (e.g., diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to plant cells, e.g., as described in WO 2008042156, US20130185823, and WO2015089419. In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured, and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi: 10.1038/nnano.2014.84, describing particles, methods of making and using them and measurements thereof.

### Vector Based Delivery Vehicles

Vectors and Vector systems that can be used to deliver programmable nuclease-peptidase composition or system polypeptides and/or polynucleotides are described in greater detail elsewhere herein.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389(1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200 December 2011).

In some embodiments, an LNP delivery vehicle can be used to deliver a virus particle containing a CRISPR-Cas system and/or component(s) thereof. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US20160174546, US20140301951, US20150105538, US20150250725, Wang et al., J. Control Release, 2017 Jan. 31. pii: S0168-3659(17)30038-X. doi: 10.1016/j.jconrel.2017.01.037. [Epub ahead of print]; Altιnoğlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015 Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi: 10.1038/nnano.2014.84; Coelho et al., N Engl J Med 2013; 369:819-29; Aleku et al., Cancer Res., 68(23):9788-98 (Dec. 1, 2008), Strumberg et al., Int. J. Clin. Pharmacol. Ther., 50 (1):76-8 (January 2012), Schultheis et al., J. Clin. Oncol., 32 (36):4141-48 (Dec. 20, 2014), and Fehring et al., Mol. Ther., 22 (4):811-20 (Apr. 22, 2014); Novobrantseva, Molecular Therapy-Nucleic Acids (2012)₁, e4; doi: 10.1038/mtna.2011.3; WO2012135025; US20140348900; US20140328759; US 20140308304; WO 2005/105152; WO 2006/069782; WO 2007/121947; US 2015/082080; US 20120251618; U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing a CRISPR-Cas system and/or component(s) thereof. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the CRISPR-Cas systems described herein.

Other non-limiting, exemplary liposomes can be those as set forth in Wang et al., ACS Synthetic Biology, 1, 403-07 (2012); Wang et al., PNAS, 113(11) 2868-2873(2016); Spuch and Navarro, Journal of Drug Delivery, vol. 2011, Article ID 469679, 12 pages, 2011. doi: 10.1155/2011/469679; WO 2008/042973; U.S. Pat. No. 8,071,082; WO 2014/186366; 20160257951; US20160129120; US20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMAo).

Other non-limiting, exemplary SNALPs that can be used to deliver the CRISPR-Cas systems described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375:1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2b (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US20020150626; Nair, J K et al., 2014, Journal of the American Chemical Society 136 (49), 16958-16961; Østergaard et al., Bioconjugate Chem., 2015, 26 (8), pp 1451-1455;

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US20100129793.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi:10.13140/RG.2.2.23912.16642. Other exemplary and non-limiting polymeric particles are described in US20170079916, US20160367686, US 20110212179, US20130302401, U.S. Pat. Nos. 6,007,845, 5,855,913, 5,985,309, 5,543,158, WO2012135025, US20130252281, US20130245107, US20130244279; US20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7 (12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29:341; El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, “Current trends in the use of liposomes for tumor targeting,” Nanomedicine (Lond). 8(9), doi: 10.2217/nnm.13.118(2013), and the documents it cites, all of which are incorporated herein by reference and the teachings of which can be applied and/or adapted for targeted delivery of one or more CRISPR-Cas molecules described herein. Mention is also made of International Patent Publication No. WO 2016/027264, and the documents it cites, all of which are incorporated herein by reference, the teachings of which can be applied and/or adapted for targeted delivery of one or more CRISPR-Cas molecules described herein. And mention is made of Lorenzer et al, “Going beyond the liver: Progress and challenges of targeted delivery of siRNA therapeutics,” Journal of Controlled Release, 203:1-15 (2015), and the documents it cites, all of which are incorporated herein by reference, the teachings of which can be applied and/or adapted for targeted delivery of one or more CRISPR-Cas molecules described herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bylayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody (ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG (SEQ ID NO: 73) such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA (SEQ ID NO: 74), cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein-discussed, there is an embodiment of the invention wherein the delivery system comprises such a targeting or active targeting moiety. Likewise, Table 2 provides exemplary targeting moieties that can be used in the practice of the invention an as to each an embodiment of the invention provides a delivery system that comprises such a targeting moiety.

TABLE 2

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine- | avβ3 | glioblastoma cells, human |
| aspartic acid-tyrosine- | | umbilical vein endothelial cells, |
| cysteine peptide | | tumor angiogenesis |
| (c(RGDyC)-LP) | | |
| ASSHN peptide | | endothelial progenitor cells; |
| | | anti-cancer |
| PR_b peptide | α₅β₁ integrin | cancer cells |
| AG86 peptide | α₆β₄ integrin | cancer cells |
| KCCYSL (P6.1 | HER-2 receptor | cancer cells |
| peptide) | | |
| affinity peptide LN | Aminopeptidase N | APN-positive tumor |
| (YEVGHRC) | (APN/CD13) | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2):189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more CRISPR-Cas molecules described herein.

Other exemplary targeting moieties are described elsewhere herein, such as epitope tags and the like.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extracellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl) phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 75)) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

### Engineered Cells

Described herein are various aspects of engineered cells that can include one or more of the programmable nuclease-peptidase composition or system polynucleotides, polypeptides, vectors, and/or vector systems, and/or programmable nuclease-peptidase composition or system particles (e.g., those particles, such as virus particles, produced from a programmable nuclease-peptidase composition or system polynucleotide and/or vector(s)) described elsewhere herein. In some embodiments, the engineered cells can express one or more of the programmable nuclease-peptidase composition or system polynucleotides and/or can produce one or more particles, such as virus particles or exosomes, containing a programmable nuclease-peptidase composition or system, which are described in greater detail herein. Such cells are also referred to herein as “producer cells”.

Described in certain example embodiments herein are engineered cells modified to express elements (i) and (iii) of the detection composition described herein. In certain example embodiments, where the engineered cells are further modified to express element (iv) of the detection composition described herein. In certain example embodiments, where the engineered cells are further modified to express element (ii) of the detection composition described herein.

In an embodiment, the invention provides a non-human eukaryotic organism; for example, a multicellular eukaryotic organism, including a eukaryotic host cell containing one or more components of an engineered delivery system described herein according to any of the described embodiments. In other aspects, the invention provides a eukaryotic organism; preferably a multicellular eukaryotic organism, comprising a eukaryotic host cell containing one or more components of a programmable nuclease-peptidase composition or system described herein according to any of the described embodiments. In some embodiments, the organism is a host of AAV.

The engineered cell can be any eukaryotic cell, including but not limited to, human, non-human animal, plant, algae, and the like.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see e.g., the American Type Culture Collection (ATCC) (Manassas, Va.)).

Further, the engineered cell may be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastomycota. fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientali, a.k.a. Pichia kudriav” evii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus (e.g., Aspergillus niger), Trichoderma spp. (e.g., Trichoderma reesei), Rhizopus spp. (e.g., “Rhizopus oryza”), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered delivery vesicle is produced it can package one or more molecules that are within the producer cell that can be related to the desired physiological and/or biological characteristic. In this context, the cargo molecules incorporated into the delivery vesicles can be capable of transferring the desired characteristic to a recipient cell.

In some embodiments, a cell can be obtained from a subject, modified such that it is an engineered delivery vesicle producer cell, and administered back to the subject from which it was obtained (autologous) or delivered to an allogenic subject. In other words, a producer cell described herein can be used in an autologous or allogenic context, such as in a cell therapy. In these embodiments, the cells can deliver a cargo, such as a therapeutic cargo or a cargo that can manipulate a cellular microenvironment within the subject.

Conventional viral and non-viral based gene transfer methods can be used to introduce nucleic acids (e.g., such as one or more of the polynucleotides of the engineered delivery system described herein) in cells or target tissues. Such methods can be used to administer nucleic acids encoding components of a nucleic acid-targeting system to cells in culture, or in a host organism. In some embodiments, a delivery is via a polynucleotide molecule (e.g., a DNA or RNA molecule) not contained in a vector. In some embodiments, delivery is via a vector. In some embodiments, delivery, is via viral particles. In aspects delivery is via a particle, (e.g., a nanoparticle) carrying one or more engineered delivery system polynucleotides, vectors, or viral particles. Particles, including nanoparticles, are discussed in greater detail elsewhere herein.

Vector delivery can be appropriate in some embodiments, where in vivo expression is envisaged. It will be appreciated that the engineered cells can be generated in vitro, ex vivo, in situ, or in vivo by delivery of one or more components of the engineered delivery systems as described elsewhere herein.

Suitable conventional viral and non-viral based methods of engineering cells to contain and/or express the engineered delivery system polynucleotides and/or vectors described herein are generally known in the art and/or described elsewhere herein.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a programmable nuclease-peptidase composition or system or component thereof described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including, but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, imaging agents, radiation sensitizers, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰ cells/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, radiation sensitizer, and any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Devices

Described in various embodiments herein are devices that are configured to carry out e.g., one or more of the assays, such as a detection, labeling, or screening, assay described herein. The devices can contain one or more of the programmable nuclease-peptidase compositions and/or systems or one or more components thereof. The assays or component thereof can be carried out on a device, such as tube, capillary, lateral flow strip, chip, cartridge or another device. The systems and/or assays described herein can be embodied on diagnostic devices. Devices can include very simple devices such as tubes for containing a single sample that contains all the reagents necessary to carry out a programmable nuclease-peptidase and/or CRISPR-Cas collateral activity reaction described herein and provide a result (such as a colometric, turbidity shift, or fluorescent signal) all within the single tube. Other devices can be complex fully automated devices that are capable of handling tens to thousands of samples at time. As is described in greater detail elsewhere herein, one or more compositions (e.g., sample preparation, target amplification reaction, and/or programmable nuclease-peptidase and/or CRISPR-Cas collateral activity detection reagents) can be included in the device. In some embodiments, they are included in one or more compartments and/or locations within the device in a free-dried, lyophilized or some other form. Devices can contain or be configured for optical-based readouts, lateral flow readouts, electrical readouts or others that are described herein and will be appreciated in view of the description provided herein.

### Discrete Volumes

In some embodiments the devices can include individual discrete volumes. In certain embodiments, an effector protein of the compositions or systems of the present invention is bound to each discrete volume in the device. Each discrete volume may comprise a different guide RNA specific for a different target molecule. In certain embodiments, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, an effector protein compositions or systems of the present invention is bound by an antibody specific for the effector protein compositions or systems of the present invention. Methods of binding a CRISPR enzyme has been described previously (see, e.g., US20140356867A1) and can be adapted for use with the present invention.

Several substrates and configurations of devices capable of defining multiple individual discrete volumes within the device may be used. As used herein “individual discrete volume” refers to a discrete space, such as a container, receptacle, or other arbitrary defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electromagnetic, or light illumination, or any combination thereof that can contain a target molecule and a indexable nucleic acid identifier (for example nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In specific embodiments, any of the applications, methods, or systems described herein requiring exact or uniform volumes may employ the use of an acoustic liquid dispenser.

### Samples

The device can be configured to hold, store, collect, receive, process and/or otherwise manipulate a sample and/or detect a component thereof. In some embodiments, the sample is a solid, semisolid, or liquid. In some embodiments, the sample is a biological sample. In some embodiments, the sample is obtained from a subject. In some embodiments, the sample is a bodily fluid. In some embodiments, the bodily fluid is saliva or nasal secretions. In some embodiments, the sample is not a bodily fluid but contains one or more cells from the subject, such as hair cells, skin cells, solid tissue or tumor cells. In some embodiments, the sample is obtained from a plant. In some embodiments, the sample is an environmental sample, such as air, soil, water, or a sample of molecules, organisms, viruses, and other particles present on an object surface. In some embodiments, the sample is a feedstuff or foodstuff or component thereof. Other exemplary samples that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

A sample for use with the invention may be a biological or environmental sample, such as a surface sample, a fluid sample, or a food sample (fresh fruits or vegetables, meats). Food samples may include a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, spinal fluid, cerebrospinal fluid, ascites, pleural effusion, seroma, pus, bile, aqueous or vitreous humor, transudate, exudate, or swab of skin or a mucosal membrane surface. In some embodiments, the biological sample is a bodily fluid. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In particular embodiments, the methods and systems can be utilized for direct detection from patient samples. In an aspect, the methods and systems can further allow for direct detection from patient samples with a visual readout to further facilitate field-deployability. In an aspect, a field deployable version can include, for example the lateral flow devices and systems as described herein, and/or colorimetric detection. The methods and systems can be utilized to distinguish multiple viral species and strains and identify clinically relevant mutations, important with viral outbreaks such as the coronavirus outbreak in Wuhan (2019-nCOV). In an aspect, the sample is from a nasophyringeal swab or a saliva sample. See, e.g., Wyllie et al., “Saliva is more sensitive for SARS-COV-2 detection in COVID-19 patients than nasopharyngeal swabs,” DOI: 10.1101/2020.04.16.20067835.

### Flexible Substrates

In certain example embodiments, the device comprises a flexible material substrate on which a number of spots or discrete volumes may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g., multiple clinical samples from different individuals) for the presence of the same target, or a limited number of target, or aliquots of a single sample (or multiple samples from the same source) for the presence of multiple different targets in the sample. In certain example embodiments, the elements of the systems described herein are freeze dried onto the paper or cloth substrate. Example flexible material based substrates that may be used in certain example devices are disclosed in Pardee et al. Cell. 2016, 165(5):1255-66 and Pardee et al. Cell. 2014, 159(4):950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719(2015). Further flexible based materials, including those suitable for use in wearable diagnostic devices are disclosed in Wang et al. “Flexible Substrate-Based Devices for Point-of-Care Diagnostics” Cell 34(11):909-21 (2016). Further flexible based materials may include nitrocellulose, polycarbonate, methylethyl cellulose, polyvinylidene fluoride (PVDF), polystyrene, or glass (see e.g., US20120238008). In certain embodiments, discrete volumes are separated by a hydrophobic surface, such as but not limited to wax, photoresist, or solid ink.

In some embodiments, the substrate, such as a flexible substrate, is a single use substrate, such as swab, strip, or cloth that is used to swab a surface or sample fluid or is placed in a prepared sample for detection by an assay described herein. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the compositions and systems of the present invention are designed to detect, for example identifying DNA SNPs that may be used to identify a suspect, or certain tissue or cell markers to determine the type of biological matter present in a sample. Likewise, the single use substrate could be used to collect a sample from a patient-such as a saliva sample from the mouth- or a swab of the skin. In other embodiments, a sample or swab may be taken of a meat product on order to detect the presence of absence of contaminants on or within the meat product.

### Microfluidic Devices

In certain example embodiments, the device is configured as a microfluidic device. It will be appreciated that the microfluidic device can incorporate a chip, cartridge, flexible substrate, lateral flow strip, and/or other components described elsewhere herein. In some embodiments, the microfluidic device can be configured to drive a sample through the device such that it contacts one or more detection reaction reagents (such as those that may be present on a flexible substrate within the device) and thus carries out a polypeptide cleavage detection reaction. In some embodiments, the microfluidic device is configured to generate and/or merge different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout in or allow to sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiators) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hou et al. “Direct Detection and drug-resistance profiling of bacteremias using inertial microfluidics” Lap Chip. 15(10):2297-2307 (2016). Further LOC embodiments are described elsewhere herein.

In one aspect, the embodiments disclosed herein are directed to a nucleic acid detection system comprising a programmable nuclease-peptidase composition or system of the present invention, one or more guide RNAs designed to bind to corresponding target molecules (e.g., a target nucleic acid), a reporter construct (also referred to herein as a detection construct in this context), and optional amplification reagents (discussed in greater detail elsewhere herein) to amplify target nucleic acid molecules and/or detectable signals in a sample. Detection compositions and detection constructs of the present invention are described in greater detail elsewhere herein.

### Lateral Flow Devices

In certain embodiments, the device is a lateral flow device. In certain embodiments, the detection assay can be provided on a lateral flow device, as described in International Publication WO 2019/071051, incorporated herein by reference. The lateral flow device can be adapted to detect one or more coronaviruses and/or other viruses in combination of the coronavirus. The lateral flow device may comprise a flexible substrate, such as a paper substrate or a flexible polymer-based substrate, which can include freeze-dried reagents for detection assays with a visual readout of the assay results. See, WO 2019/071051 at [0145]-[0151] and Example 2, specifically incorporated herein by reference. In an aspect, lyophilized reagents can include preferred excipients that aid in rate of reaction, specificity, or other variables. The excipients may comprise trehalose, histidine, and/or glycine. In certain embodiments, the coronavirus assay can be utilized with isothermal amplification reagents, allowing amplification without complex instrumentation that may be unavailable in the field, as described in WO 2019/071051. Accordingly, the assay can be adapted for field diagnostics, including use of visual readout on a lateral flow device, rapid, sensitive detection and can be deployed for early and direct detection. Colorimetric detection can be utilized and may be particularly suited for field deployable applications, as described in International Application PCT/US2019/015726, published as WO2019/148206. In particular, colorimetric detection can be as described in WO2019/148206 at FIGS. 102, 105, 107-111 and [00306]-[00324], incorporated herein by reference.

In one embodiment, the invention provides a lateral flow device comprising a substrate comprising a first end and a second end. The first end may comprise a sample loading portion, a first region comprising a detectable ligand, two or more effector systems of the present invention (e.g., programmable nuclease-peptidase compositions), two or more detection constructs, and one or more first capture regions, each comprising a first binding agent. The substrate may also comprise two or more second capture regions between the first region of the first end and the second end, each second capture region comprising a different binding agent. Each of the two or more effector systems of the present invention may comprise one or more effector proteins and one or more guide sequences, each guide sequence configured to bind one or more target molecules.

The device may comprise a lateral flow substrate for detecting a collateral polypeptide cleavage detection reaction. Substrates suitable for use in lateral flow assays are known in the art. These may include but are not necessarily limited to membranes or pads made of cellulose and/or glass fiber, polyesters, nitrocellulose, or absorbent pads (J Saudi Chem Soc 19(6):689-705; 2015), and other embodiments further described herein. The detection system, i.e., one or more programmable nuclease-peptidase compositions or systems and corresponding detection constructs are added to the lateral flow substrate at a defined reagent portion of the lateral flow substrate, typically on one end of the lateral flow substrate. Detection constructs used within the context of the present invention are described in greater detail elsewhere herein. The lateral flow substrate further comprises a sample portion. The sample portion may be equivalent to, continuous with, or adjacent to the reagent portion. In an aspect, the lateral flow substrate can be utilized for visual readout of a detectable signal in one-pot reactions, e.g., wherein steps of extracting nucleic acids, amplifying nucleic acids, and detecting are performed in the same or single individual discrete volume.

### Lateral Flow Substrate

In some embodiments, the device is a lateral flow device. In some embodiments, the lateral flow device can be composed of a composition or system and detection construct of the present invention described elsewhere herein and a lateral flow substrate for carrying out the detection reaction and/or nucleic acid release from the sample.

In certain example embodiments, a lateral flow device comprises a lateral flow substrate on which detection can be performed. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6):689-705; 2015).

Lateral support substrates comprise a first and second end, and one or more capture regions that each comprise binding agents. The first end may comprise a sample loading portion, a first region comprising a detectable ligand, two or more effector compositions or systems of the present invention, two or more detection constructs, and one or more first capture regions, each comprising a first binding agent. The substrate may also comprise two or more second capture regions between the first region of the first end and the second end, each second capture region comprising a different binding agent. Each of the two or more of the effector compositions or systems of the present invention may comprise one or more effector proteins (e.g., a RAMP and peptidase) and one or more guide sequences, each guide sequence configured to bind one or more target molecules. The lateral flow substrates may be configured to detect a peptidase activity detection reaction.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The embodiments disclosed herein can be prepared in freeze-dried format for convenient distribution and point-of-care (POC) applications. Such embodiments are useful in multiple scenarios in human health including, for example, viral detection, bacterial strain typing, sensitive genotyping, and detection of disease-associated cell free DNA. Accordingly, the lateral substrate comprising one or more of the elements of the system, including detectable ligands, effector systems, detection constructs and binding agents may be freeze-dried to the lateral flow substrate and packaged as a ready to use device. Alternatively, all or a portion of the elements of the system may be added to the reagent portion of the lateral flow substrate at the time of using the device.

### First End and Second End of the Substrate

The substrate of the lateral flow device comprises a first and second end. The effector composition or system of the present invention described herein (including any corresponding detection constructs) are added to the lateral flow substrate at a defined reagent portion of the lateral flow substrate, typically on a first end of the lateral flow substrate. Detection constructs used within the context of the present invention are described in greater detail elsewhere herein. The lateral flow substrate can further include a sample portion. The sample portion may be equivalent to, continuous with, or adjacent to the reagent portion.

In certain example embodiments, the first end comprises a first region. The first region comprises a detectable ligand, two or more effector systems of the present invention, two or more detection constructs, and one or more first capture regions, each comprising a first binding agent.

### Capture Regions

The lateral flow substrate can comprise one or more capture regions. In embodiments the first end of the lateral flow substrate comprises one or more first capture regions, with two or more second capture regions between the first region of the first end of the substrate and the second end of the substrate. The capture regions may be provided as a capture line, typically a horizontal line running across the device, but other configurations are possible. The first capture region is proximate to and on the same end of the lateral flow substrate as the sample loading portion.

### Binding Agents

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

A first binding agent that specifically binds the first molecule of the reporter construct is fixed or otherwise immobilized to the first capture region. The second capture region is located towards the opposite end of the lateral flow substrate from the first capture region. A second binding agent is fixed or otherwise immobilized at the second capture region. The second binding agent specifically binds the second molecule of the reporter construct, or the second binding agent may bind a detectable ligand. For example, the detectable ligand may be a particle, such as a colloidal particle, that when it aggregates can be detected visually, and generates a detectable positive signal. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding region comprises a second binding agent capable of specifically or non-specifically binding the detectable ligand on the antibody of the detectable ligand. Binding agents can be, for example, antibodies, that recognize a particular affinity tag. Such binding agents can further contain, for example, detectable labels, such as isotope labels and/or nucleic acid barcodes. A barcode is a short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the effector compositions and systems of the present invention may be used to detect the barcode.

### Detectable Ligands

The first region is loaded with a detectable ligand, such as those disclosed herein, for example a gold nanoparticle. The detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G. In some examples, the detectable ligand is a gold nanoparticle, which may be modified with a first antibody, such as an anti-FITC antibody.

### Lateral Flow Detection Constructs

The first region also comprises a detection construct. In one example embodiment, and for purposes of further illustration, the detection construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the detection construct is present it its initial state, i.e., in the absence of target, the FAM molecule on the first end will bind the anti-FITC antibody on the gold nanoparticle, and the biotin on the second end of the construct will bind the biotin ligand allowing for the detectable ligand to accumulate at the first test, generating a detectable signal. Generation of a detectable signal at the first band indicates the absence of the target ligand. In the presence of target, an effector complex of the present invention forms and an effector protein is activated resulting in cleavage of the detection construct containing a target polypeptide. In the absence of an intact detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FITC antibody on the colloidal gold. Therefore, in the presence of one or more targets, the detectable ligand will accumulate at the second band, indicating the presence of the one or more targets in the sample. Other detection constructs besides the one utilizing colloidal gold may be used in connection with the lateral flow devices herein. Other detection construct are described elsewhere herein.

In some embodiments, the first end of the lateral flow device comprises two detection constructs and each of the two detection constructs comprises a target polypeptide, comprising a first molecule on a first end and a second molecule on a second end. The first molecule and the second molecule may be linked by a polypeptide linker, such as a target polypeptide.

In some embodiments, the first molecule on the first end of the first detection construct may be FAM (or a first detection molecule) and the second molecule on the second end of the first detection construct may be biotin (or second detection molecule), or vice versa. In some embodiments, the first molecule on the first end of the second detection construct may be FAM and the second molecule on the second end of the second detection construct may be Digoxigenin (DIG), or vice versa.

In some embodiments, the first end may comprise three detection constructs, wherein each of the three detection constructs comprises a target polypeptide, comprising a first molecule on a first end and a second molecule on a second end. In specific embodiments, the first and second molecules on the detection constructs comprise Tye 665 and Alexa 488; Tye 665 and FAM, and Tye 665 and Digoxigenin (DIG), respectively. Other detection molecules are described elsewhere herein and can be used in connection with the lateral flow device described herein in view of the guiding principles above.

In some embodiments, the first end of the lateral flow device comprises two or more effector compositions or systems of the present invention. In some embodiments, such an effector system may include a one or more effector proteins (such as a RAMP and/or peptidase) and one or more guide sequences configured to bind to one or more target sequences.

### Samples

When utilizing the detection systems with a lateral flow substrate, samples to be screened are loaded at the sample loading portion of the lateral flow substrate. The samples must be liquid samples or samples dissolved in an appropriate solvent, usually aqueous. The liquid sample reconstitutes the detection reagents such that a detection reaction can occur. The liquid sample begins to flow from the sample portion of the substrate towards the first and second capture regions. Exemplary samples are described in greater detail elsewhere herein. See also WO 2019/071051, which is incorporated by reference herein.

### Cartridges and Chips

The cartridge, also referred to herein as a chip, according to the present invention comprises a series of components of ampoules and chambers that are communicatively coupled with one or more other components on the cartridge. The coupling is typically a fluidic communication, for example, via channels. The cartridge may comprise a membrane that seals one or more of the chambers and/or ampoules. In an aspect, the membrane allows for storage of reagents, buffers and other solid or fluid components which cover and seal the cartridge. The membrane can be configured to be punctured, pierced or otherwise released from sealing or covering one or more components of the cartridge by a means for releasing reagents. In some embodiments, the cartridge contains one or more wells, substrates (e.g., a flexible substrate), or other discrete volumes.

In some embodiments, the device is configured as lab-on-chip (LOC) diagnostic system. In some embodiments, the LOC is configured as a wireless lab-on-chip (LOC) diagnostic sensor system (see e.g., U.S. Pat. No. 9,470,699). In certain embodiments, RAMP and/or peptidase activity detection assay is performed in a LOC controlled and/or read by a wireless device (e.g., a cell phone, a personal digital assistant (PDA), a tablet) and results and/or reaction are reported to and/or measured by said device. In some embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, effector protein of the composition or system of the present invention, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA or polypeptide molecule. The conductive RNA or polypeptide molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process. Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

As noted above, certain embodiments enable the use of nucleic acid binding beads to concentrate target nucleic acid but that do not require elution of the isolated nucleic acid. Thus, in certain example embodiments, the cartridge may further comprise an activatable magnet, such as an electro-magnet. A means for activating the magnet may be located on the device, or the means for supplying the magnet or activating the magnet on the cartridge may be provided by a second device, such as those disclosed in further detail below.

The overall size of the device may be between 10, 15, 20, 25, 30, 35, 40, 45, 55, 60, 65, 70, 75, 80, 85, 90, 95, or 100 mm in width, and 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mm. The sizing of ampoules, chambers, and channels can be selected to be in line with the reaction volumes discussed herein and to fit within the general size parameters of the overall cartridge.

### Ampoules

The ampoules, also referred to as blisters, allow for storage and release of reagents throughout the cartridge. Ampoules can include liquid or solid reagents, for example, lysis reagents in one ampoule and reaction reagents in another ampoule. The reagents can be as described elsewhere herein and can be adapted for the use in the cartridge or microfluidic or other device. The ampoule may be sealed by a film that allows for the bursting, puncture or other release of the contents of the ampoules. See, e.g., Becker, H. & Gärtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Considerations for ampoules can include as discussed in, for example, Smith, S., et al., Blister pouches for effective reagent storage on microfluidic chips for blood cell counting. Microfluid Nanofluid 20, 163 (2016). DOI: 10.1007/s10404-016-1830-2. In an aspect, the seal is a frangible seal formed of a composite-layer film that is assembled to the cartridge main body or other part of the device. While referred to herein as an ampoule, the ampoule may comprise a cavity on a chip which comprises a sealed film that is opened by the release means.

### Chambers

The chip, microfluidic device, and/or other device described herein can have one or more chambers. The chambers on the chip may located and sized for fluidic communication via channels or other communication means with ampoules and/or other chambers on the chip. A chamber for receiving a sample can be provided. The sample can be injected, placed in a receptacle into the chamber for receiving a sample, or otherwise transferred to the chamber. A lysis chamber may comprise, for example, capture beads, that may be used for concentration and/or extraction of the desired target material from the sample. Alternatively, the beads may be comprised in an ampoule comprising lysis reagents that are in fluidic communication with the lysis chamber. An amplification chamber may also be provided with, for example, one or more lyophilized components of the system in the amplification chamber and/or communicatively connected to an ampoule comprising one or more components of the amplification reaction.

When the cartridge comprises a magnet, it may be configured near one or more of the chambers. In an aspect, the magnet is near the lysis well, and may be configured such that the device has a means for activating the magnet. Embodiments comprising a magnet in the cartridge may be utilized with methodologies using magnetic beads for extraction of particular target molecules.

### System for Detection Assays

A system configured for use with the cartridge and to perform an assay, also referred to as a sample analysis apparatus, detection system or detection device, is configured system to receive the cartridge and conduct an assay comprising isothermal amplification of nucleic acids and detection of target nucleic acids on the cartridge. The system may comprise: a body; a door housing which may be provided in an opened state or a closed state and configured to be coupled to the body of the sample analysis apparatus by a hinge or other closure means; a cartridge accommodating unit included in the detection system and configured to accommodate the cartridge. The system may further comprise one or more means for releasing reagents for extractions, amplification and/or detection; one or more heating means for extractions, amplification and/or detection, a means for mixing reagents for extraction, amplification, and/or detections, and/or a means for reading the results of the assay. The device may further comprise a user interface for programming the device and/or readout of the results of the assay.

### Means for Release of Reagents

The system may comprise means for releasing reagents for extraction, amplification and/or detection. Release of reagents can be performed by a crushing, puncturing, applying heat or pressure until burst, cutting, or other means for the opening of the ampoule and release of contents. e.g., Becker, H. & Gärtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Mechanical actuators

### Heating Means

The heating means or heating element can be provided, for example, by electrical or chemical elements. One or more heating means can be utilized, or circuits providing regulation of temperature to one or more locations within the detection device can be utilized. In an embodiment, the device is configured to comprise a heating means for heating the lysis (extraction) chamber and at the amplification chamber of the cartridge, sample vessel or other part of the device. In an aspect, the heating element is disposed under the extraction well. The system can be designed with one or more heating means for extraction, amplification and/or detection. In some embodiments, the device does not include a power source. In some embodiments, the heating element provides heat to a of about 65, 60, 55, 50, 45, 40, 39, 38, 37, 36, 35, 34, 33, 32, 31, 30, 29, 28, 27, 26, 25 degrees C. or less. In some embodiments, the device does not contain any heating element.

### Power Sources

In some embodiments, the device can include a power source. The power source can be coupled to one or more of the components of the device. In some embodiments, the power source is electrically coupled to one or more components of the device so as to provide electrical energy to the cone or more components. Suitable power sources that can be incorporated with the device are batteries (single use and rechargeable), solar powered power sources and batteries. In some embodiments, the power source can be coupled to an outside power source (e.g., an electric power grid) so as to recharge the on-board power source. In some embodiments, the device does not include a power source.

### Mixing Means

A means for mixing reagents for extraction, amplification and/or detection can be provided. A means for mixing reagents may comprise a means for mixing one or more fluids, or a fluid with a solid or lyophilized reaction mixture can also be provided. Means for mixing that disturb the laminar flow can be provided. In an aspect, the mixing means is a passive mixer, in another aspect, the mixing means is an active mixer. See, e.g., Nam-Trung Nguyen and Zhigang Wu 2005 J. Micromech. Microeng. 15 R1, doi: 10.1088/0960-1317/15/2/R01 for discussion of mixing approaches. In an aspect, the active mixer can be based on external sources such as pressure, temperature, hydrodynamics (with electrical or magnetic forces), dielectrophoresis, electrokinetics, or acoustics. Examples of passive mixing means can be provided by use of geometric approaches, such as a curved path or channel, see, e.g., U.S. Pat. No. 7,160,025, or an expansion/contraction of a channel cross section or diameter. When the cartridge is utilized with beads, channels and wells are configured and sized for the flow of beads.

### Means for Reading the Results of the Assay

A means for reading the results of the assay can be provided in the system. The means for reading the results of the assay will depend in part on the type of detectable signal generated by the assay. In particular embodiments, the assay generates a detectable fluorescent or color readout. In these instances, the means for reading the results of the assay will be an optic means, for example a single channel or multi-channel optical means such as a fluorimeter, colorimeter or other spectroscopic sensor.

A combination of means for reading the results of the assay can be utilized, and may include readings such as turbidity, temperature, magnetic, radio, or electrical properties and or optical properties, including scattering, polarization effects, etc.

The system may further comprise a user interface for programming the device and/or readout of the results of the assay. The user interface may comprise an LED screen. The system can be further configured for a USB port that can allow for docking of four or more devices.

In an aspect, the system comprises a means for activating a magnet that is disposed within or on the cartridge.

### Wearable Devices

The systems described herein, may further be incorporated into wearable medical devices that assess biological samples, such as biological fluids or an environmental sample, of a subject or in a subject's environment outside the clinic setting and report the outcome of the assay remotely to a central server accessible by a medical care professional. In some embodiments the device may include the ability to self-sample blood, saliva, sweat, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresies” to Andrew Conrad.

In some embodiments, the device is configured as a dosimeter or badge that serves as a sensor or indicator such that the wearer is notified of exposure to certain microbes or other agents. For example, the systems described herein may be used to detect a particular pathogen. Likewise, aptamer-based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous microbes as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

### Other Device Features

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried or lyophilized, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3):155-167).

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based detection constructs, use of a handheld UV light, or other suitable device, may be successfully used to detect a signal owing to the near complete quantum yield provided by quantum dots.

### Kits

Any of the compounds, compositions, formulations, particles, cells, devices, and combinations thereof, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, dipsticks, substrates, bottles, and the like. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, devices, described herein or a combination thereof contained therein, safety information regarding the content of the compounds, compositions, formulations, particles, devices, and cells described herein or a combination thereof contained therein, information regarding the dosages, working amounts, indications for use, and/or recommended treatment regimen(s) for the compound(s) formulations, devices, and combinations thereof contained therein. In some embodiments, the instructions can provide directions for sample collection, sample preparation, and/or use of the compounds, compositions, formulations, particles, devices and cells described herein or a combination thereof. In some embodiments, the instructions can be specific to the target(s) being detected by an effector composition or system of the present invention (e.g., a programmable nuclease-peptidase composition or system of the present invention).

### Methods of Use

### Methods of Modifying a Polypeptide

The compositions and systems of the present invention can be used to modify a polypeptide, such as a target polypeptide. In some embodiments, the target polypeptide is exogenous to a cell or organism. In some embodiments, the target polypeptide is endogenous or native to the cell or organism to which is introduced. In some embodiments, the exogenous target polypeptide is or is part of a detection construct of a system of a present invention. In some embodiments, such as in those methods where an endogenous or exogenous polypeptide is to be modified, compositions and systems of the present invention are configured to detect an exogenous target polynucleotide and thus activation of the system (and thus target polypeptide modification) can be controlled, at least in part, by controlling delivery of the target polynucleotide. In some embodiments, such as in those methods where an endogenous or exogenous polypeptide is to be modified, compositions and systems of the present invention are configured to detect an endogenous target polynucleotide, activation of the system and thus target polypeptide modification, occurs only in cells that contain the target polynucleotide, such as target RNA. In some embodiments target polypeptide modification is cleavage of the target polypeptide. In some embodiments, the target polypeptide is or is part of a detection construct. Such embodiments are described in greater detail elsewhere herein.

Described in certain example embodiments herein are methods of modifying a polypeptide comprising introducing into a sample having one or more target polynucleotide and target polypeptides, the programmable nuclease-peptidase compositions of the present invention; and activating the peptidase via sequence specific binding of the complex to the one or more target polynucleotides such that the peptidase then binds or interacts with the one or more target polypeptides resulting in modification of the one or more target polypeptides.

In certain example embodiments, the target polypeptide modification is cleavage of the target polypeptide. In certain example embodiments, the one or more target polypeptides are proenzymes, proproteins, and/or prodrugs, and the modification results in conversion of the proenzyme into an active enzyme, active protein, or active prodrug, respectively.

In certain example embodiments, introducing into the sample comprises in vitro, ex vivo, or in vivo delivery of the programable nuclease-peptidase composition into a cell or cell population.

In certain example embodiments, modification of the one or more target polypeptides results in activation or deactivation of one or more cell-signaling proteins and/or pathways. In some embodiments the cell-signaling protein is a protein involved in any one or more of the following pathways: Akt signaling pathway, AMPK signaling pathway, apoptosis signaling pathway, estrogen signaling pathway, insulin signaling pathway, JAK-STAT signaling pathway, MAPK signaling pathway, mTOR signaling pathway, NF-kappaB signaling pathway, Notch signaling pathway, p53 signaling pathway, TGF-beta signaling pathway, Toll-like receptor signaling pathway, VEGF signaling pathway, Wnt signaling pathway, hedgehog signaling pathway, a cytokine signaling pathway, a growth factor signaling pathway, a PI3K signaling pathway, a PKC signaling pathway, a MEK signaling pathway, a GSK3 beta signaling pathway, and/or the like. In some embodiments the cell-signaling protein is a protein involved in a cytokine receptor mediated pathway, a survival factor receptor mediated signaling pathway, a G-protein coupled receptor mediated signaling pathway, a growth factor receptor, mediated signaling pathway, an integrin mediated signaling pathway, a Frizzled receptor mediated signaling pathway, a Fas receptor mediated signaling pathway, a Patched/SMO receptor mediated signaling pathway.

In some embodiments, the cell signaling protein is JAK, STAT3, STAT5, Bcl-xL, cytochrome C, caspase 9, caspase 8, FADD, Bad, Bim, Bcl-2, PI3K, Akt, Akkalpha, IkapppaB, PLC, PKC, NFkappaB, G-protein, adenylate cyclase, PKA, Grb2, SOS, Ras, Raf, MEK, MEKK, MAPK, MKK, Myc, Mad, Max, CREB, ARF, mdm2, Mt, Bax, p53, ERK, Fos, a JNK, Jun, beta cadherin, TCF, a disheveled protein, GSK3beta, APC, Gli, p16, p15, p21, CycIE, CDK2, CycID, CDK4, Rb, E2F, a heat shock protein, insulin, ghrelin, preproghrelin, obestatin, neuropeptideY, erythropoietin, growth hormone, glucagon, vasopressin, calcitonin, adrenocortical hormone, amylin, angiotensin, atrial natriuretic peptide, cholecystokinin, gastrin, secretin, C-peptide, relaxin, pancreatic polypeptide, follicle-stimulating hormone, leptin, luteinizing hormone, melanocyte stimulating hormone, melanotropin, oxytocin, parathyroid hormone, prolactin, renin, somatostatin, thyroid-stimulating hormone, thyrotropin-releasing hormone, substance P, vasoactive intestinal peptide, IFN-gamma, MHC, TCRs, BCRs, activin, inhibin, bone-morophogeneitc proteins, TGF-beta, Smad transcription factors, RXR, IL-1, TNF, and/or the like.

In certain example embodiments, the one or more target polynucleotides are a specific transcript or set of transcripts and wherein modification of the one or more target polypeptides triggers cell death upon activating the peptidase in response to binding of the nuclease-peptidase to the specific transcript or set of transcripts. In certain embodiments, the guide molecule is configured to detect one or more mutations in the specific transcript or set of transcripts.

In some embodiments, the method of modifying a polypeptide can be used for, e.g., treating a disease or eliminating a pathogenic microorganism, by triggering apoptosis in the cell or otherwise disrupting signaling, or other function activity of the cell by modifying a polypeptide within said cell. Other applications of the methods of modifying a polypeptide will be appreciated in view of the description herein and, in particular, the polypeptides modified.

### Methods of Effector Activation and Biological Activity Modulation In Vivo/Ex Vivo

The programmable nuclease-peptidase compositions and components thereof can be included in an effector system as previously described. As previously described, the effector systems generally include a substrate for the peptidase of the programmable nuclease-peptidase composition that is coupled to an effector of interest. Cleavage of the substrate for the peptidase substrate directly or indirectly results in effector activity. Effector activity can result in a biological activity or modulation of a biological activity.

In some embodiments, one or more components of the effector system is expressed in an organism or a cell or cell population thereof. Activity of the effector of interest is stimulated and/or increased when the programmable nuclease-peptidase composition is activated by complexing, binding, and/or cleaving a target polynucleotide (e.g., a target RNA). In some embodiments, the target polynucleotide is endogenous to the cell in which the effector system is expressed. In some embodiments, the target polynucleotide is exogenous to the cell in which the effector system is expressed.

In some embodiments, the peptidase substrate-effector component of the effector system is separately expressed from the programmable nuclease-peptidase, the targeting polynucleotide, the target polynucleotide, or any combination thereof. Thus, in some embodiments, effector activity is controlled by controlling the timing of co-expression of the peptidase substrate-effector component of the effector system, the programmable nuclease-peptidase, the targeting polynucleotide, and the target polynucleotide.

The effector system can be used to modify a biological activity in a cell or cells so as to impart a functionality to an organism or cell(s) thereof and/or treat and/or prevent a disease, condition, infection, disorder, or any combination thereof in an organism or cell(s) thereof.

Exemplary effector systems and biological activities that can be modulated by the effector systems are described in greater detail elsewhere herein.

### Methods of Perturbation Screening

The programmable nuclease-peptidase and effector systems of the present invention can be used for functional screening, such as a method of perturbation screening. Described in several exemplary embodiments herein are methods for screening cell perturbations comprising introducing a perturbation to a cell population comprising engineered cells as described in greater detail elsewhere herein, along with any elements of the detection composition not already expressed by the engineered cells, and wherein the guide molecules are configured to detect one or more target transcripts associated with a specific cell type or cell state activating the peptidase via binding of the complex to one or more target polynucleotides such that the detection construct is modified by the activated peptidase to produce a detectable product and/or signal; and detecting an ability of the perturbation to modify expression of the one or more target transcripts by measuring a change in the detectable product and/or signal relative to a control. As is described in greater detail elsewhere herein, the engineered cells into which one or more perturbations are introduced contain a programmable nuclease-peptidase composition or system, such as a detection composition system, of the present invention. Detection constructs and detection assays and devices are described in greater detail elsewhere herein.

In general perturbation screening is a method of introducing one or more modifications (e.g., perturbations) into the genome and evaluating any change in gene and/or protein expression, phenotype, characteristic, functionality, and/or the like. Methods and tools for genome-scale screening of perturbations in cells, including single cells, using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; and International publication serial number WO/2017/075294). A similar approach may be used with the compositions and systems of the present invention provided herein.

The compositions and systems present invention is compatible with a detection reaction utilizing a detection composition of the present invention, such that genes, such as signature and/or target, genes may be perturbed, and the perturbation may be identified and assigned to the proteomic and gene expression readouts of single cells or cell populations. In certain embodiments, genes, such as signature or target genes, may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in certain embodiments, perturbation is used to discover novel drug and other targets to allow treatment of specific diseases, conditions, etc. at the population, subpopulation, and/or individual patient level.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a cell or single cell is determined by performing a detection reaction utilizing a detection composition described herein. In some embodiments, barcodes such as nucleic acid barcodes, can be included in the detection composition and/or detection construct such that single cells, or cell populations, detection compositions, detection constructs, target molecules, target polypeptides of the compositions of the present invention, can be distinguished and/or associated with a particular perturbation and/or result. In some embodiments, the barcode comprises a Unique Molecular Identifier (UMI).

Perturbations may be introduced into an engineered cell described herein using any suitable method or technique. In some embodiments, perturbations are introduced using a CRISPR-Cas system. In certain embodiments, a CRISPR system is used to create an INDEL at one or more target genes. In other embodiments, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/nature14136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimizing deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). Numerous genetic variants associated with disease phenotypes are found to be in non-coding region of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation. In one embodiment, a CRISPR system may be used to activate gene transcription. A nuclease-dead RNA-guided DNA binding domain, dCas9, tethered to transcriptional repressor domains that promote epigenetic silencing (e.g., KRAB) may be used for “CRISPRi” that represses transcription. To use dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required. In certain embodiments, the CRISPR-Cas system used to introduce the perturbation(s) includes a Cpf1.

The engineered cells into which the perturbation(s) are introduced may comprise a cell in a model non-human organism, a model non-human mammal, such as a mouse, non-human primate, and/or the like, that expresses a composition or system of the present invention or component(s) thereof, a mouse that expresses a composition or system of the present invention or component(s) thereof, a cell in vivo, or a cell ex vivo, or a cell in vitro (see e.g., WO 2014/093622 (PCT/US13/074667); US Patent Publication Nos. 20120017290 and 20110265198 assigned to Sangamo BioSciences, Inc.; US Patent Publication No. 20130236946 assigned to Cellectis; Platt et al., “CRISPR-Cas9 Knockin Mice for Genome Editing and Cancer Modeling” Cell (2014), 159(2): 440-455; “Oncogenic models based on delivery and use of the crispr-cas systems, vectors and compositions” WO2014204723A1 “Delivery and use of the crispr-cas systems, vectors and compositions for hepatic targeting and therapy” WO2014204726A1; “Delivery, use and therapeutic applications of the crispr-cas systems and compositions for modeling mutations in leukocytes” WO2016049251; and Chen et al., “Genome-wide CRISPR Screen in a Mouse Model of Tumor Growth and Metastasis” 2015, Cell 160, 1246-1260), which can be adapted for use with the present invention described herein.

In some embodiments, the cell or cells are tumor cells, such as tumor cells obtained from a subject in need of treatment. In some embodiments, the subject has or is suspected of having a cancer.

In one embodiment, one or more perturbations are introduced into one or more protein-coding genes or non-protein-coding DNA. In some embodiments a CRISPR system may be used to knockout protein-coding genes by frameshifts, point mutations, inserts, or deletions. An extensive toolbox may be used for efficient and specific CRISPR system mediated knockout as described herein, including a double-nicking CRISPR to efficiently modify both alleles of a target gene or multiple target loci and a smaller Cas protein for delivery on smaller vectors (Ran, F. A., et al., In vivo genome editing using Staphylococcus aureus Cas9. Nature. 520, 186-191 (2015)). A genome-wide sgRNA mouse library (˜10 sgRNAs/gene) may also be used in a mouse that expresses a suitable Cas protein (see, e.g., WO2014204727A1).

In one embodiment, perturbation is by deletion of regulatory elements. Non-coding elements may be targeted by using pairs of guide RNAs to delete regions of a defined size, and by tiling deletions covering sets of regions in pools.

In one embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus (AAV). Other suitable vectors are provided elsewhere herein.

In some embodiments, perturbations are introduced into primary mouse T-cells such as by viral vector delivery of a CRISPR system or by a method described by Hendel et al, (Nature Biotechnology 33, 985-989 (2015) doi: 10.1038/nbt.3290). Such methods may be adapted to other cell types.

In certain embodiments, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed.

Not being bound by a theory, perturbation studies targeting the genes and gene signatures described herein could (1) generate new insights regarding regulation and interaction of molecules within the system that contribute to suppression of an immune response, such as in the case within the tumor microenvironment, and (2) establish potential therapeutic targets or pathways that could be translated into clinical application.

### Methods of Detecting Target Polynucleotides

The programmable nuclease-peptidase compositions and detection compositions described herein can be used in a method of detecting target polynucleotides, such as those present in a sample. Such methods employ one or more of the detection compositions described herein, systems, cells, described herein, and/or devices described herein. Exemplary aspects of the method, e.g., detection constructs and detectable signal generation, are also described in greater detail elsewhere herein. Generally, a method of detection includes complexing of a programmable nuclease-peptidase composition (such as a detection composition) of the present invention with a guide molecule and specifically binding a target polynucleotide. Without being bound by theory, binding of a target polynucleotide activates a peptidase of the system, which cleaves or otherwise modifies a target polypeptide of a detection construct to produce a detectable signal thereby indicated detection of a target polynucleotide. Detection can occur, in vitro, in vivo, in situ, or ex vivo. The system can be configured to detect one or more, e.g., 1, 2, 3, 4, 5, 6, 7, 8, 9, 10 or more different target polynucleotides.

Described in certain example embodiments herein are methods of detecting target polynucleotides in samples comprising combining a sample or a component thereof with the detection composition as described in greater detail elsewhere herein; and activating the peptidase via binding of the complex to one or more target polynucleotides such that the detection construct is modified by the activated peptidase such that a detectable product and/or signal is produced, thereby detecting the target polynucleotide in the sample. In some embodiments, the method further comprising amplifying and/or enriching the target polynucleotide. In some embodiments, activating the peptidase further results in activation or generation of one or more signal amplification molecules.

Methods employing Cas13 or Cas12 based detection can be used as a general guide for configuration and design of a method, including sample processing, for target nucleic acid detection methods employing the programmable nuclease-peptidase compositions of the present invention as they related to target nucleic acid preparation and processing (see e.g., Jong et al. N Engl J Med. 2020. 383(15):1492-1494; Broughton, et al. CRISPR-Cas12-based detection of SARS-COV-2. Nat Biotechnol (2020), doi: 10.1038/s41587-020-0513-4 (DETECTR detection); Gootenberg et al., Science. 2018 Apr. 27; 360(6387):439-444. doi: 10.1126/science.aaq0179 (multiplexing lateral flow platform for point-of-care diagnostics); and Chen, et al., Science. 2018 Apr. 27; 360(6387):436-439. doi: 10.1126/science.aar6245 (Cas12 detection), Myrhvold et al., Science 27 Apr. 2018:360:6387, pp. 444-448; doi: 10.1126/science.aas8836 (field deployable viral diagnostics), Joung et al., Point-of-care testing for COVID-19 using SHERLOCK diagnostics” doi: 10.1101/2020.05.04.20091231; Schmid-Burgk, et al., “LAMP-Seq: Population-Scale COVID-19 Diagnostics Using Combinatorial Barcoding,” doi: 10.1101/2020.04.06.025635, Gootenberg, 2018; Gootenberg, et al, Science. 2017 Apr. 28; 356(6336):438-442 (2017); Myhrvold, et al., Science 360, 444-448 (2018)). Nucleic acid detection with SHERLOCK relies on the collateral activity of Type VI and Type V Cas proteins, such as Cas13 and Cas12, which unleashes promiscuous cleavage of reporters upon target detection (Gooteneberg et al., 2018) (Abudayyeh, et al., Science. 353(6299)(2016); East-Seletsky et al. Nature 538:270-273 (2016); Smargon et al. Mol Cell 65(4):618-630 (2017)), Gootenberg, 2018; Myhrvold et al. Science 360(6387):444-448 (2018); Gootenberg, 2017; Chen et al. Science 360(6387):436-439(2018); Li et al. Cell Rep 25(12):3262-3272 (2018); Li et al. Nat Protoc 13(5):899-914 (2018), WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. Pat. No. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6, Science. 2018 Apr. 27; 360(6387):439-444; Gootenberg J S, et al., Nucleic acid detection with CRISPR-Cas13a/C2c2, Science. 2017 Apr. 28; 356(6336):438-442; Abudayyeh O O, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh O O, et al., C2c2 is a single-component programmable RNA-guided RNA-targeting CRISPR effector, Science. 2016 Aug. 5; 353(6299): aaf5573; Yang L, et al., Engineering and optimising deaminase fusions for genome editing. Nat Commun. 2016 Nov. 2; 7:13330, Myhrvold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety. Differences in the mechanism of nucleic acid detection and signal generation by a detection construct from such guiding methods and systems will be readily apparent in view of the description herein.

The low cost and adaptability of the assay platform described herein lends itself to a number of applications including (i) general viral RNA/DNA quantitation, (ii) rapid, multiplexed RNA/DNA expression detection, and (iii) sensitive detection of target nucleic acids in both clinical and environmental samples. Additionally, the systems disclosed herein may be adapted for detection of transcripts within biological settings, such as cells. Given the highly specific nature of the effectors described herein, it may be possible to track allelic specific expression of transcripts or disease-associated mutations and/or the presence of microorganisms in live cells.

In certain example embodiments, a single guide RNA specific to a single target is placed in separate volumes. Each volume may then receive a different sample or aliquot of the same sample. In certain example embodiments, multiple guide RNA each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, U, or T. Accordingly, guide RNAs that are all, or comprise a substantial portion, of a single nucleotide may be generated, each with a different fluorophore. In this way up to four different targets may be screened in a single individual discrete volume.

In some embodiments, the CRISPR effector systems and methods herein are capable of detecting down to at least attomolar concentrations of target molecules, such as viral polynucleotides. In some embodiments, the CRISPR effector systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of viral DNA or RNA per microliter (cp/μL). In some embodiments, the CRISPR effector systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of viral DNA or RNA per microliter (cp/μL) using a fluorescent or colorimetric readout.

In some embodiments, the detection reaction can occur as a two-step reaction in which amplification of target(s) and target detection via the effector composition/system of the present invention occur in separate reactions. In some embodiments, the detection reaction (including any target and/or signal amplification) can occur as a single, one-pot reaction. In some embodiments where the detection reaction is a one-pot reaction, target amplification is achieved using LAMP or RPA (see also below).

In some embodiments, the total time to perform the detection method (from sample preparation to detection) can be greater than 0 hours but less than about 4, 3.5, 3, 2.5, 2, 1.5, 1, or 0.5 hours. In some embodiments, the total time to perform the detection method (from sample preparation to detection) can occur within about 20 to 120 minutes, such as within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, to/or 120 minutes. In some embodiments, the total time to perform the detection method (from sample preparation to detection) can occur within about 20 to about 60 minutes, e.g. within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or/to 60 minutes. In some embodiments, the total time to perform the detection method (from sample preparation to detection) can occur within about 20 to about 45 minutes, e.g. within about 20, 21, 22, 23, 24, 25, 26, 27, 28, 29, 30, 31, 32, 33, 34, 35, 36, 37, 38, 39, 40, 41, 42, 43, 44, and/or 45 minutes. In some embodiments, the total time to perform the detection method (from sample preparation to detection) can occur within about 20 to about 30 minutes, e.g., within about 20, 21, 22, 23, 24, 25, 26, 27, 28, 29, and/or 30 minutes.

In some embodiments, the detection reaction can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the detection reaction can occur within about 1 to about 45 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the reaction can occur within about 1 to about 30 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the detection reaction can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the detection reaction can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the detection reaction can occur within about 1 to about 15 minutes, e.g., within about 1, 2, 3, 4, 5, 6, 7, 8, 9, 10, 11, 12, 13, to/or about 15 minutes. In some embodiments, the detection reaction can occur within about 1 to about 10 minutes, e.g., within about 1, 2, 3, 4, 5, 6, 7, 8, 9, to/or about 10 minutes. In some embodiments, the detection reaction can occur within about 1 to about 5 minutes, e.g., within about 1, 2, 3, 4, to/or about 5 minutes.

### Sample and Target Nucleic Acid Processing, Isolation, Amplification, and Enrichment

In some embodiments, a sample and/or target polynucleotides is/are isolated, amplified, and/or enriched, and/or otherwise processed prior to amplification, enrichment, and/or detection. Such processing can include lysis of one or more cells or particles (e.g., viruses, exosomes, virus like particles, and/or the like) present in the sample to release target nucleic acids. In some embodiments, nucleic acids are isolated or otherwise separated from the one or more cells or particles (e.g., viruses, exosomes, virus like particles, and/or the like) present in the sample or sample lysate. In some embodiments, the method does not require or include extraction of the nucleic acids from the sample prior to amplification and/or target detection. In some embodiments, the sample preparation (e.g., lysis) and amplification occur in the same reaction vessel or location.

In some embodiments, the sample preparation (e.g., lysis), target amplification, and detection occur in the same reaction vessel or location. In some embodiments, the reaction vessel or location contains the sample preparation, amplification, and/or detection compositions and/or systems. In these embodiments, the sample can be added to the vessel and processing, amplification and detection can occur in the same vessel with no requirement to remove or add reagents to the vessel prior to obtaining a result. In some embodiments, the reagents, compositions, and systems are included in a vessel in a dehydrated (e.g., freeze dried, lyophilized, etc.) form and can be reconstituted when ready to use.

In some embodiments, the method includes preparation of the reagents for one or more steps, such as sample preparation, amplification, and/or detection, for storage. Such storage preparation can include, but is not limited to lyophilizing, freeze drying, or otherwise dehydrating them. They can be prepared for storage inside of individual reaction vessels or locations within a device or other vessel. In some of these embodiments, the reagents, compositions, systems or combinations thereof are e.g., lyophilized or freeze dried inside of the reaction vessel or at the specific discreet locations on a substrate or otherwise in a device. They can be stored at a suitable temperature ranging from ambient temperature (e.g., about 25-32 degrees C.) to about −20 or −80 degrees Celsius. In some embodiments, they are stored for 1, 2, 3, 4, 5, 6, 7, 8, 9, 10, 11, or 12 days, weeks, months or years. In some embodiments, the reagents, compositions, systems or combinations thereof are prepared and stored at about 4 degrees C. for about, 2, 3, 4, 5, 6, 7, 8, 9, 10, 11, or 12 days, weeks, months or years or more.

Due to the sensitivity of said systems, a number of applications that require from the rapid and sensitive detection may benefit from the embodiments disclosed herein and are contemplated to be within the scope of the invention. Further, any of the sample and/or nucleic acid processing methods described in this section can be applied, as relevant, to other methods employing the programmable nuclease-peptidase and detection compositions of the present invention herein. It is not intended to limit these features to just methods specifically designed to detect target polynucleotides.

### Sample Preparation

In some embodiments, the sample preparation can include release of polynucleotides (e.g., DNA and/or RNA) from cells and/or microorganisms, such as viruses, bacteria, engineered or other cells, particles (e.g., exosomes) etc., present in the sample. In some embodiments, the sample preparation can include virus, bacteria, inactivation and/or nuclease inactivation. The step of sample preparation can occur prior to any target amplification and/or detection. In some embodiments, sample preparation can include nuclease inactivation and/or viral inactivation by 1, 2, 3, 4 or more thermal (heat or cold) inactivation steps, chemical inactivation steps, biologic inactivation, physiologic inactivation, physical inactivation steps, or any combination thereof. The phrase “physiological inactivation” refers to conditions that deviate from the normal working physiological conditions (e.g., pH, osmolarity, temperature, salinity, etc.) necessary for causing or maintaining the activation of a component (e.g., an enzyme) present in a sample that result in the inactivation or inhibition of the function or activity of the component. Inactivation can, in some embodiments, result in lysis of the cells, microorganisms, viruses, and/or particles. In some embodiments, the same methods and reagents can be applied to other microbes (e.g., bacteria and eukaryotic cells).

Amplification and Enrichment of Target and/or Signal

### Target Amplification

In certain example embodiments, target RNAs and/or DNAs may be amplified prior to activating the effector protein of the composition and/or system of the present invention. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In certain embodiments, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), or a helicase-based amplification method (see e.g. WO 2020/006036, which is incorporated by reference herein as if expressed in its entirety). In some embodiments, amplification is via LAMP. In some embodiments, amplification is via RPA.

In certain example embodiments, the RNA or DNA amplification is nucleic acid sequence-based amplification is NASBA, which is initiated with reverse transcription of target RNA by a sequence-specific reverse primer to create an RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the effector protein effector protein of the composition and/or system of the present invention and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and an RNA polymerase promoter. After, or during, the RPA reaction, a RNA polymerase is added that will produce RNA from the double-stranded DNA templates. The amplified target RNA can then in turn be detected by the effector system effector protein of the composition and/or system of the present invention. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or apatamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification reagents can include one or more primers and/or probes optimized for amplification of a target sequence by one or more of the amplification methods previously described. Primer and probe design for the methods described herein will be within the purview of one of ordinary skill in the art in view of the context and disclosure only provided herein.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

In some embodiments, the amplification reagent or component thereof is shelf-stable. In some embodiments, the amplification reagent or component thereof is shelf-stable at ambient temperature.

### Target Polynucleotide Enrichment

In certain example embodiments, target RNA or DNA may first be enriched prior to detection or amplification of the target RNA or DNA. In certain example embodiments, this enrichment may be achieved by binding of the target nucleic acids by a CRISPR effector system or other suitable affinity based capture strategy capable of specifically capturing target nucleic acids so as to allow separation from non-target nucleic acids.

Current target-specific enrichment protocols require single-stranded nucleic acid prior to hybridization with probes. Among various advantages, the present embodiments can skip this step and enable direct targeting to double-stranded DNA (either partly or completely double-stranded). In addition, the embodiments disclosed herein are enzyme-driven targeting methods that offer faster kinetics and easier workflow allowing for isothermal enrichment. In certain example embodiments, a set of guide RNAs to different target nucleic acids are used in a single assay, allowing for detection of multiple targets an/or multiple variants of a single target.

In certain example embodiments, a dead CRISPR effector protein may bind the target nucleic acid in solution and then subsequently be isolated from said solution. For example, the dead CRISPR effector protein bound to the target nucleic acid, may be isolated from the solution using an antibody or other molecule, such as an aptamer, that specifically binds the dead CRISPR effector protein.

In other example embodiments, the dead CRISPR effector protein may bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the solid support comprises a patterned surface suitable for immobilization of molecules in an ordered pattern. In certain embodiments a patterned surface refers to an arrangement of different regions in or on an exposed layer of a solid support. In some embodiments, the solid support comprises an array of wells or depressions in a surface. The composition and geometry of the solid support can vary with its use. In some embodiments, the solids support is a planar structure such as a slide, chip, microchip and/or array. As such, the surface of the substrate can be in the form of a planar layer. In some embodiments, the solid support comprises one or more surfaces of a flowcell. The term “flowcell” as used herein refers to a chamber comprising a solid surface across which one or more fluid reagent can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In some embodiments, the solid support or its surface is non-planar, such as the inner or outer surface of a tube or vessel. In some embodiments, the solid support comprise microspheres or beads. “Microspheres,” “bead,” “particles,” are intended to mean within the context of a solid substrate to mean small discrete particles made of various material including, but not limited to, plastics, ceramics, glass, and polystyrene. In certain embodiments, the microspheres are magnetic microspheres or beads. Alternatively or additionally, the beads may be porous. The bead sizes range from nanometers, e.g., 100 nm, to millimeters, e.g., 1 mm.

A sample containing, or suspected of containing, the target nucleic acids may then be exposed to the substrate to allow binding of the target nucleic acids to the bound dead CRISPR effector protein. Non-target molecules may then be washed away. In certain example embodiments, the target nucleic acids may then be released from the CRISPR effector protein/guide RNA complex for further detection using the methods disclosed herein. In certain example embodiments, the target nucleic acids may first be amplified as described herein.

In certain example embodiments, the CRISPR effector may be labeled with a binding tag. In certain example embodiments the CRISPR effector may be chemically tagged. For example, the CRISPR effector may be chemically biotinylated. In another example embodiment, a fusion may be created by adding additional sequence encoding a fusion to the CRISPR effector. One example of such a fusion is an AviTag™, which employs a highly targeted enzymatic conjugation of a single biotin on a unique 15 amino acide peptide tag. In certain embodiments, the CRISPR effector may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the CRISPR effector system once it has bound a target nucleic acid or to fix the CRISPR effector system on the solid substrate.

In certain example embodiments, the guide RNA may be labeled with a binding tag. In certain example embodiments, the entire guide RNA may be labeled using in vitro transcription (IVT) incorporating one or more biotinylated nucleotides, such as, biotinylated uracil. In some embodiments, biotin can be chemically or enzymatically added to the guide RNA, such as, the addition of one or more biotin groups to the 3′ end of the guide RNA. The binding tag may be used to pull down the guide RNA/target nucleic acid complex after binding has occurred, for example, by exposing the guide RNA/target nucleic acid to a streptavidin coated solid substrate.

Accordingly, in certain example embodiments, an engineered or non-naturally-occurring CRISPR effector may be used for enrichment purposes. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of the RNA strand at the target locus of interest. In a preferred embodiment, the one or more mutations may comprise two mutations. In a preferred embodiment the one or more amino acid residues are modified in a C2c2 effector protein, e.g., an engineered or non-naturally-occurring effector protein or C2c2. In particular embodiments, the one or more modified of mutated amino acid residues are one or more of those in C2c2 corresponding to R597, H602, R1278 and H1283 (referenced to Lsh C2c2 amino acids), such as mutations R597A, H602A, R1278A and H1283A, or the corresponding amino acid residues in Lsh C2c2 orthologues.

In particular embodiments, the one or more modified of mutated amino acid residues are one or more of those in C2c2 corresponding to K2, K39, V40, E479, L514, V518, N524, G534, K535, E580, L597, V602, D630, F676, L709, I713, R717 (HEPN), N718, H722 (HEPN), E773, P823, V828, 1879, Y880, F884, Y997, L1001, F1009, L1013, Y1093, L1099, L1111, Y1114, L1203, D1222, Y1244, L1250, L1253, K1261, I1334, L1355, L1359, R1362, Y1366, E1371, R1372, D1373, R1509 (HEPN), H1514 (HEPN), Y1543, D1544, K1546, K1548, V1551, I1558, according to C2c2 consensus numbering. In certain embodiments, the one or more modified of mutated amino acid residues are one or more of those in C2c2 corresponding to R717 and R1509. In certain embodiments, the one or more modified of mutated amino acid residues are one or more of those in C2c2 corresponding to K2, K39, K535, K1261, R1362, R1372, K1546 and K1548. In certain embodiments, said mutations result in a protein having an altered or modified activity. In certain embodiments, said mutations result in a protein having a reduced activity, such as reduced specificity. In certain embodiments, said mutations result in a protein having no catalytic activity (i.e., “dead” C2c2). In an embodiment, said amino acid residues correspond to Lsh C2c2 amino acid residues, or the corresponding amino acid residues of a C2c2 protein from a different species.

The above enrichment systems may also be used to deplete a sample of certain nucleic acids. For example, guide RNAs may be designed to bind non-target RNAs to remove the non-target RNAs from the sample. In one example embodiment, the guide RNAs may be designed to bind nucleic acids that do carry a particular nucleic acid variation. For example, in a given sample a higher copy number of non-variant nucleic acids may be expected. Accordingly, the embodiments disclosed herein may be used to remove the non-variant nucleic acids from a sample, to increase the efficiency with which the detection effector system effector protein of the composition and/or system of the present invention can detect the target variant sequences in a given sample.

Amplification and/or Enhancement of Detectable Signal

In certain example embodiments, further modification or reagents may be introduced that further amplify the detectable positive signal. For example, activated effector protein peptidase activation may be used to generate a secondary target or additional guide sequence, or both. In one example embodiment, the reaction solution would contain a secondary target polypeptide that is spiked in at high concentration. The secondary target polypeptide may be distinct from the primary target polypeptide (i.e., the first target polypeptide e for which the assay is designed to detect) and in certain instances may be common across all reaction volumes. A secondary polypeptide may include a protecting group such that is not active until acted upon by the effector protein. Cleavage of the protecting group by an activated effector protein (i.e., after activation by formation of complex with the primary target(s) in solution) and formation of a complex with free effector protein in solution and activation from the spiked in secondary target polypeptide.

In some embodiments another CRISPR system can be used to enrich or amplify the detectable signal. In some embodiments the effector system(s) of the present invention that is/are activated upon target binding can produce, such as via collateral (e.g., peptidase) activity, species that can activate (or be targets of) a second CRISPR system (such as a Cas-12 or Cas-13 detection system) thus amplifying the signal for detection. In some embodiments a CRISPR type-III effector can be used as the signal amplifying system. In some embodiments, the type III effector is Csm6, which is which is activated by cyclic adenylate molecules or linear adenine homopolymers terminated with a 2′,3′-cyclic phosphate. In some embodiments, the first CRISPR system includes a Cas13 (e.g., Cas 13a, 13b, 13c, or 13d) and/or a Cas 12a effector(s) and the amplification system or molecule is or includes Csm6. See also Gootenberg et al. 2018. Science. 360:439-44 and WO 2019/051318, which are incorporated by reference herein as if expressed in their entireties.

As demonstrated in the Working Examples, Up1 can bind transcription initiation factor Up3. In some embodiments, Up3 or fragment thereof is used as the secondary polypeptide to amplify the signal by the Up1. In some embodiments, Up3 is coupled to one or more signal molecules (e.g., molecules capable of producing a detectable signal).

### Exemplary Applications of the Target Polynucleotide Detection Methods

### Microbe and Virus Detection and Applications

In certain example embodiments, the systems, devices, and methods, disclosed herein are directed to detecting the presence of one or more microbial agents in a sample, such as a biological sample obtained from a subject. In certain example embodiments, the microbe may be a bacterium, a fungus, a yeast, a protozoa, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g., bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a POC device, the embodiments disclosed herein may be used guide therapeutic regimens, such as selection of the appropriate antibiotic or antiviral. The embodiments disclosed herein may also be used to screen environmental samples (air, water, surfaces, food etc.) for the presence of microbial contamination.

Disclosed is a method to identify microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multi-level analysis can be performed for a particular subject in which any number of microbes can be detected at once. In some embodiments, simultaneous detection of multiple microbes may be performed using a set of probes that can identify one or more microbial species.

Multiplex analysis of samples enables large-scale detection of samples, reducing the time and cost of analyses. However, multiplex analyses are often limited by the availability of a biological sample. In accordance with the invention, however, alternatives to multiplex analysis may be performed such that multiple effector proteins can be added to a single sample and each detection construct may be combined with a separate quencher dye. In this case, positive signals may be obtained from each quencher dye separately for multiple detection in a single sample.

Disclosed herein are methods for distinguishing between two or more species of one or more organisms in a sample. The methods are also amenable to detecting one or more species of one or more organisms in a sample.

### Microbe Detection

In some embodiments, a method for detecting microbes in samples is provided comprising distributing a sample or set of samples into one or more individual discrete volumes, the individual discrete volumes comprising a CRISPR system as described herein; incubating the sample or set of samples under conditions sufficient to allow binding of the one or more guide RNAs to one or more microbe-specific targets; activating the CRISPR effector protein via binding of the one or more guide RNAs to the one or more target molecules, wherein activating the CRISPR effector protein results in modification of the RNA-based detection construct such that a detectable positive signal is generated; and detecting the detectable positive signal, wherein detection of the detectable positive signal indicates a presence of one or more target molecules in the sample. The one or more target molecules may be mRNA, gDNA (coding or non-coding), trRNA, or RNA comprising a target nucleotide tide sequence that may be used to distinguish two or more microbial species/strains from one another. The guide RNAs may be designed to detect target sequences. The embodiments disclosed herein may also utilize certain steps to improve hybridization between guide RNA and target RNA sequences. Methods for enhancing ribonucleic acid hybridization are disclosed in WO 2015/085194, entitled “Enhanced Methods of Ribonucleic Acid Hybridization” which is incorporated herein by reference. The microbe-specific target may be RNA or DNA or a protein. If DNA method may further comprise the use of DNA primers that introduce a RNA polymerase promoter as described herein. If the target is a protein then aptamers can be utilized and the method includes one or more specific to protein detection described herein.

### Detection of Single Nucleotide Variants

In some embodiments, one or more identified target sequences may be detected using guide RNAs that are specific for and bind to the target sequence as described herein. The systems and methods of the present invention can distinguish even between single nucleotide polymorphisms present among different microbial species and therefore, use of multiple guide RNAs in accordance with the invention may further expand on or improve the number of target sequences that may be used to distinguish between species. For example, in some embodiments, the one or more guide RNAs may distinguish between microbes at the species, genus, family, order, class, phylum, kingdom, or phenotype, or a combination thereof. This application can also apply to non-microbial cells, such as human cells in detection of disease or genotyping.

Detection Based on rRNA Sequences

In certain example embodiments, the devices, systems, and methods disclosed herein may be used to distinguish multiple microbial species in a sample. In certain example embodiments, identification may be based on ribosomal RNA sequences, including the 16S, 23S, and 5S subunits. Methods for identifying relevant rRNA sequences are disclosed in U.S. Patent Application Publication No. 2017/0029872. In certain example embodiments, a set of guide RNA may designed to distinguish each species by a variable region that is unique to each species or strain. Guide RNAs may also be designed to target RNA genes that distinguish microbes at the genus, family, order, class, phylum, kingdom levels, or a combination thereof. In certain example embodiments where amplification is used, a set of amplification primers may be designed to flanking constant regions of the ribosomal RNA sequence and a guide RNA designed to distinguish each species by a variable internal region. In certain example embodiments, the primers and guide RNAs may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv: 1307.8690 [q-bio.GN].

In certain example embodiments, a method or diagnostic is designed to screen microbes across multiple phylogenetic and/or phenotypic levels at the same time. For example, the method or diagnostic may comprise the use of multiple detection compositions or systems of the present invention with different guide RNAs. A first set of guide RNAs may distinguish, for example, between mycobacteria, gram positive, and gram-negative bacteria. These general classes can be even further subdivided. For example, guide RNAs could be designed and used in the method or diagnostic that distinguish enteric and non-enteric within gram negative bacteria. A second set of guide RNA can be designed to distinguish microbes at the genus or species level. Thus, a matrix may be produced identifying all mycobacteria, gram positive, gram negative (further divided into enteric and non-enteric) with each genus of species of bacteria identified in a given sample that fall within one of those classes. The foregoing is for example purposes only. Other means for classifying other microbe types are also contemplated and would follow the general structure described above.

### Screening for Drug Resistance

In certain example embodiments, the devices, systems and methods disclosed herein may be used to screen for microbial genes of interest, for example antibiotic and/or antiviral resistance genes. Guide RNAs may be designed to distinguish between known genes of interest. Samples, including clinical samples, may then be screened using the embodiments disclosed herein for detection of such genes. The ability to screen for drug resistance at POC would have tremendous benefit in selecting an appropriate treatment regime. In certain example embodiments, the antibiotic resistance genes are carbapenemases including KPC, NDM1, CTX-M15, OXA-48. Other antibiotic resistance genes are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

Ribavirin is an effective antiviral that hits a number of RNA viruses. Several clinically important viruses have evolved ribavirin resistance including Foot and Mouth Disease Virus doi: 10.1128/JVI.03594-13; polio virus (Pfeifer and Kirkegaard. PNAS, 100(12):7289-7294, 2003); and hepatitis C virus (Pfeiffer and Kirkegaard, J. Virol. 79(4):2346-2355, 2005). A number of other persistant RNA viruses, such as hepatitis and HIV, have evolved resitance to existing antiviral drugs: hepatitis B virus (lamivudine, tenofovir, entecavir) doi: 10/1002/hep22900; hepatits C virus (telaprevir, BILN2061, ITMN-191, SCh6, boceprevir, AG-021541, ACH-806) doi: 10.1002/hep.22549; and HIV (many drug resistance mutations) hivb.standford.edu. The embodiments disclosed herein may be used to detect such variants among others.

Aside from drug resistance, there are a number of clinically relevant mutations that could be detected with the embodiments disclosed herein, such as persistent versus acute infection in LCMV (doi: 10.1073/pnas.1019304108), and increased infectivity of Ebola (Diehl et al. Cell. 2016, 167(4):1088-1098.

As described herein elsewhere, closely related microbial species (e.g., having only a single nucleotide difference in a given target sequence) may be distinguished by introduction of a synthetic mismatch in the gRNA.

### Set Cover Approaches

In particular embodiments, a set of guide RNAs is designed that can identify, for example, all microbial species within a defined set of microbes. In certain example embodiments, the methods for generating guide RNAs as described herein may be compared to methods disclosed in WO 2017/040316, incorporated herein by reference. As described in WO 2017040316, a set cover solution may identify the minimal number of target sequences probes or guide RNAs needed to cover an entire target sequence or set of target sequences, e.g., a set of genomic sequences. Set cover approaches have been used previously to identify primers and/or microarray probes, typically in the 20 to 50 base pair range. See, e.g., Pearson et al., cs.virginia.edu/˜robins/papers/primers_dam11_final.pdf., Jabado et al. Nucleic Acids Res. 2006 34(22):6605-11, Jabado et al. Nucleic Acids Res. 2008, 36(1):e3 doi10.1093/nar/gkm1106, Duitama et al. Nucleic Acids Res. 2009, 37(8):2483-2492, Phillippy et al. BMC Bioinformatics. 2009, 10:293 doi: 10.1186/1471-2105-10-293. However, such approaches generally involved treating each primer/probe as k-mers and searching for exact matches or allowing for inexact matches using suffix arrays. In addition, the methods generally take a binary approach to detecting hybridization by selecting primers or probes such that each input sequence only needs to be bound by one primer or probe and the position of this binding along the sequence is irrelevant. Alternative methods may divide a target genome into pre-defined windows and effectively treat each window as a separate input sequence under the binary approach—i.e., they determine whether a given probe or guide RNA binds within each window and require that all of the windows be bound by the state of some probe or guide RNA. Effectively, these approaches treat each element of the “universe” in the set cover problem as being either an entire input sequence or a pre-defined window of an input sequence, and each element is considered “covered” if the start of a probe or guide RNA binds within the element. These approaches limit the fluidity to which different probe or guide RNA designs are allowed to cover a given target sequence.

In contrast, the embodiments disclosed herein are directed to detecting longer probe or guide RNA lengths, for example, in the range of 70 bp to 200 bp that are suitable for hybrid selection sequencing. In addition, the methods disclosed WO 2017/040316 herein may be applied to take a pan-target sequence approach capable of defining a probe or guide RNA sets that can identify and facilitate the detection sequencing of all species and/or strains sequences in a large and/or variable target sequence set. For example, the methods disclosed herein may be used to identify all variants of a given virus, or multiple different viruses in a single assay. Further, the method disclosed herein treat each element of the “universe” in the set cover problem as being a nucleotide of a target sequence, and each element is considered “covered” as long as a probe or guide RNA binds to some segment of a target genome that includes the element. This type of set cover methods that may be used instead of the binary approach of previous methods, the methods disclosed in herein better model how a probe or guide RNA may hybridize to a target sequence. Rather than only asking if a given guide RNA sequence does or does not bind to a given window, such approaches may be used to detect a hybridization pattern—i.e. where a given probe or guide RNA binds to a target sequence or target sequences—and then determines from those hybridization patterns the minimum number of probes or guide RNAs needed to cover the set of target sequences to a degree sufficient to enable both enrichment from a sample and sequencing of any and all target sequences. These hybridization patterns may be determined by defining certain parameters that minimize a loss function, thereby enabling identification of minimal probe or guide RNA sets in a way that allows parameters to vary for each species, e.g. to reflect the diversity of each species, as well as in a computationally efficient manner that cannot be achieved using a straightforward application of a set cover solution, such as those previously applied in the probe or guide RNA design context.

The ability to detect multiple transcript abundances may allow for the generation of unique microbial signatures indicative of a particular phenotype. Various machine learning techniques may be used to derive the gene signatures. Accordingly, the guide RNAs of the detection compositions/systems of the present invention may be used to identify and/or quantitate relative levels of biomarkers defined by the gene signature in order to detect certain phenotypes. In certain example embodiments, the gene signature indicates susceptibility to an antibiotic, resistance to an antibiotic, or a combination thereof.

In one aspect of the invention, a method comprises detecting one or more pathogens. In this manner, differentiation between infection of a subject by individual microbes may be obtained. In some embodiments, such differentiation may enable detection or diagnosis by a clinician of specific diseases, for example, different variants of a disease. Preferably the pathogen sequence is a genome of the pathogen or a fragment thereof. The method further may comprise determining the evolution of the pathogen. Determining the evolution of the pathogen may comprise identification of pathogen mutations, e.g., nucleotide deletion, nucleotide insertion, nucleotide substitution. Amongst the latter, there are non-synonymous, synonymous, and noncoding substitutions. Mutations are more frequently non-synonymous during an outbreak. The method may further comprise determining the substitution rate between two pathogen sequences analyzed as described above. Whether the mutations are deleterious or even adaptive would require functional analysis, however, the rate of non-synonymous mutations suggests that continued progression of this epidemic could afford an opportunity for pathogen adaptation, underscoring the need for rapid containment. Thus, the method may further comprise assessing the risk of viral adaptation, wherein the number non-synonymous mutations is determined. (Gire, et al., Science 345, 1369, 2014).

### Monitoring Microbe Outbreaks

In some embodiments, a detection composition of the present invention or methods of use thereof as described herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, or a mechanism involved in a disease outbreak caused by a pathogen.

The pattern of pathogen transmission may comprise continued new transmissions from the natural reservoir of the pathogen or subject-to-subject transmissions (e.g., human-to-human transmission) following a single transmission from the natural reservoir or a mixture of both. In one embodiment, the pathogen transmission may be bacterial or viral transmission, in such case, the target sequence is preferably a microbial genome or fragments thereof. In one embodiment, the pattern of the pathogen transmission is the early pattern of the pathogen transmission, i.e., at the beginning of the pathogen outbreak. Determining the pattern of the pathogen transmission at the beginning of the outbreak increases likelihood of stopping the outbreak at the earliest possible time thereby reducing the possibility of local and international dissemination.

Determining the pattern of the pathogen transmission may comprise detecting a pathogen sequence according to the methods described herein. Determining the pattern of the pathogen transmission may further comprise detecting shared intra-host variations of the pathogen sequence between the subjects and determining whether the shared intra-host variations show temporal patterns. Patterns in observed intrahost and interhost variation provide important insight about transmission and epidemiology (Gire, et al., 2014).

Detection of shared intra-host variations between the subjects that show temporal patterns is an indication of transmission links between subject (in particular between humans) because it can be explained by subject infection from multiple sources (superinfection), sample contamination recurring mutations (with or without balancing selection to reinforce mutations), or co-transmission of slightly divergent viruses that arose by mutation earlier in the transmission chain (Park, et al., Cell 161(7):1516-1526, 2015). Detection of shared intra-host variations between subjects may comprise detection of intra-host variants located at common single nucleotide polymorphism (SNP) positions. Positive detection of intra-host variants located at common (SNP) positions is indicative of superinfection and contamination as primary explanations for the intra-host variants. Superinfection and contamination can be parted on the basis of SNP frequency appearing as inter-host variants (Park, et al., 2015). Otherwise, superinfection and contamination can be ruled out. In this latter case, detection of shared intra-host variations between subjects may further comprise assessing the frequencies of synonymous and nonsynonymous variants and comparing the frequency of synonymous and nonsynonymous variants to one another. A nonsynonymous mutation is a mutation that alters the amino acid of the protein, likely resulting in a biological change in the microbe that is subject to natural selection. Synonymous substitution does not alter an amino acid sequence. Equal frequency of synonymous and nonsynonymous variants is indicative of the intra-host variants evolving neutrally. If frequencies of synonymous and nonsynonymous variants are divergent, the intra-host variants are likely to be maintained by balancing selection. If frequencies of synonymous and nonsynonymous variants are low, this is indicative of recurrent mutation. If frequencies of synonymous and nonsynonymous variants are high, this is indicative of co-transmission (Park, et al., 2015).

Like Ebola virus, Lassa virus (LASV) can cause hemorrhagic fever with high case fatality rates. Andersen et al. generated a genomic catalog of almost 200 LASV sequences from clinical and rodent reservoir samples (Andersen, et al., Cell Volume 162, Issue 4, p 738-750, 13 Aug. 2015). Andersen et al. show that whereas the 2013-2015 EVD epidemic is fueled by human-to-human transmissions, LASV infections mainly result from reservoir-to-human infections. Andersen et al. elucidated the spread of LASV across West Africa and show that this migration was accompanied by changes in LASV genome abundance, fatality rates, codon adaptation, and translational efficiency. The method may further comprise phylogenetically comparing a first pathogen sequence to a second pathogen sequence, and determining whether there is a phylogenetic link between the first and second pathogen sequences. The second pathogen sequence may be an earlier reference sequence. If there is a phylogenetic link, the method may further comprise rooting the phylogeny of the first pathogen sequence to the second pathogen sequence. Thus, it is possible to construct the lineage of the first pathogen sequence. (Park, et al., 2015).

The method may further comprise determining whether the mutations are deleterious or adaptive. Deleterious mutations are indicative of transmission-impaired viruses and dead-end infections, thus normally only present in an individual subject. Mutations unique to one individual subject are those that occur on the external branches of the phylogenetic tree, whereas internal branch mutations are those present in multiple samples (i.e., in multiple subjects). Higher rate of nonsynonymous substitution is a characteristic of external branches of the phylogenetic tree (Park, et al., 2015).

In internal branches of the phylogenetic tree, selection has had more opportunity to filter out deleterious mutants. Internal branches, by definition, have produced multiple descendent lineages and are thus less likely to include mutations with fitness costs. Thus, lower rate of nonsynonymous substitution is indicative of internal branches (Park, et al., 2015).

Synonymous mutations, which likely have less impact on fitness, occurred at more comparable frequencies on internal and external branches (Park, et al., 2015).

By analyzing the sequenced target sequence, such as viral genomes, it is possible to discover the mechanisms responsible for the severity of the epidemic episode such as during the 2014 Ebola outbreak. For example, Gire et al. made a phylogenetic comparison of the genomes of the 2014 outbreak to all 20 genomes from earlier outbreaks suggests that the 2014 West African virus likely spread from central Africa within the past decade. Rooting the phylogeny using divergence from other ebolavirus genomes was problematic (6, 13). However, rooting the tree on the oldest outbreak revealed a strong correlation between sample date and root-to-tip distance, with a substitution rate of 8×10-4 per site per year (13). This suggests that the lineages of the three most recent outbreaks all diverged from a common ancestor at roughly the same time, around 2004, which supports the hypothesis that each outbreak represents an independent zoonotic event from the same genetically diverse viral population in its natural reservoir. They also found out that the 2014 EBOV outbreak might be caused by a single transmission from the natural reservoir, followed by human-to-human transmission during the outbreak. Their results also suggested that the epidemic episode in Sierra Leon might stem from the introduction of two genetically distinct viruses from Guinea around the same time (Gire, et al., 2014).

It has been also possible to determine how the Lassa virus spread out from its origin point, in particular thanks to human-to-human transmission and even retrace the history of this spread 400 years back (Andersen, et al., Cell 162 (4):738-50, 2015).

In relation to the work needed during the 2013-2015 EBOV outbreak and the difficulties encountered by the medical staff at the site of the outbreak, and more generally, the method of the invention makes it possible to carry out sequencing using fewer selected probes such that sequencing can be accelerated, thus shortening the time needed from sample taking to results procurement. Further, kits and systems can be designed to be usable on the field so that diagnostics of a patient can be readily performed without need to send or ship samples to another part of the country or the world.

In any method described above, sequencing the target sequence or fragment thereof may use any of the sequencing processes described above. Further, sequencing the target sequence or fragment thereof may be a near-real-time sequencing. Sequencing the target sequence or fragment thereof may be carried out according to previously described methods (Experimental Procedures: Matranga et al., 2014; and Gire, et al., 2014). Sequencing the target sequence or fragment thereof may comprise parallel sequencing of a plurality of target sequences. Sequencing the target sequence or fragment thereof may comprise Illumina sequencing.

Analyzing the target sequence or fragment thereof that hybridizes to one or more of the selected probes may be an identifying analysis, wherein hybridization of a selected probe to the target sequence or a fragment thereof indicates the presence of the target sequence within the sample.

Currently, primary diagnostics are based on the symptoms a patient has. However, various diseases may share identical symptoms so that diagnostics rely much on statistics. For example, malaria triggers flu-like symptoms: headache, fever, shivering, joint pain, vomiting, hemolytic anemia, jaundice, hemoglobin in the urine, retinal damage, and convulsions. These symptoms are also common for septicemia, gastroenteritis, and viral diseases. Amongst the latter, Ebola hemorrhagic fever has the following symptoms fever, sore throat, muscular pain, headaches, vomiting, diarrhea, rash, decreased function of the liver and kidneys, internal and external hemorrhage.

When a patient is presented to a medical unit, for example in tropical Africa, basic diagnostics will conclude to malaria because statistically, malaria is the most probable disease within that region of Africa. The patient is consequently treated for malaria although the patient might not actually have contracted the disease and the patient ends up not being correctly treated. This lack of correct treatment can be life-threatening especially when the disease the patient contracted presents a rapid evolution. It might be too late before the medical staff realizes that the treatment given to the patient is ineffective and comes to the correct diagnostics and administers the adequate treatment to the patient.

The method of the invention provides a solution to this situation. Indeed, because the number of guide RNAs can be dramatically reduced, this makes it possible to provide on a single chip selected probes divided into groups, each group being specific to one disease, such that a plurality of diseases, e.g., viral infection, can be diagnosed at the same time. Thanks to the invention, more than 3 diseases can be diagnosed on a single chip, preferably more than 4, 5, 6, 7, 8, 9, 10, 11, 12, 13, 14, 15, 16, 17, 18, 19, 20 diseases at the same time, preferably the diseases that most commonly occur within the population of a given geographical area. Since each group of selected probes is specific to one of the diagnosed diseases, a more accurate diagnostics can be performed, thus diminishing the risk of administering the wrong treatment to the patient.

In other cases, a disease such as a viral infection may occur without any symptoms, or had caused symptoms but they faded out before the patient is presented to the medical staff. In such cases, either the patient does not seek any medical assistance or the diagnostics is complicated due to the absence of symptoms on the day of the presentation.

The present invention may also be used in concert with other methods of diagnosing disease, identifying pathogens and optimizing treatment based upon detection of nucleic acids, such as mRNA in crude, non-purified samples.

The method of the invention also provides a powerful tool to address this situation. Indeed, since a plurality of groups of selected guide RNAs, each group being specific to one of the most common diseases that occur within the population of the given area, are comprised within a single diagnostic, the medical staff only need to contact a biological sample taken from the patient with the chip. Reading the chip reveals the diseases the patient has contracted.

In some cases, the patient is presented to the medical staff for diagnostics of particular symptoms. The method of the invention makes it possible not only to identify which disease causes these symptoms but at the same time determine whether the patient suffers from another disease he was not aware of.

This information might be of utmost importance when searching for the mechanisms of an outbreak. Indeed, groups of patients with identical viruses also show temporal patterns suggesting a subject-to-subject transmission links.

In some embodiments, a CRISPR system or methods of use thereof as described herein may be used to predict disease outcome in patients suffering from viral diseases. In specific embodiments, such viral diseases may include, but are not necessarily limited to, Lassa fever. Specific factors related to Lassa fever disease outcome may include but are not necessarily limited to, age, extent of kidney injury, and/or CNS injury.

### Screening Microbial Genetic Perturbations

In certain example embodiments, the detection compositions and systems of the present invention disclosed herein may be used to screen microbial genetic perturbations. Such methods may be useful, for example to map out microbial pathways and functional networks. Microbial cells may be genetically modified and then screened under different experimental conditions. As described above, the embodiments disclosed herein can screen for multiple target molecules in a single sample, or a single target in a single individual discrete volume in a multiplex fashion. Genetically modified microbes may be modified to include a nucleic acid barcode sequence that identifies the particular genetic modification carried by a particular microbial cell or population of microbial cells. A barcode is s short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the effector compositions and systems of the present invention described herein may be used to detect the barcode. Detection of the positive detectable signal indicates the presence of a particular genetic modification in the sample. The methods disclosed herein may be combined with other methods for detecting complimentary genotype or phenotypic readouts indicating the effect of the genetic modification under the experimental conditions tested. Genetic modifications to be screened may include, but are not limited to, a gene knock-in, a gene knock-out, inversions, translocations, transpositions, or one or more nucleotide insertions, deletions, substitutions, mutations, or addition of nucleic acids encoding an epitope with a functional consequence such as altering protein stability or detection. In a similar fashion, the methods described herein may be used in synthetic biology application to screen the functionality of specific arrangements of gene regulatory elements and gene expression modules.

In certain example embodiments, the methods may be used to screen hypomorphs. Generation of hypomorphs and their use in identifying key bacterial functional genes and identification of new antibiotic therapeutics as disclosed in PCT/US2016/060730 entitled “Multiplex High-Resolution Detection of Micro-organism Strains, Related Kits, Diagnostic Methods and Screening Assays” filed Nov. 4, 2016, which is incorporated herein by reference.

The different experimental conditions may comprise exposure of the microbial cells to different chemical agents, combinations of chemical agents, different concentrations of chemical agents or combinations of chemical agents, different durations of exposure to chemical agents or combinations of chemical agents, different physical parameters, or both. In certain example embodiments the chemical agent is an antibiotic or antiviral. Different physical parameters to be screened may include different temperatures, atmospheric pressures, different atmospheric and non-atmospheric gas concentrations, different pH levels, different culture media compositions, or a combination thereof.

### Screening Environmental Samples

The methods disclosed herein may also be used to screen environmental samples for contaminants by detecting the presence of target nucleic acids. For example, in some embodiments, the invention provides a method of detecting microbes, comprising: exposing a detection composition of the present invention as described herein to a sample; activating an RNA effector protein via binding of one or more guide RNAs to one or more microbe-specific target RNAs or one or more trigger RNAs such that a detectable positive signal is produced. The positive signal can be detected and is indicative of the presence of one or more microbes in the sample. In some embodiments, the detection composition or system of the present invention or component thereof may be on a substrate as described herein, and the substrate may be exposed to the sample. In other embodiments, the same detection composition or system of the present invention, and/or a different detection composition or system of the present invention may be applied to multiple discrete locations on the substrate. In further embodiments, the different detection composition or system of the present invention may detect a different microbe at each location. As described in further detail above, a substrate may be a flexible materials substrate, for example, including, but not limited to, a paper substrate, a fabric substrate, or a flexible polymer-based substrate.

In accordance with the invention, the substrate may be exposed to the sample passively, by temporarily immersing the substrate in a fluid to be sampled, by applying a fluid to be tested to the substrate, or by contacting a surface to be tested with the substrate. Any means of introducing the sample to the substrate may be used as appropriate.

As described herein, a sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In some embodiments, Checking for food contamination by bacteria, such as E. coli, in restaurants or other food providers; food surfaces; Testing water for pathogens like Salmonella, Campylobacter, or E. coli, also checking food quality for manufacturers and regulators to determine the purity of meat sources; identifying air contamination with pathogens such as legionella; Checking whether beer is contaminated or spoiled by pathogens like Pediococcus and Lactobacillus; contamination of pasteurized or un-pasteurized cheese by bacteria or fungi during manufacture.

A microbe in accordance with the invention may be a pathogenic microbe or a microbe that results in food or consumable product spoilage. A pathogenic microbe may be pathogenic or otherwise undesirable to humans, animals, or plants. For human or animal purposes, a microbe may cause a disease or result in illness. Animal or veterinary applications of the present invention may identify animals infected with a microbe. For example, the methods and systems of the invention may identify companion animals with pathogens including, but not limited to, kennel cough, rabies virus, and heartworms. In other embodiments, the methods and systems of the invention may be used for parentage testing for breeding purposes. A plant microbe may result in harm or disease to a plant, reduction in yield, or alter traits such as color, taste, consistency, odor. For food or consumable contamination purposes, a microbe may adversely affect the taste, odor, color, consistency or other commercial properties of the food or consumable product. In certain example embodiments, the microbe is a bacterial species. The bacteria may be a psychrotroph, a coliform, a lactic acid bacteria, or a spore-forming bacteria. In certain example embodiments, the bacteria may be any bacterial species that causes disease or illness, or otherwise results in an unwanted product or trait. Bacteria in accordance with the invention may be pathogenic to humans, animals, or plants.

### Example Microbes

The embodiment disclosed herein may be used to detect a number of different microbes. The term microbe as used herein includes bacteria, fungus, protozoa, parasites and viruses.

### Bacteria

The following provides an example list of the types of microbes that might be detected using the embodiments disclosed herein. In certain example embodiments, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Near-real-time microbial diagnostics are needed for food, clinical, industrial, and other environmental settings (see e.g., Lu T K, Bowers J, and Koeris M S., Trends Biotechnol. 2013 June; 31(6):325-7). In certain embodiments, the assay described herein is configured for detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnificus, Yersinia enterocolitica and Yersinia pseudotuberculosis, Brucella spp., Corynebacterium ulcerans, Coxiella burnetii, or Plesiomonas shigelloides).

### Fungi

In certain example embodiments, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

### Protozoa

In certain example embodiments, the microbe is a protozoan. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.Babesia microti, Cryptosporidium parvum, Cyclospora cayetanensis, Plasmodium falciparum, P. vivax, P. ovale, P. malariae, and Toxoplasma gondii.

### Parasites

In certain example embodiments, the microbe is a parasite. Examples of parasites that can be detected in accordance with disclosed methods include without limitation one or more of (or any combination of), an Onchocerca species and a Plasmodium species.

### Viruses

In certain example embodiments, the systems, devices, and methods, disclosed herein are directed to detecting viruses in a sample. The embodiments disclosed herein may be used to detect viral infection (e.g., of a subject or plant), or determination of a viral strain, including viral strains that differ by a single nucleotide polymorphism. The virus may be a DNA virus, an RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\0.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e., after infection has occurred or prophylactically, i.e., before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

### SARS-COV-2

The present disclosure relates to and/or involves detection of SARS-COV-2.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from. While there are many thousands of variants of SARS-COV-2, (Koyama, Takahiko Koyama; Platt, Daniela; Parida, Laxmi (June 2020). “Variant analysis of SARS-COV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-COV-2 as hCoV-19 identified seven clades (O, S, L, V, G, GH, and GR) (Alm E, Broberg E K, Connor T, et al. Geographical and temporal distribution of SARS-COV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, 1378 and D392) (Guan Q, Sadykov M, Mfarrej S, et al. A genetic barcode of SARS-COV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, Á.; et al. “A dynamic nomenclature proposal for SARS-COV-2 lineages to assist genomic epidemiology”. 5:1403-1407).

Genetic variants of SARS-COV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-COV-2, particularly those having substitutions of therapeutic concern. Table 3 below shows exemplary, non-limiting genetic substitutions in SARS-COV-2 variants.

TABLE 3

| | Common Pango Lineages |
|---|---|
| | with Spike Protein |
| Spike Protein Substitution | Substitutions |
| L452R | A.2.5, B.1, B.1.429, |
| | B.1.427, B.1.617.1, |
| | B.1.526.1, B.1.617.2, |
| | C.36.3 |
| E484K | B.1.1.318, B.1.1.7, |
| | B.1.351, B.1.525, |
| | B.1.526, B.1.621, |
| | B.1.623, P.1, P.1.1, |
| | P.1.2, R.1 |
| K417N, E484K, N501Y | B.1.351, B.1.351.3 |
| K417T, E484K, N501Y | P.1, P.1.1, P.1.2 |
| A67V, del69-70, T95I, del142-144, | B.1.1.529 and |
| Y145D, del211, L212I, ins214EPE, | BA lineages |
| G339D, S371L, S373P, S375F, | |
| K417N, N440K, G446S, S477N, | |
| T478K, E484A, Q493R, G496S, | |
| Q498R, N501Y, Y505H, T547K, | |
| D614G, H655Y, N679K, P681H, | |
| N764K, D796Y, N856K, Q954H, | |
| N969K, L981F | |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-COV-2 lineages, known as the PANGO nomenclature. It is available at cov-lineages.org.

In some embodiments, the SARS-COV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T716I, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156del, 157del, R158G, L452R, T478K, D614G, P681R, and D950N; P.1, also known as Gamma (WHO) or Japan/Brazil variant, having the following spike protein substitutions: L18F, T20N, P26S, D138Y, R190S, K417T, E484K, N501Y, D614G, H655Y, and T1027I; and B.1.1.529 also known as Omicron (WHO), having the following spike protein substitutions: A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, or any combination thereof.

In some embodiments, the SARS-COV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In some embodiments, the SARS-Cov-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-Cov-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments the SARS-Cov-2 variant is a VOC. In some embodiments, the SARS-COV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), a Omicon variant (B.1.1.529) or any combination thereof.

In some embodiments the SARS-Cov-2 variant is a VOI. In some embodiments, the SARS-COV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T95I), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); or any combination thereof.

In some embodiments SARS-Cov-2 variant is a VON. In some embodiments, the SARS-Cov-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: https://doi.org/10.1101/2021.02.08.21251393) (spike protein substitutions: F157L, V367F, Q613H, P681R); or any combination thereof.

### Drug Resistant Viruses

In certain embodiments, the virus is a drug resistant virus. By means of example, and without limitation, the virus may be a ribavirin resistant virus. Ribavirin is a very effective antiviral that hits a number of RNA viruses. Below are a few important viruses that have evolved ribavirin resistance. Foot and Mouth Disease Virus: doi: 10.1128/JVI.03594-13. Polio virus: www.pnas.org/content/100/12/7289.full.pdf. Hepatitis C Virus: jvi.asm.org/content/79/4/2346.full. A number of other persistent RNA viruses, such as hepatitis and HIV, have evolved resistance to existing antiviral drugs. Hepatitis B Virus (lamivudine, tenofovir, entecavir): doi: 10.1002/hep.22900. Hepatitis C Virus (Telaprevir, BILN2061, ITMN-191, SCH6, Boceprevir, AG-021541, ACH-806): doi: 10.1002/hep.22549. HIV has many drug resistant mutations, see hivdb.stanford.edu/for more information. Aside from drug resistance, there are a number of clinically relevant mutations that could be targeted with the CRISPR systems according to the invention as described herein. For instance, persistent versus acute infection in LCMV: doi: 10.1073/pnas.1019304108; or increased infectivity of Ebola: http://doi.org/10.1016/j.cell.2016.10.014 and http://doi.org/10.1016/j.cell.2016.10.013.

### Malaria Detection and Monitoring

Malaria is a mosquito-borne pathology caused by Plasmodium parasites. The parasites are spread to people through the bites of infected female Anopheles mosquitoes. Five Plasmodium species cause malaria in humans: Plasmodium falciparum, Plasmodium vivax, Plasmodium ovale, Plasmodium malariae, and Plasmodium knowlesi. Among them, according to the World Health Organization (WHO), Plasmodium falciparum and Plasmodium vivax are responsible for the greatest threat. P. falciparum is the most prevalent malaria parasite on the African continent and is responsible for most malaria-related deaths globally. P. vivax is the dominant malaria parasite in most countries outside of sub-Saharan Africa.

Treatment against Plasmodium sp. include aryl-amino alcohols such as quinine or quinine derivatives such as chloroquine, amodiaquine, mefloquine, piperaquine, lumefantrine, primaquine; lipophilic hydroxynaphthoquinone analog, such as atovaquone; antifolate drugs, such as the sulfa drugs sulfadoxine, dapsone and pyrimethamine; proguanil; the combination of atovaquone/proguanil; atemisins drugs; and combinations thereof. In some embodiments. The method includes screening for resistance against one or more of these compounds.

Target sequences for the assays described herein include those that are diagnostic for the presence of a mosquito-borne pathogen include a sequence that diagnostic for the presence of Plasmodium, notably Plasmodia species affecting humans such as Plasmodium falciparum, Plasmodium vivax, Plasmodium ovale, Plasmodium malariae, and Plasmodium knowlesi, including sequences from the genomes thereof.

Target sequences for the assays described herein include those that are diagnostic for monitoring drug resistance to treatment against Plasmodium, including, but not limited to, Plasmodia species affecting humans such as Plasmodium falciparum, Plasmodium vivax, Plasmodium ovale, Plasmodium malariae, and Plasmodium knowlesi.

Further target sequences include sequences include target molecules/nucleic acid molecules coding for proteins involved in essential biological process for the Plasmodium parasite and notably transporter proteins, such as protein from drug/metabolite transporter family, the ATP-binding cassette (ABC) protein involved in substrate translocation, such as the ABC transporter C subfamily or the Na+/H+ exchanger, membrane glutathione S-transferase; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional target may also include the gene(s) coding for the heme polymerase.

Further target sequences include target molecules/nucleic acid molecules coding for proteins involved in essential biological process may be selected from the P. falciparum chloroquine resistance transporter gene (pfcrt), the P. falciparum multidrug resistance transporter 1 (pfmdr1), the P. falciparum multidrug resistance-associated protein gene (Pfmrp), the P. falciparum Na+/H+ exchanger gene (pfnhe), the gene coding for the P. falciparum exported protein 1, the P. falciparum Ca2+ transporting ATPase 6 (pfatp6); the P. falciparum dihydropteroate synthase (pfdhps), dihydrofolate reductase activity (pfdhpr) and dihydrofolate reductase-thymidylate synthase (pfdhfr) genes, the cytochrome b gene, gtp cyclohydrolase and the Kelch13 (K13) gene as well as their functional heterologous genes in other Plasmodium species.

A number of mutations, notably single point mutations, have been identified in the proteins which are the targets of the current malaria treatments and associated with specific resistance phenotypes. Accordingly, the invention allows for the detection of various resistance phenotypes of mosquito-borne parasites, such as plasmodium by detection of those targets that are associated with the specific resistance phenotypes.

In some embodiments, the method detects one or more mutation(s) and/or one or more single nucleotide polymorphisms in target nucleic acids/molecules. In some embodiments, any one of the mutations below, or their combination thereof, can be used as drug resistance marker and can be detected using the methods, assays, devices, compositions, and/or devices described herein.

Single point mutations in P. falciparum K13 that can be detected by an assay described herein include the following single point mutations in positions 252, 441, 446, 449, 458, 493, 539, 543, 553, 561, 568, 574, 578, 580, 675, 476, 469, 481, 522, 537, 538, 579, 584 and 719 and notably mutations E252Q, P441L, F446I, G449A, N458Y, Y493H, R539T, I543T, P553L, R561H, V568G, P574L, A578S, C580Y, A675V, M476I; C469Y; A481V; S522C; N537I; N537D; G538V; M579I; D584V; and H719N. These mutations are generally associated with artemisins drugs resistance phenotypes (Artemisinin and artemisinin-based combination therapy resistance, April 2016 WHO/HTM/GMP/2016.5).

Mutations in the P. falciparum dihydrofolate reductase (DHFR) (PfDHFR-TS, PFD0830w) that can be detected by the assays described herein include mutations in positions 108, 51, 59 and 164, notably 108 D, 164L, 51I and 59R which modulate resistance to pyrimethamine. Other polymorphisms that can be detected by the methods described herein include 437G, 581G, 540E, 436A and 613S, which are associated with resistance to sulfadoxine. Additional mutations that can be detected by the assays described herein include Ser108Asn, Asn51Ile, Cys59Arg, Ile164Leu, Cys50Arg, Ile164Leu, Asn188Lys, Ser189Arg and Val213Ala, Ser108Thr and Ala16Val. Mutations Ser108Asn, Asn51Ile, Cys59Arg, Ile164Leu, Cys50Arg, Ile164Leu are notably associated with pyrimethamine based therapy and/or chloroguanine-dapsone combination therapy resistances and can be detected by the assays described herein. Cycloguanil resistance appears to be associated with the double mutations Ser108Thr and Ala16Val, which can be detected by the assays described herein. Amplification of dhfr may also be of high relevance for therapy resistance notably pyrimethamine resistance and can be detected by the assays described herein.

Mutations in the P. falciparum dihydropteroate synthase (DHPS) (PfDHPS, PF08_0095) can be detected by the assays described herein, and include, without limitation, mutations in positions 436, 437, 581 and 613 Ser436Ala/Phe, Ala437Gly, Lys540Glu, Ala581Gly and Ala613Thr/Ser. Polymorphism in position 581 and/or 613 have also been associated with resistance to sulfadoxine-pyrimethamine base therapies and can be detected by an assay described herein.

Mutations in the P. falciparum chloroquine-resistance transporter (PfCRT) can be detected by the assays described herein. In some embodiments, the polymorphism in position 76, notably the mutation Lys76Thr, is associated with resistance to chloroquine and can be detected by an assay described herein. Further polymorphisms include Cys72Ser, Met74Ile, Asn75Glu, Ala220Ser, Gln271Glu, Asn326Ser, Ile356Thr and Arg371Ile which may be associated with chloroquine resistance can be detected by an assay described herein. PfCRT is also phosphorylated at the residues S33, S411 and T416, which may regulate the transport activity or specificity of the protein, which can be detected by an assay described herein.

Mutations in the P. falciparum multidrug-resistance transporter 1 (PfMDR1) (PFE1150w) can be detected by an assay described herein. For example, polymorphisms in positions 86, 184, 1034, 1042, notably Asn86Tyr, Tyr184-Phe, Ser1034Cys, Asn1042Asp and Asp1246Tyr have been identified and reported to influence have been reported to influence susceptibilities to lumefantrine, artemisinin, quinine, mefloquine, halofantrine and chloroquine and can be detected by an assay described herein. Additionally, amplification of PfMDR1 is associated with reduced susceptibility to lumefantrine, artemisinin, quinine, mefloquine, and halofantrine and can be detected by an assay described herein. Deamplification of PfMDR1 leads to an increase in chloroquine resistance and can be detected by an assay described herein. Amplification of pfmdr1 may also be detected. The phosphorylation status of PfMDR1 is also of high relevance and can be detected by an assay described herein.

Mutations in the P. falciparum multidrug-resistance associated protein (PfMRP) (gene reference PFA0590w) can be detected by an assay described herein. For example, polymorphisms in positions 191 and/or 437, such as Y191H and A437S have been identified and associated with chloroquine resistance phenotypes and can be detected by an assay described herein.

Mutations in the P. falciparum NA+/H+ enchanger (PfNHE) (ref PF13_0019) can be detected by an assay described herein. For example, increased repetition of the DNNND in microsatellite ms4670 may be a marker for quinine resistance and can be detected by an assay described herein.

Mutations altering the ubiquinol binding site of the cytochrome b protein encoded by the cytochrome be gene (cytb, mal_mito_3) are associated with atovaquone resistance and can be detected by an assay described herein. Mutations in positions 26, 268, 276, 133 and 280 and notably Tyr26Asn, Tyr268Ser, M1331 and G280D may be associated with atovaquone resistance and can be detected by an assay described herein.

In P Vivax, mutations in PvMDR1, the homolog of Pf MDR1 have been associated with chloroquine resistance, notably polymorphism in position 976 such as the mutation Y976F and can be detected by an assay described herein.

The above mutations are defined in terms of protein sequences. However, the skilled person is able to determine the corresponding mutations, including SNPs, to be identified as a nucleic acid target sequence.

Other identified drug-resistance markers are known in the art, for example as described in “Susceptibility of Plasmodium falciparum to antimalarial drugs (1996-2004)”; WHO; Artemisinin and artemisinin-based combination therapy resistance (April 2016 WHO/HTM/GMP/2016.5); “Drug-resistant malaria: molecular mechanisms and implications for public health” FEBS Lett. 2011 Jun. 6; 585 (11):1551-62. doi: 10.1016/j.febslet.2011.04.042. Epub 2011 Apr. 23. Review. PubMed PMID: 21530510; the contents of which are herewith incorporated by reference and can be detected by an assay described herein.

As to polypeptides that may be detected in accordance with the present invention, gene products of all genes mentioned herein may be used as targets. Correspondingly, it is contemplated that such polypeptides could be used for species identification, typing and/or detection of drug resistance.

In certain example embodiments, the systems, devices, and methods, disclosed herein are directed to detecting the presence of one or more mosquito-borne parasite in a sample, such as a biological sample obtained from a subject. In certain example embodiments, the parasite may be selected from the species Plasmodium falciparum, Plasmodium vivax, Plasmodium ovale, Plasmodium malariae or Plasmodium knowlesi. Accordingly, the methods disclosed herein can be adapted for use in other methods (or in combination) with other methods that require quick identification of parasite species, monitoring the presence of parasites and parasite forms (for example corresponding to various stages of infection and parasite life-cycle, such as exo-erythrocytic cycle, erythrocytic cycle, sporpogonic cycle; parasite forms include merozoites, sporozoites, schizonts, gametocytes); detection of certain phenotypes (e.g., pathogen drug resistance), monitoring of disease progression and/or outbreak, and treatment (drug) screening. Further, in the case of malaria, a long time may elapse following the infective bite, namely a long incubation period, during which the patient does not show symptoms. Similarly, prophylactic treatments can delay the appearance of symptoms, and long asymptomatic periods can also be observed before a relapse. Such delays can easily cause misdiagnosis or delayed diagnosis, and thus impair the effectiveness of treatment.

Because of the rapid and sensitive diagnostic capabilities of the embodiments disclosed here, detection of parasite type, down to a single nucleotide difference, and the ability to be deployed as a POC device, the embodiments disclosed herein may be used guide therapeutic regimens, such as selection of the appropriate course of treatment. The embodiments disclosed herein may also be used to screen environmental samples (mosquito population, etc.) for the presence and the typing of the parasite. The embodiments may also be modified to detect mosquito-borne parasites and other mosquito-borne pathogens simultaneously. In some instances, malaria and other mosquito-borne pathogens may present initially with similar symptoms. Thus, the ability to quickly distinguish the type of infection can guide important treatment decisions. Other mosquito-born pathogens that may be detected in conjunction with malaria include dengue, West Nile virus, chikungunya, yellow fever, filariasis, Japanese encephalitis, Saint Louis encephalitis, western equine encephalitis, eastern equine encephalitis, Venezuelan equine encephalitits, La Crosse encephalitis, and zika.

In certain example embodiments, the devices, systems, and methods disclosed herein may be used to distinguish multiple mosquito-borne parasite species in a sample. In certain example embodiments, identification may be based on ribosomal RNA sequences, including the 18S, 16S, 23S, and 5S subunits. In certain example embodiments, identification may be based on sequences of genes that are present in multiple copies in the genome, such as mitochondrial genes like CYTB. In certain example embodiments, identification may be based on sequences of genes that are highly expressed and/or highly conserved such as GAPDH, Histone H2B, enolase, or LDH. Methods for identifying relevant rRNA sequences are disclosed in U.S. Patent Application Publication No. 2017/0029872. In certain example embodiments, a set of guide RNA may be designed to distinguish each species by a variable region that is unique to each species or strain. Guide RNAs may also be designed to target RNA genes that distinguish microbes at the genus, family, order, class, phylum, kingdom levels, or a combination thereof. In certain example embodiments where amplification is used, a set of amplification primers may be designed to flanking constant regions of the ribosomal RNA sequence and a guide RNA designed to distinguish each species by a variable internal region. In certain example embodiments, the primers and guide RNAs may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv: 1307.8690 [q-bio.GN].

In certain example embodiments, species identification can be performed based on genes that are present in multiple copies in the genome, such as mitochondrial genes like CYTB. In certain example embodiments, species identification can be performed based on highly expressed and/or highly conserved genes such as GAPDH, Histone H2B, enolase, or LDH.

In certain example embodiments, a method or diagnostic is designed to screen mosquito-borne parasites across multiple phylogenetic and/or phenotypic levels at the same time. For example, the method or diagnostic may comprise the use of multiple CRISPR systems with different guide RNAs. A first set of guide RNAs may distinguish, for example, between Plasmodium falciparum or Plasmodium vivax. These general classes can be even further subdivided. For example, guide RNAs could be designed and used in the method or diagnostic that distinguish drug-resistant strains, in general or with respect to a specific drug or combination of drugs. A second set of guide RNA can be designed to distinguish microbes at the species level. Thus, a matrix may be produced identifying all mosquito-borne parasites species or subspecies, further divided according to drug resistance. The foregoing is for example purposes only. Other means for classifying other types of mosquito-borne parasites are also contemplated and would follow the general structure described above.

In certain example embodiments, the devices, systems and methods disclosed herein may be used to screen for mosquito-borne parasite genes of interest, for example drug resistance genes. Guide RNAs may be designed to distinguish between known genes of interest. Samples, including clinical samples, may then be screened using the embodiments disclosed herein for detection of one or more such genes. The ability to screen for drug resistance at POC would have tremendous benefit in selecting an appropriate treatment regime. In certain example embodiments, the drug resistance genes are genes encoding proteins such as transporter proteins, such as protein from drug/metabolite transporter family, the ATP-binding cassette (ABC) protein involved in substrate translocation, such as the ABC transporter C subfamily or the Na+/H+ exchanger; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional targets may also include the gene(s) coding for the heme polymerase. In certain example embodiments, the drug resistance genes are selected from the P. falciparum chloroquine resistance transporter gene (pfcrt), the P. falciparum multidrug resistance transporter 1 (pfmdr1), the P. falciparum multidrug resistance-associated protein gene (Pfmrp), the P. falciparum Na+/H+ exchanger gene (pfnhe), the P. falciparum Ca2+ transporting ATPase 6 (pfatp6), the P. falciparum dihydropteroate synthase (pfdhps), dihydrofolate reductase activity (pfdhpr) and dihydrofolate reductase-thymidylate synthase (pfdhfr) genes, the cytochrome b gene, gtp cyclohydrolase and the Kelch13 (K13) gene as well as their functional heterologous genes in other Plasmodium species. Other identified drug-resistance markers are known in the art, for example as described in “Susceptibility of Plasmodium falciparum to antimalarial drugs (1996-2004)”; WHO; Artemisinin and artemisinin-based combination therapy resistance (April 2016 WHO/HTM/GMP/2016.5); “Drug-resistant malaria: molecular mechanisms and implications for public health” FEBS Lett. 2011 Jun. 6; 585 (11):1551-62. doi: 10.1016/j.febslet.2011.04.042. Epub 2011 Apr. 23. Review. PubMed PMID: 21530510; the contents of which are herewith incorporated by reference.

In some embodiments, a CRISPR system, detection system or methods of use thereof as described herein may be used to determine the evolution of a mosquito-borne parasite outbreak. The method may comprise detecting one or more target sequences from a plurality of samples from one or more subjects, wherein the target sequence is a sequence from a mosquito-borne parasite spreading or causing the outbreaks. Such a method may further comprise determining a pattern of mosquito-borne parasite transmission, or a mechanism involved in a disease outbreak caused by a mosquito-borne parasite. The samples may be derived from one or more humans, and/or be derived from one or more mosquitoes.

The pattern of pathogen transmission may comprise continued new transmissions from the natural reservoir of the mosquito-borne parasite or other transmissions (e.g., across mosquitoes) following a single transmission from the natural reservoir or a mixture of both. In one embodiment, the target sequence is preferably a sequence within the mosquito-borne parasite genome or fragments thereof. In one embodiment, the pattern of the mosquito-borne parasite transmission is the early pattern of the mosquito-borne parasite transmission, i.e., at the beginning of the mosquito-borne parasite outbreak. Determining the pattern of the mosquito-borne parasite transmission at the beginning of the outbreak increases likelihood of stopping the outbreak at the earliest possible time thereby reducing the possibility of local and international dissemination.

Determining the pattern of the mosquito-borne parasite transmission may comprise detecting a mosquito-borne parasite sequence according to the methods described herein. Determining the pattern of the pathogen transmission may further comprise detecting shared intra-host variations of the mosquito-borne parasite sequence between the subjects and determining whether the shared intra-host variations show temporal patterns. Patterns in observed intrahost and interhost variation provide important insight about transmission and epidemiology (Gire, et al., 2014).

In addition to other sample types disclosed herein, the sample may be derived from one or more mosquitoes, for example the sample may comprise mosquito saliva.

### Biomarker Detection and Applications

In certain example embodiments, the systems, devices, and methods disclosed herein may be used for biomarker detection. For example, the systems, devices and method disclosed herein may be used for SNP detection and/or genotyping. The systems, devices and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular diseases or disorders, immune diseases or disorders, autoimmune diseases or disorders, neurodegenerative diseases or disorders, neuro motor diseases or disorders, metabolic diseases or disorders, graft-versus-host disease, transplant rejection, ischemic injury (e.g., stroke, heart attack, etc.) spinal cord injury, pathogenic infection, and cancer among other diseases may be detected. In certain example embodiments, the embodiments disclosed herein may be used for cell free DNA detection of diseases that involve lysis, such as liver fibrosis and restrictive/obstructive lung disease. In certain example embodiments, the embodiments could be utilized for faster and more portable detection for pre-natal testing of cell-free DNA. The embodiments disclosed herein may be used for screening panels of different SNPs associated with, among others, cardiovascular health, lipid/metabolic signatures, ethnicity identification, paternity matching, human ID (e.g., matching suspect to a criminal database of SNP signatures). The embodiments disclosed herein may also be used for cell free DNA detection of mutations related to and released from cancer tumors. The embodiments disclosed herein may also be used for detection of meat quality, for example, by providing rapid detection of different animal sources in a given meat product. Embodiments disclosed herein may also be used for the detection of GMOs or gene editing related to DNA. As described herein elsewhere, closely related genotypes/alleles or biomarkers (e.g. having only a single nucleotide difference in a given target sequence) may be distinguished by introduction of a synthetic mismatch in the gRNA.

In an aspect, the invention relates to a method for detecting target nucleic acids in samples, comprising distributing a sample or set of samples into one or more individual discrete volumes, the individual discrete volumes comprising a detection composition according to the invention as described herein; incubating the sample or set of samples under conditions sufficient to allow binding of the one or more guide RNAs to one or more target molecules; activating the effector protein of the detection composition via binding of the one or more guide RNAs to the one or more target molecules, wherein activating the detection composition effector protein results in modification of the detection construct such that a detectable signal is generated; and detecting the detectable signal, wherein detection of the detectable e signal indicates a presence of one or more target molecules in the sample.

In some embodiments, the target polynucleotide encodes a SNAP Receptor (SNARE) protein. In some embodiments, the target polynucleotide encodes is a vehicle associated membrane protein (VAMP). In some embodiments, the VAMP proteins is VAMP1, VAMP2, VAMP3, VAMP4, VAMP5, VAMP7, and/or VAMP8. In some embodiments, the target polynucleotide encodes a SNAP23 polypeptide and/or SNAP25 polypeptide. In some embodiments, the target polynucleotide encodes phosphatase and tensin homolog (PTEN).

In some embodiments, the target polynucleotide encodes an antibody associated with an autoimmune disease. In some embodiments, the target polynucleotide encodes an autoantigen.

### Detecting Circulating Tumor Cells

In one embodiment, circulating cells (e.g., circulating tumor cells (CTC)) can be assayed with the present invention. Isolation of circulating tumor cells (CTC) for use in any of the methods described herein may be performed. Exemplary technologies that achieve specific and sensitive detection and capture of circulating cells that may be used in the present invention have been described (Mostert B, et al., Circulating tumor cells (CTCs): detection methods and their clinical relevance in breast cancer. Cancer Treat Rev. 2009; 35:463-474; and Talasaz A H, et al., Isolating highly enriched populations of circulating epithelial cells and other rare cells from blood using a magnetic sweeper device. Proc Natl Acad Sci USA. 2009; 106:3970-3975). As few as one CTC may be found in the background of 105-106 peripheral blood mononuclear cells (Ross A A, et al., Detection and viability of tumor cells in peripheral blood stem cell collections from breast cancer patients using immunocytochemical and clonogenic assay techniques. Blood. 1993, 82:2605-2610). The CellSearch® platform uses immunomagnetic beads coated with antibodies to Epithelial Cell Adhesion Molecule (EpCAM) to enrich for EPCAM-expressing epithelial cells, followed by immunostaining to confirm the presence of cytokeratin staining and absence of the leukocyte marker CD45 to confirm that captured cells are epithelial tumor cells (Momburg F, et al., Immunohistochemical study of the expression of a Mr 34,000 human epithelium-specific surface glycoprotein in normal and malignant tissues. Cancer Res. 1987; 47:2883-2891; and Allard W J, et al., Tumor cells circulate in the peripheral blood of all major carcinomas but not in healthy subjects or patients with nonmalignant diseases. Clin Cancer Res. 2004; 10:6897-6904). The number of cells captured have been prospectively demonstrated to have prognostic significance for breast, colorectal and prostate cancer patients with advanced disease (Cohen S J, et al., J Clin Oncol. 2008; 26:3213-3221; Cristofanilli M, et al. N Engl J Med. 2004; 351:781-791; Cristofanilli M, et al., J Clin Oncol. 2005; 23:1420-1430; and de Bono J S, et al. Clin Cancer Res. 2008; 14:6302-6309).

The present invention also provides for isolating CTCs with CTC-Chip Technology. CTC-Chip is a microfluidic based CTC capture device where blood flows through a chamber containing thousands of microposts coated with anti-EpCAM antibodies to which the CTCs bind (Nagrath S, et al. Isolation of rare circulating tumour cells in cancer patients by microchip technology. Nature. 2007; 450:1235-1239). CTC-Chip provides a significant increase in CTC counts and purity in comparison to the CellSearch® system (Maheswaran S, et al. Detection of mutations in EGFR in circulating lung-cancer cells, N Engl J Med. 2008; 359:366-377), both platforms may be used for downstream molecular analysis.

### Cell-Free Chromatin

In certain embodiments, cell free chromatin fragments are isolated and analyzed according to the present invention. Nucleosomes can be detected in the serum of healthy individuals (Stroun et al., Annals of the New York Academy of Sciences 906:161-168(2000)) as well as individuals afflicted with a disease state. Moreover, the serum concentration of nucleosomes is considerably higher in patients suffering from benign and malignant diseases, such as cancer and autoimmune disease (Holdenrieder et al (2001) Int J Cancer 95, 1 14-120, Trejo-Becerril et al (2003) Int J Cancer 104, 663-668; Kuroi et al 1999 Breast Cancer 6, 361-364; Kuroi et al (2001) Int j Oncology 19, 143-148; Amoura et al (1997) Arth Rheum 40, 2217-2225; Williams et al (2001) J Rheumatol 28, 81-94). Not being bound by a theory, the high concentration of nucleosomes in tumor bearing patients derives from apoptosis, which occurs spontaneously in proliferating tumors. Nucleosomes circulating in the blood contain uniquely modified histones. For example, U.S. Patent Publication No. 2005/0069931 (Mar. 31, 2005) relates to the use of antibodies directed against specific histone N-terminus modifications as diagnostic indicators of disease, employing such histone-specific antibodies to isolate nucleosomes from a blood or serum sample of a patient to facilitate purification and analysis of the accompanying DNA for diagnostic/screening purposes. Accordingly, the present invention may use chromatin bound DNA to detect and monitor, for example, tumor mutations. The identification of the DNA associated with modified histones can serve as diagnostic markers of disease and congenital defects.

Thus, in another embodiment, isolated chromatin fragments are derived from circulating chromatin, preferably circulating mono and oligonucleosomes. Isolated chromatin fragments may be derived from a biological sample. The biological sample may be from a subject or a patient in need thereof. The biological sample may be sera, plasma, lymph, blood, blood fractions, urine, synovial fluid, spinal fluid, saliva, circulating tumor cells or mucous.

Cell-Free DNA (cfDNA)

In certain embodiments, the present invention may be used to detect cell free DNA (cfDNA). Cell free DNA in plasma or serum may be used as a non-invasive diagnostic tool. For example, cell free fetal DNA has been studied and optimized for testing on-compatible RhD factors, sex determination for X-linked genetic disorders, testing for single gene disorders, indentificaiton of preeclampsia. For example, sequencing the fetal cell fraction of cfDNA in maternal plasma is a reliable approach for detecting copy number changes associated with fetal chromosome aneuploidy. For another example, cfDNA isolated from cancer patients has been used to detect mutations in key genes relevant for treatment decisions.

In certain example embodiments, the present disclosure provides detecting cfDNA directly from a patient sample. In certain other example embodiments, the present disclosure provides enriching cfDNA using the enrichment embodiments disclosed above and prior to detecting the target cfDNA.

### Exosomes

In one embodiment, exosomes can be assayed with the present invention. Exosomes are small extracellular vesicles that have been shown to contain RNA. Isolation of exosomes by ultracentrifugation, filtration, chemical precipitation, size exclusion chromatography, and microfluidics are known in the art. In one embodiment exosomes are purified using an exosome biomarker. Isolation and purification of exosomes from biological samples may be performed by any known methods (see e.g., WO2016172598A1).

### SNP Detection and Genotyping

In certain embodiments, the present invention may be used to detect the presence of single nucleotide polymorphisms (SNP) in a biological sample. The SNPs may be related to maternity testing (e.g., sex determination, fetal defects). They may be related to a criminal investigation. In one embodiment, a suspect in a criminal investigation may be identified by the present invention. Not being bound by a theory nucleic acid based forensic evidence may require the most sensitive assay available to detect a suspect or victim's genetic material because the samples tested may be limiting.

In other embodiments, SNPs associated with a disease are encompassed by the present invention. SNPs associated with diseases are well known in the art and one skilled in the art can apply the methods of the present invention to design suitable guide RNAs (see e.g., www.ncbi.nlm.nih.gov/clinvar?term=human%5Borgn%5D).

In an aspect, the invention relates to a method for genotyping, such as SNP genotyping, comprising: distributing a sample or set of samples into one or more individual discrete volumes, the individual discrete volumes comprising a detection composition or system according to the invention as described herein; incubating the sample or set of samples under conditions sufficient to allow binding of the one or more guide RNAs to one or more target molecules; activating the detection composition effector protein via binding of the one or more guide RNAs to the one or more target molecules, wherein activating the detection composition effector protein results in modification of the detection construct such that a detectable signal is generated; and detecting the detectable signal, wherein detection of the detectable signal indicates a presence of one or more target molecules characteristic for a particular genotype in the sample.

In certain embodiments, the detectable signal is compared to (e.g., by comparison of signal intensity) one or more standard signal, preferably a synthetic standard signal). In certain embodiments, the standard is or corresponds to a particular genotype. In certain embodiments, the standard comprises a particular SNP or other (single) nucleotide variation. In certain embodiments, the standard is a (PCR-amplified) genotype standard. In certain embodiments, the standard is or comprises DNA. In certain embodiments, the standard is or comprises RNA. In certain embodiments, the standard is or comprised RNA which is transcribed from DNA. In certain embodiments, the standard is or comprises DNA which is reverse transcribed from RNA. In certain embodiments, the detectable signal is compared to one or more standard, each of which corresponds to a known genotype, such as a SNP or other (single) nucleotide variation. In certain embodiments, the detectable signal is compared to one or more standard signal and the comparison comprises statistical analysis, such as by parametric or non-parametric statistical analysis, such as by one- or two-way ANOVA, etc. In certain embodiments, the detectable signal is compared to one or more standard signal and when the detectable signal does not (statistically) significantly deviate from the standard, the genotype is determined as the genotype corresponding to said standard.

In other embodiments, the present invention allows rapid genotyping for emergency pharmacogenomics. In one embodiment, a single point of care assay may be used to genotype a patient brought into the emergency room. The patient may be suspected of having a blood clot and an emergency physician needs to decide a dosage of blood thinner to administer. In exemplary embodiments, the present invention may provide guidance for administration of blood thinners during myocardial infarction or stroke treatment based on genotyping of markers such as VKORC1, CYP2C9, and CYP2C19. In one embodiment, the blood thinner is the anticoagulant warfarin (Holford, NH (December 1986). “Clinical Pharmacokinetics and Pharmacodynamics of Warfarin Understanding the Dose-Effect Relationship”. Clinical Pharmacokinetics. Springer International Publishing. 11 (6): 483-504). Genes associated with blood clotting are known in the art (see e.g., US20060166239A1; Litin S C, Gastineau D A (1995) “Current concepts in anticoagulant therapy”. Mayo Clin. Proc. 70 (3):266-72; and Rusdiana et al., Responsiveness to low-dose warfarin associated with genetic variants of VKORC1, CYP2C9, CYP2C19, and CYP4F2 in an Indonesian population. Eur J Clin Pharmacol. 2013 March; 69(3):395-405). Specifically, in the VKORC1 1639 (or 3673) single-nucleotide polymorphism, the common (“wild-type”) G allele is replaced by the A allele. People with an A allele (or the “A haplotype”) produce less VKORCI than do those with the G allele (or the “non-A haplotype”). The prevalence of these variants also varies by race, with 37% of Caucasians and 14% of Africans carrying the A allele. The end result is a decreased number of clotting factors and therefore, a decreased ability to clot.

In certain example embodiments, the availability of genetic material for detecting a SNP in a patient allows for detecting SNPs without amplification of a DNA or RNA sample. In the case of genotyping, the biological sample tested is easily obtained. In certain example embodiments, the incubation time of the present invention may be shortened. The assay may be performed in a period of time required for an enzymatic reaction to occur. One skilled in the art can perform biochemical reactions in 5 minutes (e.g., 5 minute ligation). The present invention may use an automated DNA extraction device to obtain DNA from blood. The DNA can then be added to a reaction that generates a target molecule for the effector protein. Immediately upon generating the target molecule the masking agent can be cut and a signal detected. In exemplary embodiments, the present invention allows a POC rapid diagnostic for determining a genotype before administering a drug (e.g., blood thinner). In the case where an amplification step is used, all of the reactions occur in the same reaction in a one step process. In preferred embodiments, the POC assay may be performed in less than an hour, preferably 10 minutes, 20 minutes, 30 minutes, 40 minutes, or 50 minutes.

In certain embodiments, the systems, devices, and methods disclosed herein may be used for detecting the presence or expression level of long non-coding RNAs (lncRNAs). Expression of certain lncRNAs are associated with disease state and/or drug resistance. In particular, certain lncRNAs (e.g., TCONS_00011252, NR_034078, TCONS_00010506, TCONS_00026344, TCONS_00015940, TCONS_00028298, TCONS_00026380, TCONS_0009861, TCONS_00026521, TCONS_00016127, NR_125939, NR_033834, TCONS_00021026, TCONS_00006579, NR_109890, and NR_026873) are associated with resistance to cancer treatment, such as resistance to one or more BRAF inhibitors (e.g., Vemurafenib, Dabrafenib, Sorafenib, GDC-0879, PLX-4720, and LGX818) for treating melanoma (e.g., nodular melanoma, lentigo maligna, lentigo maligna melanoma, acral lentiginous melanoma, superficial spreading melanoma, mucosal melanoma, polypoid melanoma, desmoplastic melanoma, amelanotic melanoma, and soft-tissue melanoma). The detection of lncRNAs using the various embodiments described herein can facilitate disease diagnosis and/or selection of treatment options.

In one embodiment, the present invention can guide DNA- or RNA-targeted therapies (e.g., CRISPR, TALE, Zinc finger proteins, RNAi), particularly in settings where rapid administration of therapy is important to treatment outcomes.

### LOH Detection

Cancer cells undergo a loss of genetic material (DNA) when compared to normal cells. This deletion of genetic material which almost all, if not all, cancers undergo is referred to as “loss of heterozygosity” (LOH). Loss of heterozygosity (LOH) is a gross chromosomal event that results in loss of the entire gene and the surrounding chromosomal region. The loss of heterozygosity is a common occurrence in cancer, where it can indicate the absence of a functional tumor suppressor gene in the lost region. However, a loss may be silent because there still is one functional gene left on the other chromosome of the chromosome pair. The remaining copy of the tumor suppressor gene can be inactivated by a point mutation, leading to loss of a tumor suppressor gene. The loss of genetic material from cancer cells can result in the selective loss of one of two or more alleles of a gene vital for cell viability or cell growth at a particular locus on the chromosome.

An “LOH marker” is DNA from a microsatellite locus, a deletion, alteration, or amplification in which, when compared to normal cells, is associated with cancer or other diseases. An LOH marker often is associated with loss of a tumor suppressor gene or another, usually tumor related, gene.

The term “microsatellites” refers to short repetitive sequences of DNA that are widely distributed in the human genome. A microsatellite is a tract of tandemly repeated (i.e., adjacent) DNA motifs that range in length from two to five nucleotides, and are typically repeated 5-50 times. For example, the sequence TATATATATA (SEQ ID NO: 76) is a dinucleotide microsatellite, and GTCGTCGTCGTCGTC (SEQ ID NO: 77) is a trinucleotide microsatellite (with A being Adenine, G Guanine, C Cytosine, and T Thymine). Somatic alterations in the repeat length of such microsatellites have been shown to represent a characteristic feature of tumors. Guide RNAs may be designed to detect such microsatellites. Furthermore, the present invention may be used to detect alterations in repeat length, as well as amplifications and deletions based upon quantitation of the detectable signal. Certain microsatellites are located in regulatory flanking or intronic regions of genes, or directly in codons of genes. Microsatellite mutations in such cases can lead to phenotypic changes and diseases, notably in triplet expansion diseases such as fragile X syndrome and Huntington's disease.

Frequent loss of heterozygosity (LOH) on specific chromosomal regions has been reported in many kinds of malignancies. Allelic losses on specific chromosomal regions are the most common genetic alterations observed in a variety of malignancies, thus microsatellite analysis has been applied to detect DNA of cancer cells in specimens from body fluids, such as sputum for lung cancer and urine for bladder cancer. (Rouleau, et al. Nature 363, 515-521 (1993); and Latif, et al. Science 260, 1317-1320 (1993)). Moreover, it has been established that markedly increased concentrations of soluble DNA are present in plasma of individuals with cancer and some other diseases, indicating that cell free serum or plasma can be used for detecting cancer DNA with microsatellite abnormalities. (Kamp, et al. Science 264, 436-440 (1994); and Steck, et al. Nat Genet. 15(4), 356-362 (1997)). Two groups have reported microsatellite alterations in plasma or serum of a limited number of patients with small cell lung cancer or head and neck cancer. (Hahn, et al. Science 271, 350-353(1996); and Miozzo, et al. Cancer Res. 56, 2285-2288(1996)). Detection of loss of heterozygosity in tumors and serum of melanoma patients has also been previously shown (see, e.g., United States patent number U.S. Pat. No. 6,465,177B1).

Thus, it is advantageous to detect of LOH markers in a subject suffering from or at risk of cancer. The present invention may be used to detect LOH in tumor cells. In one embodiment, circulating tumor cells may be used as a biological sample. In preferred embodiments, cell free DNA obtained from serum or plasma is used to noninvasively detect and/or monitor LOH. In other embodiments, the biological sample may be any sample described herein (e.g., a urine sample for bladder cancer). Not being bound by a theory, the present invention may be used to detect LOH markers with improved sensitivity as compared to any prior method, thus providing early detection of mutational events. In one embodiment, LOH is detected in biological fluids, wherein the presence of LOH is associated with the occurrence of cancer. The method and systems described herein represents a significant advance over prior techniques, such as PCR or tissue biopsy by providing a non-invasive, rapid, and accurate method for detecting LOH of specific alleles associated with cancer. Thus, the present invention provides a methods and systems which can be used to screen high-risk populations and to monitor high risk patients undergoing chemoprevention, chemotherapy, immunotherapy or other treatments.

Because the method of the present invention requires only DNA extraction from bodily fluid such as blood, it can be performed at any time and repeatedly on a single patient. Blood can be taken and monitored for LOH before or after surgery; before, during, and after treatment, such as chemotherapy, radiation therapy, gene therapy or immunotherapy; or during follow-up examination after treatment for disease progression, stability, or recurrence. Not being bound by a theory, the method of the present invention also may be used to detect subclinical disease presence or recurrence with an LOH marker specific for that patient since LOH markers are specific to an individual patient's tumor. The method also can detect if multiple metastases may be present using tumor specific LOH markers.

### Detection of Epigenetic Modifications

Histone variants, DNA modifications, and histone modifications indicative of cancer or cancer progression may be used in the present invention. For example, U.S. patent publication 20140206014 describes that cancer samples had elevated nucleosome H2AZ, macroH2A1.1, 5-methylcytosine, P-H2AX (Ser139) levels as compared to healthy subjects. The presence of cancer cells in an individual may generate a higher level of cell free nucleosomes in the blood as a result of the increased apoptosis of the cancer cells. In one embodiment, an antibody directed against marks associated with apoptosis, such as H2B Ser 14 (P), may be used to identify single nucleosomes that have been released from apoptotic neoplastic cells. Thus, DNA arising from tumor cells may be advantageously analyzed according to the present invention with high sensitivity and accuracy.

### Pre-natal Screening

In certain embodiments, the method and systems of the present invention may be used in prenatal screening. In certain embodiments, cell-free DNA is used in a method of prenatal screening. In certain embodiments, DNA associated with single nucleosomes or oligonucleosomes may be detected with the present invention. In preferred embodiments, detection of DNA associated with single nucleosomes or oligonucleosomes is used for prenatal screening. In certain embodiments, cell-free chromatin fragments are used in a method of prenatal screening.

Prenatal diagnosis or prenatal screening refers to testing for diseases or conditions in a fetus or embryo before it is born. The aim is to detect birth defects such as neural tube defects, Down syndrome, chromosome abnormalities, genetic disorders and other conditions, such as spina bifida, cleft palate, Tay Sachs disease, sickle cell anemia, thalassemia, cystic fibrosis, Muscular dystrophy, and fragile X syndrome. Screening can also be used for prenatal sex discernment. Common testing procedures include amniocentesis, ultrasonography including nuchal translucency ultrasound, serum marker testing, or genetic screening. In some cases, the tests are administered to determine if the fetus will be aborted, though physicians and patients also find it useful to diagnose high-risk pregnancies early so that delivery can be scheduled in a tertian,' care hospital where the baby can receive appropriate care.

It has been realized that there are fetal cells which are present in the mother's blood, and that these cells present a potential source of fetal chromosomes for prenatal DNA-based diagnostics. Additionally, fetal DNA ranges from about 2-10% of the total DNA in maternal blood. Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses. Cell-free fetal DNA has been shown to exist in plasma and serum of pregnant women as early as the sixth week of gestation, with concentrations rising during pregnancy and peaking prior to parturition. Because these cells appear very early in the pregnancy, they could form the basis of an accurate, noninvasive, first trimester test. Not being bound by a theory, the present invention provides unprecedented sensitivity in detecting low amounts of fetal DNA. Not being bound by a theory, abundant amounts of maternal DNA is generally concomitantly recovered along with the fetal DNA of interest, thus decreasing sensitivity in fetal DNA quantification and mutation detection. The present invention overcomes such problems by the unexpectedly high sensitivity of the assay.

The H3 class of histones consists of four different protein types: the main types, H3.1 and H3.2; the replacement type, H3.3; and the testis specific variant, H3t. Although H3.1 and H3.2 are closely related, only differing at Ser96, H3.1 differs from H3.3 in at least 5 amino acid positions. Further, H3.1 is highly enriched in fetal liver, in comparison to its presence in adult tissues including liver, kidney and heart. In adult human tissue, the H3.3 variant is more abundant than the H3.1 variant, whereas the converse is true for fetal liver. The present invention may use these differences to detect fetal nucleosomes and fetal nucleic acid in a maternal biological sample that comprises both fetal and maternal cells and/or fetal nucleic acid.

In one embodiment, fetal nucleosomes may be obtained from blood. In other embodiments, fetal nucleosomes are obtained from a cervical mucus sample. In certain embodiments, a cervical mucus sample is obtained by swabbing or lavage from a pregnant woman early in the second trimester or late in the first trimester of pregnancy. The sample may be placed in an incubator to release DNA trapped in mucus. The incubator may be set at 37° C. The sample may be rocked for approximately 15 to 30 minutes. Mucus may be further dissolved with a mucinase for the purpose of releasing DNA. The sample may also be subjected to conditions, such as chemical treatment and the like, as well known in the art, to induce apoptosis to release fetal nucleosomes. Thus, a cervical mucus sample may be treated with an agent that induces apoptosis, whereby fetal nucleosomes are released. Regarding enrichment of circulating fetal DNA, reference is made to U.S. patent publication Nos. 20070243549 and 20100240054. The present invention is especially advantageous when applying the methods and systems to prenatal screening where only a small fraction of nucleosomes or DNA may be fetal in origin.

Prenatal screening according to the present invention may be for a disease including, but not limited to Trisomy 13, Trisomy 16, Trisomy 18, Klinefelter syndrome (47, XXY), (47, XYY) and (47, XXX), Turner syndrome, Down syndrome (Trisomy 21), Cystic Fibrosis, Huntington's Disease, Beta Thalassaemia, Myotonic Dystrophy, Sickle Cell Anemia, Porphyria, Fragile-X-Syndrome, Robertsonian translocation, Angelman syndrome, DiGeorge syndrome and Wolf-Hirschhorn Syndrome.

Several further aspects of the invention relate to diagnosing, prognosing and/or treating defects associated with a wide range of genetic diseases which are further described on the website of the National Institutes of Health under the topic subsection Genetic Disorders (website at health.nih.gov/topic/Genetic Disorders).

### Cancer and Cancer Drug Resistance Detection

In certain embodiments, the present invention may be used to detect genes and mutations associated with cancer. In certain embodiments, mutations associated with resistance are detected. The amplification of resistant tumor cells or appearance of resistant mutations in clonal populations of tumor cells may arise during treatment (see, e.g., Burger J A, et al., Clonal evolution in patients with chronic lymphocytic leukaemia developing resistance to BTK inhibition. Nat Commun. 2016 May 20; 7:11589; Landau D A, et al., Mutations driving CLL and their evolution in progression and relapse. Nature. 2015 Oct. 22; 526(7574):525-30; Landau D A, et al., Clonal evolution in hematological malignancies and therapeutic implications. Leukemia. 2014 January; 28(1):34-43; and Landau D A, et al., Evolution and impact of subclonal mutations in chronic lymphocytic leukemia. Cell. 2013 Feb. 14; 152(4):714-26). Accordingly, detecting such mutations requires highly sensitive assays and monitoring requires repeated biopsy. Repeated biopsies are inconvenient, invasive and costly. Resistant mutations can be difficult to detect in a blood sample or other noninvasively collected biological sample (e.g., blood, saliva, urine) using the prior methods known in the art. Resistant mutations may refer to mutations associated with resistance to a chemotherapy, targeted therapy, or immunotherapy.

In certain embodiments, mutations occur in individual cancers that may be used to detect cancer progression. In one embodiment, mutations related to T cell cytolytic activity against tumors have been characterized and may be detected by the present invention (see e.g., Rooney et al., Molecular and genetic properties of tumors associated with local immune cytolytic activity, Cell. 2015 Jan. 15; 160(1-2): 48-61). Personalized therapies may be developed for a patient based on detection of these mutations (see e.g., WO2016100975A1). In certain embodiments, cancer specific mutations associated with cytolytic activity may be a mutation in a gene selected from the group consisting of CASP8, B2M, PIK3CA, SMCIA, ARID5B, TET2, ALPK2, COL5A1, TP53, DNER, NCOR1, MORC4, CIC, IRF6, MYOCD, ANKLE1, CNKSR1, NF1, SOS1, ARID2, CUL4B, DDX3X, FUBP1, TCP11L2, HLA-A, B or C, CSNK2A1, MET, ASXL1, PD-L1, PD-L2, IDO1, IDO2, ALOX12B and ALOX15B, or copy number gain, excluding whole-chromosome events, impacting any of the following chromosomal bands: 6q16.1-q21, 6q22.31-q24.1, 6q25.1-q26, 7p11.2-q11.1, 8p23.1, 8p11.23-p11.21 (containing IDO1, IDO2), 9p24.2-p23 (containing PDL1, PDL2), 10p15.3, 10p15.1-p13, 11p14.1, 12p13.32-p13.2, 17p13.1 (containing ALOX12B, ALOX15B), and 22q11.1-q11.21.

In certain embodiments, the present invention is used to detect a cancer mutation (e.g., resistance mutation) during the course of a treatment and after treatment is completed. The sensitivity of the present invention may allow for noninvasive detection of clonal mutations arising during treatment and can be used to detect a recurrence in the disease.

In certain example embodiments, detection of microRNAs (miRNA) and/or miRNA signatures of differentially expressed miRNA, may be used to detect or monitor progression of a cancer and/or detect drug resistance to a cancer therapy. As an example, Nadal et al. (Nature Scientific Reports, (2015) doi: 10.1038/srep12464) describe mRNA signatures that may be used to detect non-small cell lung cancer (NSCLC).

In certain example embodiments, the presence of resistance mutations in clonal subpopulations of cells may be used in determining a treatment regimen. In other embodiments, personalized therapies for treating a patient may be administered based on common tumor mutations. In certain embodiments, common mutations arise in response to treatment and lead to drug resistance. In certain embodiments, the present invention may be used in monitoring patients for cells acquiring a mutation or amplification of cells harboring such drug resistant mutations.

Treatment with various chemotherapeutic agents, particularly with targeted therapies such as tyrosine kinase inhibitors, frequently leads to new mutations in the target molecules that resist the activity of the therapeutic. Multiple strategies to overcome this resistance are being evaluated, including development of second generation therapies that are not affected by these mutations and treatment with multiple agents including those that act downstream of the resistance mutation. In an exemplary embodiment, a common mutation to ibrutinib, a molecule targeting Bruton's Tyrosine Kinase (BTK) and used for CLL and certain lymphomas, is a Cysteine to Serine change at position 481 (BTK/C481S). Erlotinib, which targets the tyrosine kinase domain of the Epidermal Growth Factor Receptor (EGFR), is commonly used in the treatment of lung cancer and resistant tumors invariably develop following therapy. A common mutation found in resistant clones is a threonine to methionine mutation at position 790.

Non-silent mutations shared between populations of cancer patients and common resistant mutations that may be detected with the present invention are known in the art (see e.g., WO/2016/187508). In certain embodiments, drug resistance mutations may be induced by treatment with ibrutinib, erlotinib, imatinib, gefitinib, crizotinib, trastuzumab, vemurafenib, RAF/MEK, check point blockade therapy, or antiestrogen therapy. In certain embodiments, the cancer specific mutations are present in one or more genes encoding a protein selected from the group consisting of Programmed Death-Ligand 1 (PD-L1), androgen receptor (AR), Bruton's Tyrosine Kinase (BTK), Epidermal Growth Factor Receptor (EGFR), BCR-Abl, c-kit, PIK3CA, HER2, EML4-ALK, KRAS, ALK, ROS1, AKT1, BRAF, MEK1, MEK2, NRAS, RAC1, and ESR1.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Recently, gene expression in tumors and their microenvironments have been characterized at the single cell level (see e.g., Tirosh, et al. Dissecting the multicellular ecosystem of metastatic melanoma by single cell RNA-seq. Science 352, 189-196, doi: 10.1126/science.aad0501 (2016)); Tirosh et al., Single-cell RNA-seq supports a developmental hierarchy in human oligodendroglioma. Nature. 2016 Nov. 10; 539(7628):309-313. doi: 10.1038/nature20123. Epub 2016 Nov. 2; and International patent publication serial number WO 2017004153 A1). In certain embodiments, gene signatures may be detected using the present invention. In one embodiment complement genes are monitored or detected in a tumor microenvironment. In one embodiment MITF and AXL programs are monitored or detected. In one embodiment, a tumor specific stem cell or progenitor cell signature is detected. Such signatures indicate the state of an immune response and state of a tumor. In certain embodiments, the state of a tumor in terms of proliferation, resistance to treatment and abundance of immune cells may be detected.

Thus, in certain embodiments, the invention provides low-cost, rapid, multiplexed cancer detection panels for circulating DNA, such as tumor DNA, particularly for monitoring disease recurrence or the development of common resistance mutations.

In some embodiments, the cancer biomarker detected is a nucleic acid sequence encoding PTEN.

### Immunotherapy Applications

The embodiments disclosed herein can also be useful in further immunotherapy contexts. For instance, in some embodiments methods of diagnosing, prognosing and/or staging an immune response in a subject comprise detecting a first level of expression, activity and/or function of one or more biomarker and comparing the detected level to a control level wherein a difference in the detected level and the control level indicates that the presence of an immune response in the subject.

In certain embodiments, the present invention may be used to determine dysfunction or activation of tumor infiltrating lymphocytes (TIL). TILs may be isolated from a tumor using known methods. The TILs may be analyzed to determine whether they should be used in adoptive cell transfer therapies. Additionally, chimeric antigen receptor T cells (CAR T cells) may be analyzed for a signature of dysfunction or activation before administering them to a subject. Exemplary signatures for dysfunctional and activated T cell have been described (see e.g., Singer M, et al., A Distinct Gene Module for Dysfunction Uncoupled from Activation in Tumor-Infiltrating T Cells. Cell. 2016 Sep. 8; 166(6):1500-1511.e9. doi: 10.1016/j.cell.2016.08.052).

In some embodiments, the programmable nuclease-peptidase compositions of the present invention is used to evaluate that state of immune cells, such as T cells (e.g., CD8+ and/or CD4+ T cells). In particular, T cell activation and/or dysfunction can be determined, e.g., based on genes or gene signatures associated with one or more of the T cell states. In this way, c2c2 can be used to determine the presence of one or more subpopulations of T cells.

In some embodiments, the programmable nuclease-peptidase compositions of the present invention can be used in a diagnostic assay or may be used as a method of determining whether a patient is suitable for administering an immunotherapy or another type of therapy. For example, detection of gene or biomarker signatures may be performed via the programmable nuclease-peptidase system of the present invention to determine whether a patient is responding to a given treatment or, if the patient is not responding, if this may be due to T cell dysfunction. Such detection is informative regarding the types of therapy the patient is best suited to receive. For example, whether the patient should receive immunotherapy.

In some embodiments, the systems and assays disclosed herein may allow clinicians to identify whether a patient's response to a therapy (e.g., an adoptive cell transfer (ACT) therapy) is due to cell dysfunction, and if it is, levels of up-regulation and down-regulation across the biomarker signature will allow problems to be addressed. For example, if a patient receiving ACT is non-responsive, the cells administered as part of the ACT may be assayed by an assay disclosed herein to determine the relative level of expression of a biomarker signature known to be associated with cell activation and/or dysfunction states. If a particular inhibitory receptor or molecule is up-regulated in the ACT cells, the patient may be treated with an inhibitor of that receptor or molecule. If a particular stimulatory receptor or molecule is down-regulated in the ACT cells, the patient may be treated with an agonist of that receptor or molecule.

In certain example embodiments, the systems, methods, and devices described herein may be used to screen gene signatures that identify a particular cell type, cell phenotype, or cell state. Likewise, through the use of such methods as compressed sensing, the embodiments disclosed herein may be used to detect transcriptomes. Gene expression data are highly structured, such that the expression level of some genes is predictive of the expression level of others. Knowledge that gene expression data are highly structured allows for the assumption that the number of degrees of freedom in the system are small, which allows for assuming that the basis for computation of the relative gene abundances is sparse. It is possible to make several biologically motivated assumptions that allow Applicants to recover the nonlinear interaction terms while under-sampling without having any specific knowledge of which genes are likely to interact. In particular, if Applicants assume that genetic interactions are low rank, sparse, or a combination of these, then the true number of degrees of freedom is small relative to the complete combinatorial expansion, which enables Applicants to infer the full nonlinear landscape with a relatively small number of perturbations. Working around these assumptions, analytical theories of matrix completion and compressed sensing may be used to design under-sampled combinatorial perturbation experiments. In addition, a kernel-learning framework may be used to employ under-sampling by building predictive functions of combinatorial perturbations without directly learning any individual interaction coefficient Compresses sensing provides a way to identify the minimal number of target transcripts to be detected in order obtain a comprehensive gene-expression profile. Methods for compressed sensing are disclosed in PCT/US2016/059230 “Systems and Methods for Determining Relative Abundances of Biomolecules” filed Oct. 27, 2016, which is incorporated herein by reference. Having used methods like compressed sensing to identify a minimal transcript target set, a set of corresponding guide RNAs may then be designed to detect said transcripts. Accordingly, in certain example embodiments, a method for obtaining a gene-expression profile of cell comprises detecting, using the embodiments disclosed, herein a minimal transcript set that provides a gene-expression profile of a cell or population of cells.

### Detecting Nucleic Acid Tagged Molecules

In some embodiments, the programmable nuclease-peptidase and/or detection compositions of the present invention described herein may be used to detect nucleic acid identifiers. Nucleic acid identifiers are non-coding nucleic acids that may be used to identify a particular article. Example nucleic acid identifiers, such as DNA watermarks, are described in Heider and Barnekow. “DNA watermarks: A proof of concept” BMC Molecular Biology 9:40 (2008). The nucleic acid identifiers may also be a nucleic acid barcode. A nucleic-acid based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify target molecules and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

### Methods of Cell Labeling

The programmable nuclease-peptidase and/or detection compositions of the present invention can be used, for example, to label a cell. As previously described in relation to e.g., methods of detecting target polynucleotides, when a detection composition of the present invention is activated by binding a target polynucleotide a detectable signal or product is produced. In some embodiments, the detectable signal or product is such that it allows a cell to which the system is delivered to and activated in to be “labeled” via the detectable signal or product. For example, if the detectable signal is an optical signal (e.g., fluorescence) produced from a protein, then the cell is effectively labeled with fluorescence that can be tracked, imaged, and used for e.g., fluorescence-based sorting or separation techniques. Other signals and products that can be used as labels are described in greater detail elsewhere herein and will be appreciated in view of the description provided herein. In this way, cells containing a target polynucleotide can be effectively labeled. Labeling via a method described herein can occur in vivo, ex vivo, in vitro, or in situ. Such methods can be applied to various cell detection, imaging, diagnostic, prognostic, screening, functionality, cell isolation and separation, and other assays and techniques where cell labeling is traditionally employed. Such labeling approaches can be helpful for cell type and cell state evaluation, particularly at the single cell level.

Described in certain example embodiments herein are methods of labeling cells comprising introducing a detection composition as described in greater detail elsewhere herein into a population of cells, wherein the guide molecule is configured to detect one or more target transcripts associated with a particular cell type or cell state; and activating the peptidase via binding of the complex to the one or more target transcripts such that the detection construct is modified by the activated peptidase such that a detectable product and/or signal is generated, thereby labeling cells within the cell population expressing the one or more target transcripts.

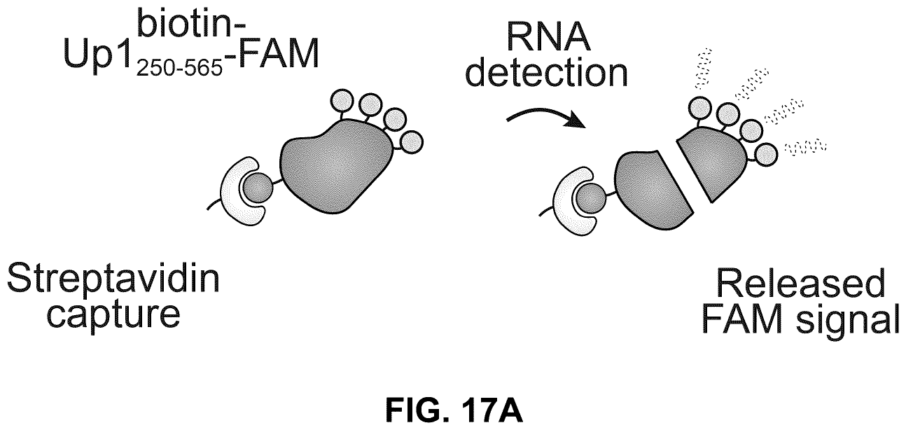

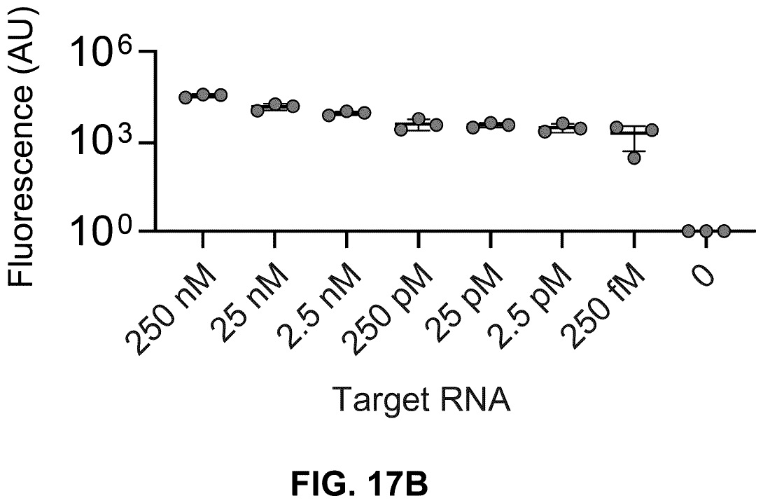

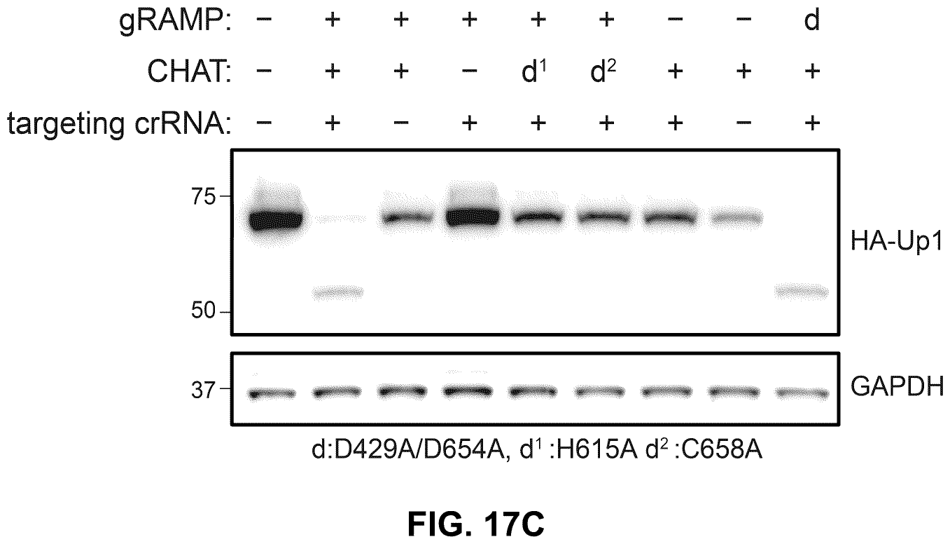

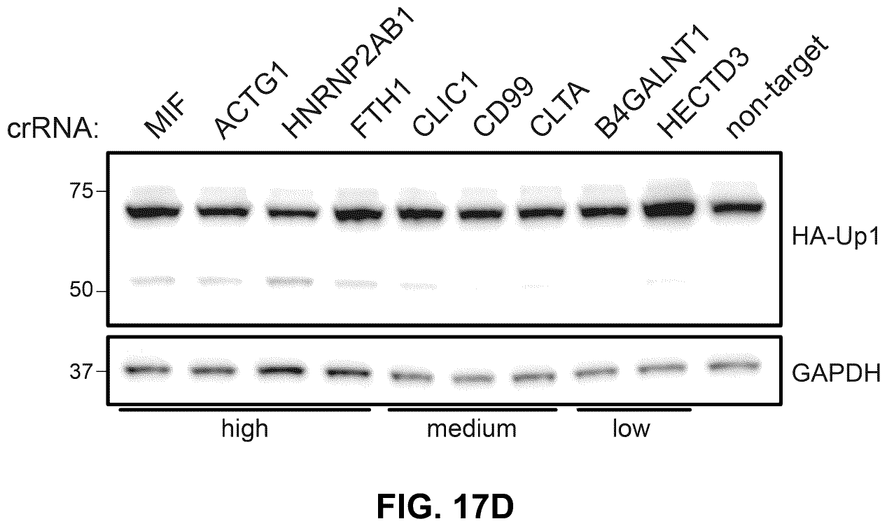

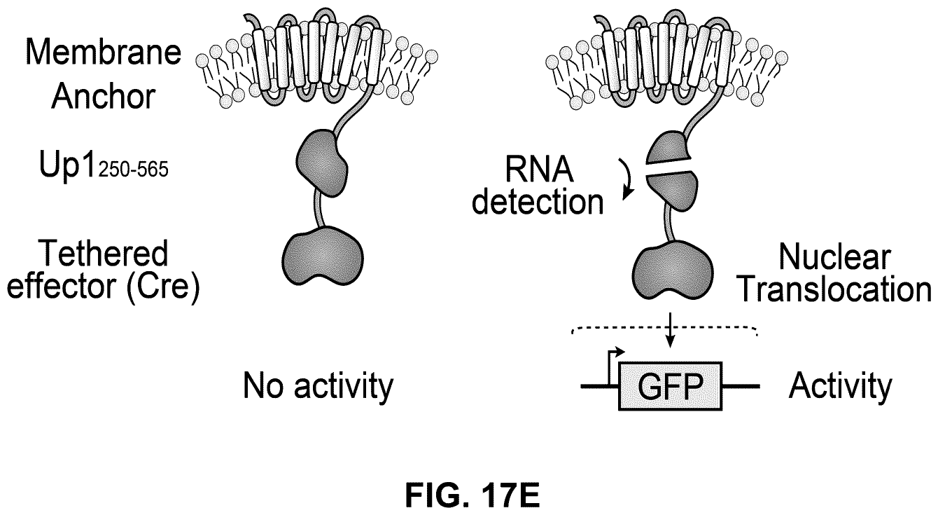

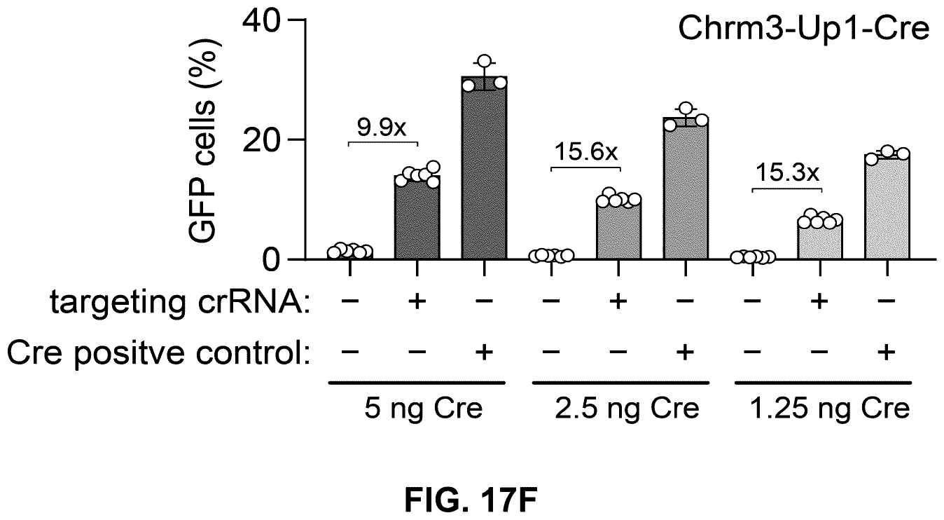

In some embodiments, the peptidase substrate is tethered or anchored to a structure within the cell. Exemplary cell structures to which the peptidase substrate can be anchored is the cell or nuclear membrane, mitochondria membrane, endoplasmic reticulum, lysosome, Golgi apparatus, microtubules or other cytoskeleton components, and/or the like. In some embodiments the substrate is coupled to a signal producing molecule or product producing molecule that is inactive until released from the peptidase substrate or is otherwise modified by activity of the peptidase on the substrate upon binding a target nucleic acid (e.g., a target RNA). See e.g., FIG. 17E and the Working Examples herein.

In Vivo Delivery and/or Effector Function

Similar to embodiments of cell labeling, the programmable nuclease-peptidase system can be configured for in vivo effector function and/or delivery of a molecule, such as a therapeutic molecule. As shown in e.g., FIG. 17E, a substrate for the peptidase can be tethered or otherwise anchored to a cellular structure. Exemplary cell structures to which the peptidase substrate can be anchored is the cell or nuclear membrane, mitochondria membrane, endoplasmic reticulum, lysosome, Golgi apparatus, microtubules or other cytoskeleton components, and/or the like. The substrate can also be coupled to (either directly or via a linker), to an effector molecule (e.g., a Cre recombinase, CRISPR-Cas system, or other effector molecule) or to a therapeutic molecule. In some embodiments, the effector or other molecule (e.g., a therapeutic molecule), is inactive while coupled to the substrate. When a target RNA is present, in cell that also contain the programmable nuclease-peptidase, the peptidase is activated upon binding the target RNA and acts to cleave the tethered substrate. Cleaving of the substrate releases and/or otherwise activates the effector or therapeutic molecule that was linked to the peptidase substrate. Target RNA can be endogenous to the cell that expresses the programmable nuclease-peptidase system and/or tethered substrate-effector (or therapeutic) complex. In other embodiments, Target RNA is exogenous to the cell. Exogenous target RNA can provide an additional measure of temporal and/or spatial control of effector function and/or therapeutic delivery. Exemplary effectors that can be included in these embodiments are described in greater detail elsewhere herein and will be appreciated by those of ordinary skill in the art in view of the description herein.

In some embodiments, a method of in vivo effector activation or delivery includes introducing a programmable nuclease system of the present invention into a cell comprising a substrate of the peptidase, wherein the substrate of the peptidase is optionally tethered to a cellular structure and wherein the substrate the peptidase is coupled to an effector. In some embodiments, the effector is capable of producing a detectable signal when activated, is a therapeutic molecule or prodrug, is a genetic modifying molecule, or any combination thereof. In some embodiments, the effector is inactive when coupled to an uncleaved substrate. In some embodiments, the effector is inactive when coupled to a cleaved substrate portion (and thus is active when coupled to an uncleaved substrate). In some embodiments, the method further comprises cleaving the substrate in response to a target RNA and activation of the peptidase of the programmable nuclease system. In some embodiments, the target RNA is endogenous to the cell or is exogenous to the cell. In some embodiments, the substrate is tethered to a cell membrane or a nuclear membrane.

### EXAMPLES

### Example 1-Determination of a CHAT Domain Containing Protein

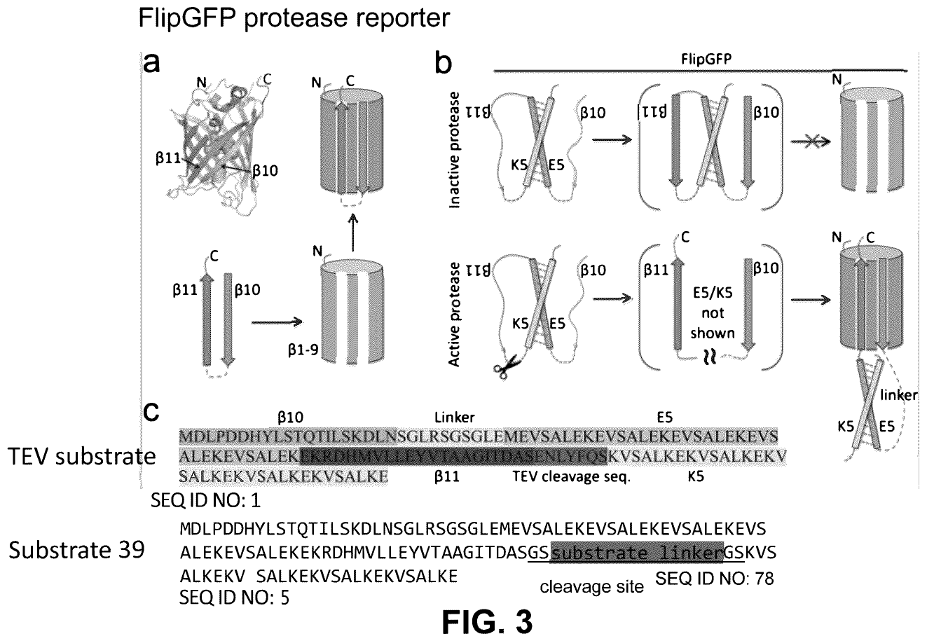

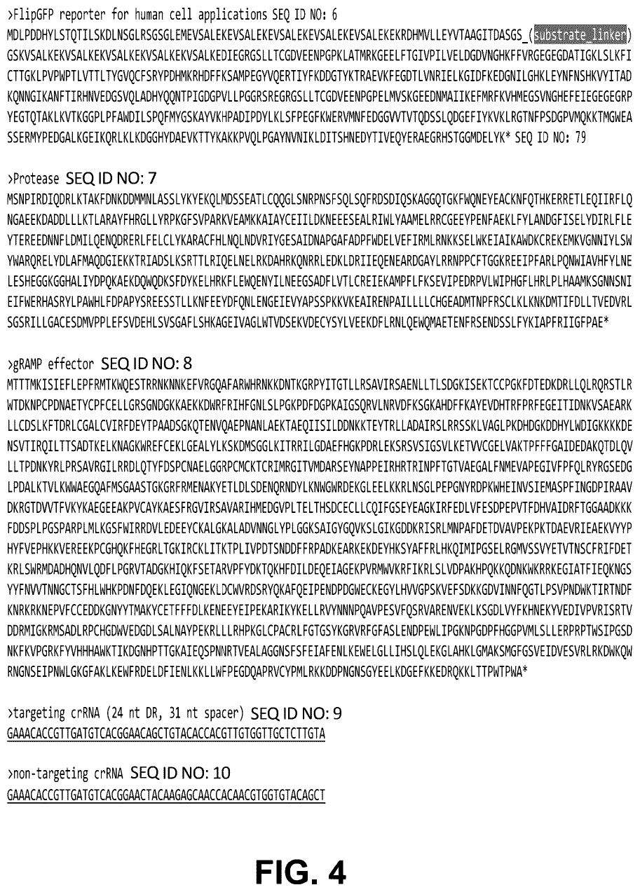

A 3D ribbon model of the predicted structure of a D. ishimotonii CHAT domain containing protein was developed using Alphafold2 (FIG. 1). The putative active site was also identified on in the 3D ribbon model. A putative natural target protein for the CHAT domain containing protein of FIG. 1 was also identified. A 3D ribbon model of the natural target protein was generated using Alphafold2 (FIG. 2). A Flip protease reporter construct and assay (FIGS. 3-4) was developed to analyze the protease/peptidase recognition site of the putative natural target for the CHAT domain containing protein of FIG. 1. The Flip protease reporter assay and construct was based upon the construct described in Zhang et al., J Am Chem Soc. 2019 Mar. 20; 141(11):4526-4530. doi: 10.1021/jacs.8b13042. Epub 2019 Mar. 6. PMID: 30821975; PMCID: PMC6486793. The construct contains a putative protease/peptidase substrates as well as a control (TEV) site. If the putative substrate is indeed a substrate of the protease or peptidase, the reporter is cleaved at or in effective proximity to the substrate sequence and a signal or loss of signal is generated due to flipping of one or more of the domains of the reporter construct. Candidate substrates are incorporated into the FLIP-reporter construct at the position designated “substrate linker”.

An in vitro experiment was performed to examine in vitro reconstitution of the system and RNA-guided protein cleavage. Briefly, a gRAMP-protease-crRNA complex was purified from E. coli and incubated with purified WP_124327587.1 protein. Reactions were incubated at 37 degrees C. for 1 hour in the presence of Mg²⁺ and ATP. Representative results are shown in FIG. 5, which demonstrates in vitro reconstitution of RNA-guided protein cleavage. This also revealed that the substrate is neighboring protein WP_124327587.1 (FIG. 2), that cleavage of the substrate is dependent on presence of a target RNA, and that the protease is a multi-turnover enzyme as it can process (e.g., cleave) an excess of substrate.

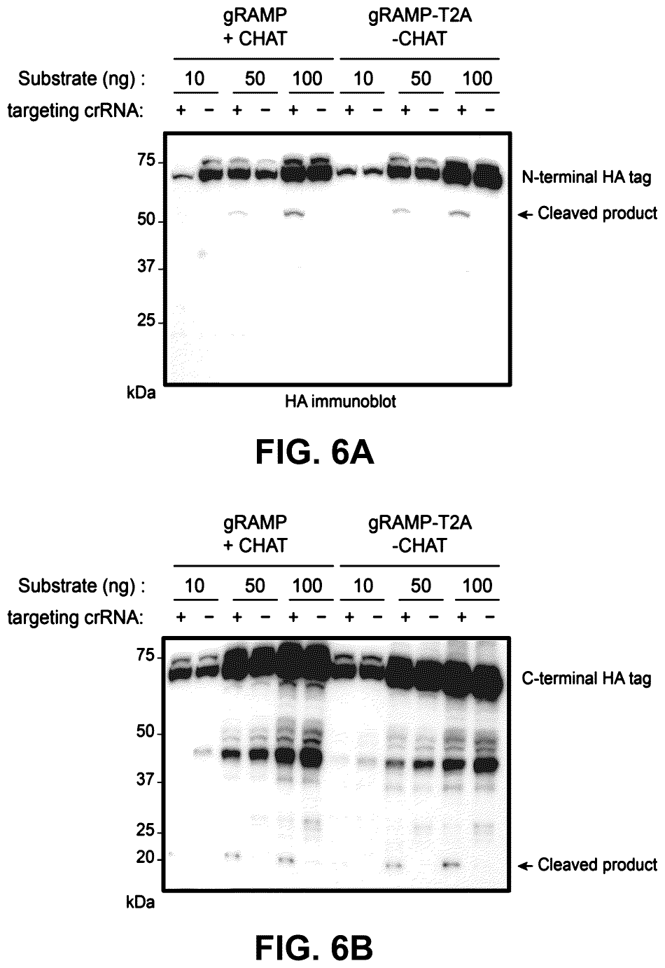

Further, protein substrate cleavage following RNA targeting by the gRAMP-CHAT complex was also demonstrated in cells. Briefly, HEK-293 cells were transfected with separate gRAMP and CHAT expression plasmids or a combination of the two proteins with a T2A linker, a targeting or non-targeting crRNA, a plasmid expressing the target RNA, and an HA-tagged protein substrate on the N-terminus (FIG. 6A) or C-terminus (FIG. 6B). Immunoblot analysis using an anti-HA-antibody of the cell lysates was performed after 3 days of incubation. Cleavage of substrate occurred in a manner dependent on a targeting crRNA as shown in FIGS. 6A-6B.

### Example 2

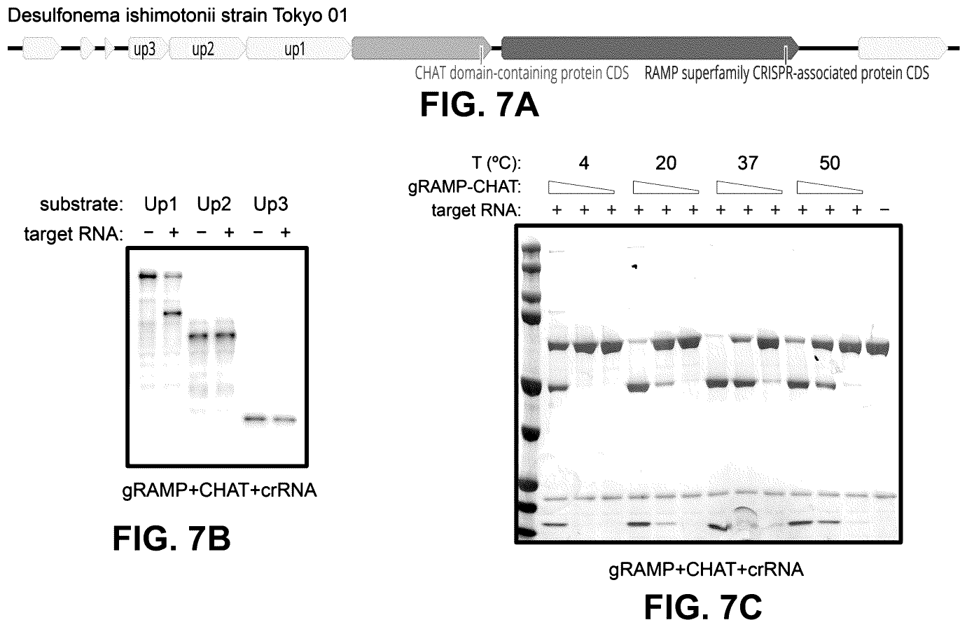

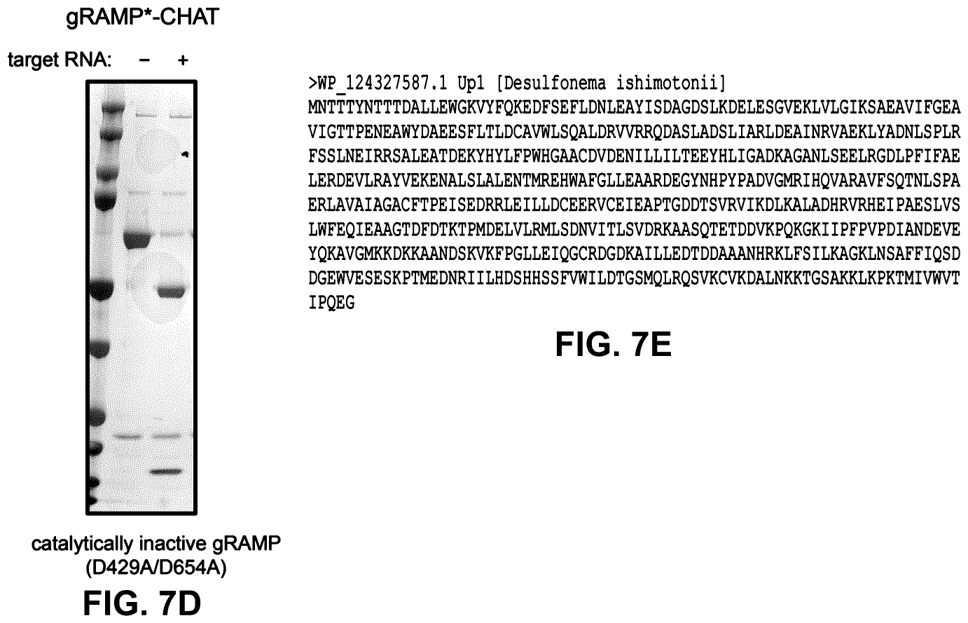

In vitro experiments were performed to examine the gRAMP-CHAT locus and the Up1 gRAMP-CHAT substrate. FIGS. 7A-7E demonstrate the gRAMP-CHAT locus from Desulfonema ishimotonii strain Tokyo 01 and that Upstream protein 1 (Up1, WP_12327587.1) is cleaved by the gRAMP-CHAT in response to target RNA. The gRAMP-CHAT complex exhibited protease activity across a wide range of temperatures ranging from 4-50 degrees C. Further, RNA cleavage by gRAMP is not required for protease activity as inactivating the nuclease with the D429A/D654A mutations has no effect on protease activity. Without being bound by theory, this can facilitate applications for sensing RNA without their destruction.

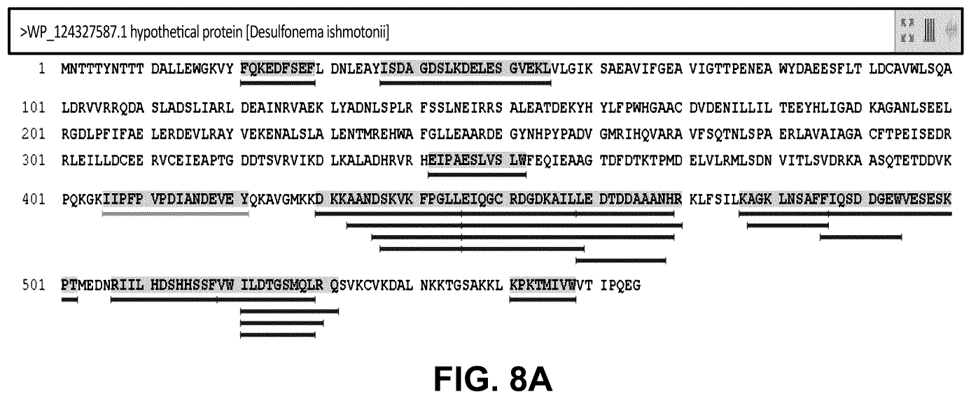

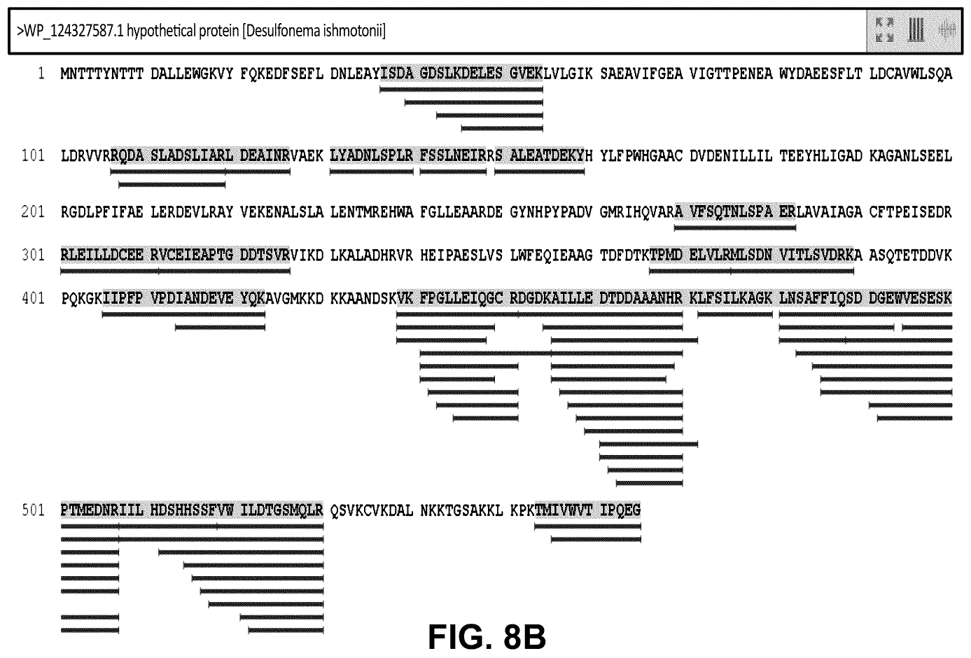

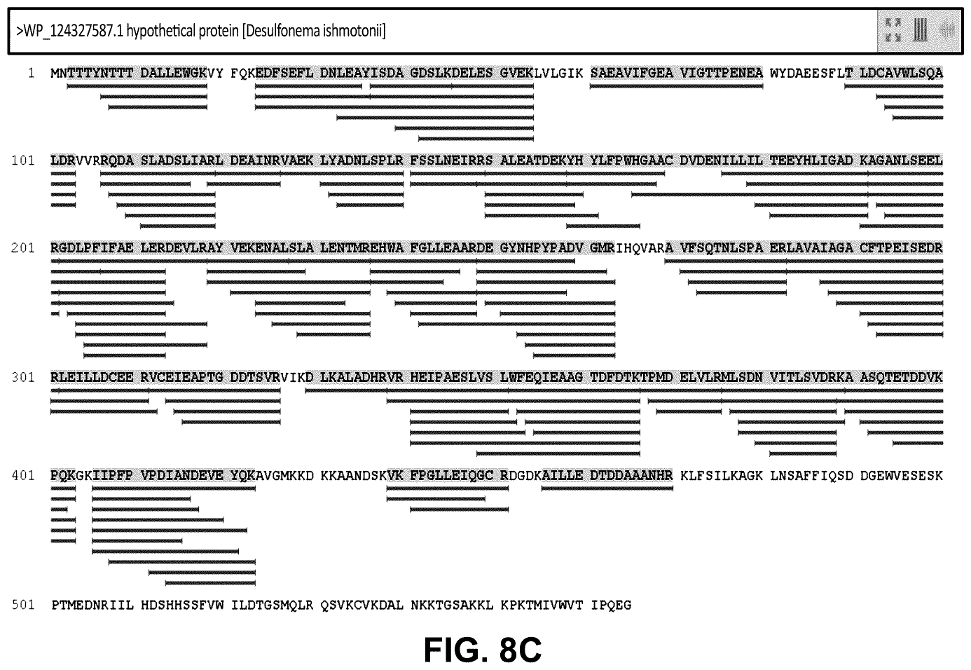

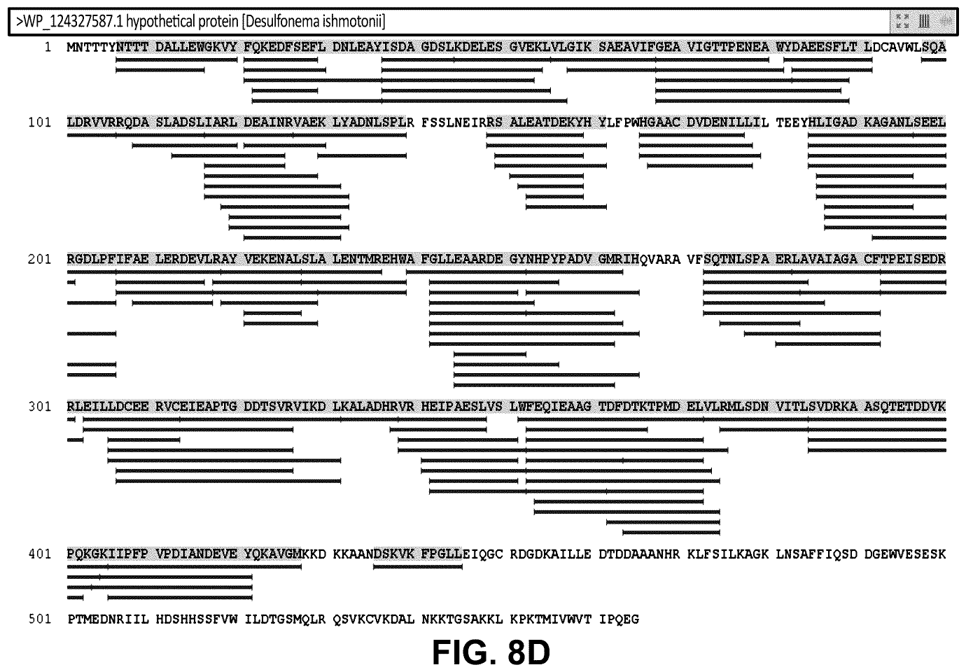

Enzyme digest mapping of peptides from the two fragments (N-terminal and C-terminal) produced from Up1 cleavage with the Desulfonema ishimotonii strain Tokyo 01 gRAMP-CHAT. Without being bound by theory, enzyme digest mapping revealed an approximate breakage point around M427-D430. See FIGS. 8A-8D.

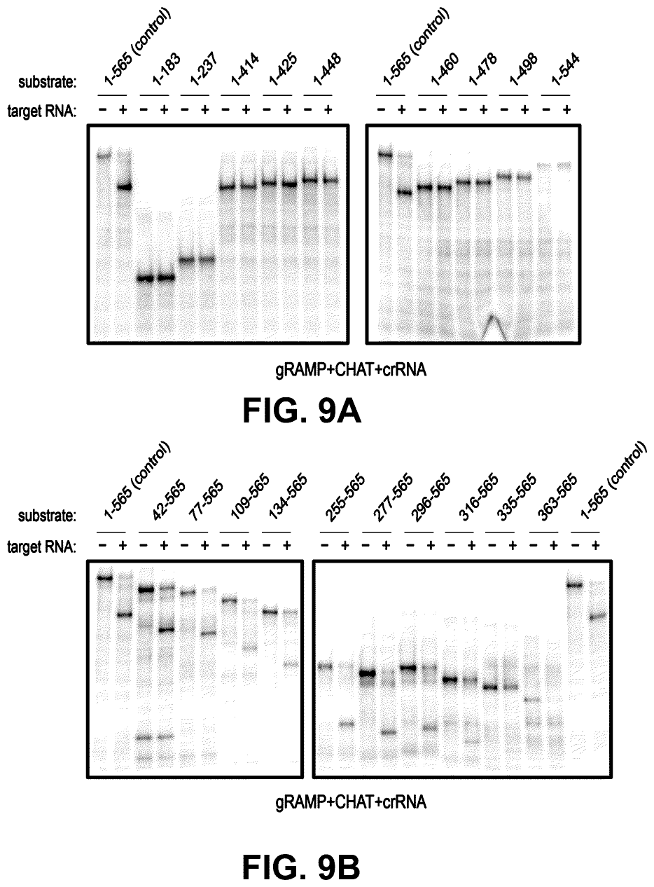

Truncation mapping of the Up1 substrate demonstrated that the C-terminal end of Up1 is required for cleavage but that the N-terminal end can be truncated. Smaller versions of Up1 containing amino acids 296-565 retained full activity for processing and can be used in applications to reduce the size of the protein substrate. See FIGS. 9A-9B.

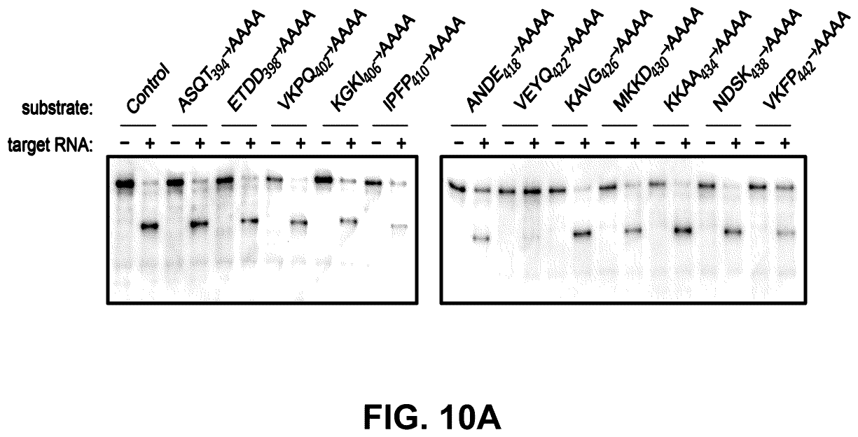

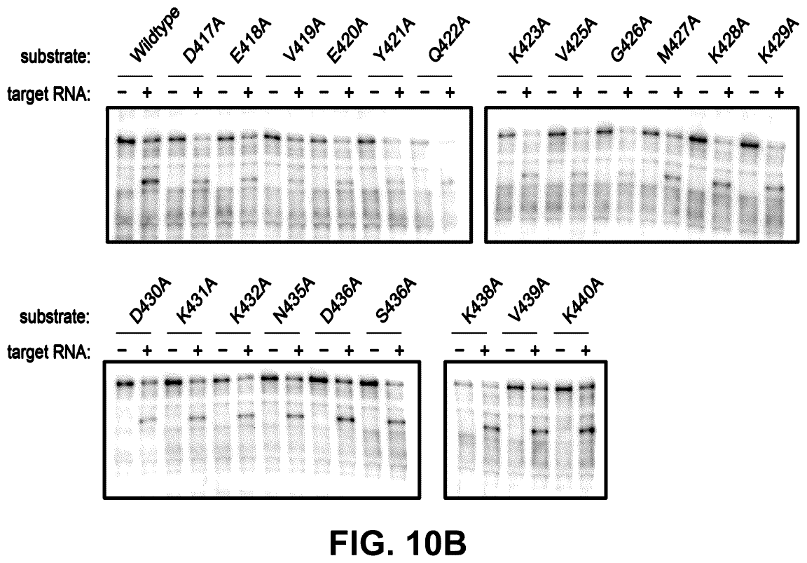

Alanine substitution mutation analysis in the Up1 protein substrate examined the effect of different amino acids have on gRAMP-CHAT mediated protein cleavage. No single alanine mutation blocks CHAT protease activity, which suggested that cleavage is not dependent on a specific residue and potentially that the shape of the substrate is being recognized. See FIGS. 10A-10B.

### Example 3

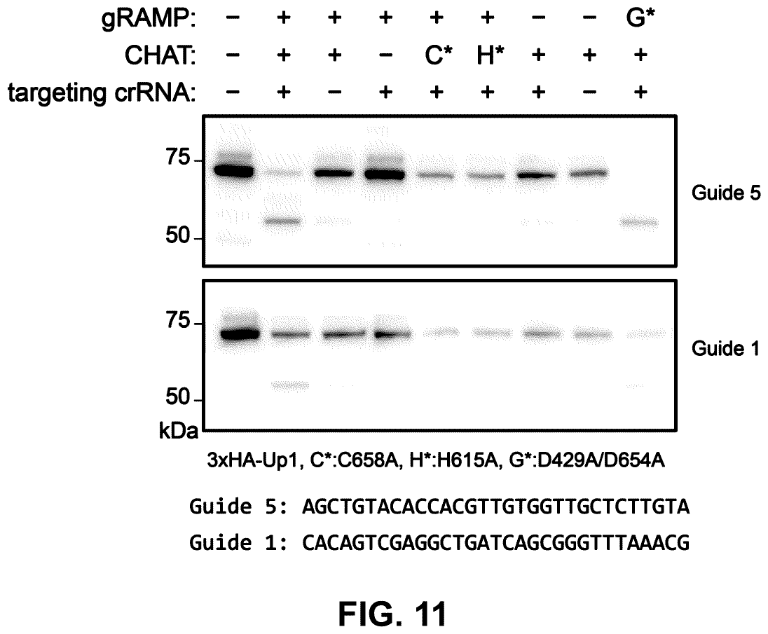

In vivo experiments were performed in human cells that demonstrated processing of 3×HA-tagged Up1, which is dependent on gRAMP, CHAT, and a targeting crRNA. See FIG. 11. This activity was abolished in the C658A and H615A CHAT mutations, which disrupted the catalytic site. Consistent with the in vitro data, inactivating the gRAMP nuclease residues with D429A/D654A mutations does not prevent cleavage of Up1 indicating that target RNA binding alone is required. This work was performed with two separate spacer sequences as shown in FIG. 11.

### Example 4

The gRAMP-CHAT substrate (e.g., Up1) and/or gRAMP-CHAT can be incorporated into an in vitro nucleic acid detection assay. FIG. 12 shows an exemplary schematic for an in vitro nucleic acid detection with gRAMP-CHAT. A gRAMP-CHAT substrate (e.g., Up1) containing an N-terminal avidin tag, which can be biotinylated, and a C-terminal FAM. Cleavage of the biotin-Up1-FAM substrate in response to target RNA can allow for visual detection on a standard biotin/FAM flow strip.

### Example 5

The gRAMP-CHAT substrate (e.g., Up1) and/or gRAMP-CHAT can be incorporated into an in vivo effector system. FIG. 13 shows an exemplary schematic for an in vivo effector system in which proteins are tethered to a cell membrane using transmembrane domains (e.g., gap43: LCCMRRTKQVEKNDEDQKI (SEQ ID NO: 34), L10: GCVCSSNPENNNN (SEQ ID NO: 35), S15: GSSKSKPKDPSQRRNNNN (SEQ ID NO: 36)) with a linker sequence containing a minimal Up1 substrate (amino acids 297-565). Following RNA detection and Up1 cleavage, the effector domain can move into the nucleus and perform different biological activities. For example, dCas9-VPR effector can be used to allow for the activation of genes, and a Cre effector to activate GFP expression.

### Example 6

The gRAMP-CHAT substrate (e.g., Up1) and/or gRAMP-CHAT can be incorporated into a degron. FIG. 14 Shows an exemplary schematic for a degron in which a degron tag is fused to an effector of interest via a linker sequence containing a minimal Up1 substrate (297-565). For example, a dihydrofolate reductase (DHFR) sequence (ISLIAALAVDHVIGMETVMPWNLPADLAWFKRNTLNKPVIMGRHTWESIGRPLPGR KNIILSSQPSTDDRVTWVKSVDEAIAACGDVPEIMVIGGGRVYEQFLPKAQKLYLTHI DAEVEGDTHFPDYEPDDWESVFSEFHDADAQNSHSYCFEILERR (SEQ ID NO: 37)), which destabilizes the protein resulting in degradation. Following RNA detection and Up1 cleavage, the degron tag is removed from the effector thereby stabilizing the effector and allowing for its activity. Exemplary effectors include reporters (e.g., fluorescent proteins (e.g., GFP)), a Cas (e.g., Cas 9), Cre, and others. Such an approach can be applied to any effector of interest.

### Example 7-RNA-Activated Protein Cleavage with a CRISPR-Associated Endopeptidase

Prokaryotes possess a multitude of defense systems against foreign genetic elements, including clustered regularly interspaced short palindromic repeats (CRISPR) and CRISPR-associated proteins (Cas) systems^{4,5}. While the predominant function of CRISPR-Cas systems is to provide adaptive immunity via RNA-guided DNA or RNA nuclease activity, additional proteins have been identified in genetic association with CRISPR loci⁶. One example are the CRISPR-associated transposase (CAST) systems^{7,8}, which perform RNA-guided DNA insertion whereby nuclease inactive CRISPR effectors guide Tn7-like mobile genetic elements to specific DNA sequences^{9,10}. However, additional enzymatic functions linked to CRISPR-Cas systems remain to be discovered and characterized.

The identification and development of diverse nucleic-acid guided enzymes remains an ongoing goal in biology and an exciting area of investigation. Although advances in genomic technologies have unveiled tremendous insight into gene function, mutations that cause disease, and gene-expression differences between cell types, our ability to target and manipulate cells based on this information remains limited. While it is possible to disrupt^{11,12}, activate¹³, and edit genes¹⁴⁻¹⁶, there is a lack tools for more sophisticated cellular control based on the presence of certain mutations or cell-type specific gene expression signatures.

Previous work has uncovered several fascinating RNA-targeting type III CRISPR systems linked to proteases^{5,6}, including a Lon protease which responds to cyclic oligoadenylate second messengers to cleave the CRISPR-T protein¹⁷. In addition, a recently characterized subtype III-E single component effector gRAMP^{2,3} (also referred to as Cas7-11) is also associated with a protease, a CHAT family member containing tetratricopeptide repeats (TPR-CHAT). The CHAT family of proteases harbor catalytic cysteine residues and contain eukaryotic caspases involved in programmed cell death, and gRAMP-CHAT was previously hypothesized to act as a bacterial caspase³. Notably, gRAMP and TPR-CHAT from Candidatus Scalindua brodae were shown to form a stable protein complex³, however, the substrate and function of associated protease is unknown.

Here, Applicant determines the protein substrate and mechanism of a type III-E CRISPR-associated protease (CASP) system from Desulfonema ishimotonii, reveal insight into its natural function, and how it can be engineered for novel RNA sensing applications in vitro and in human cells.

A gRAMP-CHAT Complex Cleaves the Neighboring Gene Product Up1

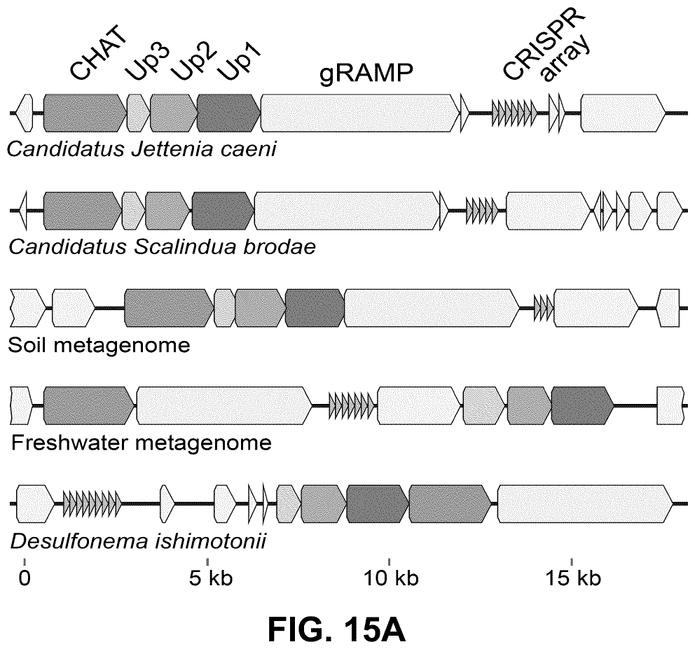

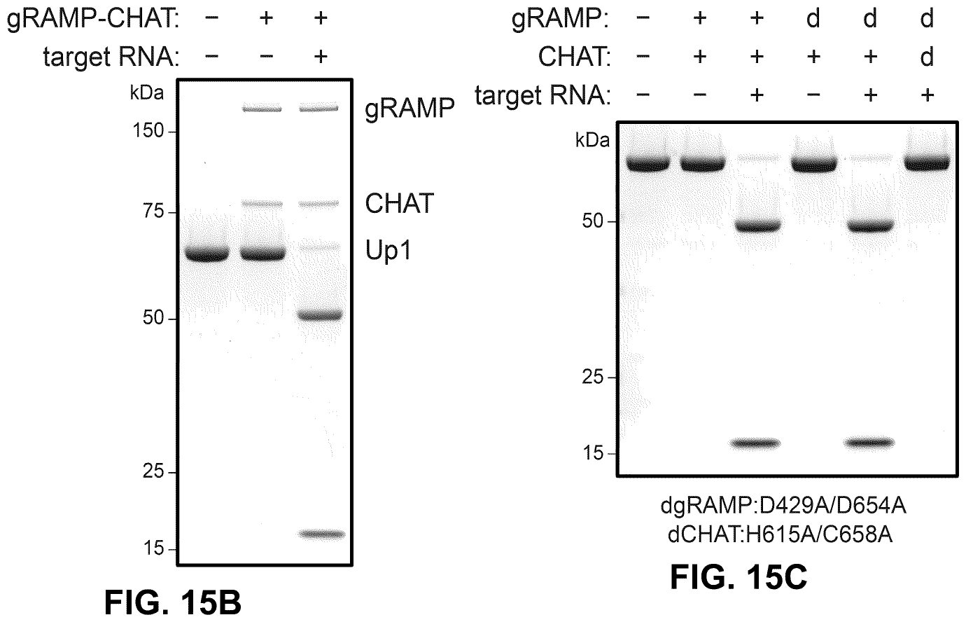

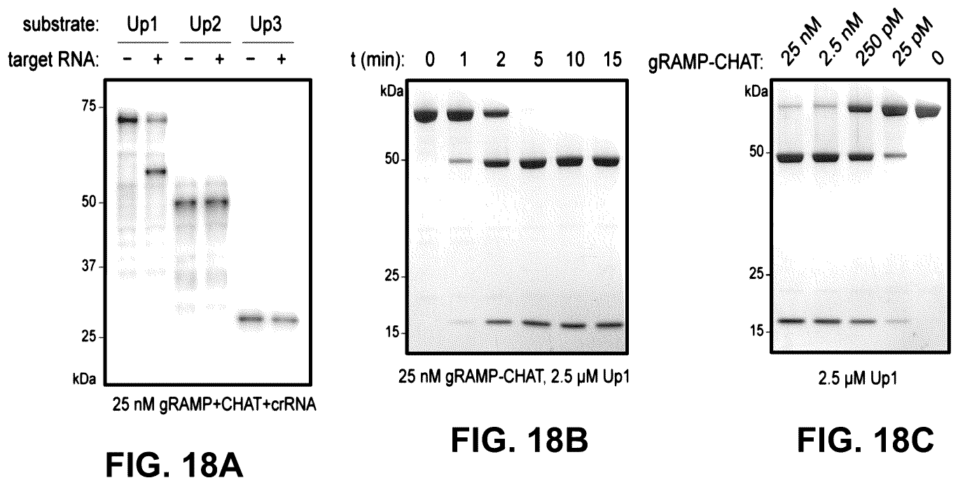

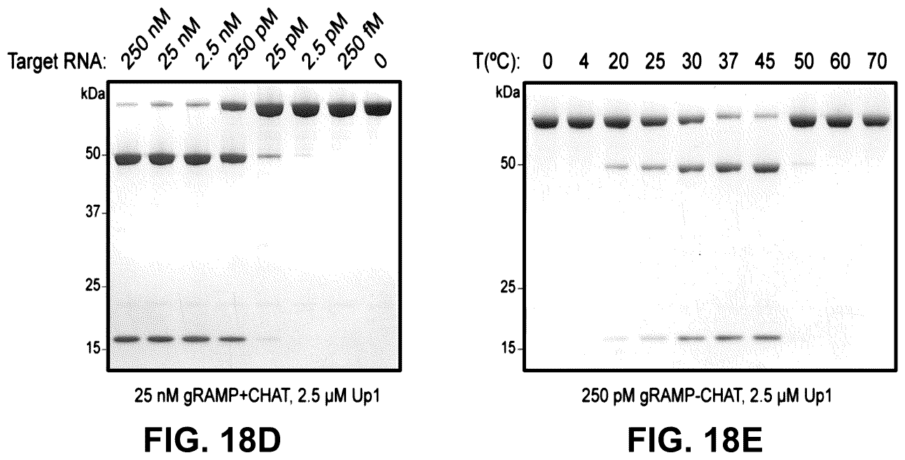

In contrast to prototypical type III CRISPR systems consisting of multi-subunit Csm/Cmr complexes, the subtype III-E family consists of a single component gRAMP effector containing naturally fused Cas7 domains¹⁸. In addition to the associated TPR-CHAT protease, these loci frequently contain three additional genes located in an operon (FIG. 15A), suggesting that they are likely involved in the natural function of CASP systems. Starting from a system in D. ishimotonii2 (DiCASP), Applicant was able to purify a stable gRAMP-CHAT-crRNA complex as previously reported with Candidatus S. brodae³. Applicant next performed in vitro reactions by adding the proteins expressed from the three upstream genes (Up1-3) in the presence or absence of a complementary RNA and identified that the largest protein, Up1, is specifically cleaved in response to target RNA (FIG. 15B, and FIG. 18A). These in vitro reactions yielded two precise protein products indicating a single cleavage event within Up1 as opposed to protein degradation.

Applicant determined the requirements of Up1 cleavage and found that while mutating the catalytic residues of the CHAT protease (H615A/C658A) abolished activity, disrupting the catalytic sites of gRAMP (D429A/D654A) did not (FIG. 15C). This result indicates that target RNA binding alone is sufficient for CHAT activation and that RNA cleavage is not required. In vitro characterization revealed that DiCASP is a highly processive ATP-independent protease cleaving 100-fold excess of Up1 substrate in minutes, and with an optimal activity at 37-45° C. (FIG. 18B-18E).

### Characterization of Up1 Proteolytic Processing

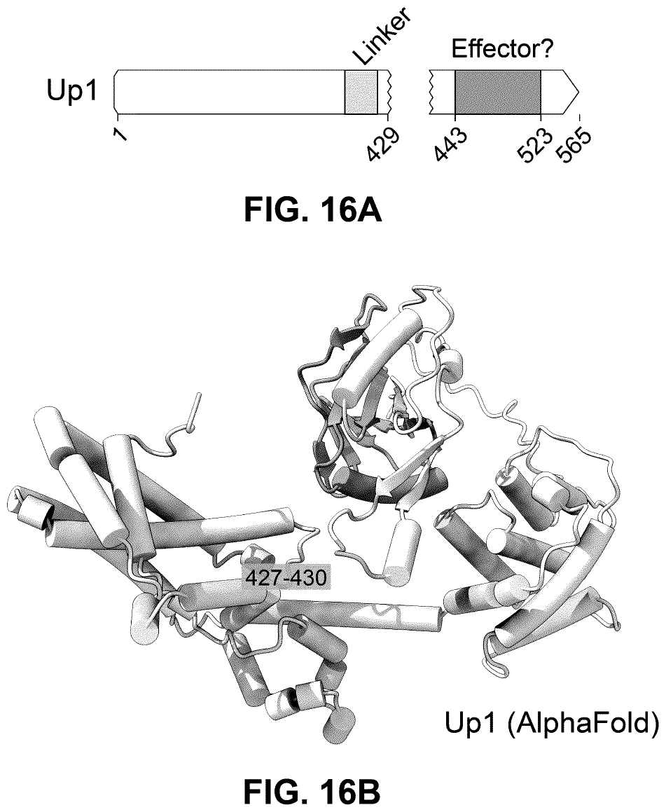

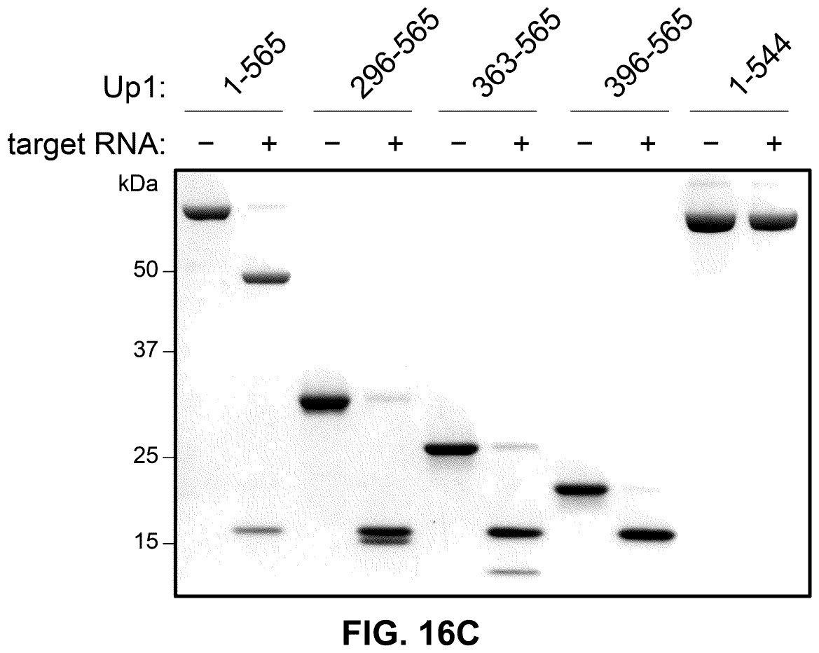

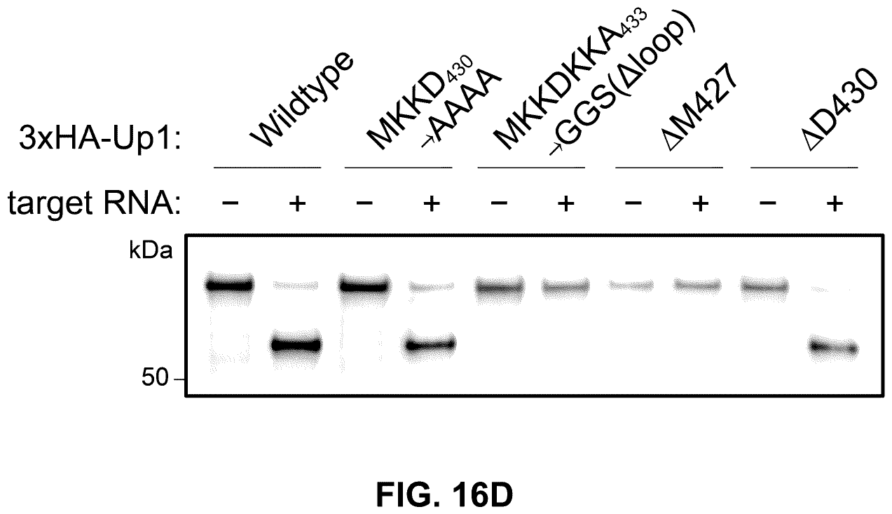

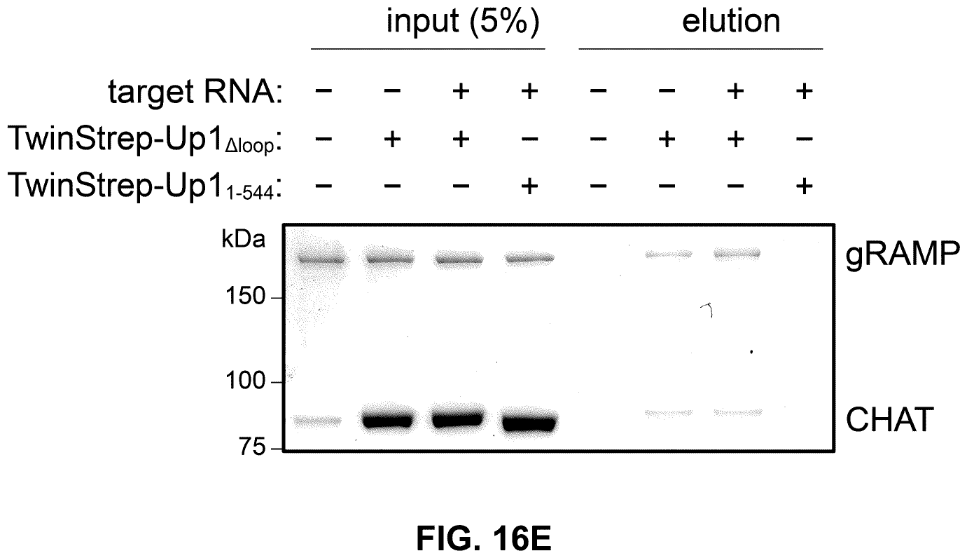

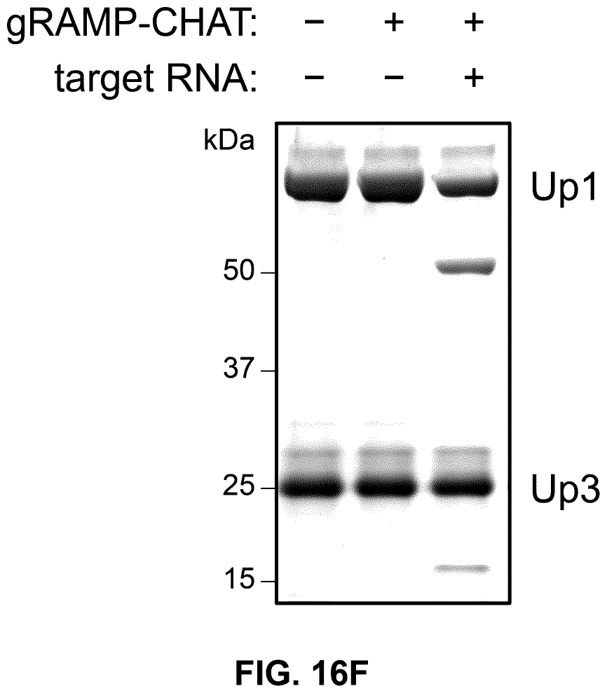

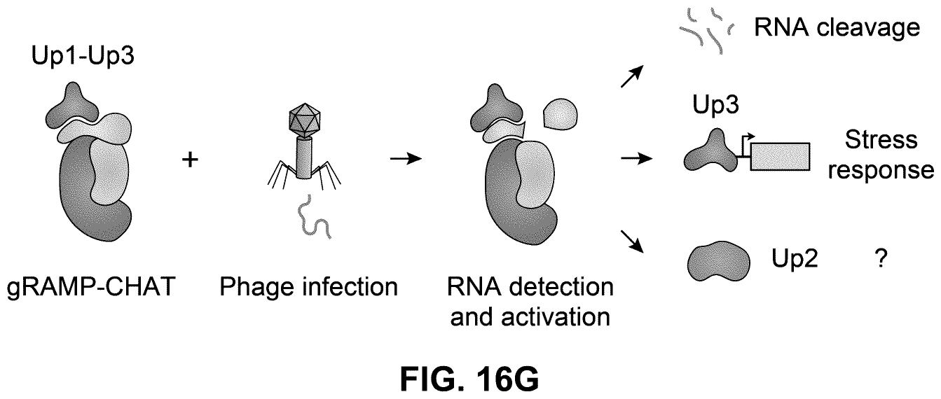

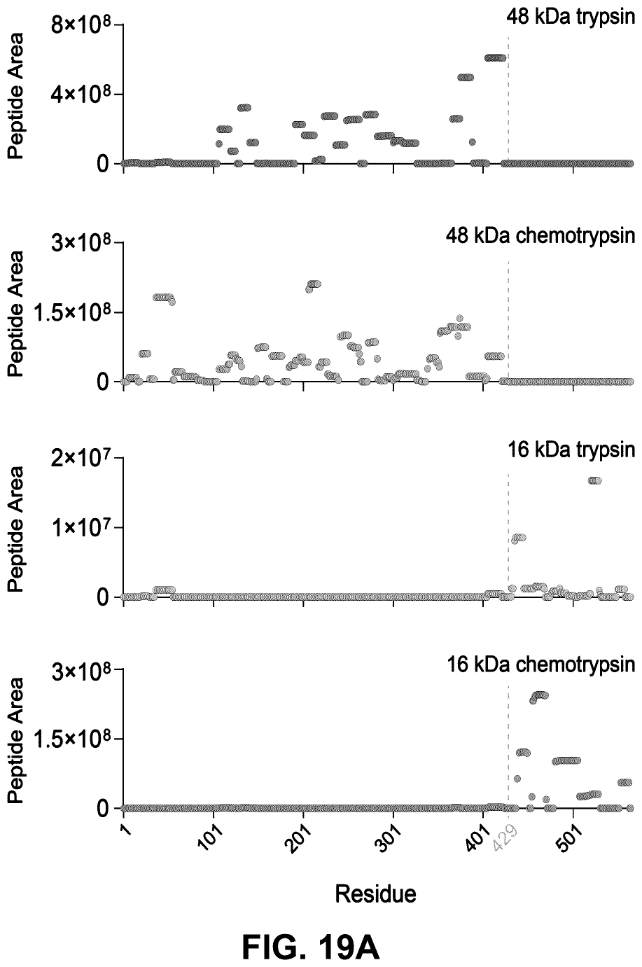

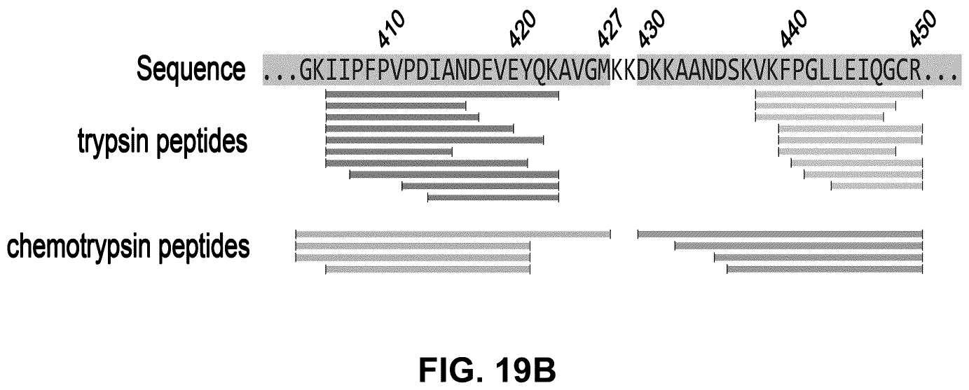

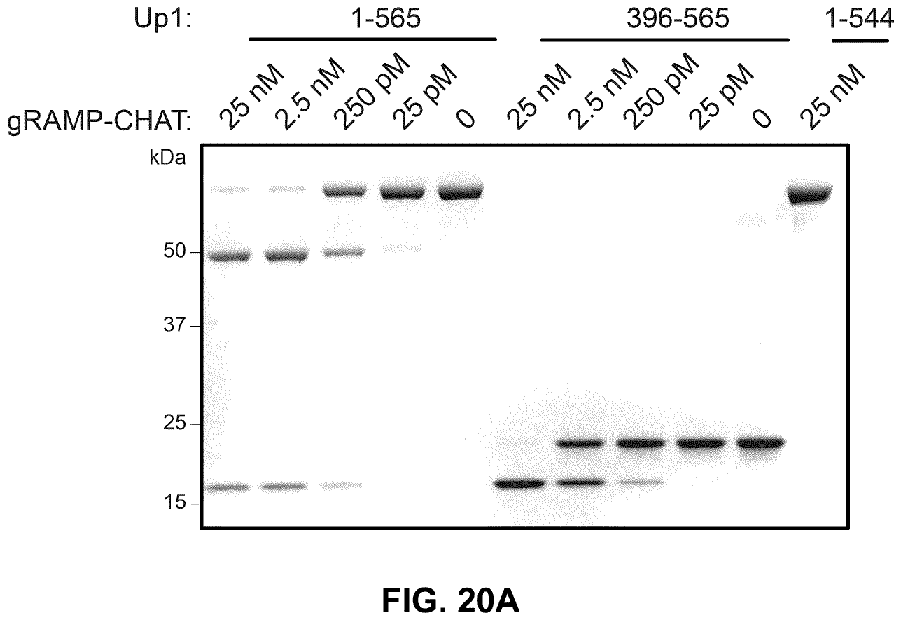

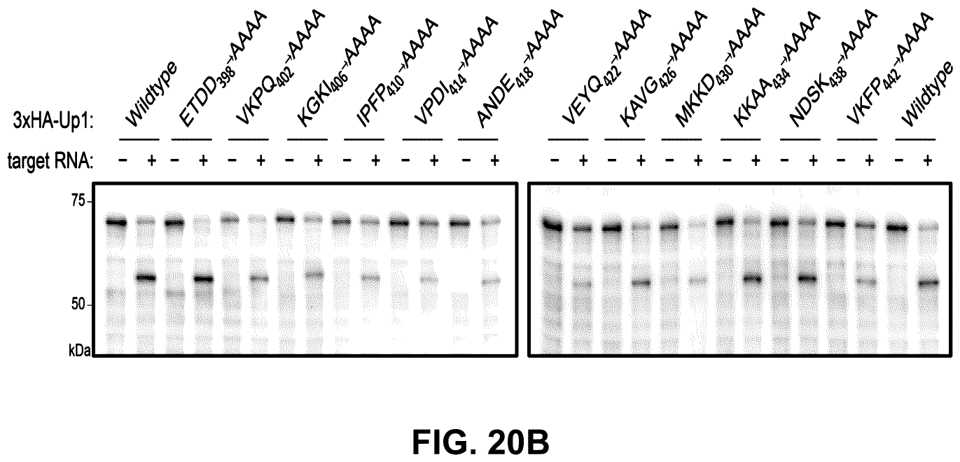

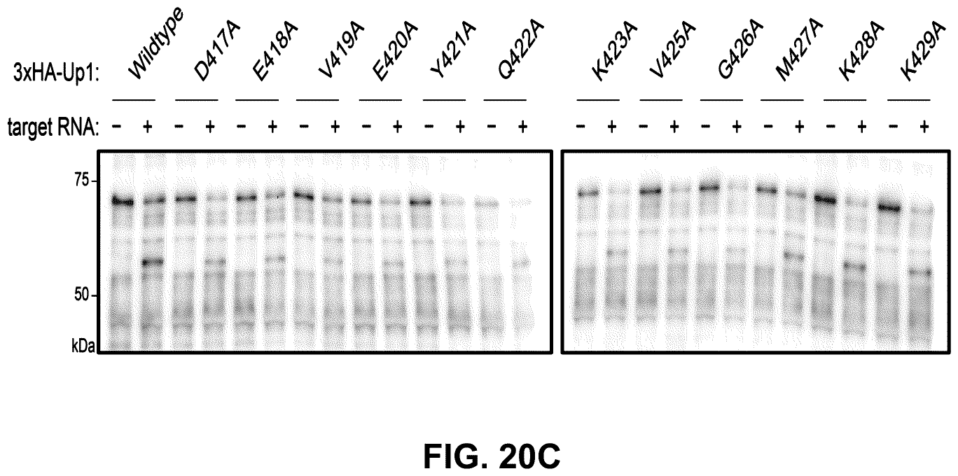

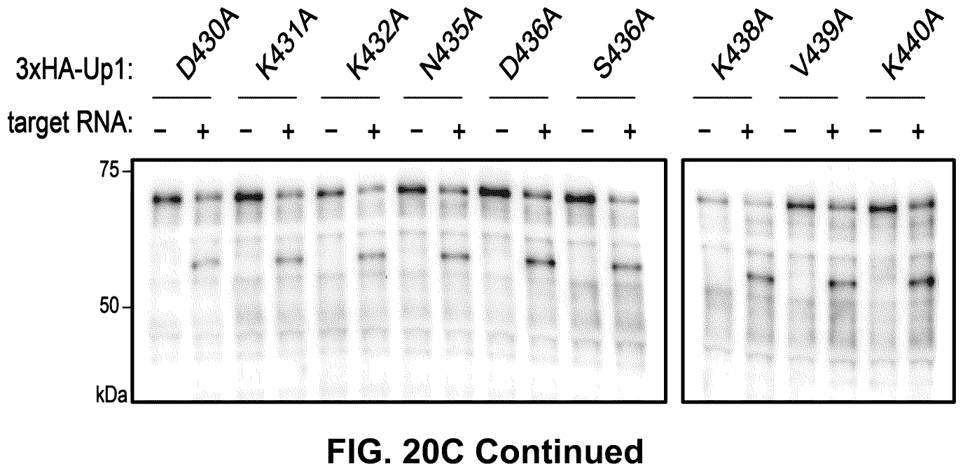

Structural prediction of the Up1 protein revealed two domains separated by a long flexible linker (FIG. 16A-16B) which Applicant hypothesized to be liberated following protein cleavage. However, mass spectrometry analysis (and the estimated 48 kDa and 16 kDa products) indicate that Up1 is cleaved further downstream between residues 427 and 430 (FIG. 19A-19B), placing the cleavage site within a small flexible loop in the C-terminal domain of the Up1 structural model. By generating truncation mutations of Up1, Applicant determined that the N-terminal sequence is dispensable for processing by gRAMP-CHAT as Up1 fragments containing residues 396-565 were fully active in vitro (FIG. 16C, and FIG. 20A). In contrast, Applicant observed that Up1 C-terminal residues are strictly required and that even a twenty amino acid truncation abolished activity (FIG. 16C).

Interestingly, mutational analysis by alanine substitutions revealed no Up1 residues critical for cleavage (FIG. 20B-20C), and instead that the size of the loop at position 427-430 is important for processing. Applicant observed that truncating the loop by four residues, or deleting M427 alone, prevented in vitro cleavage, while the deletion of D430 had no effect (FIG. 16D). Using an uncleavable Up1_{Δloop} mutant as bait, Applicant was able to pulldown active gRAMP-CHAT complex both in the presence and absence of target RNA, but not with a C-terminal truncation mutant (Up1₁₋₅₄₄), indicating that Up1 binding to gRAMP-CHAT is not dependent on activation of the protease (FIG. 16E).

### Up1 Binds the Transcription Initiation Factor Up3

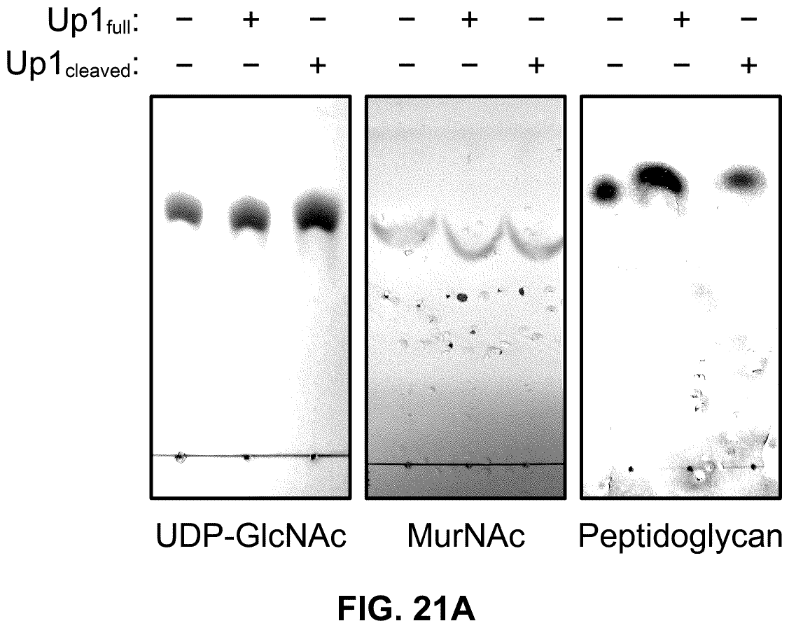

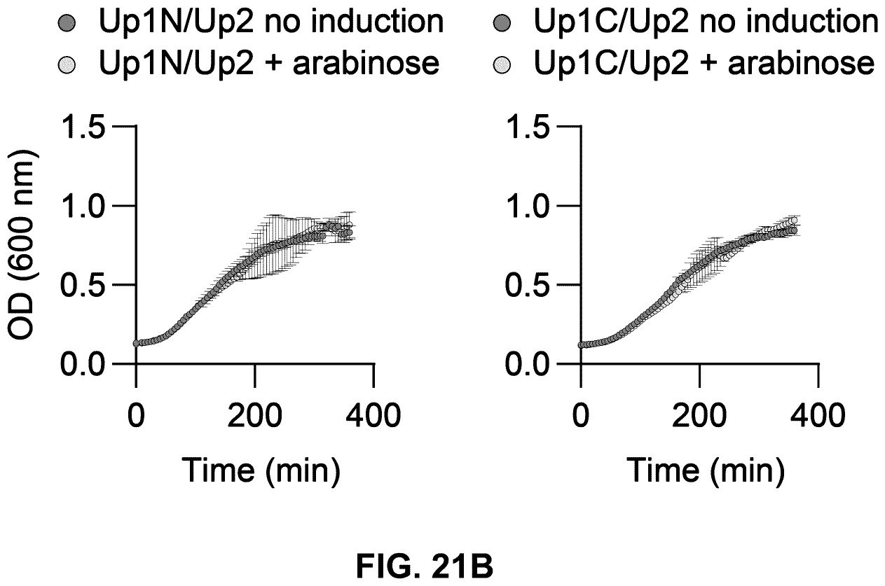

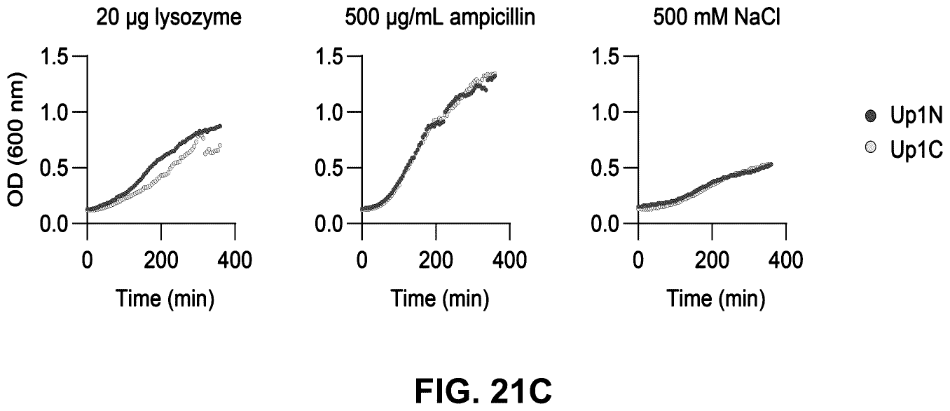

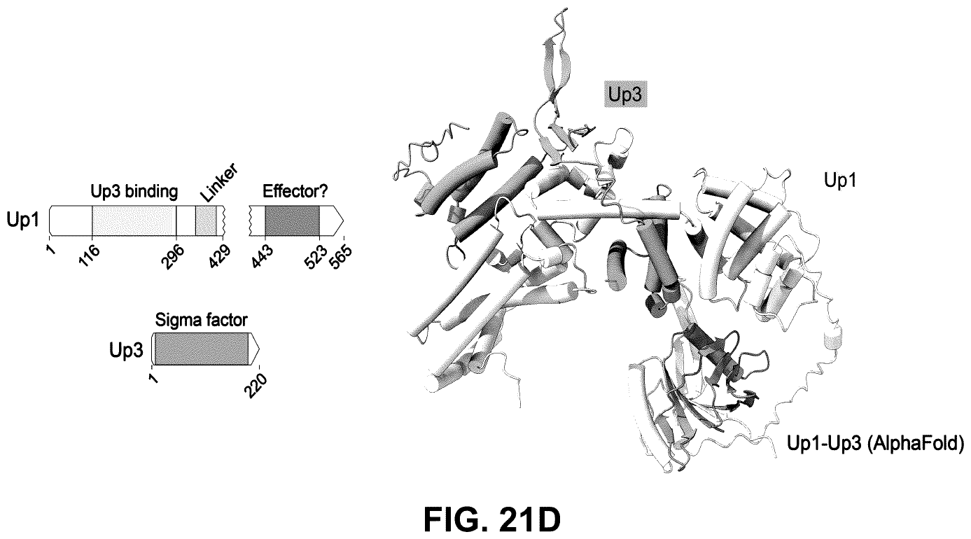

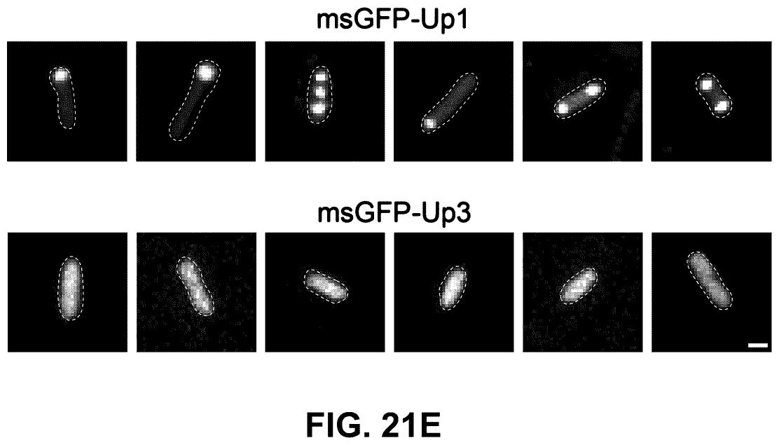

A fascinating question is the biological role of Up1 and how proteolytic processing regulates its activity. One intriguing possibility is that processed Up1 fragments, Up1N (residues 1-428) or Up1C (residues 429-565), might promote an abortive infection response to prevent phage propagation. Homology searches revealed a weak match of Up1C to a peptidoglycan deacetylase (HHpred¹⁹ probability: 92.4%, e-value: 0.66), however, Applicant did not detect processing of cell wall components by thin layer chromatography following in vitro reactions (FIG. 21A), and overexpression of neither fragment was toxic to E. coli (FIG. 21B). In contrast to a cell death response, processed Up1 might instead promote cell survival, but Applicant also did not detect any growth advantage under various cell wall stresses (FIG. 21C).

Rather, Applicant predicted a strong binding interaction between the N-terminal domain of Up1 and the adjacent Up3 protein, which strongly resembles a sigma factor (HHpred¹⁹ probability: 100%, e-value: 2.9e-31, FIG. 21D). Sigma factors are transcription initiation proteins that recruit RNA polymerase to specific sites, hinting that Up1 might be involved in regulating a transcriptional response to infection. Consistent with our computational binding prediction, purification of Up3 in the presence of untagged Up1 yielded an Up1-Up3 complex which could be cleaved by gRAMP-CHAT in the presence of target RNA (FIG. 16F). The Up1-Up3 interaction is predicted to block Up3 DNA binding suggesting that Up1 could be a sigma factor inhibitor.

Sigma factors are frequently regulated by inhibitors (anti-sigma factors) and there are several examples in bacteria in which a protease cleaves an anti-sigma factor to activate a transcriptional stress response including the anti-sigma factor RseA in E. coli²⁰, and the RsiW anti-sigma factor in B. subtilis²¹. In E. coli, the DegS protease senses cell envelope stress and cleaves RseA²², a transmembrane component, to release the bound sigma factor and Applicant was curious whether Up proteins are similarly spatially regulated in E. coli. Applicant generated fusions to monomeric superfolder green fluorescent protein (GFP) and visualized live cells by confocal microscopy. In contrast to msGFP-Up3 which was evenly distributed throughout the cell, msGFP-Up1 revealed distinct clustering at the cell poles, often with 1 or 2 foci per cell, but occasionally more (FIG. 21E). This phenotype is reminiscent of cell division proteins like FtsZ, or those with the ability to self-assemble²³, and Applicant hypothesizes that spatial clustering of Up1 could assist the inhibition of Up3 by physical sequestration from the bacterial chromosome, similar to DegS and RseA. Together, our data supports a model whereby Up1 is a inhibitor of the sigma factor Up3 and that Up1 cleavage could trigger transcriptional changes as one arm of the defense response (FIG. 17D).

RNA Sensing Applications with CASP Systems

The high enzymatic turnover of Up1 in response to a target RNA enables numerous biological applications. In addition, the ability to uncouple RNA cleavage from activation of the CHAT protease allows for non-destructive sensing of RNA. While the collateral nuclease activity of CRISPR effectors has been used to cleave nucleic acid substrates in diagnostic applications²⁴, CASP systems allow for a new modality of substrates using engineered Up1 proteins. As a proof of concept, Applicant purified an avidin-tagged form of Up1₂₅₀₋₅₆₅, biotinylated in vitro with BirA, and fluorescently labeled with NHS-fluorescein (FIG. 17A). To prevent labeling of Up1N amine side chains, Applicant mutated eight lysine residues to arginine, and four lysines within the cleavage loop to alanine (FIG. 22A). By immobilizing Up1 substrates and measuring released fluorescence activity, Applicant could perform in vitro detection of RNA across a wide range of RNA concentrations without nucleic acid amplification (FIG. 17B).

The ability to sense mRNA within live cells remains an unmet goal in biology and Applicant envisions that RNA-activated proteases could be useful for a variety of cellular functions. To determine if DiCASP can mediate RNA-guided protein cleavage in human cells Applicant transfected HEK293T cells with plasmids expressing gRAMP, CHAT, crRNA, a synthetic target RNA, and Up1 fused to an 3×HA epitope tag. Immunoblot of cell lysates revealed processing of Up1 that was dependent on a targeting crRNA, and the catalytic residues of the CHAT protease, but not gRAMP (FIG. 17C), consistent with our in vitro results.

Truncation analysis of Up1 also confirmed that N-terminal residues are dispensable for human cell activity facilitating the design of protein reporters containing minimal fragments of Up1 (FIG. 22B). Testing DiCASP activity and Up1 cleavage across a panel of endogenous transcripts revealed efficiencies ranging from 3 to 22% (FIG. 17D), with moderate correlation to RNA expression level (R²=0.624, FIG. 22C). To convert Up1 cleavage into a discrete and readily detectable signal Applicant constructed reporters in which the Cre recombinase is tethered to membrane anchors and sequestered from the nucleus (FIG. 17E). Applicant transfected mouse Neuro-2A cells harboring an inactive loxP-GFP reporter cassette which is expressed only upon Cre activity. Flow cytometry analysis revealed crRNA-dependent GFP expression in 10% of cells, and a 15-fold increase over non-targeting controls in the best conditions (FIG. 17F and FIG. 22D).

### DISCUSSION

Here Applicant demonstrates that the TPR-CHAT protease associated with the type III-E RNA-targeting gRAMP effector mediates RNA-activated endopeptidase activity and elucidate its substrate and mechanism. Our results support a model whereby an Up1-Up3 complex can bind to the CHAT protease, and that target RNA recognition mediated by gRAMP and a crRNA, but not RNA cleavage, is required for protease activation.

Although the full biological consequence of Up1 processing in the native host D. ishimotonii is unknown, our work points to a function in regulating the sigma factor Up3. Together, Applicant proposes a three-pronged strategy of defense that type III-E CASP systems use against phage including targeted RNA cleavage via the RNA endonuclease gRAMP, an Up1-Up3 regulated transcriptional stress response, and a potential third arm mediated through Up2 (FIG. 16G). The clear conservation of Up2 across CASP systems is a strong indication of its biological involvement and future work will be required to determine its role in the defense response.

Up3 is similar to the sigma-70 family of transcription initiation factors, including RpoE which controls an envelope stress response and can be activated by various stresses including phage infection. The parallels between DiCASP and other protease-regulated anti-sigma factors, like DegS and the transmembrane anti-sigma factor RseA²², are incredible, and reveal convergent mechanisms to elegantly modulate gene expression in response to cellular threats. The discovery that Up1 localizes to the cellular poles in a heterologous host suggests that this is likely an intrinsic property of Up1 to self-assemble and could have implications for applications with Up1-based reporters. Applicant hypothesizes this activity is mediated by the C-terminal domain.

Applicant predicts that Up1 interacts with Up3 through its N-terminal residues (FIG. 21D), and therefore it remains unclear how proteolytic cleavage within the Up1 C-terminal domain releases Up3. While changes in spatial localization could be involved, it is possible that additional host proteins are required for the full degradation of Up1 following initial cleavage by CHAT. Applicant notes that DegS cleavage of RseA is also insufficient to release sigma factor and the remaining RseA fragment is further processed by Rsep^{25,26} and the ClpXP protease²⁷ to allow transcriptional activation.

The parallels between the subtype III-E CASP systems investigated here and the type III CRISPR-associated Lon protease¹⁷ are fascinating and further investigation into the function of processed CRISPR-T and diverse Up1 proteins will be required to determine if convergent evolution is at play. The ability of independent type III CRISPR systems to co-opt these enzymes raises the likelihood that additional RNA-activated proteases exist in nature awaiting discovery.

While there are numerous technologies to detect RNA in fixed cells, the ability to sense transcripts in live cells should enable powerful new technologies to target and manipulate specific cell types. While our work provides a method to label specific cell types, for example to identify and isolate specific cell types from a loxP:GFP mouse, additional applications could enable cell-type specific genome editing or gene expression by tethering other effectors to the cell membrane, or via the removal of protein degron tags.

Although Up1 can be substantially truncated for applications, the relatively large size of the minimal fragment (˜160 amino acids) provides both advantages and challenges. While this likely affords high specificity and a low chance of nonspecific protein cleavage within cells, it could hinder the ability to engineer new substrate specificities including against endogenous human proteins. The ability to sense lowly expressed genes with DiCASP also remains limited and future engineering and protein evolution will also be required to realize the full potential of this system in cells. Despite these challenges, the ability to sense RNA and activate a new enzymatic function will provide new possibilities in biology. This work reveals an exciting example of CRISPR systems coordinating a wider cellular response beyond nuclease activity, and Applicant expects that the continued investigation of CRISPR-associated enzymes will provide interesting and useful RNA-activated functions moving forward.

### Material and Methods

### Gene Synthesis and Cloning

The TPR-CHAT protease and Up1-3 genes from D. ishimotonii were codon optimized for human cell expression (GenScript) and synthesized and assembled from gene fragments. Additional materials were cloned by Gibson Assembly (New England Biolabs). pDF0159 (pCMV-huDisCas7-11, Addgene #172507), pDF0118 (TwinStrp-SUMO-DisCas7-11, Addgene #172503), and pDF0114 (pU6-crRNA, Addgene #172508) were gifts from Omar Abudayyeh & Jonathan Gootenberg.

### In Vitro RNA Synthesis

In vitro transcribed RNA was generated by annealing a DNA oligonucleotide containing the reverse complement of the desired RNA with a short T7 oligonucleotide. In vitro transcription reactions were performed using the HiScribe T7 High Yield RNA synthesis kit (NEB) at 37° C. for 8-12 h and RNA was purified using Agencourt AMPure RNA Clean beads (Beckman Coulter).

### Cell-Free Transcription-Translation

3×HA tagged forms of Up1-3 were cloned into pCDNA3.1 vectors and amplified by PCR using oligos containing the T7 promoter and terminator. Cell-free transcription-translation was performed using PURExpress (New England Biolabs) in 5 μL reactions containing 2 μL buffer A, 1.5 μL buffer B, 0.25 μL of Superase RNAse Inhibitor (Invitrogen), and 50-100 ng of PCR template. Reactions were incubated for 2 h at 37° C. and directly transferred to in vitro reactions.

### Protein Purification

All proteins were expressed in BL21 E. coli (Sigma Aldrich, CMC0016). Cells were grown in Terrific Broth (TB) to mid-log phase and the temperature lowered to 18° C. Expression was induced at OD₆₀₀ 0.6 with 0.25 mM IPTG for 16-20 h before harvesting and freezing cells at −80° C. The gRAMP-CHAT complex was purified following co-expression of plasmids containing TwinStrep-SUMO-gRAMP and a mature crRNA, and pCDF-6×HIS-CHAT. Cell paste was resuspended in lysis buffer (50 mM Tris pH 7.5, 250 mM NaCl, and 5% glycerol) supplemented with EDTA-free complete protease inhibitor (Roche). Cells were lysed using a microfluidizer and cleared lysate was bound to Strep-Tactin Superflow Plus (Qiagen) using the gRAMP affinity tag. The resin was extensively washed and bound protein eluted by cleaving the TwinStrep-SUMO tag with Ulp1 protease overnight digest at 4° C. (1:100 ratio). The eluted protein was bound to Ni-NTA Superflow (Qiagen) in 15 mM imidazole using the CHAT affinity tag, the resin extensively washed with lysis buffer plus 40 mM imidazole, and the complex eluted with 300 mM imidazole buffer. The eluted complex was diluted to 100 mM NaCl and purified on a HiTrap Heparin (Cytiva) column with a 100 mM to 1 M NaCl gradient. Fractions containing the gRAMP-CHAT complex were pooled, concentrated, and run on a Superose 6 Increase column (Cytiva) with a final storage buffer of 25 mM Tris pH 7.5, 250 mM NaCl, 10% glycerol, 1 mM DTT.

Up1 was purified using a TwinStrep-SUMO tag and lysis buffer containing 50 mM Tris pH 7.5, 250 mM NaCl, and 5% glycerol. Following Ulp1 digest, Up1 protein was diluted to 100 mM NaCl and purified using a Resource Q anion exchange column (Cytiva) with a 100 mM to 1 M NaCl gradient before gel filtration chromatography on a Superose 6 Increase column (Cytiva) with a final storage buffer of 25 mM Tris pH 7.5, 250 mM NaCl, 10% glycerol, 1 mM DTT. For pulldown experiments, Up1 protein was eluted with 5 μM desthiobiotin instead of Ulp1 cleavage before ion exchange chromatography.

Up3 was purified using a pCDF-6×HIS-Up1 plasmid and Ni-NTA Superflow resin (Qiagen) in lysis buffer containing 50 mM Tris pH 7.5, 250 mM NaCl, 1 mM MgCl₂, 5% glycerol and 15 mM imidazole. The resin was extensively washed with lysis buffer plus 40 mM imidazole, and Up3 eluted with 300 mM imidazole buffer. The Up1-Up3 complex was purified in a similar way with the addition of a pUC19 plasmid containing untagged Up1. The complex was purified using a Resource Q anion exchange column (Cytiva) following Up3 elution and moved to storage buffer (25 mM Tris pH 7.5, 250 mM NaCl, 10% glycerol, 1 mM DTT).

### Up1 In Vitro Reactions

Typical in vitro reactions were performed in 20 μL containing 4 μL of 5× reaction buffer (100 mM HEPES pH 7.5, 500 mM NaCl, 5 mM DTT, 25% glycerol), 0.5 μL of 150 mM MgCl2, 1 μL of Up1 substrate (2.5 μM final concentration), 2 μL of gRAMP-CHAT-crRNA complex (25 nM final concentration), and 2 μL of purified target RNA (250 nM final concentration). Reactions were incubated at 37° C. for 1 hour before the addition of Laemmli buffer. Samples were boiled for 5 minutes and run on 12-well Nupage 4-12% Bis-Tris gels (Invitrogen) and stained with Coomassie dye before imaging on a Chemi-Doc (Bio-Rad).

### Thin Layer Chromatography

Uridine 5′-diphospho-N-acetylglucosamine (UDP-GlcNAc, Sigma Aldrich U4375), N-acetylemuramic acid (MurNAc, Sigma Aldrich A3007), and peptidoglycan from Bacillus subtilis (Sigma Aldrich, 69554) were resuspended in dimethyl sulfoxide at 10 mg/mL. Full length or cleaved Up1 protein was added and the reactions incubated at 37° C. for 2 hours in the presence of 1 mM MgCl₂, 1 mM ZnCl₂, and 5 mM DTT. Oligosaccharides were separated by thin layer chromatography on silica gel 60 F254 LuxPlates (Millipore Sigma) in 30% propanol for 1 hour, and charred with 30% ammonium bisulfate at 150° C. for visualization. UDP-GlcNAc was visualized under 254 nm UV light.

### Up1 Labeling and In Vitro Diagnostics

Mutated and truncated Up1 was purified as previously described except with HEPES buffer in all steps instead of Tris. Up1 was biotinylated in vitro using the BirA biotin ligase (Avidity). Up1 was incubated with with NHS-Fluorescein (Thermo Fisher Scientific, #46409) on ice for 1 h before quenching 200 mM Tris pH 7.5. Labeled Up1 was purified using a Resource Q anion exchange column as before. Purified biotin-Up1-FAM substrate was bound to MyOne Streptavidin T1 dynabeads (Thermo Fisher Scientific) in phosphate buffered saline for 30 min at room temperature. The beads were washed 10 times with PBS supplemented with 0.1% bovine serum albumin and resuspended in PBS. In vitro reactions were performed as before and Dyneabeads were removed from the reaction using a magnetic. The supernatant, containing cleaved Up1C, was transferred to 96-well plates and fluorescence measured using a Synergy Neo2 plate reader (BioTek) and subtracting the background signal from a well with no target RNA.

### Structural Predictions

Up1 and Up1-Up3 structures were predicted using Colabfold²⁸, an interface for Alphafold²⁹ and MMSeqs2 (UniRef+environmental).

### Microscopy

E. coli harboring pCDF-msGFP-Up1 and -Up3 were grown in LB to mid-log phase. Cells were centrifuged at 1000 g for 2 min, resuspended in PBS, and imaged using a STELLARIS 5 confocal microscope (Leica Microsystems). Images were acquired as Z-stacks and representative images show as maximum projections.

### Cell Culture and Transfection

HEK293T and Neuro2A cells were cultured in Dulbecco's modified Eagle medium with high glucose, sodium pyruvate, and GlutaMAX (Thermo Fisher Scientific), 1× penicillin-streptomycin (Thermo Fisher Scientific), and 10% fetal bovine serum (Seradigm). Cells were maintained at a confluency below 90%. For immunoblot analysis, 24-well plates were seeded with 87,500 cells/well approximately 16 h before transfection. Cell were typically transfected with 50 ng of 3×HA-Up1, 400 ng gRAMP, 400 ng CHAT, 100 ng target, and 500 ng crRNA in Opti-MEM (Thermo Fisher Scientific) with 4.5 L TransIt-LT1 transfection reagent (Mirus).

For flow cytometry experiments, 96-well plates were seeded with 17,500 cells/well. Cell were typically transfected with 60 ng gRAMP, 60 ng CHAT, 20 ng target, 60 ng crRNA, and 0.5-5 ng of Cre constructs in Opti-MEM (Thermo Fisher Scientific) with 0.6 μL TransIt-LT1 transfection reagent (Mirus).

### Western Blot and Flow Cytometry

Cells were typically harvested 96 h post-transfection. Cells were washed with ice-cold PBS and lysed in 75 μL of NP-40 lysis buffer (50 mM Tris pH 8, 150 mM NaCl, 1% NP-40). Cell suspensions were kept on ice for 10 min and cleared by centrifugation at 4 C for 10 min at 21,000 g. Lysates were stored at −80 before western blot analysis. Lysates were mixed with 4× Lammlae buffer (Bio-Rad) run on 12-well Nupage 4-12% Bis-Tris gels (Invitrogen). Proteins were transferred to PDVF membranes using an iBlot2 at 23V for 6 min. Membranes were blocked for 30 min at room temperature with TBST (Tris-buffer saline with 0.1% Tween 20) with 5% bovine serum albumin (Rockland). anti-HA:HRP (Cell Signaling Technologies, #2999) and anti-GAPDH: HRP (Cell Signaling Technologies #3683) were added at 1:5000 dilution and incubated for 30-60 min at room temperature. Membranes were washed 5× with TBST, incubated with Pierce ECL Western Blotting Substrate (Thermo Fisher Scientific) and imaged using a Chemi-Doc (Bio-Rad).

For flow cytometry analysis, cells were trypsinized 96 h post-transfection and resuspended in PBS supplemented with 5% FBS. Cells were analyzed using a CytoFLEX S flow cytometer (Beckman Coulter),

### REFERENCES FOR EXAMPLE 7

1. Anzalone, A. V., Koblan, L. W. & Liu, D. R. Genome editing with CRISPR-Cas nucleases, base editors, transposases and prime editors. Nat. Biotechnol. 38, 824-844 (2020).
2. Özcan, A. et al. Programmable RNA targeting with the single-protein CRISPR effector Cas7-11. Nature 597, 720-725 (2021).
3. van Beljouw, S. P. B. et al. The gRAMP CRISPR-Cas effector is an RNA endonuclease complexed with a caspase-like peptidase. Science 373, 1349-1353(2021).
4. Bernheim, A. & Sorek, R. The pan-immune system of bacteria: antiviral defence as a community resource. Nat. Rev. Microbiol. 18, 113-119(2020).
5. Makarova, K. S. et al. Evolutionary classification of CRISPR-Cas systems: a burst of class 2 and derived variants. Nat. Rev. Microbiol. 18, 67-83(2020).
6. Shmakov, S. A., Makarova, K. S., Wolf, Y. I., Severinov, K. V. & Koonin, E. V. Systematic prediction of genes functionally linked to CRISPR-Cas systems by gene neighborhood analysis. Proc. Natl. Acad. Sci. U.S.A. 115, E5307-E5316 (2018).
7. Peters, J. E., Makarova, K. S., Shmakov, S. & Koonin, E. V. Recruitment of CRISPR-Cas systems by Tn7-like transposons. Proc. Natl. Acad. Sci. U.S.A. 114, E7358-E7366 (2017).
8. Faure, G. et al. CRISPR-Cas in mobile genetic elements: counter-defence and beyond. Nat. Rev. Microbiol. 17, 513-525 (2019).
9. Strecker, J. et al. RNA-guided DNA insertion with CRISPR-associated transposases. Science 365, 48-53 (2019).
10. Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019).
11. Cong, L. et al. Multiplex genome engineering using CRISPR/Cas systems. Science 339, 819-823(2013).
12. Mali, P. et al. RNA-guided human genome engineering via Cas9. Science 339, 823-826 (2013).
13. Konermann, S. et al. Genome-scale transcriptional activation by an engineered CRISPR-Cas9 complex. Nature 517, 583-588(2015).
14. Komor, A. C., Kim, Y. B., Packer, M. S., Zuris, J. A. & Liu, D. R. Programmable editing of a target base in genomic DNA without double-stranded DNA cleavage. Nature 533, 420-424(2016).
15. Anzalone, A. V. et al. Search-and-replace genome editing without double-strand breaks or donor DNA. Nature 576, 149-157 (2019).
16. Gaudelli, N. M. et al. Programmable base editing of A·T to G·C in genomic DNA without DNA cleavage. Nature 551, 464-471 (2017).
17. Rouillon, C. et al. SAVED by a toxin: Structure and function of the CRISPR Lon protease. doi: 10.1101/2021.12.06.471393.
18. Kato, K. et al. Structure and engineering of the type III-E CRISPR-Cas7-11 effector complex. Cell (2022) doi: 10.1016/j.cell.2022.05.003.
19. Söding, J., Biegert, A. & Lupas, A. N. The HHpred interactive server for protein homology detection and structure prediction. Nucleic Acids Res. 33, W244-8 (2005).
20. OMP Peptide Signals Initiate the Envelope-Stress Response by Activating DegS Protease via Relief of Inhibition Mediated by Its PDZ Domain. Cell 113, 61-71 (2003).
21. Schöbel, S., Zellmeier, S., Schumann, W. & Wiegert, T. The Bacillus subtilis sigmaW anti-sigma factor RsiW is degraded by intramembrane proteolysis through YluC. Mol. Microbiol. 52, 1091-1105 (2004).
22. Ades, S. E., Connolly, L. E., Alba, B. M. & Gross, C. A. The Escherichia coli sigma (E)-dependent extracytoplasmic stress response is controlled by the regulated proteolysis of an anti-sigma factor. Genes Dev. 13, 2449-2461 (1999).
23. Rudner, D. Z. & Losick, R. Protein Subcellular Localization in Bacteria. Cold Spring Harbor Perspectives in Biology vol. 2 a000307-a000307 (2010).
24. Gootenberg, J. S. et al. Nucleic acid detection with CRISPR-Cas13a/C2c2. Science 356, 438-442 (2017).
25. Alba, B. M., Leeds, J. A., Onufryk, C., Lu, C. Z. & Gross, C. A. DegS and YaeL participate sequentially in the cleavage of RseA to activate the ζ^{E}-dependent extracytoplasmic stress response. Genes & Development vol. 16 2156-2168(2002).
26. Kanehara, K., Ito, K. & Akiyama, Y. YaeL (EcfE) activates the ζ^{E} pathway of stress response through a site-2 cleavage of anti-ζ^{E}, RseA. Genes & Development vol. 16 2147-2155 (2002).
27. Flynn, J. M., Levchenko, I., Sauer, R. T. & Baker, T. A. Modulating substrate choice: the SspB adaptor delivers a regulator of the extracytoplasmic-stress response to the AAA+ protease ClpXP for degradation. Genes Dev. 18, 2292-2301 (2004).
28. Mirdita, M. et al. ColabFold: making protein folding accessible to all. Nat. Methods 19, 679-682 (2022).
29. Jumper, J. et al. Highly accurate protein structure prediction with AlphaFold. Nature 596, 583-589(2021).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A programmable nuclease-peptidase composition comprising:
   a. a repeat-associated mysterious protein (RAMP) polypeptide;
   b. a guide molecule capable of forming a RAMP-guide molecule complex with the RAMP polypeptide and directing sequence specific binding of the complex to a target polynucleotide; and
   c. a peptidase capable of binding to the RAMP polypeptide, the guide molecule, or further complexing with the RAMP-guide molecule complex, wherein binding of the RAMP-guide molecule complex to the target polynucleotide initiates binding and/or interaction of the peptidase with a target polypeptide.
2. The programmable nuclease-peptidase composition of aspect 1, wherein target polypeptide interaction and/or binding occurs at, or in effective proximity to, a peptidase recognition motif in the target polypeptide.
3. The programmable nuclease-peptidase composition of aspect 2, wherein the peptidase recognition motif comprises or consists of SEQ ID NO: 3 or a sequence therein, optionally MKKD (SEQ ID NO: 25).
4. The programmable nuclease-peptidase composition of any one of aspects 1-3, wherein the peptidase is a TPR-CHAT peptidase.
5. The programmable nuclease-peptidase composition of aspect 4, wherein the TPR-CHAT peptidase is derived from Desulfonema ishimotonii, or a homolog, ortholog, or variant thereof.
6. The programmable nuclease-peptidase composition of any one of aspects 1-5, wherein the RAMP polypeptide is derived from Desulfonema ishimotonii, or a homolog, ortholog or variant thereof.
7. The programmable nuclease-peptidase composition of aspect 6, wherein the RAMP polypeptide comprises a Cas11 domain and multiple Cas7 domains.
8. The programmable nuclease-peptidase composition of aspect 7, wherein the RAMP polypeptide further comprises a Csm3, Csm4, or Csm6 domain.
9. The programmable nuclease-peptidase composition of aspect 6, wherein the RAMP polypeptide is a Type III-E Cas polypeptide.
10. The programmable nuclease-peptidase composition of any one of aspects 1-9, wherein the target polypeptide comprises, consists of, or is coupled to an effector, wherein the effector is optionally
   a. a reporter polypeptide;
   b. a signal amplification polypeptide;
   c. an engineered prodrug;
   d. a cargo polypeptide; or
   e. a pathogenic polypeptide.
11. The programmable nuclease-peptidase composition of any one of aspects 1-10, wherein the target polypeptide is a disease-associated polypeptide.
12. The programmable nuclease-peptidase composition of aspect 11, wherein the disease-associated polypeptide is associated with a neurodegenerative disease, a metabolic disease, an autoimmune disease, or a cancer.
13. A polynucleotide encoding a programmable nuclease-peptidase composition or component thereof as in any one of aspects 1-12.
14. The polynucleotide of aspect 13, further comprising one or more regulatory elements and wherein the polynucleotide encoding a programmable nuclease-peptidase composition or component thereof is operatively coupled to one or more of the one or more regulatory elements.
15. A vector or vector system comprising one or more polynucleotides according to any one of aspects 13 or 14.
16. The vector or vector system of aspect 15, wherein the vector or vector system is a viral vector or vector system, optionally an adeno-associated virus vector or vector system.
17. A cell or cell population comprising a programmable nuclease-peptidase composition of any one of aspects 1 to 12.
18. A pharmaceutical formulation comprising:
   a. a programmable nuclease-peptidase composition or component thereof as in any one of claims 1-12, a polynucleotide as in claim 13-14, a vector or vector system as in any one of claims 15-16, a cell or cell population as in claim 17, or any combination thereof; and
   b. a pharmaceutically acceptable carrier.
19. A method of modifying a polypeptide comprising:
   a. introducing into a sample having one or more target polynucleotides and one or more target polypeptides, the programmable nuclease-peptidase compositions of any one of aspects 1-12; and
   b. activating the peptidase via sequence specific binding of the complex to the one or more target polynucleotides such that the peptidase then binds or interacts with the one or more target polypeptides resulting in modification of the one or more target polypeptides.
20. The method of aspect 19, wherein the target polypeptide modification is cleavage of the target polypeptide.
21. The method of any one of aspects 19-20, wherein introducing into the sample comprises in vitro, ex vivo, or in vivo delivery of the programable nuclease-peptidase composition into a cell or cell population.
22. The method of any one of aspects 19-21, wherein the one or more target polypeptides comprise proenzymes and the modification results in conversion of the proenzyme into an active enzyme.
23. The method of any one of aspects 19-22, wherein modification of the one or more target polypeptides results in activation or deactivation of one or more cell-signaling proteins.
24. The method of any one of aspects 19-23, wherein the one or more target polynucleotides comprise a specific transcript or set of transcripts and wherein modification of the one or more target polypeptides triggers cell death upon activating the peptidase in response to binding of the nuclease-peptidase to the specific transcript or set of transcripts.
25. The method of aspect 24, wherein the guide molecule is configured to detect one or more mutations in the specific transcript or set of transcripts.
26. A detection composition comprising:
   (i) a RAMP polypeptide;
   (ii) a guide molecule capable of forming a RAMP-guide molecule complex with the RAMP polypeptide and directing sequence-specific binding of the complex to a target polynucleotide;
   (iii) a peptidase capable of binding the RAMP polypeptide, the guide molecule, or further complexing with the RAMP-guide complex; and
   (iv) a detection construct,
   wherein binding of the RAMP-guide molecule complex to the target polynucleotide initiates peptidase mediated modification of the detection construct resulting in generation of a detectable signal.
27. The composition of aspect 26, wherein the detection construct comprises a peptidase recognition motif recognized by the peptidase.
28. The composition of any one of aspects 26-27, wherein the peptidase recognition motif comprises or consists of SEQ ID NO: 3 or a sequence therein, optionally MKKD (SEQ ID NO: 25).
29. The composition of any one of aspects 26-28, wherein the peptidase is a TPR-CHAT peptidase.
30. The composition of aspect 29, wherein the TPR-CHAT peptidase is derived from Desulfonema ishimotonii or a homolog, ortholog, or variant thereof.
31. The composition of any one of aspects 26-30, wherein the RAMP polypeptide is derived from Desulfonema ishimotonii, or a homolog, ortholog or variant thereof.
32. The composition of any one of aspects 26-31, wherein the RAMP polypeptide comprises a Cas11 domain and multiple Cas7 domains.
33. The composition of aspect 32, wherein the RAMP polypeptide further comprises a Csm3, Csm4, or Csm6 domain.
34. The composition of any one of aspects 26-33, wherein the RAMP polypeptide is a Type III-E Cas polypeptide.
35. The composition of any one of aspects 26-34, wherein the detection construct comprises a polypeptide comprising a protease recognition motif recognized by the peptidase.
36. The composition of aspect 35, wherein the polypeptide is a fluorescent protein protease reporter.
37. A polynucleotide encoding one or more elements (i)-(iv) of the detection composition of any one of aspects 26-36.
38. A vector system comprising one or more vectors encoding one or more of elements (i)-(iv) of the detection composition of any one of aspects 26-36.
39. An engineered cell modified to express elements (i) and (iii), of the detection composition of any one of aspects 26-36.
40. The engineered cell of aspect 39, further modified to express element (iv).
41. The engineered cell of aspect 39 or 40, further modified to express element (ii).
42. A method for screening cell perturbations comprising;
   a. introducing a perturbation to a cell population comprising engineered cells of any one of aspects 39 to 41, along with any elements of the detection composition not already expressed by the engineered cells, and wherein the guide molecules are configured to detect one or more target transcripts associated with a specific cell type or cell state;
   b. activating the peptidase via binding of the complex to one or more target polynucleotides such that the detection construct is modified by the activated peptidase to produce a detectable product and/or signal;
   c. detecting an ability of the perturbation to modify expression of the one or more target transcripts by measuring a change in the detectable product and/or signal relative to a control.
43. A method of detecting target polynucleotides in samples comprising:
   a. combining a sample or a component thereof with the detection composition as in any one of aspects 26-36; and
   b. activating the peptidase via binding of the complex to one or more target polynucleotides such that the detection construct is modified by the activated peptidase such that a detectable product and/or signal is produced, thereby detecting the target polynucleotide in the sample.
44. The method of aspect 43, further comprising amplifying and/or enriching the target polynucleotide.
45. The method of aspect 43, wherein activating the peptidase further results in activation or generation of one or more signal amplification molecules.
46. A method of labeling cells comprising:
   a. introducing the detection composition an in any one of aspects 26-36 into a population of cells, wherein the guide molecule is configured to detect one or more target transcripts associated with a particular cell type or cell state; and
   b. activating the peptidase via binding of the complex to the one or more target transcripts such that the detection construct is modified by the activated peptidase such that a detectable product and/or signal is generated, thereby labeling cells within the cell population expressing the one or more target transcripts.
47. The method of aspect 46, wherein labeled cells are further sorted or isolated based on production of the detectable product and/or signal.
48. A method of in vivo effector activation or delivery comprising:
   introducing a programmable nuclease system of any one of aspects 1-12 into a cell comprising a substrate of the peptidase, wherein the substrate of the peptidase is optionally tethered to a cellular structure and wherein the substrate the peptidase is coupled to an effector.
49. The method of aspect 48, wherein the effector
   a. is capable of producing a detectable signal when activated
   b. is a therapeutic molecule or prodrug;
   c. is a genetic modifying molecule;
   d. or any combination thereof.
50. The method of any one of aspects 48-49, wherein the effector is inactive when coupled to an uncleaved substrate.
51. The method of any one of aspects 48-49, wherein the effector is inactive when coupled to a cleaved substrate portion.
52. The method of any one of claims 48-51, further comprising cleaving the substrate by the peptidase in response to a target RNA and activation of the peptidase of the programmable nuclease system.
53. The method of aspect 52, wherein the target RNA is endogenous to the cell or is exogenous to the cell.
54. The method of any one of aspects 48-53, wherein the substrate is tethered to a cell membrane or a nuclear membrane.

1. A programmable nuclease-peptidase composition comprising:
a repeat-associated mysterious protein (RAMP) polypeptide;
a guide molecule capable of forming a RAMP-guide molecule complex with the RAMP polypeptide and directing sequence specific binding of the complex to a target polynucleotide; and
a peptidase capable of binding to the RAMP polypeptide, the guide molecule, or further complexing with the RAMP-guide molecule complex, wherein binding of the RAMP-guide molecule complex to the target polynucleotide initiates binding and/or interaction of the peptidase with a target polypeptide.

2. The programmable nuclease-peptidase composition of claim 1, wherein target polypeptide interaction and/or binding occurs at, or in effective proximity to, a peptidase recognition motif in the target polypeptide.

3. The programmable nuclease-peptidase composition of claim 2, wherein the peptidase recognition motif comprises or consists of SEQ ID NO: 3 or a sequence therein, optionally MKKD (SEQ ID NO: 25).

4. The programmable nuclease-peptidase composition of claim 1, wherein the peptidase is a TPR-CHAT peptidase.

5. The programmable nuclease-peptidase composition of claim 1, wherein (a) the peptidase is a TPR-CHAT peptidase and wherein the TPR-CHAT peptidase is derived from Desulfonema ishimotonii, or a homolog, ortholog, or variant thereof; (b) the RAMP polypeptide is derived from Desulfonema ishimotonii, or a homolog, ortholog or variant thereof; or both (a) and (b).

6. (canceled)

7. The programmable nuclease-peptidase composition of claim 5, wherein (a) the RAMP polypeptide comprises a Cas11 domain and multiple Cas7 domains or (b) the RAMP polypeptide is a Type III-E Cas polypeptide.

8. The programmable nuclease-peptidase composition of claim 7, wherein the RAMP polypeptide comprises a Cas11 domain and multiple Cas 7 domains and further comprises a Csm3, Csm4, or Csm6 domain.

9. (canceled)

10. The programmable nuclease-peptidase composition of claim 1, wherein the target polypeptide comprises, consists of, or is coupled to an effector, wherein the effector is optionally
a. a reporter polypeptide;
b. a signal amplification polypeptide;
c. an engineered prodrug;
d. a cargo polypeptide; or
e. a pathogenic polypeptide.

11. The programmable nuclease-peptidase composition of claim 1, wherein the target polypeptide is a disease-associated polypeptide, optionally wherein the disease-associated polypeptide is associated with a neurodegenerative disease, a metabolic disease, an autoimmune disease, or a cancer.

12. (canceled)

13. A polynucleotide encoding a programmable nuclease-peptidase composition or component thereof as in claim 1, optionally further comprising one or more regulatory elements and wherein the polynucleotide encoding a programmable nuclease-peptidase composition or component thereof is operatively coupled to one or more of the one or more regulatory elements.

14. (canceled)

15. A vector or vector system comprising one or more polynucleotides according to claim 13, optionally wherein the vector or vector system is a viral vector or vector system, optionally an adeno-associated virus vector or vector system.

16. (canceled)

17. A cell or cell population comprising a programmable nuclease-peptidase composition of claim 1.

18. A pharmaceutical formulation comprising:
(a) a programmable nuclease-peptidase composition or component thereof as in claim 1;
(b) a polynucleotide encoding (a);
(c) a vector or vector system comprising one or more polynucleotides of (b);
(d) a cell or cell population comprising (a), (b), (c), or any combination thereof; or
(e) any combination of (a)-(d);
and
a pharmaceutically acceptable carrier.

19. A method of modifying a polypeptide comprising:
introducing into a sample having one or more target polynucleotides and target polypeptides, the programmable nuclease-peptidase compositions of claim 1; and
activating the peptidase via sequence specific binding of the complex to the one or more target polynucleotides such that the peptidase then binds or interacts with the one or more target polypeptides resulting in modification of the one or more target polypeptides.

20. The method of claim 19, wherein (a) the target polypeptide modification is cleavage of the target polypeptide; (b) introducing into the sample comprises in vitro, ex vivo, or in vivo delivery of the programable nuclease-peptidase composition into a cell or cell population; or (c) both (a) and (b).

21. (canceled)

22. The method of claim 20, wherein
(a) the one or more target polypeptides are proenzymes and the modification results in conversion of the proenzyme into an active enzyme;
(b) modification of the one or more target polypeptides results in activation or deactivation of one or more cell-signaling proteins;
(c) wherein the one or more target polynucleotides are a specific transcript or set of transcripts and wherein modification of the one or more target polypeptides triggers cell death upon activating the peptidase in response to binding of the nuclease-peptidase to the specific transcript or set of transcripts; or
(d) any combination of (a)-(c).

23. (canceled)

24. (canceled)

25. The method of claim 22, wherein the guide molecule is configured to detect one or more mutations in the specific transcript or set of transcripts.

26. A detection composition comprising:
(i) a RAMP polypeptide;
(ii) a guide molecule capable of forming a RAMP-guide molecule complex with the RAMP polypeptide and directing sequence-specific binding of the complex to a target polynucleotide;
(iii) a peptidase capable of binding the RAMP polypeptide, the guide molecule, or further complexing with the RAMP-guide complex; and
(iv) a detection construct,
wherein binding of the RAMP-guide molecule complex to the target polynucleotide initiates peptidase mediated modification of the detection construct resulting in generation of a detectable signal.

27. The composition of claim 26, wherein the detection construct comprises a peptidase recognition motif recognized by the peptidase.

28. The composition of claim 27, wherein the peptidase recognition motif comprises or consists of SEQ ID NO: 3 or a sequence therein, optionally MKKD (SEQ ID NO: 25).

29. The composition of claim 26, wherein the peptidase is a TPR-CHAT peptidase.

30. The composition of claim 29, wherein (a) the peptidase is a TPR-CHAT peptidase and wherein the TPR-CHAT peptidase is derived from Desulfonema ishimotonii or a homolog, ortholog, or variant thereof; (b) wherein the RAMP polypeptide is derived from Desulfonema ishimotonii, or a homolog, ortholog or variant thereof; or both (a) and (b).

31. (canceled)

32. The composition of claim 31, wherein (a) the RAMP polypeptide comprises a Cas11 domain and multiple Cas7 domains or (b) the RAMP polypeptide is a Type III-E Cas polypeptide.

33. The composition of claim 32, wherein the RAMP polypeptide comprises a Cas11 domain and multiple Cas 7 domains and further comprises a Csm3, Csm4, or Csm6 domain.

34. (canceled)

35. The composition of claim 26, wherein the detection construct comprises a polypeptide comprising a protease recognition motif recognized by the peptidase, optionally wherein the polypeptide is a fluorescent protein protease reporter.

36. (canceled)

37. A polynucleotide encoding one or more elements (i)-(iv) of the detection composition of claim 26.

38. A vector system comprising one or more vectors encoding one or more of elements (i)-(iv) of the detection composition of claim 26.

39. An engineered cell modified to express elements (i) and (iii), of the detection composition of claim 26.

40. The engineered cell of claim 39, wherein the engineered cell is (a) further modified to express element (iv); (b) further modified to express element (ii); or (c) both (a) and (b).

41. (canceled)

42. A method for screening cell perturbations comprising;
introducing a perturbation to a cell population comprising engineered cells of claim 39, along with any elements of the detection composition not already expressed by the engineered cells, and wherein the guide molecules are configured to detect one or more target transcripts associated with a specific cell type or cell state;
activating the peptidase via binding of the complex to one or more target polynucleotides such that the detection construct is modified by the activated peptidase to produce a detectable product and/or signal;
detecting an ability of the perturbation to modify expression of the one or more target transcripts by measuring a change in the detectable product and/or signal relative to a control.

43. A method of detecting target polynucleotides in samples comprising:
combining a sample or a component thereof with the detection composition as in claim 26; and
activating the peptidase via binding of the complex to one or more target polynucleotides such that the detection construct is modified by the activated peptidase such that a detectable product and/or signal is produced, thereby detecting the target polynucleotide in the sample.

44. The method of claim 43, further comprising amplifying and/or enriching the target polynucleotide, optionally wherein activating the peptidase further results in activation or generation of one or more signal amplification molecules.

45. (canceled)

46. A method of labeling cells comprising:
introducing the detection composition as in claim 26 into a population of cells, wherein the guide molecule is configured to detect one or more target transcripts associated with a particular cell type or cell state; and
activating the peptidase via binding of the complex to the one or more target transcripts such that the detection construct is modified by the activated peptidase such that a detectable product and/or signal is generated, thereby labeling cells within the cell population expressing the one or more target transcripts.

47. The method of claim 46, wherein labeled cells are further sorted or isolated based on production of the detectable product and/or signal.

48. A method of in vivo effector activation or delivery comprising:
introducing a programmable nuclease system of claim 1 into a cell comprising a substrate of the peptidase, wherein the substrate of the peptidase is optionally tethered to a cellular structure and wherein the substrate the peptidase is coupled to an effector.

49. The method of claim 48, wherein the effector
a. is capable of producing a detectable signal when activated
b. is a therapeutic molecule or prodrug;
c. is a genetic modifying molecule;
d. or any combination thereof.

50. The method of claim 48, wherein the effector is inactive when coupled to an uncleaved substrate or wherein the effector is inactive when coupled to a cleaved substrate portion.

51. (canceled)

52. The method of claim 48, further comprising cleaving the substrate by the peptidase in response to a target RNA and activation of the peptidase of the programmable nuclease system, optionally wherein the target RNA is endogenous to the cell or is exogenous to the cell.

53. (canceled)

54. The method of claim 48, wherein the substrate is tethered to a cell membrane or a nuclear membrane.
